(19) United States
(12) Patent Application Publication
(10) Pub. No.: US 2024/0165066 A1
(43) Pub. Date: May 23, 2024

(54) LYMPH-RELEASING COMPOSITIONS OF FATTY ACIDS AND USES THEREOF FOR LYMPHATIC INCORPORATION AND SYSTEMIC DISEASE TREATMENT

(71) Applicant: Amarin Pharmaceuticals Ireland Limited, Dublin (IE)

(72) Inventors: Richard Louis Dunbar, Bridgewater, NJ (US); James Moberly, Bridgewater, NJ (US); Richard Preston Mason, Bridgewater, NJ (US)

(21) Appl. No.: 18/472,875

(22) Filed: Sep. 22, 2023

Related U.S. Application Data
(63) Continuation of application No. 18/160,286, filed on Jan. 26, 2023, now abandoned.
(60) Provisional application No. 63/303,365, filed on Jan. 26, 2022.
(60) Provisional application No. 63/303,383, filed on Jan. 26, 2022.
(60) Provisional application No. 63/304,042, filed on Jan. 28, 2022.
(60) Provisional application No. 63/334,065, filed on Apr. 22, 2022.
(60) Provisional application No. 63/334,071, filed on Apr. 22, 2022.
(60) Provisional application No. 63/340,292, filed on May 10, 2022.
(60) Provisional application No. 63/340,304, filed on May 10, 2022.
(60) Provisional application No. 63/342,509, filed on May 16, 2022.
(60) Provisional application No. 63/348,908, filed on Jun. 3, 2022.

(51) Int. Cl. A61K 31/202; A61K 31/5575; A61K 31/685; A61P 11/00

(52) U.S. Cl. CPC A61K 31/202; A61K 31/5575; A61K 31/685; A61P 11/00

(57) ABSTRACT

Provided are compositions comprising one or more polyunsaturated fatty acids or derivatives thereof, a source of phospholipid, and optionally one or more additional emulsifiers, as well as methods of using the same to treat various diseases. In some embodiments, provided is a lymph-releasing composition of eicosapentaenoic acid ethyl ester (LR-EtEPA) and methods of using the same to increase EPA uptake in tissues and to treat various diseases including cardiopulmonary diseases, renal diseases, neurological diseases, and cancer.

### CROSS-REFERENCE TO RELATED APPLICATION(S)

This application is a continuation of U.S. patent application Ser. No. 18/160,286 filed Jan. 26, 2023, which claims the benefit of U.S. Provisional Patent Application No. 63/303,365, filed on Jan. 26, 2022; U.S. Provisional Patent Application No. 63/303,383, filed on Jan. 26, 2022; U.S. Provisional Patent Application No. 63/304,042, filed on Jan. 28, 2022; U.S. Provisional Patent Application No. 63/334,065, filed on Apr. 22, 2022; U.S. Provisional Patent Application No. 63/334,071, filed on Apr. 22, 2022; U.S. Provisional Patent Application No. 63/340,292, filed on May 10, 2022; U.S. Provisional Patent Application No. 63/340,304, filed on May 10, 2022; U.S. Provisional Patent Application No. 63/342,509, filed on May 16, 2022; and U.S. Provisional Patent Application No. 63/348,908, filed on Jun. 3, 2022. The contents of each of these provisional applications are incorporated by reference in their entirety.

### SUMMARY

Provided are compositions comprising one or more polyunsaturated fatty acids or derivatives thereof, a source of phospholipid, and optionally one or more additional emulsifiers, as well as methods of using the same to treat various diseases. For example, in some embodiments, provided is a lymph-releasing composition of eicosapentaenoic acid ethyl ester (LR-EtEPA) and methods of using the same to increase EPA uptake in tissues and to treat various diseases including cardiopulmonary diseases, renal diseases, neurological diseases, and cancer.

In one aspect, provided is a composition comprising: (a) at least 15%, by weight, one or more polyunsaturated fatty acids (PUFAs) or derivatives thereof; and (b) 1% to 85%, by weight, a source of phospholipid. In some embodiments, the composition further comprises (c) 1% to 20%, by weight, one or more emulsifiers.

In some embodiments, the one or more PUFAs or derivatives thereof are selected from the group consisting of linoleic acid (LA), gamma-linoleic acid (GLA), dihomo-gamma-linoleic acid (DGLA), arachidonic acid (AA), adrenic acid (AdA), omega-6 docosapentaenoic acid (DPA6), alpha-lineoleic acid (ALA), stearidonic acid (SDA), omega-3 eicosatetraenoic acid (ETA), eicosapentaenoic acid (EPA), docosapentaenoic acid (DPA), docosahexaenoic acid (DHA), an LA derivative, a GLA derivative, a DGLA derivative, an AA derivative, an AdA derivative, a DPA6 derivative, an ALA derivative, an SDA derivative, an ETA derivative, an EPA derivative, a DPA derivative, and a DHA derivative.

In some embodiments, the one or more PUFAs or derivatives thereof are selected from the group consisting of tetracosatetraenoic acid (TTE), tetracosapentaenoic acid (TPA), tetracosahexaenoic acid (THA), a TTE derivative, a TPA derivative, and a THA derivative.

In some embodiments, the PUFA derivative comprises an oxylipin.

the one or more PUFAs or derivatives thereof comprises EPA in free acid form or a pharmaceutically acceptable ester, conjugate, or salt thereof. In some embodiments, the EPA is eicosapentaenoic acid ethyl ester (EtEPA).

In some embodiments, the EPA or EtEPA comprises at least 66%, 75%, 80%, 90%, 95%, or 96%, by weight, of all PUFAs present in the composition.

In some embodiments, the composition comprises no more than 20%, by weight of all PUFAs present in the composition, one or more of the following: (a) one or more omega-6 PUFAs or derivatives thereof selected from the group consisting of LA, GLA, DGLA, AdA, DPA6, an LA derivative, a GLA derivative, a DGLA derivative, an AdA derivative, and a DPA6 derivative; (b) one or more omega-3 PUFAs or derivatives thereof selected from the group consisting of ALA, SDA, ETA, DPA, an ALA derivative, an SDA derivative, an ETA derivative, and a DPA derivative; and (c) one or more oxylipins.

In some embodiments, the composition comprises about 500 mg to about 1 g of the EPA or EtEPA.

In some embodiments, the source of phospholipid comprises a glycerophospholipid, a lysophospholipid, or a mixture thereof.

In some embodiments, the source of phospholipid is lecithin. In some embodiments, the lecithin comprises up to 40%, up to 60%, up to 80%, up to 90%, up to 95%, or up to 97%, by weight of the lecithin, phosphatidylethanolamine, and no more than 10%, no more than 5%, no more than 4%, no more than 3%, no more than 2%, or no more than 1%, by weight of the lecithin, phosphatidylinositol.

In some embodiments, the lecithin comprises: (a) 19%-27%, by weight, phosphatidylcholine; (b) no more than 4%, by weight, lysophosphatidylcholine; (c) 16%-22%, by weight, phosphatidylethanolamine; (d) 11%-18%, by weight, phosphatidylinositol; and (e) 1%-9%, by weight, phosphatidic acid.

In some embodiments, a weight ratio of the one or more PUFAs or derivatives thereof and the source of phospholipid ranges from about 5:1 to about 1:5, from about 3.75:1 to about 1:5, or from about 1:1 to about 1:5, or a weight ratio of the EPA or EtEPA and the source of phospholipid ranges from about 1:1 to about 1:5.

In some embodiments, the one or more emulsifiers comprise polysorbate 80, polyoxyl-35, or both. In some embodiments, the one or more emulsifiers comprise one or more glycerol derivatives selected from the group consisting of triacylglycerol, diacylglycerol, and monoacylglycerol. In some embodiments, the glycerol derivative is castor oil. In some embodiments, the glycerol derivative is re-esterified triglyceride (rTG) enriched with the PUFA.

In another aspect, provided is a kit comprising: (a) a first composition comprising one or more polyunsaturated fatty acids (PUFAs) or derivatives thereof; and (b) a second composition comprising a source of phospholipid. In some embodiments, the first and/or second composition further comprises one or more emulsifiers.

In another aspect, provided is a lymph-releasing eicosapentaenoic acid ethyl ester (LR-EtEPA) composition comprising: (a) at least 15%, by weight, EtEPA; and (b) 1% to 85%, by weight, a source of phospholipid. In some embodiments, the LR-EtEPA composition further comprises (c) 1% to 20%, by weight, one or more emulsifiers.

In another aspect, provided is a method of treating or preventing a disease in a subject in need thereof, the method comprising administering to the subject a therapeutically effective amount of a composition, kit, or LR-EtEPA composition according to various embodiments of the present technology.

In another aspect, provided is a composition, kit, or LR-EtEPA composition according to various embodiments of the present technology for use in a method of treating or preventing a disease in a subject in need thereof, wherein a therapeutically effective amount of the composition, kit, or LR-EtEPA composition is administered to the subject.

In some embodiments, the disease is a cardiovascular disease. In some embodiments, the cardiovascular disease is selected from the group consisting of hypertriglyceridemia, hypercholesterolemia, mixed dyslipidemia, coronary heart disease, stroke, atherosclerosis, arrhythmia, hypertension, myocardial infarction, vasculitis, cardiomyopathy (e.g., viral cardiomyopathy including related to COVID-19), pericarditis, congestive heart failure, myocardial necrosis, vascular ischemia, vascular disease beyond the cardiopulmonary system, thrombotic disease, post-myocardial infarction cardiac remodeling, giant cell arteritis, polyarteritis nodosa, cryoglobulinemia, episodic small-vessel ischemia (Raynaud's disease), deep venous thrombosis, disseminated intravascular coagulation, and erectile dysfunction.

In some embodiments, the subject has a fasting baseline triglyceride level of about 135 mg/dL to about 500 mg/dL.

In some embodiments, the subject has one or more of: a baseline non-high-density lipoprotein cholesterol (HDL-C) value of about 200 mg/dL to about 300 mg/dL; a baseline total cholesterol (TC) value of about 250 mg/dL to about 300 mg/dL; a baseline very low-density lipoprotein cholesterol (VLDL-C) value of about 140 mg/dL to about 200 mg/dL; a baseline HDL-C value of about 10 mg/dL to about 30 mg/dL; a baseline low-density lipoprotein cholesterol (LDL-C) value of about 40 mg/dL to about 100 mg/dL; and a baseline high-sensitivity C-reactive protein (hsCRP) level of about 2 mg/dL or less.

In some embodiments, the subject is on stable statin therapy. In some embodiments, the stable statin therapy comprises a statin and optionally ezetimibe. In some embodiments, the statin is selected from the group consisting of atorvastatin, fluvastatin, lovastatin, pitavastatin, pravastatin, rosuvastatin, and simvastatin.

In some embodiments, the disease is a pulmonary disease. In some embodiments, the pulmonary disease is selected from the group consisting of community-acquired pneumonia, COVID-19 pneumonia, systemic inflammatory response syndrome (SIRS), sepsis, SIRS, acute respiratory distress syndrome (ARDS), pulmonary embolism, diffuse interstitial pneumonia, radiation pneumonitis, pleuritis, acute eosinophilic pneumonia, chronic eosinophilic pneumonia, Loffler syndrome, sarcoidosis, interstitial lung disease, chronic obstructive pulmonary disease (COPD), reactive airway disease, asthma, bronchiectasis, bronchiolitis, cystic fibrosis, bronchial carcinoid, pulmonary arterial hypertension, pulmonary vasculitis, microscopic polyangiitis, granulomatosis with polyangiitis (Wegener's disease), eosinophilic granulomatosis with polyangiitis (Churg-Strauss), nasopharyngitis, Goodpasture's syndrome, cryoglobulinemia, systemic lupus erythematosus (SLE), systemic sclerosis, and antiphospholipid syndrome.

In some embodiments, the disease is a neurological disease. In some embodiments, the neurological disease is selected from the group consisting of Huntington's disease, sleep disorders, dementia, psychosis, anxiety, treatment-resistant depression, neuropathic pain, schizophrenia, bipolar disorder, dyslexia, dyspraxia, attention deficit hyperactivity disorder (ADHD), epilepsy, autism, Alzheimer's disease, Parkinson's Disease, senile dementia, multiple sclerosis, diabetes-induced neuropathy, macular degeneration, retinopathy of prematurity, amyotrophic lateral sclerosis (ALS), retinitis pigmentosa, cerebral palsy, muscular dystrophy, neurological cancer, cystic fibrosis, and neural tube defects.

In some embodiments, the disease is cancer. In some embodiments, the cancer is a hematological malignancy selected from the group consisting of monoclonal B cell lymphocytosis, multiple myeloma, myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), blast crisis chronic myelogenous leukemia (bcCML), B cell acute lymphoid leukemia (B-ALL), T cell acute lymphoid leukemia (T-ALL), T cell lymphoma, and B cell lymphoma. In some embodiments, the cancer is a solid tumor selected from the group consisting of lung cancer, breast cancer, liver cancer, stomach cancer, colon cancer, rectal cancer, colorectal cancer, kidney cancer, gastric cancer, gallbladder cancer, cancer of the small intestine, esophageal cancer, melanoma, bone cancer, pancreatic cancer, skin cancer, uterine cancer, ovarian cancer, testicular cancer, cancer of the thyroid gland, cancer of the adrenal gland, bladder cancer, and glioma.

In some embodiments, the disease is a disease associated with kidney selected from the group consisting of post-infectious glomerulonephritis, IgA nephropathy (Berger's disease), Henoch-Schönlein purpura, systemic IgA vasculitis, microscopic polyangiitis, granulomatosis with polyangiitis (Wegener's), eosinophilic granulomatosis with polyangiitis (Churg-Strauss), polyarteritis, idiopathic crescentic glomerulonephritis, anti-GBM glomerulonephritis, Goodpasure syndrome, cryoglobulin-associated glomerulonephritis, idiopathic membranoproliferative glomerulopnephritis (MPGN), hepatitis C-associated glomerulonephritis, systemic lupus erythematosus (SLE) associated glomerulonephritis, minimal change disease (nill disease, lipoid nephrosis), membranous nephropathy, focal and segmental glomerulosclerosis, amyloidosis, diabetic nephropathy, HIV-associated nephropathy, membranoproliferative glomerlonephropathy, mitigating proteinuria, mitigating chronic renal failure, and/or mitigating mortality/morbidity in severe chronic kidney disease (CKD)/end-stage renal disease (ESRD).

In some embodiments, the disease is a disease associated with pancreas selected from the group consisting of hyperglycemia, pre-diabetes, diabetes (Type 1 and/or Type 2), and pancreatitis.

In some embodiments, the disease is a disease associated with liver selected from the group consisting of chronic viral hepatitis, autoimmune hepatitis, alcoholic liver disease, non-alcoholic fatty liver disease, hemochromatosis, Wilson disease, primary biliary cholangitis, primary sclerosing cholangitis, and cholelithiasis.

In some embodiments, the disease is a disease associated with intestines selected from the group consisting of gastroesophageal reflux disease (GERD), gastritis, peptic ulcer disease, obesity, cachexia, intestinal angina, Crohn disease, ulcerative colitis, antibiotic-associated colitis, irritable bowel syndrome, colon cancer, colon polyposis, and carcinoid.

In some embodiments, the disease is a disease associated with blood cells selected from the group consisting of iron deficiency anemia, anemia of chronic disease, hemolytic anemia, thalassemia, polycythemia vera, sickle cell disease anemia, sickle cell disease pain, immune thrombocytopenia, leukemias, Non-Hodgkin lymphoma, and Hodgkin lymphoma.

In some embodiments, the disease is a disease associated with oxidative stress, glutathione (GSH) depletion, Nrf2 activation, and/or heme-oxygenase activation. In some embodiments, the disease is anemia, sickle cell disease, and/or glomerulonephritis. In some embodiments, the method further comprises administering to the subject a N-acetylcysteine (NAC) related agent. In some embodiments, the NAC related agent is selected from the group consisting of cystine, methionine, N-acetylcysteine, and L-2-oxothiazolidine-4-carboxylate.

In some embodiments, the disease is oxidative stress, endothelial dysfunction, narrowing and/or thickening of arteries, and/or inflammation induced by inhalation of particulate matter. In some embodiments, the disease is oxidative stress, endothelial dysfunction, narrowing and/or thickening of arteries, and/or inflammation induced by long-term and/or short-term exposure to air pollution.

In some embodiments, the composition, kit, or LR-EtEPA composition is administered to the subject to provide a daily dose of about 1 g to about 20 g of EtEPA, for example, about 4 g of EtEPA.

In some embodiments, the composition, kit, or LR-EtEPA composition is administered to the subject once or twice per day, with or without food.

### BRIEF DESCRIPTION OF THE DRAWINGS

FIG. 1A shows comparison of lymph-releasing eicosapentaenoic acid ethyl ester (LR-EtEPA) versus plain EtEPA at equal doses of EtEPA for tissue EPA enrichment in the lymph, heart, lungs, brain, and lung alveolar macrophages (AVMs). For each tissue, the fold equivalent of LR-EtEPA compared to plain EtEPA for EPA level and EPA:AA ratio is shown.

FIG. 1B shows preferred routing of EPA through the lymphatic system over the portal vein by co-administration of lymph-releasing compounds (e.g., lecithin (LC) and/or emulsifiers) with EtEPA, thereby avoiding visceral EPA loss (e.g., visceral adipose EPA sequestration and hepatic first pass EPA loss) and increasing systemic and tissue EPA levels, for example, in the kidney, jejunum, and pancreas. For each of the exemplary tissues, the percentage increases in EPA level and EPA:AA ratio by LR-EtEPA over plain EtEPA are shown.

FIG. 2 shows that LR-EtEPA is superior to plain EtEPA at enriching lymph fluid with EPA after a single dose, and such enrichment is more enhanced when the relative ratio of the lymph-releasing compounds to EtEPA (LC:EtEPA) is increased from 1:4 to 1:1. IPE, icosapent ethyl, also referred to as EtEPA; FAME, fatty acid methyl ester, which is an indicator of the total amount of a fatty acid measured (in this case it is EPA).

FIGS. 3-4 show that LR-EtEPA is superior to EtEPA at increasing tissue EPA levels in the lung and heart (FIG. 3) and at improving EPA:AA ratio in lung immune cells (FIG. 4) after only 7 Days.

FIG. 5 shows LR-EtEPA vs EtEPA for various phospholipid-EPA (PL-EPA) fractional amounts (FrAmt, 100th) on day 7 in cellular tissues.

FIG. 6 shows LR-EtEPA:EtEPA ratio vs pool size, comparing various cellular PL-EPAs.

FIGS. 7-12 show vector plots of LR-EtEPA:EtEPA ratio, comparing various PL-EPAs in the lungs (FIG. 7), AVMs (FIG. 8), heart (FIG. 9), blood matrix (FIG. 10), blood cells (FIG. 11), and liver (FIG. 12) at day 7.

FIG. 13 shows LR-EtEPA vs EtEPA for various PL-EPA/ARA ratio fractional amounts (FrAmt, 100th) on day 7 in cellular tissues.

FIG. 14 shows LR-EtEPA:EtEPA ratio vs pool size, comparing various cellular PL-EPA/ARA ratios at day 7.

FIGS. 15-20 show vector plots of LR-EtEPA:EtEPA ratio, comparing various PL-EPA/ARA ratios in the lungs (FIG. 15), alveolar macrophages (FIG. 16), heart (FIG. 17), blood matrix (FIG. 18), blood cells (FIG. 19), and liver (FIG. 20) at day 7.

FIG. 21 shows that LR-EtEPA (1× (IPE+LC 4:1)) is superior to plain EtEPA (IPE) in improving EPA:ARA ratio in lung immune cells after 7 days and after 21 days. OA, oleic acid; IPE, icosapent ethyl, also referred to as EtEPA.

FIG. 22 is a summary plot showing that at equal doses, LR-EtEPA outperforms plain EtEPA at increasing EPA in lymph, lungs, lung AVMs, heart, and brain in long-Evans rats after 7 and/or 21 days of multiple dosing.

FIGS. 23A and 23B show potential mechanisms of different cell types as indicated in disease conditions including atherosclerosis, pulmonary diseases, renal diseases, and injury, as well as how EPA (and its derived oxylipins) may alleviate these disease conditions.

FIGS. 24A-24E show dose response plots of EPA/AA (FIGS. 24A-24B), OXygenation-Promoters (OXP, including EPA, GLA, and DHA)/AA (FIGS. 24C-24D), and Medicinal Oxylipin Precursors (MOP, including DGLA, EPA, DPA, and DHA)/AA (FIG. 24E) ratios in the lung tissue in rats dosed with five treatment arms presented in the order of, from left to right: (1) oleic acid (OA); (2) EPA+GLA+DHA; plain EtEPA (EtEPA or IPE); (3) LR-EtEPA (EtEPA plus lecithin (LC)) with EtEPA equimolar to plain EtEPA (1× LR-EtEPA), and (5) LR-EtEPA at 1.5-fold the molar dose of plain EtEPA (1.5× LR-EtEPA). FIGS. 24A and 24C is a “violin” plot, while FIGS. 24B, 24D, and 24E show stacked dose response curve in untransformed (top panel) and semi-log (bottom panel) plots. ME, methyl ester; FAME, fatty acid methyl ester, which measures the total amount of a fatty acid of interest; Intercept, interception; Rel. Potency, relative potency; Sigma represents residual standard error.

Similar to FIGS. 24A-24E, FIGS. 25A-25D show dose response plots of EPA/AA (FIGS. 25A-25B), OXP/AA (FIG. 25C), and MOP/AA (FIG. 25D) ratios in the AVMs in rats dosed with five treatment arms presented in the order of, from left to right: (1) OA; (2) EPA+GLA+DHA; (3) EtEPA; (4) 1× LR-EtEPA, and (5) 1.5× LR-EtEPA.

FIGS. 26A-26D show dose response plots of EPA/AA (FIGS. 26A-26B), OXP/AA (FIG. 26C), and MOP/AA (FIG. 26D) ratios in the heart tissue in rats dosed with five treatment arms presented in the order of, from left to right: (1) OA; (2) EPA+GLA+DHA; (3) EtEPA; (4) 1× LR-EtEPA, and (5) 1.5× LR-EtEPA.

FIGS. 27A-27D show dose response plots of EPA/AA (FIGS. 27A-27B), OXP/AA (FIG. 27C), and MOP/AA (FIG. 27D) ratios in the kidney tissue in rats dosed with five treatment arms presented in the order of, from left to right: (1) OA; (2) EPA+GLA+DHA; (3) EtEPA; (4) 1× LR-EtEPA, and (5) 1.5× LR-EtEPA.

FIGS. 28A-28B show dose response plots of EPA/AA ratio in the brain tissue in rats dosed with five treatment arms presented in the order of, from left to right: (1) OA; (2) EPA+GLA+DHA; (3) EtEPA; (4) 1× LR-EtEPA, and (5) 1.5× LR-EtEPA.

FIGS. 29A-29D show dose response plots of EPA/AA (FIGS. 29A-29B), OXP/AA (FIG. 29C), and MOP/AA (FIG. 29D) ratios in the pancreas tissue in rats dosed with five treatment arms presented in the order of, from left to right: (1) OA; (2) EPA+GLA+DHA; (3) EtEPA; (4) 1× LR-EtEPA, and (5) 1.5× LR-EtEPA.

FIGS. 30A-30D show dose response plots of EPA/AA (FIGS. 30A-30B), OXP/AA (FIG. 30C), and MOP/AA (FIG. 30D) ratios in the jejunum tissue in rats dosed with five treatment arms presented in the order of, from left to right: (1) OA; (2) EPA+GLA+DHA; (3) EtEPA; (4) 1× LR-EtEPA, and (5) 1.5× LR-EtEPA.

FIGS. 31A-31D show how EtEPA affects functional indices of key enzymes that convert LC-PUFAs to other LC-PUFAs, a process that is rate-limited by desaturase enzymes but is also facilitated by elongase enzymes. FIG. 31A shows the functional impact of various treatments on Δ5-desaturation of the ω-6 LC-PUFA DGLA (20:3ω-6), yielding ARA (20:4ω-6). The product/precursor ratio ARA:DGLA is called the Δ5-desaturation index at ω-6 (Δ5D-1 ω-6). The x-axis is dose proportional to the EtEPA dose, ranging from 0 to 4.7 mmol EPA/kg/d. As an exception, at 3.1 mmol EPA/kg/d, plain EtEPA is slightly offset to the left whereas LR-EtEPA is slightly offset to the right to avoid overprinting. As shown, EtEPA substantially suppresses Δ5D-I ω-6 compared to EPA+GLA+DHA and oleic acid (OA) control. FIG. 31B shows a contrasting, ascending progression for the functional impact of various treatments on Δ8-desaturation of the ω-6 LC-PUFA eicosadienoic acid (EDA 20:2ω-6) yielding DGLA (20:3ω-6). The product/precursor ratio DGLA:EDA is called the Δ8-desaturation index at ω-6 ω-6). As shown, EtEPA substantially induces Δ8D-I ω-6 compared to EPA+GLA+DHA and oleic acid (OA) control. FIG. 31C is a schematic that summarizes data on the mechanism by which EtEPA (IPE) alters DGLA kinetics thus raising the DGLA pool, in contrast to a key long chain PUFA composition Oxepa®, namely, GLA which is included in Oxepa® as a DGLA precursor. Specifically, Oxepa® consists of three LC-PUFAs: (1) EPA, (2) GLA, and (3) DHA; in the experiment the combination of these three LC-PUFAs is referred to as EPA+GLA+DHA. After dosing Long-Evans rats EtEPA at an equimolar dose as the total LC-PUFAs in [EPA+GLA+DHA], EtEPA altered DGLA kinetics “coming and going,” meaning (1) EtEPA promoted DGLA production by enhancing conversion of eicosadienoic acid (EDA) to DGLA, assessed by the Δ8-desaturation index ω-6 (product/precursor ratio=DGLA/EDA), and (2) EtEPA inhibited DGLA catabolism by suppressing conversion of DGLA to arachidonic acid (ARA), assessed by the Δ5-desaturase index ω-6 (product/precursor ratio=ARA/DGLA). In contrast, the data suggest the GLA of EPA+GLA+DHA featured in Oxepa® has limited potential to alter DGLA kinetics, which reduces to providing substrate for DGLA production from GLA, rather than substantially altering the 08 desaturase or Δ5-desaturase enzymes. IPE, icosapent ethyl, also referred to as EtEPA. FIG. 31D shows a descending progression for the functional impact of various treatments on Δ5-elongation of the ω-6 LC-PUFA eicosapentaenoic acid (EPA, 20:5ω-6) yielding the longer omega-3 LC-PUFA DPA (22:5ω-3). The product/precursor ratio DPA:EPA is called the Δ5-elongation index at ω-3 ω-3). As shown, EtEPA substantially suppresses Δ5D-I ω-3 compared to EPA+GLA+DHA and oleic acid (OA) control.

FIGS. 32A-32J show the effect on various formulations on oxylipins present in lung tissue. The oxylipins are listed in the graph titles, along with an arrow (↓) indicating the parent LC-PUFA that was oxygenated to yield said oxylipin. Remarkably, EtEPA not only raised several EPA-derived oxylipins considered to have therapeutic potential, but it also raised DGLA-derived oxylipins and oxylipins derived from linoleic acid (LA, 18:2ω-6). Lung oxylipins were assayed from the rats with and without hydrolysis, thus yielding total oxylipins (i.e., free/non-esterified plus bound/esterified oxylipins) and free/non-esterified oxylipins. Bound/esterified oxylipins were determined by subtracting free/non-esterified oxylipins from total oxylipins. In many cases, esterified oxylipins are the dominant form, at times outnumbering free/non-esterified oxylipins by ten-fold. The oxylipin studies confirm that the large increases in tissue EPA achieved from LR-EtEPA vs. plain EtEPA vs. EPA+GLA+DHA translate into substantial increases in EPA-derived oxylipins of therapeutic potential. This reassures that the pharmacokinetic effects translate to pharmacodynamic effects. Not only so, but EPA also exerts functional effects on oxylipins derived from other LC-PUFAs, such as DGLA and LA shown here.

FIG. 33 shows a model to determine whether the high relative potencies (8) seen across a spectrum of tissue perfusion rates would extend to tissues that typically have the lowest perfusion rates (e.g., inactive muscle and adipose). To model this, the left panel shows linear regression results for five tissues that are in the lower half of the tissue perfusion spectrum typical of a healthy human adult on the x-axis. The y-axis is the relative potency, 8, as determined by dose-response modeling, as detailed elsewhere. The best-fit line had a good fit per R2 (p=0.0153). The slope and y-intercept of this line are given at the top of the graph. This line was used to model lower levels of tissue perfusion, shown in the right panel, whose x-axis includes lower-perfusion tissues. The 90% confidence intervals exclude 1, and the 8 remained close to 3×, reassuring that even lower-perfusion tissues are likely to exhibit a roughly 3-fold advantage for LR-EtEPA compared to plain EtEPA in 21 days. If the confidence intervals had included 1, it would indicate that more time than 21 days might be required for a therapeutic effect to manifest in lower-perfusion tissues.

FIG. 34A presents a schematic for how EtEPA induces the HMOX-1 gene, which promotes heme-oxygenase 1 protein (HO-1). Not shown is that upregulation of HMOX-1 is induced through the nuclear factor erythroid 2-related factor 2 (Nrf2) pathway. LC-PUFAs and their oxylipins, particularly EPA and its oxylipins stimulate Nrf2, an essential transcription factor, which regulates oxidative stress by inducing the antioxidant response element (ARE). In turn, ARE induces production of HMOX1 yielding HO-1, NQ01 yielding NAD(P)H dehydrogenase [quinone] 1, and GST yielding glutathione S-transferase. All of these have salutary effects, exemplified by HO-1's vasodilatory, anti-inflammatory, anti-apoptotic, anti-thrombotic, and angiogenic effects, mediated by effects on carbon monoxide, the antioxidants biliverdin/bilirubin, and via antioxidant effects of ferritin.

FIG. 34B presents results from a provoked-inflammation cell-culture study with proteomics outcomes, wherein endothelial cells from various tissues were exposed to the inflammatory cytokine IL-6 in the absence or presence of EPA. Notably, heme-oxygenase-1 was significantly increased in endothelial cells from pulmonary, vascular, and brain endothelial cells in the presence of EPA. Also of interest are related increases in proteins involved in the related antioxidant ferritin in vascular endothelial cells. Finally fatty acid desaturase 1 (FADS1) encodes the Δ5-desaturase enzyme and fatty acid desaturase 2 (FADS2) encodes the 06-desaturase enzyme. FADS2 was significantly suppressed in vascular endothelial cells and brain endothelial cells, and FADS1 was significantly suppressed in brain endothelial cells, which protein suppression accords with functional results from Long-Evans rats discussed elsewhere, particularly showing functional suppression of Δ5-desaturase.

FIGS. 34C-34D illustrate that endothelial nitric oxide bioavailability depends on eNOS coupling efficiency.

FIG. 35 is a volcano plot of modulated proteins: IL-6 vs EPA+IL-6 (all points above the horizontal line are considered significant (p<0.05)).

FIG. 36 is a volcano Plot of modulated proteins: IL-6 vs DHA+IL-6 (all points above the horizontal line are considered significant (p<0.05)).

FIG. 37 shows the effects of EPA and DHA on relative expression levels (log₂ intensity) of eNOS challenged with IL-6. Graphs show normalized intensity values of each replicate in the treatment groups (n=3).

FIG. 38 illustrates air pollution particulate matters (PMs) cause multi-organ damage and systemic vascular inflammation associated with both the innate and adaptive immune responses. These vascular changes lead to increased cardiovascular risk and ischemic injury, including myocardial infarction and stroke.

FIGS. 39A-39B show distribution of particulate size in urban particulate matter (FIG. 39A) and fine particulate matter (FIG. 39B).

FIG. 40 is a schematic illustration of proteomic analysis protocol.

FIG. 41A is a volcano plot of all detected proteins from fine PMs vs EPA+fine PMs.

FIG. 41B is a volcano plot of all detected proteins from urban PMs vs EPA+urban PMs.

FIGS. 42A-42C show effects of EPA on relative expression levels (log₂ intensity) of inflammatory protein CXCL6 (FIG. 42A), HSP90B1 (FIG. 42B), and GSTP1 (FIG. 42C) following challenges with urban and fine PMs, respectively.

### DETAILED DESCRIPTION

While the present invention is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” In this manner, slight variations from a stated value can be used to achieve substantially the same results as the stated value. Also, the disclosure of ranges is intended as a continuous range including every value between the minimum and maximum values recited as well as any ranges that can be formed by such values. Also disclosed herein are any and all ratios (and ranges of any such ratios) that can be formed by dividing a recited numeric value into any other recited numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

### Compositions

In some embodiments, provided are compositions comprising one or more polyunsaturated fatty acid (PUFA)s or derivatives thereof, and a source of phospholipid. In some embodiments, the composition may further comprise one or more additional emulsifiers. In certain of these embodiments, the PUFAs or derivatives thereof can function as the active ingredient, and the phospholipid and/or additional emulsifiers can be referred to as additives or excipients.

In some embodiments, provided is a kit comprising a first composition comprising one or more PUFAs or derivatives thereof, and a second composition comprising a source of phospholipid. In some embodiments, the first and/or second composition may further comprise one or more additional emulsifiers.

Polyunsaturated Fatty Acids or Derivatives Thereof

In some embodiments, the PUFA or derivative thereof comprises a long chain PUFA (LC-PUFA), which are typically fatty acids having at least 18 carbon atoms, or a derivative thereof. In some embodiments, the PUFA or derivative thereof comprises a very long chain PUFA (VLC-PUFA), which are typically fatty acids having at least 24 carbon atoms, or a derivative thereof.

The one or more PUFAs or derivatives thereof of the composition may be selected from the group consisting of omega-6 fatty acids including linoleic acid (FA 18:2ω-6, or LA), γ-linoleic acid (FA 18:3ω-6, or GLA), dihomo-γ-linoleic acid (FA 20:3ω-6, or DGLA), arachidonic acid (FA 20:4ω-6, AA, or ARA), adrenic acid (FA 22:4ω-6, or AdA, also known as docosatetraenoic acid, or DTA), and omega-6 docosapentaeinoic acid (FA 22:5ω-6, or DPA6); and omega-3 fatty acids including α-linoleic acid (FA 18:3ω3, or ALA), stearidonic acid (FA 18:4ω-3, or SDA), omega-3-eicosatetraenoic acid (FA 20:4ω-3, or ETA), eicosapentaenoic acid (FA 20:5ω-3, or EPA), docosapentaenoic acid (FA 22:5ω-3, or DPA), and docosahexaenoic acid (FA 22:6ω-3, or DHA); and derivatives thereof including an LA derivative, a GLA derivative, a DGLA derivative, an AA or ARA derivative, an AdA or DTA derivative, a DPA6 derivative, an ALA derivative, an SDA derivative, an ETA derivative, an EPA derivative, a DPA derivative, and a DHA derivative. The term “AA” and “ARA” are used interchangeably in the present disclosure to refer to arachidonic acid.

As used herein, a “PUFA” especially those written in short form (e.g., EPA) refers to the PUFA in free acid form and/or a pharmaceutically acceptable ester, or conjugate, or salt thereof, or mixtures of any of the foregoing, as long as the acyl group or the carbon chain portion of the molecule remains intact. The term “pharmaceutically acceptable” in the present context means that the substance in question does not produce unacceptable toxicity to the subject or interaction with other components of the composition. For example, the term “EPA” encompasses eicosapentaenoic acid and a pharmaceutically acceptable ester, conjugate, or salt thereof, or mixtures of any of the foregoing. A “derivative” of a PUFA include molecules where the acyl group or the carbon chain portion of the molecule has one or more modifications.

In some embodiments, the PUFA derivative is an oxylipin. Oxylipins are bioactive lipids generated from oxygenation of PUFAs by enzymes such as cyclooxygenases (COX), lipoxygenases (LOX), and cytochrome P450 epoxygenase (CYP), or by non-enzymatic processes. Oxylipins especially those derived from EPA may have vaso-protective activities, including protection from septic and hypertensive vascular diseases. See Newman et al., PLOS ONE (2014) 9:11. For example, major oxylipin classes include prostacyclins (potent vasodilators), thromboxanes (potent pro-coagulants and vasoconstrictors), prostaglandins (vasodilators), leukotrienes (inflammatory mediators), epoxides (potent vasodilators), and resolvins (potent inflammation resolvers). PUFA-derived oxylipins are thought to mediate the effects of the PUFAs in many biological conditions. In certain of these embodiments, the PUFA derivative has at least one hydroxyl group, an epoxide group, a non-vicinal di-hydroxyl group, a vicinal di-hydroxyl group, a tri-hydroxyl group, and/or a ketone group. See Gabbs et al., Adv. Nutr. (2015) 6:513-40 for descriptions of oxylipin metabolites and potential therapeutic applications, which is incorporated herein by reference in its entirety.

In some embodiments, the LA derivative is at least one selected from the group consisting of 9-hydroperoxy-octadecadienoic acid (9-HpODE), 13-hydroperoxy-octadecadienoic acid (13-HpODE), 9-hydroxy-octadecadienoic acid (9-HODE), 13-hydroxy-octadecadienoic acid (13-HODE), 9,10,13 trihydroxy-octadecenoic acid (9,10,13 TriHOME), 9,12,13 trihydroxy-octadecenoic acid (9,12,13 TriHOME), 9-oxo-octadecadienoic acid (9-oxo-ODE), 13-oxo-octadecadienoic acid (13-oxo-ODE), 9,10-epoxy-octadecenoic acid (9,10-EpOME), 12,13-epoxy-octadecenoic acid (12,13-EpOME), 9,10-dihydroxy-octadecenoic acid (9,10-DiHOME), and 12,13-dihydroxy-octadecenoic acid (12,13-DiHOME). As examples of potential benefits, selected LA derivatives may be used as therapeutics to promote vasodilation/arterial relaxation (e.g., one or more HpODEs, EpOMEs), inhibit platelet aggregation (e.g., one or more HODEs), induce peroxisome proliferator-activated receptor (PPAR) (e.g., one or more HODEs, oxo-ODEs), inhibit leukotriene production (e.g., one or more HODEs), suppress triglyceride-rich lipoproteins (TRLs) assembly and secretion (e.g., one or more HODEs), and/or suppress tumor cell adhesion or proliferation (e.g., one or more HODEs, EpOMEs).

In some embodiments, the GLA derivative is at least one selected from the group consisting of 6-hydroxy-octatrienoic acid (6-HOTrE or 6-hydroxy-GLA), 7-hydroxy-octatrienoic acid (7-HOTrE or 7-hydroxy-GLA), 9-hydroxy-octatrienoic acid (9-HOTrE or 9-hydroxy-GLA), 10-hydroxy-octatrienoic acid (10-HOTrE or 10-hydroxy-GLA), 12-hydroxy-octatrienoic acid (12-HOTrE or 12-hydroxy-GLA), 13-hydroxy-octatrienoic acid (13-HOTrE or 13-hydroxy-GLA), 6,13-dihydroxy-octadienoic acid (6,13-DiHODE or 6,13-dihydroxy-GLA), and trihydroxy GLA derivatives (trihydroxy-GLAs). As examples of potential benefits, selected GLA derivatives may be used as therapeutics to promote vasodilation (e.g., one or more HOTrEs, DiHODEs, trihydroxy-GLAs), suppress reactive oxygen species generation (e.g., one or more HOTrEs, DiHODEs, trihydroxy-GLAs), mitigate neurodegenerative conditions (e.g., one or more HOTrEs, DiHODEs, trihydroxy-GLAs), and/or as anti-inflammatory therapeutics, for example, to reduce the production of tumor necrosis factor-α (TNF-α), to reduce the migration of neutrophils and macrophages into a site of inflammation, to reduce interleukin-1β (IL-1β) production in the individual, and/or to reduce macrophage chemotactic protein-1 (MCP-1) (e.g., one or more HOTrEs, DiHODEs, trihydroxy-GLAs). See U.S. Patent Application No. 2007/0248586 A1, which is incorporated herein by reference in its entirety.

In some embodiments, the DGLA derivative is at least one selected from the group consisting of prostaglandin D1 (PGD1), prostaglandin E1 (PGE1), 15-hydroxy-PGE1, 19-hydroxy-PGE1, 13,14-dihydroxy-PGE1, 13,14-dihydroxy-15-keto-PGE1, prostaglandin F1α (PGF1α), 6-keto-PGF1α, 15-keto-PGF1α, 13,14-dihydroxy-PGF1α, 15,19-dihydroxy-PGF1α, 13,14-dihydroxy-15-keto PGF1α, prostacyclin 11 (prostaglandin I1 or PGI1), thromboxane A1 (TXA1), thromboxane B1 (TXB1), leukotriene B3 (LTB3), leukotriene C3 (LTC3), leukotriene D3 (LTD3), leukotriene E3 (LTE3), 5-hydroperoxy-eicosatrienoic acid (5-HpETrE), 8-hydroperoxy-eicosatrienoic acid (8-HpETrE), 12-hydroperoxy-eicosatrienoic acid (12-HpETrE), 15-hydroperoxy-eicosatrienoic acid (15-HpETrE), 5-hydroxy-eicosatrienoic acid (5-HETrE), 8-hydroxy-eicosatrienoic acid (8-HETrE), 12-hydroxy-eicosatrienoic acid (12-HETrE), 15-hydroxy-eicosatrienoic acid (15-HETrE), 8,9-epoxy-eicosadienoic acid (8,9-EpEDE), 11,12-epoxy-eicosadienoic acid (11,12-EpEDE), 14,15-epoxy-eicosadienoic acid (14,15-EpEDE), 8,9-dihydroxy-eicosadienoic acid (8,9-DiHEDE), 11,12-dihydroxy-eicosadienoic acid (11,12-DiHEDE), and 14,15-dihydroxy-eicosadienoic acid (14,15-DiHEDE). As examples of potential benefits, selected DGLA derivatives may be used as therapeutics to promote vasodilation, including mitigating pulmonary hypertension or enhancing peripheral blood flow (e.g., one or more PGs or derivatives), inhibit platelet aggregation (e.g., one or more PGs or derivatives, HETrEs), alleviate neuropathy (e.g., one or more PGs or derivatives), promote wound healing (e.g., one or more PGs or derivatives), leukotriene production (e.g., one or more HETrEs), and/or suppress cellular hyperproliferation (e.g., one or more HETrEs). See U.S. Patent Application No. 2007/0248586 A1, which is incorporated herein by reference in its entirety.

In some embodiments, the AA or ARA derivative is at least one selected from the group consisting of 6-keto-prostaglandin F1 alpha (6k-PGF1α), thromboxane B2 (TXB2), 11-dehydro-thromboxane B2 (11-dTXB2), prostaglandin F2 alpha (PGF2a), prostaglandin E2 (PGE2), prostaglandin A2 (PGA2), prostaglandin D2 (PGD2), 2,3-dinor 11 beta-prostaglandin F2 alpha (2,3-dinor11bPGF2a), prostaglandin J2 (PGJ2), 15-deoxy-delta-12,14-prostaglandin J2 (15d-PGJ2), leukotriene B4 (LTB4), 20-hydroxy-leukotriene B4 (20-OH-LTB4), leukotriene C4 (LTC4), leukotriene D4 (LTD4), leukotriene E4 (LTE4), 5-hydroperoxy-eicosatetraenoic acid (5-HpETE), 8-hydroperoxy-eicosatetraenoic acid (8-HpETE), 9-hydroperoxy-eicosatetraenoic acid (9-HpETE), 11-hydroperoxy-eicosatetraenoic acid (11-HpETE), 12-hydroperoxy-eicosatetraenoic acid (12-HpETE), 15-hydroperoxy-eicosatetraenoic acid (15-HpETE), 5-hydroxy-eicosatetraenoic acid (5-HETE), 8-hydroxy-eicosatetraenoic acid (8-HETE), 9-hydroxy-eicosatetraenoic acid (9-HETE), 11-hydroxy-eicosatetraenoic acid (11-HETE), 12-hydroxy-eicosatetraenoic acid (12-HETE), 15-hydroxy-eicosatetraenoic acid (15-HETE), 18-hydroxy-eicosatetraenoic acid (18-HETE), 19-hydroxy-eicosatetraenoic acid (19-HETE), 20-hydroxy-eicosatetraenoic acid (20-HETE), 5-oxo-eicosatetraenoic acid (5-oxo-ETE), 8-oxo-eicosatetraenoic acid (8-oxo-ETE), 11-oxo-eicosatetraenoic acid (11-oxo-ETE), 12-oxo-eicosatetraenoic acid (12-oxo-ETE), 15-oxo-eicosatetraenoic acid (15-oxo-ETE), lipoxin A4 (LXA4), lipoxin A5 (LXA5), hepoxilin A3 (HxA3), hepoxilin B3 (HxB3), trioxilin A3 (TrxA3), trioxilin B3 (TrxB3), eoxin A4 (ExA4), eoxin C4 (ExC4), eoxin D4 (ExD4), eoxin E4 (ExE4), 5,6-epoxy-eicosatrienoic acid (5,6-EpETrE, or 5,6-EET), 8,9-epoxy-eicosatrienoic acid (8,9-EpETrE, or 8,9-EET), 11,12-epoxy-eicosatrienoic acid (11,12-EpETrE, or 11,12-EET), 14,15-epoxy-eicosatrienoic acid (14,15-EpETrE, or 14,15-EET), 5,12-dihydroxy-eicosatetraenoic acid (5,12-DiHETE), 5,6-dihydroxy-eicosatrienoic acid (5,6-DiHETrE, or 5,6-DiHET), 8,9-dihydroxy-eicosatrienoic acid (8,9-DiHETrE, or 8,9-DiHET), 11,12-dihydroxy-eicosatrienoic acid (11,12-DiHETrE, or 11,12-DiHET), 14,15-dihydroxy-eicosatrienoic acid (14,15-DiHETrE, or 14,15-DiHET), and 12-hydroxyheptadecatrenoic acid (12-HHTrE). As examples of potential benefits, selected AA derivatives may be used as therapeutics to promote vasodilation/arterial relaxation (e.g., one or more PGs or derivatives, HpETEs, HETEs, EpETrEs, DiHETrEs), promote angiogenesis (e.g., one or more HETEs, EpETrEs, DiHETrEs), lower intracranial or renal pressure (e.g., one or more HETEs), attenuate myocyte apoptosis following reperfusion (e.g., one or more EpETrEs), inhibit platelet aggregation (e.g., one or more PGs and derivatives, HpETEs), attenuate inflammation (e.g., one or more PGs and derivatives, HETEs, lipoxins), suppress reactive oxygen species generation/oxidative injury (e.g., one or more lipoxins), induce PPAR (e.g., one or more PG derivatives, HETEs, DiHETrEs), promote adipocyte differentiation (e.g., one or more hepoxilins), attenuate cancer (e.g., one or more oxo-ETEs), promote wound healing (e.g., one or more HETEs), and/or attenuate LOX activity (e.g., one or more oxo-ETEs). See Gabbs et al., Adv. Nutr. (2015) 6:513-40, which is incorporated herein by reference in its entirety.

In some embodiments, the AdA or DTA derivative is at least one selected from the group consisting of dihomo-prostaglandin E2 (dihomo-PGE2), dihomo-prostaglandin D2 (dihomo-PGD2), dihomo-prostaglandin F1α (dihomo-PGF2a), dihomo-prostacyclin 12 (dihomo-prostaglandin 12 or dihomo-PGI2), dihomo-thromboxane A2 (dihomo-TXA2), dihomo-thromboxane B2 (dihomo-TXB2), 7-hydroperoxy-docosatetraenoic acid (dihomo-7-HpETE), 10-hydroperoxy-docosatetraenoic acid (dihomo-10-HpETE), 11-hydroperoxy-docosatetraenoic acid (dihomo-11-HpETE), 13-hydroperoxy-docosatetraenoic acid (dihomo-13-HpETE), 14-hydroperoxy-docosatetraenoic acid (dihomo-14-HpETE), 17-hydroperoxy-docosatetraenoic acid (dihomo-17-HpETE), 7-hydroxy-docosatetraenoic acid (dihomo-7-HETE), 10-hydroxy-docosatetraenoic acid (dihomo-10-HETE), 11-hydroxy-docosatetraenoic acid (dihomo-11-HETE), 13-hydroxy-docosatetraenoic acid (dihomo-13-HETE), 14-hydroxy-docosatetraenoic acid (dihomo-14-HETE), 17-hydroxy-docosatetraenoic acid (dihomo-17-HETE), 7,11-dihydroxy-docosatetraenoic acid (dihomo-7,11-DiHETE), 7,14-dihydroxy-docosatetraenoic acid (dihomo-7,14-DiHETE), 7,17-dihydroxy-docosatetraenoic acid (dihomo-7,17-DiHETE), 10,17-dihydroxy-docosatetraenoic acid (dihomo-10,17-DiHETE), 11,17-dihydroxy-docosatetraenoic acid (dihomo-11,17-DiHETE), 13,15-dihydroxy-docosatetraenoic acid (dihomo-13,15-DiHETE), 13,17-dihydroxy-docosatetraenoic acid (dihomo-13,17-DiHETE), 16,17-dihydroxy-docosatetraenoic acid (dihomo-16,17-DiHETE), 7,8-epoxy-docosatrienoic acid (dihomo-7,8-EpETrE), 10,11-epoxy-docosatrienoic acid (dihomo-10,11-EpETrE), 13,14-epoxy-docosatrienoic acid (dihomo-13,14-EpETrE), 16,17-epoxy-docosatrienoic acid (dihomo-16,17-EpETrE), 7,8-dihydroxy-docosatrienoic acid (dihomo-7,8-DiHETrE), 10,11-dihydroxy-docosatrienoic acid (dihomo-10,11-DiHETrE), 13,14-dihydroxy-docosatrienoic acid (dihomo-13,14-DiHETrE), 16,17-dihydroxy-docosatrienoic acid (dihomo-16,17-DiHETrE), 7,16,17-trihydroxy-docosatetraenoic acid (di homo-7,16,17-trihydroxy-ETrE), and 14-hydroxy-7,10,12-nonadecatrienoic acid (14-HNTrE). As an example of potential benefits, selected AdA derivatives may be used as therapeutics to promote vasodilation and/or oppose net vasoconstrictive properties from analogous oxylipins, especially in the kidney (e.g., one or more dihomo-PGs or derivatives, dihomo-thromboxanes, dihomo-HETEs, dihomo-DiHETEs dihomo-EpETrEs), suppress reactive oxygen species generation (e.g., one or more dihomo-HETEs, dihomo-DiHETEs), mitigate neurodegenerative conditions (e.g., one or more dihomo-HETEs, dihomo-DiHETEs), and as anti-inflammatory therapeutics, for example to reduce the production of tumor necrosis factor-α (TNF-α), to reduce the migration of neutrophils and macrophages into a site of inflammation, to reduce interleukin-1β (IL-1β) production in the individual, and/or to reduce macrophage chemotactic protein-1 (MCP-1) (e.g., one or more dihomo-HETEs, dihomo-DiHETEs). See U.S. Patent Application No. 2006/0241088 A1, which is incorporated herein by reference in its entirety.

In some embodiments, the DPA6 derivative is at least one selected from the group consisting of 7-hydroperoxy-DPA6, 8-hydroperoxy-DPA6, 10-hydroperoxy-DPA6, 11-hydroperoxy-DPA6, 13-hydroperoxy-DPA6, 14-hydroperoxy-DPA6, 17-hydroperoxy-DPA6, 7-hydroxy-DPA6, 8-hydroxy-DPA6, 10-hydroxy-DPA6, 11-hydroxy-DPA6, 13-hydroxy-DPA6, 14-hydroxy-DPA6, 17-hydroxy-DPA6, 4,5-dihydroxy-DPA6, 7,14-dihydroxy-DPA6, 7,17-dihydroxy-DPA6, 8,14-dihydroxy-DPA6, 10,17-dihydroxy-DPA6, 13,17-dihydroxy-DPA6, 16,17-dihydroxy-DPA6, 4,5,17-trihydroxy-DPA6, 7,16,17-trihydroxy-DPA6, and 10,13,17-trihydroxy-DPA6. As examples of potential benefits, selected DPA6 derivatives may be used as therapeutics to promote vasodilation (e.g., one or more hydroxy-DPA6s, dihydroxy-DPA6s), suppress reactive oxygen species generation (e.g., one or more hydroxy-DPA6s, dihydroxy-DPA6s), mitigate neurodegenerative conditions (e.g., one or more hydroxy-DPA6s, dihydroxy-DPA6s), and as anti-inflammatory therapeutics, for example to reduce the production of tumor necrosis factor-α (TNF-α), to reduce the migration of neutrophils and macrophages into a site of inflammation, to reduce interleukin-1β (IL-1β) production in the individual, and/or to reduce macrophage chemotactic protein-1 (MCP-1) (e.g., one or more hydroxy-DPA6s, dihydroxy-DPA6s). See U.S. Patent Application No. 2006/0241088 A1, which is incorporated herein by reference in its entirety.

In some embodiments, the ALA derivative is at least one selected from the group consisting of 9-hydroperoxy-octatrienoic acid (9-HpOTrE), 13-hydroperoxy-octatrienoic acid (13-HpOTrE), 9-hydroxy-octatrienoic acid (9-HOTrE), 13-hydroxy-octatrienoic acid (13-HOTrE), 9,16-dihydroxy-octatrienoic acid (9,16-DiHOTrE), 9-oxo-octatrienoic acid (9-oxo-OTrE), 13-oxo-octatrienoic acid (13-oxo-OtrE), 9,10-epoxy-octadienoic acid (9,10-EpODE), 12,13-epoxy-octadienoic acid (12,13-EpODE), 15,16-epoxy-octadienoic acid (15,16-EpODE), 9,10-dihydroxy-octadienoic acid (9,10-DiHODE), 12,13-dihydroxy-octadienoic acid (12,13-DiHODE), and 15,16-dihydroxy-octadienoic acid (15,16-DiHODE). As examples of potential benefits, selected ALA derivatives may be used as therapeutics to inhibit platelet aggregation (e.g., one or more DiHOTrEs), attenuate inflammation (e.g., one or more HOTrEs), promote adipocyte differentiation (e.g., one or more oxo-OtrEs), attenuate COX activity (e.g., one or more DiHOTrEs), lower action potential in myocytes (e.g., one or more HpOTrEs), and/or increase glucose uptake (e.g., one or more oxo-OtrEs).

In some embodiments, the SDA derivative is at least one selected from the group consisting of 6-hydroperoxy-octatetraenoic acid (6-HpOTE or 6-hydroperoxy-SDA), 7-hydroperoxy-octatetraenoic acid (7-HpOTE or 7-hydroperoxy-SDA), 9-hydroperoxy-octatetraenoic acid (9-HpOTE or 9-hydroperoxy-SDA), 10-hydroperoxy-octatetraenoic acid (10-HpOTE or 10-hydroperoxy-SDA), 12-hydroperoxy-octatetraenoic acid (12-HpOTE or 12-hydroperoxy-SDA), 13-hydroperoxy-octatetraenoic acid (13-HpOTE or 13-hydroperoxy-SDA), 15-hydroperoxy-octatetraenoic acid (15-HpOTE or 15-hydroperoxy-SDA), 16-hydroperoxy-octatetraenoic acid (16-HpOTE or 16-hydroperoxy-SDA), 6-hydroxy-octatetraenoic acid (6-HOTE or 6-hydroxy-SDA), 7-hydroxy-octatetraenoic acid (7-HOTE or 7-hydroxy-SDA), 9-hydroxy-octatetraenoic acid (9-HOTE or 9-hydroxy-SDA), 10-hydroxy-octatetraenoic acid (10-HOTE or 10-hydroxy-SDA), 12-hydroxy-octatetraenoic acid (12-HOTE or 12-hydroxy-SDA), 13-hydroxy-octatetraenoic acid (13-HOTE or 13-hydroxy-SDA), 15-hydroxy-octatetraenoic acid (15-HOTE or 15-hydroxy-SDA), 16-hydroxy-octatetraenoic acid (16-HOTE or 16-hydroxy-SDA), 6,13-di hydroxy-octadecatrienoic acid (6,13-DiHOTrE or 6,13-dihydroxy-SDA), 6,16-dihydroxy-octadecatrienoic acid (6,16-DiHOTrE or 6,16-dihydroxy-SDA), 6,7-dihydroxy-octadecadienoic acid (6,7-DiHODE or 6,7-dihydroxy-SDA), 9,10-dihydroxy-octadecadienoic acid (9,10-DiHODE or 9,10-dihydroxy-SDA), 12,13-dihydroxy-octadecadienoic acid (12,13-DiHODE or 12,13-dihydroxy-SDA), 15,16-dihydroxy-octadecadienoic acid (15,16-DiHODE or 15,16-dihydroxy-SDA), and trihydroxy-SDAs bearing a hydroxyl group at any three positions among carbons C6, C7, C9, C10, C12, C13, C15 or C16 of SDA (trihydroxy-SDAs). As examples of potential benefits, selected SDA derivatives may be used as therapeutics to promote vasodilation (e.g., one or more HOTEs, DiHOTrEs, DiHODEs, trihydroxy-SDAs), suppress reactive oxygen species generation (e.g., one or more HOTEs, DiHOTrEs, DiHODEs, trihydroxy-SDAs), mitigate neurodegenerative conditions (e.g., one or more HOTEs, DiHOTrEs, DiHODEs, trihydroxy-SDAs), and as anti-inflammatory therapeutics, for example to reduce the production of tumor necrosis factor-α (TNF-α), to reduce the migration of neutrophils and macrophages into a site of inflammation, to reduce interleukin-1β (IL-1β) production in the individual, and/or to reduce macrophage chemotactic protein-1 (MCP-1) (e.g., one or more HOTEs, DiHOTrEs, DiHODEs, trihydroxy-SDAs). See U.S. Patent Application No. 2007/0248586 A1, which is incorporated herein by reference in its entirety.

In some embodiments, the ETA derivative is at least one selected from the group consisting of Δ17,18 prostaglandin D1 (Δ17,18 PGD1 or ω-3 PGD1), Δ17,18 prostaglandin E1 (Δ17,18 PGE1 or ω-3 PGE1), and Δ17,18 prostaglandin F1α (Δ17,18 PGF1α or ω-3 PGF1α), Δ17,18 prostacyclin 11 (Δ17,18 PGI1 or ω-3 PGE1), Δ17,18 12-hydroperoxy-eicosatetraenoic acid (Δ17,18 12-HpETE or ω-3 12-HpETE), Δ17,18 15-hydroperoxy-eicosatetraenoic acid (Δ17,18 15-HpETE or ω-3 15-HpETE), Δ16,17 18-hydroperoxy-eicosatetraenoic acid (Δ16,17 18-HpETE), Δ17,18 12-hydroxy-eicosatetraenoic acid (Δ17,18 12-HETE or ω-3 12-HETE), Δ17,18 15-hydroxy-eicosatetraenoic acid (Δ17,18 15-HETE or ω-3 15-HETE), Δ16,17 18-hydroxy-eicosatetraenoic acid (Δ16,17 18-HETE), 0.617,18 19-hydroxy-eicosatetraenoic acid (Δ17,18 19-HETE or ω-3 19-HETE), Δ17,18 20-hydroxy-eicosatetraenoic acid (Δ17,18 20-HETE or ω-3 20-HETE), Δ17,18 11,12 epoxy-eicosatrienoic acid (Δ17,18 11,12-EpETrE or ω-3 11,12-EpETrE), Δ17,18 14,15 epoxy-eicosatrienoic acid (Δ17,18 14,15-EpETrE or ω-3 14,15-EpETrE), and 17,18 epoxy-eicosatrienoic acid (17,18-EpETrE), Δ17,18 11,12 dihydroxy-eicosatrienoic acid (Δ17,18 11,12-DiHETrE or ω-3 11,12-DiHETrE), Δ17,18 14,15 dihydroxy-eicosatrienoic acid (Δ17,18 14,15-DiHETrE or ω-3 14,15-DiHETrE), and 17,18 dihydroxy-eicosatrienoic acid (17,18-DiHETrE). As examples of potential benefits, selected ETA derivatives may be used as therapeutics to inhibit COX and suppress conversion of AA into PGs and thromboxanes (e.g., one or more ω-3 HETEs, ω-3 DiHETrEs) and/or promote vasodilation or mitigate congestive heart failure (e.g., one or more ω-3 HETEs, ω-3 EpETrEs, ω-3 DiHETrEs).

In some embodiments, the EPA derivative is at least one selected from the group consisting of 6-keto-prostaglandin F2 alpha (6k-PGF2a), thromboxane B3 (TXB3), 11-dehydro-thromboxane B3 (11-dTXB3), prostaglandin F3 alpha (PGF3a), prostaglandin E3 (PGE3), prostaglandin A3 (PGA3), prostaglandin D3 (PGD3), 2,3-dinor 11 beta-prostaglandin F3 alpha (2,3-dinor11bPGF3a), prostaglandin J3 (PGJ3), 15-deoxy-delta-12,14-prostaglandin J3 (15d-PGJ3), leukotriene B5 (LTB5), 20-hydroxy-leukotriene B5 (20-OH-LTB5), leukotriene C5 (LTC5), leukotriene D5 (LTD5), leukotriene E5 (LTE5), 5-hydroperoxy-eicosapentaenoic acid (5-HpEPE), 8-hydroperoxy-eicosapentaenoic acid (8-HpEPE), 9-hydroperoxy-eicosapentaenoic acid (9-HpEPE), 11-hydroperoxy-eicosapentaenoic acid (11-HpEPE), 12-hydroperoxy-eicosapentaenoic acid (12-HpEPE), 15-hydroperoxy-eicosapentaenoic acid (15-HpEPE), 18-hydroperoxy-eicosapentaenoic acid (18-HpEPE), 5-hydroxy-eicosapentaenoic acid (5-HEPE), 8-hydroxy-eicosapentaenoic acid (8-HEPE), 9-hydroxy-eicosapentaenoic acid (9-HEPE), 11-hydroxy-eicosapentaenoic acid (11-HEPE), 12-hydroxy-eicosapentaenoic acid (12-HEPE), 15-hydroxy-eicosapentaenoic acid (15-HEPE), 18-hydroxy-eicosapentaenoic acid (18-HEPE), 19-hydroxy-eicosapentaenoic acid (19-HE PE), 20-hydroxy-eicosapentaenoic acid (20-HEPE), 5-oxo-eicosapentaenoic acid (5-oxo-EPE), 12-oxo-eicosapentaenoic acid (12-oxo-EPE), 15-oxo-eicosapentaenoic acid (15-oxo-EPE), 5,6-epoxy-eicosatetraenoic acid (5,6-EpETE), 8,9-epoxy-eicosatetraenoic acid (8,9-EpETE), 11,12-epoxy-eicosatetraenoic acid (11,12-EpETE), 14,15-epoxy-eicosatetraenoic acid (14,15-EpETE), 5,6-dihydroxy-eicosatetraenoic acid (5,6-diHETE), 8,9-dihydroxy-eicosatetraenoic acid (8,9-diHETE), 11,12-dihydroxy-eicosatetraenoic acid (11,12-diHETE), 14,15-dihydroxy-eicosatetraenoic acid (14,15-diHETE), 17,18-dihydroxy-eicosatetraenoic acid (17,18-diHETE), 17,18-epoxy-eicosatetraenoic acid (17,18-EpETE), lipoxin A5 (LxAS), lipoxin B5 (LxBS), 15-epi-lipoxin A4, resolvin E1 (RvE1), resolvin E2 (RvE2), resolvin E3 (RvE3), and resolvin E4 (RvE4). As examples of potential benefits, selected EPA derivatives may be used as therapeutics to promote vasodilation/arterial relaxation (e.g., one or more PGs or derivatives, lipoxins, HEPEs, EpETEs), inhibit platelet aggregation in absolute terms or relative to AA-derived oxylipins (e.g., one or more PGs and derivatives, HpEPEs, HEPEs, EpETEs, DiHETEs), attenuate inflammation (e.g., one or more PGs and derivatives, LTs, HEPEs, oxo-EPEs, EpETEs, RvEs), promote adipocyte differentiation/raise adiponectin (e.g., one or more PGs and derivatives, HEPES), attenuate cancer (e.g., one or more PGs or derivatives), attenuate COX activity (e.g., one more LTs, HpEPEs), attenuate LOX activity (e.g., one more LTs, HpEPEs, HEPEs), and/or enhance glucose-dependent insulin secretion (e.g., one more HEPEs).

In some embodiments, the DPA derivative is at least one selected from the group consisting of 7-hydroperoxy-docosapentaeonic acid (7-hydroperoxy-DPA), 10-hydroperoxy-docosapentaeonic acid (10-hydroperoxy-DPA), 11-hydroperoxy-docosapentaeonic acid (11-hydroperoxy-DPA), 13-hydroperoxy-docosapentaeonic acid (13-hydroperoxy-DPA), 14-hydroperoxy-docosapentaeonic acid (14-hydroperoxy-DPA), 16-hydroperoxy-docosapentaeonic acid (16-hydroperoxy-DPA), 17-hydroperoxy-docosapentaeonic acid (17-hydroperoxy-DPA), 7-hydroxy-docosapentaeonic acid (7-hydroxy-DPA), 10-hydroxy-docosapentaeonic acid (10-hydroxy-DPA), 11-hydroxy-docosapentaeonic acid (11-hydroxy-DPA), 13-hydroxy-docosapentaeonic acid (13-hydroxy-DPA), 14-hydroxy-docosapentaeonic acid (14-hydroxy-DPA), 16-hydroxy-docosapentaeonic acid (16-hydroxy-DPA), 17-hydroxy-docosapentaeonic acid (17-hydroxy-DPA), 7,17-di hydroxy-docosape ntaeon ic acid (7,17-di hydroxy-DPA), 8,14-dihydroxy-docosapentaeonic acid (8,14-dihydroxy-DPA), 10,17-dihydroxy-docosapentaeonic acid (10,17-dihydroxy-DPA), 10,20-dihydroxy-docosapentaeonic acid (10,20-dihydroxy-DPA), 13,20-dihydroxy-docosapentaeonic acid (13,20-dihydroxy-DPA), 16,17-dihydroxy-docosapentaeonic acid (16,17-dihydroxy-DPA), 13-oxo-docosapentaeonic acid (13-oxo-DPA or 13-EFOX-D5), MaR1n-3 DPA, MaR2n-3 DPA, MaR3n-3 DPA, PD1n-3 DPA, PD2n-3 DPA, 7,13,20-trihydroxy-n-3-docosapentaeonic acid (resolvin T1 or RvT1), 7,12,13-trihydroxy-n-3-docosapentaeonic acid (resolvin T2 or RvT2), 7,8,13-trihydroxy-n-3-docosapentaeonic acid (resolvin T3 or RvT3), 7,16,17-trihydroxy-n-3-docosapentaeonic acid (7,16,17-trihydroxy-DPA), RvD1 n-3 DPA, RvD2n-3 DPA, and RvD2n-3 DPA. As examples of potential benefits, selected DPA derivatives may be used as therapeutics to promote vasodilation (e.g., one or more hydroxy-DPAs, dihydroxy-DPAs), suppress reactive oxygen species generation (e.g., one or more hydroxy-DPAs, dihydroxy-DPAs), mitigate neurodegenerative conditions (e.g., one or more hydroxy-DPAs, dihydroxy-DPAs), and as anti-inflammatory therapeutics, for example to reduce the production of tumor necrosis factor-α (TNF-α), to reduce the migration of neutrophils and macrophages into a site of inflammation, to reduce interleukin-1β (IL-1β) production in the individual, and/or to reduce macrophage chemotactic protein-1 (MCP-1) (e.g., one or more hydroxy-DPAs, dihydroxy-DPAs). See Duan et al., Front Physiol. (2021) 12:646491; U.S. Patent Application No. 2006/0241088 A1, each of which is incorporated herein by reference in its entirety.

In some embodiments, the DHA derivative is at least one selected from the group consisting of 4-hydroperoxy-docosahexaenoic acid (4-HpDoHE), 7-hydroperoxy-docosahexaenoic acid (7-HpDoHE), 8-hydroperoxy-docosahexaenoic acid (8-HpDoHE), 10-hydroperoxy-docosahexaenoic acid (10-HpDoHE), 11-hydroperoxy-docosahexaenoic acid (11-HpDoHE), 13-hydroperoxy-docosahexaenoic acid (13-HpDoHE), 14-hydroperoxy-docosahexaenoic acid (14-HpDoHE), 16-hydroperoxy-docosahexaenoic acid (16-HpDoHE), 17-hydroperoxy-docosahexaenoic acid (17-HpDoHE), 4-hydroxy-docosahexaenoic acid (4-HDoHE), 7-hydroxy-docosahexaenoic acid (7-HDoHE), 8-hydroxy-docosahexaenoic acid (8-HDoHE), 10-hydroxy-docosahexaenoic acid (10-HDoHE), 11-hydroxy-docosahexaenoic acid (11-HDoHE), 13-hydroxy-docosahexaenoic acid (13-HDoHE), 14-hydroxy-docosahexaenoic acid (14-HDoHE), 16-hydroxy-docosahexaenoic acid (16-HDoHE), 17-hydroxy-docosahexaenoic acid (17-HDoHE), 20-hydroxy-docosahexaenoic acid (20-HDoHE), 21-hydroxy-docosahexaenoic acid (21-HDoHE), 22-hydroxy-docosahexaenoic acid (22-HDoHE), 7,14-dihydroxy-docosahexaenoic acid (7,14-DiHDoHE), 7,17-dihydroxy-docosahexaenoic acid (7,17-DiHDoHE), 8,14-dihydroxy-docosahexaenoic acid (8,14-DiHDoHE), 10,17-dihydroxy-docosahexaenoic acid (10,17-DiHDoHE), 10,20-dihydroxy-docosahexaenoic acid (10,20-DiHDoHE), 14,20-dihydroxy-docosahexaenoic acid (14,20-DiHDoHE), 14,21-dihydroxy-docosahexaenoic acid (14,21-DiHDoHE), 7-oxo-docosahexaenoic acid (7-oxo-DoHE), 4,5-epoxy-docosapentaenoic acid (4,5-EpDPE), 7,8-epoxy-docosapentaenoic acid (7,8-EpDPE), 10,11-epoxy-docosapentaenoic acid (10,11-EpDPE), 13,14-epoxy-docosapentaenoic acid (13,14-EpDPE), 16,17-epoxy-docosapentaenoic acid (16,17-EpDPE), 19,20-epoxy-docosapentaenoic acid (19,20-EpDPE), 4,5-dihydroxy-docosapentaenoic acid (4,5-DiHDPE), 7,8-dihydroxy-docosapentaenoic acid (7,8-DiHDPE), 10,11-dihydroxy-docosapentaenoic acid (10,11-DiHDPE), 13,14-dihydroxy-docosapentaenoic acid (13,14-DiHDPE), 16,17-dihydroxy-docosapentaenoic acid (16,17-DiHDPE), 19,20-dihydroxy-docosapentaenoic acid (19,20-DiHDPE), 4,5-epoxy-17-0H-docosahexaenoic acid, (4,5-epoxy-17-hydroxy-DHA), 7,8-epoxy-17-0H-docosahexaenoic acid (7,8-epoxy-17-hydroxy-DHA), maresin 1 (MaR1), maresin 2 (MaR2), protectin 1 (PD1), protectin X (PDX), aspirin-triggered PD1 (AT-PD1), resolvin D1 (RvD1), resolvin D2 (RvD2), resolvin D3 (RvD3), resolvin D4 (RvD4), resolvin D5 (RvD5), resolvin D6 (RvD6), aspirin-triggered resolvin D1 (AT-RvD1), aspirin-triggered resolvin D2 (AT-RvD2), aspirin-triggered resolvin D3 (AT-RvD3), aspirin-triggered resolvin D4 (AT-RvD4), aspirin-triggered resolvin D5 (AT-RvD5), and aspirin-triggered resolvin D6 (AT-RvD6). As examples of potential benefits, selected DHA derivatives may be used as therapeutics to promote vasodilation/arterial relaxation (e.g., one or more HDoHEs, EpDPEs, DiHDPEs), inhibit platelet aggregation (e.g., one or more HDoHE, EpEPE, DiHDPE, PDs), attenuate inflammation/inflammatory pain (e.g., one or more HDoHEs, DiHDoHEs, DiHDPEs, PDs, MaRs, RvDs, AT-RvDs), suppress reactive oxygen species generation/oxidative injury (e.g., one or more HDoHEs, PDs), induce PPAR (e.g., one or more HDoHEs), attenuate COX activity (e.g., one or more HDoHEs), attenuate LOX activity (e.g., one or more PDs), improve insulin sensitivity (e.g., one or more PDs), and/or promote wound healing (e.g., one or more DiHDoHEs, PDs).

In some embodiments, the PUFAs or derivatives thereof of the composition may comprise tetracosatetraenoic acid (TTE, a 24-carbon omega-6 VLC-PUFA with four double bonds), tetracosapentaenoic acid (TPA, a 24-carbon omega-3 VLC-PUFA with five double bonds), tetracosahexaenoic acid (THA, a 24-carbon omega-3 VLC-PUFA with six double bonds), or another VLC-PUFA or derivative thereof.

In some embodiments, compositions of the present technology comprise EPA as an active ingredient. The term “EPA” as used herein refers to eicosapentaenoic acid (e.g., all-cis eicosa-5,8,11,14,17-pentaenoic acid) in free acid form and/or a pharmaceutically acceptable ester, conjugate, or salt thereof, or mixtures of any of the foregoing. For example, in some embodiments, the EPA comprises eicosapentaenoic acid. In other embodiments, the EPA is in the form of an eicosapentaenoic acid ester, for example, a C1-C5 alkyl ester of eicosapentaenoic acid. In one embodiment, the EPA comprises eicosapentaenoic acid ethyl ester (also referred to herein as ethyl eicosapentaenoic acid, ethyl eicosapentaenoate, icosapent ethyl, ethyl-EPA, EtEPA, IPE, E-EPA or EPA-E). In another embodiment, the EPA comprises eicosapentaenoic acid methyl ester, eicosapentaenoic acid propyl ester, or eicosapentaenoic acid butyl ester. In another embodiment, the EPA comprises lithium eicosapentaenoic acid, a mono-, di- or triglyceride of eicosapentaenoic acid, or any other ester or salt of eicosapentaenoic acid.

In some embodiments, the EPA comprises an EPA-fatty acid conjugate wherein EPA is conjugated to another molecule of EPA or to another fatty acid (or a derivative thereof). In some embodiments, the EPA-fatty acid conjugate comprises a diester formed between EPA and EPA, or between EPA and a second fatty acid (or a derivative thereof), as shown in structures (I) where:

R¹O—R³—OR² (I)
R1 is an acyl group derived from EPA;
R2 is an acyl group derived from a 10 to 30 carbon fatty acid optionally with one or more cis or trans double bonds, or a derivative thereof, where R2 can be the same as or different from R1; and
R3 is an alkylene group with one or more hydrogens optionally substituted with an alkyl group, a hydroxyl group, an epoxy group, an aryl group, a phosphate group, or a phosphate group modified with small organic compound.

In certain of these embodiments, R1 and R2 may both be acyl groups derived from EPA (i.e., EPA-EPA conjugate). Alternatively, R1 may be derived from EPA, and R2 from a different fatty acid or a derivative thereof (i.e., EPA-fatty acid conjugate), for example, an omega-6 fatty acid such as LA, GLA, DGLA, AA, AdA, and DPA6, or an omega-3 fatty acid such as ALA, SDA, ETA, DPA, and DHA, or a derivative thereof.

Synthesis of a diester conjugate can be accomplished according to methods well known in the art, including for example, using metals, metal-chlorides, or organic acids as catalysts; using fatty acid chlorides such as EPA-chloride, LA-chloride, conjugated linoleic acid chloride (cLA-chloride), GLA-chloride, DGLA-chloride, AA-chloride, AdA-chloride, DPA6 ALA-chloride, SDA-chloride, ETA-chloride, DPA-chloride, and DHA-chloride; and using immobilized enzymes as catalysts.

In some embodiments, the EPA-fatty acid conjugate (e.g., EPA-fatty acid diester) comprises a phospholipid-EPA (PL-EPA) conjugate in which the fatty acid at the sn-1 and/or sn-2 position of the phospholipid molecule is replaced with EPA. In some embodiments, an acyl group derived from EPA, and a second acyl group derived from EPA or a second fatty acid (or a derivative thereof), are attached to the sn-1 and sn-2 carbons of a phospholipid molecule, as shown in structures (II)

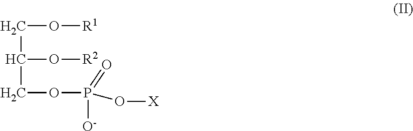

where:
each of R1 and R2 is an acyl group derived from a 10 to 30 carbon fatty acid optionally with one or more cis or trans double bonds, or a derivative thereof, wherein at least one of R1 and R2 is derived from EPA; and
X is selected from the group consisting of anion, choline, ethanolamine, glycerol, inositol, and serine.

In certain of these embodiments, the EPA-fatty acid conjugate (e.g., PL-EPA) can also serve as the source of phospholipid of the composition. Phospholipids (e.g., glycerophospholipids) with a specific fatty acid composition (e.g., enriched with EPA) can be synthesized according to methods described in WO2005/038037, Int. J. Mol. Sci. (2014) 15:15244-15258, and J. Oleo Sci. (2017) 66(11):1207-1215, the entire contents of each of which are incorporated by reference herein.

In should be noted that EPA in the embodiments discussed above is illustrative, and the EPA in those embodiments can be replaced with another PUFA or a derivative thereof as disclosed herein, for example, an omega-6 fatty acid such as LA, GLA, DGLA, AA, AdA, and DPA6, or an omega-3 fatty acid such as ALA, SDA, ETA, DPA, and DHA, or a derivative thereof.

In some embodiments, the composition contains at least 5%, by weight of the composition, of the one or more PUFAs (e.g., EPA, as the term “EPA” is defined and exemplified herein) or derivatives thereof as disclosed herein, for example, at least 5%, at least 10%, at least 15%, at least 20%, at least 30%, at least 40%, at least 50%, at least 60%, at least 70%, at least 80%, at least 90%, at least 95%, at least 96%, at least 97%, at least 98%, or at least 99% of PUFAs or derivatives thereof, by weight of the composition.

In some embodiments, the composition contains a mixture of two or more PUFAs or derivatives thereof as disclosed herein. In certain of these embodiments, each PUFA or derivative thereof comprises at least 5%, by weight, of the composition, for example, at least 5%, at least 10%, at least 15%, at least 20%, at least 30%, at least 40%, or about 50%, by weight, of the composition.

In some embodiments, the composition contains at least 5%, by weight of the composition, EPA (as the term “EPA” is defined and exemplified herein) or a derivative thereof, for example, at least 5%, at least 10%, at least 15%, at least 20%, at least 30%, at least 40%, at least 50%, at least 60%, at least 70%, at least 80%, at least 90%, at least 95%, at least 96%, at least 97%, at least 98%, or at least 99% EPA or a derivative thereof, by weight of the composition.

In some embodiments, the composition contains at least 5%, by weight of the composition, EPA-fatty acid conjugate, for example, at least 5%, at least 10%, at least 15%, at least 20%, at least 30%, at least 40%, at least 50%, at least 60%, at least 70%, at least 80%, at least 90%, at least 95%, at least 96%, at least 97%, at least 98%, or at least 99% EPA-fatty acid conjugate, by weight of the composition. In some embodiments, the composition contains no more than 10%, no more than 9%, no more than 8%, no more than 7%, no more than 6%, no more than 5%, no more than 4%, no more than 3%, no more than 2%, no more than 1%, no more than 0.6%, no more than 0.5%, no more than 0.4%, no more than 0.3%, no more than 0.2, or no more than 0.1% of any EPA-fatty acid conjugate other than EPA-EPA diester, by weight of the composition.

In some embodiments, the composition contains a mixture of EPA-fatty acid conjugates, for example, EPA-fatty acid diesters. In some embodiments, the composition contains less than 20%, less than 15%, less than 10%, less than 9%, less than 8%, less than 7%, less than 6%, less than 5%, less than 4%, less than 3%, less than 2%, less than 1%, less than 0.5%, or less than 0.1% EPA-DHA conjugate (e.g., EPA-DHA diester), by weight of the composition.

In some embodiments, EPA (as the term “EPA” is defined and exemplified herein) is present in the composition in an amount of about 50 mg to about 5000 mg, about 75 mg to about 2500 mg, or about 100 mg to about 1000 mg, for example, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, or about 5000 mg.

In some embodiments, the composition is formulated for administration to a subject in an amount sufficient to provide a daily dose of EPA (as the term “EPA” is defined and exemplified herein) about 1 mg to about 20,000 mg, about 25 mg to about 10,000 mg, about 50 mg to about 5000 mg, about 75 mg to about 2500 mg, or about 100 mg to about 1000 mg, for example, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5025 mg, about 5050 mg, about 5075 mg, about 5100 mg, about 5125 mg, about 5150 mg, about 5175 mg, about 5200 mg, about 5225 mg, about 5250 mg, about 5275 mg, about 5300 mg, about 5325 mg, about 5350 mg, about 5375 mg, about 5400 mg, about 5425 mg, about 5450 mg, about 5475 mg, about 5500 mg, about 5525 mg, about 5550 mg, about 5575 mg, about 5600 mg, about 5625 mg, about 5650 mg, about 5675 mg, about 5700 mg, about 5725 mg, about 5750 mg, about 5775 mg, about 5800 mg, about 5825 mg, about 5850 mg, about 5875 mg, about 5900 mg, about 5925 mg, about 5950 mg, about 5975 mg, about 6000 mg, about 6025 mg, about 6050 mg, about 6075 mg, about 6100 mg, about 6125 mg, about 6150 mg, about 6175 mg, about 6200 mg, about 6225 mg, about 6250 mg, about 6275 mg, about 6300 mg, about 6325 mg, about 6350 mg, about 6375 mg, about 6400 mg, about 6425 mg, about 6450 mg, about 6475 mg, about 6500 mg, about 6525 mg, about 6550 mg, about 6575 mg, about 6600 mg, about 6625 mg, about 6650 mg, about 6675 mg, about 6700 mg, about 6725 mg, about 6750 mg, about 6775 mg, about 6800 mg, about 6825 mg, about 6850 mg, about 6875 mg, about 6900 mg, about 6925 mg, about 6950 mg, about 6975 mg, about 7000 mg, about 7025 mg, about 7050 mg, about 7075 mg, about 7100 mg, about 7125 mg, about 7150 mg, about 7175 mg, about 7200 mg, about 7225 mg, about 7250 mg, about 7275 mg, about 7300 mg, about 7325 mg, about 7350 mg, about 7375 mg, about 7400 mg, about 7425 mg, about 7450 mg, about 7475 mg, about 7500 mg, about 7525 mg, about 7550 mg, about 7575 mg, about 7600 mg, about 7625 mg, about 7650 mg, about 7675 mg, about 7700 mg, about 7725 mg, about 7750 mg, about 7775 mg, about 7800 mg, about 7825 mg, about 7850 mg, about 7875 mg, about 7900 mg, about 7925 mg, about 7950 mg, about 7975 mg, about 8000 mg, about 8025 mg, about 8050 mg, about 8075 mg, about 8100 mg, about 8125 mg, about 8150 mg, about 8175 mg, about 8200 mg, about 8225 mg, about 8250 mg, about 8275 mg, about 8300 mg, about 8325 mg, about 8350 mg, about 8375 mg, about 8400 mg, about 8425 mg, about 8450 mg, about 8475 mg, about 8500 mg, about 8525 mg, about 8550 mg, about 8575 mg, about 8600 mg, about 8625 mg, about 8650 mg, about 8675 mg, about 8700 mg, about 8725 mg, about 8750 mg, about 8775 mg, about 8800 mg, about 8825 mg, about 8850 mg, about 8875 mg, about 8900 mg, about 8925 mg, about 8950 mg, about 8975 mg, about 9000 mg, about 9025 mg, about 9050 mg, about 9075 mg, about 9100 mg, about 9125 mg, about 9150 mg, about 9175 mg, about 9200 mg, about 9225 mg, about 9250 mg, about 9275 mg, about 9300 mg, about 9325 mg, about 9350 mg, about 9375 mg, about 9400 mg, about 9425 mg, about 9450 mg, about 9475 mg, about 9500 mg, about 9525 mg, about 9550 mg, about 9575 mg, about 9600 mg, about 9625 mg, about 9650 mg, about 9675 mg, about 9700 mg, about 9725 mg, about 9750 mg, about 9775 mg, about 9800 mg, about 9825 mg, about 9850 mg, about 9875 mg, about 9900 mg, about 9925 mg, about 9950 mg, about 9975 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg.

In some embodiments, EPA (as the term “EPA” is defined and exemplified herein) represents at least 50%, by weight, of all fatty acids or PUFAs present in the composition, for example, at least 50%, at least 60%, at least 66%, at least 70%, at least 75%, at least 80%, at least 85%, at least 90%, at least 95%, at least 96%, at least 97%, at least 98%, at least 99%, or 100%, by weight, of all fatty acids or PUFAs present in the composition.

In some embodiments, the composition comprises ultra-pure EPA. The term “ultra-pure” as used herein with respect to EPA refers to a composition comprising at least 96%, by weight of the composition, EPA (as the term “EPA” is defined and exemplified herein). Ultra-pure EPA can comprise even higher purity EPA, for example, at least 97%, at least 98%, or at least 99%, by weight of the composition, EPA, wherein the EPA is any form of EPA as set forth herein.

In some embodiments, the composition contains no more than 20%, no more than 15%, no more than 10%, no more than 9%, no more than 8%, no more than 7%, no more than 6%, no more than 5%, no more than 4%, no more than 3%, no more than 2%, no more than 1%, no more than 0.6%, no more than 0.5%, no more than 0.4%, no more than 0.3%, no more than 0.2, or no more than 0.1% of, by weight of total fatty acids or PUFAs present in the composition, of any PUFA or derivative thereof other than EPA. In some embodiments, the composition contains no more than 20%, no more than 15%, no more than 10%, no more than 9%, no more than 8%, no more than 7%, no more than 6%, no more than 5%, no more than 4%, no more than 3%, no more than 2%, no more than 1%, no more than 0.6%, no more than 0.5%, no more than 0.4%, no more than 0.3%, no more than 0.2, or no more than 0.1% of, by weight of total fatty acids or PUFAs present in the composition, DHA (e.g., ethyl-DHA or EDHA) or a derivative thereof, if any. In some embodiments, the composition contains substantially no DHA (e.g., EDHA) or derivatives thereof. In some embodiments, the composition contains no DHA (e.g., EDHA) or derivatives thereof.

In some embodiments, the composition contains less than 30%, less than 20%, less than 15%, less than 10%, less than 9%, less than 8%, less than 7%, less than 6%, less than 5%, less than 4%, less than 3%, less than 2%, less than 1%, less than 0.5%, or less than 0.25%, by weight of the composition, or by weight of all fatty acids or PUFAs present in the composition, of any PUFA or derivative thereof other than EPA. Illustrative examples of a “PUFA or derivative thereof other than EPA” include LA (e.g., ethyl-LA) or a derivative thereof, GLA (e.g., ethyl-GLA) or a derivative thereof, DGLA (e.g., ethyl-DGLA) or a derivative thereof, AA (e.g., ethyl-AA) or a derivative thereof, AdA (e.g., ethyl-AdA) or a derivative thereof, DPA6 (e.g., ethyl-DPA6) or a derivative thereof, ALA (e.g., ethyl-ALA) or a derivative thereof, SDA (e.g., ethyl-SDA) or a derivative thereof, ETA (e.g., ethyl-ETA) or a derivative thereof, DPA (ethyl-DPA) or a derivative thereof, and DHA (e.g., ethyl-DHA or EDHA) or a derivative thereof.

Phospholipids

In some embodiments, the source of phospholipid of the composition can comprise a glycerophospholipid, a lysophospholipid, or mixtures thereof.

In some embodiments, the source of phospholipid comprises a glycerophospholipid. Glycerophospholipids are glycerol-based phospholipids characterized by a glycerol backbone with a polar phosphodiester group attached to the sn-3 carbon and two fatty acid-derived acyl groups attached to the sn-1 and sn-2 carbons. Glycerophospholipids are the main component of biological membranes. Non-limiting examples of glycerophospholipids include phosphatidic acid or phosphatidate (PA), phosphatidylcholine (PC), phosphatidylethanolamine (PE), phosphatidylglycerol (PG), phosphatidylinositol (PI), and phosphatidylserine (PS). Glycerophospholipids may be obtained from krill oil, refined, and optionally enriched with a particular fatty acid such as EPA.

In some embodiments, the source of phospholipid comprises a lysophospholipid (LPL). LPLs are glycerophospholipids in which one acyl chain is lacking and only one hydroxyl group of the glycerol backbone is acylated. 1-Lysophospholipids (1-LPLs) maintain the acyl chain at the sn-2 position, whereas 2-lysophospholipids (2-LPLs) are only acylated at the sn-1 position. Thus, LPLs are small bioactive lipid molecules characterized by a single carbon chain and a polar head group. LPLs are bioactive signaling lipids that are generated from phospholipase-mediated hydrolyzation of membrane phospholipids and sphingolipids. Non-limiting examples of LPLs include sn-1-acyl-glycerol-3-phosphate, sn-2-acyl-glycero-3-phosphate, lysophosphatidic acid (LPA or lysoPA), lysophosphatidylcholine (LPC or lysoPC), lysophosphatidylethanolamine (LPE or lysoPE), lysophosphatidylglycerol (LPG or lysoPG), lysophosphatidylinositol (IPI or lysoPI), and lysophosphatidylserine (LPS or lysoPS).

In certain of these embodiments, the acyl chains of a glycerophospholipid or an LPL can be derived from any fatty acids, for example, ten to thirty long-chain hydrocarbon (C10-C30) fatty acids. These fatty acids can be straight-chained or branched, saturated or unsaturated (e.g., containing one or more cis or trans double bonds). In some embodiments, the acyl chains can be derived from PUFAs or derivatives thereof as described herein, including, for example, LA, GLA, DGLA, AA, AdA, DPA6, ALA, SDA, ETA, EPA, DPA, DHA, or a derivative thereof.

In some embodiments, the source of phospholipid is lecithin. Lecithin is usually a mixture of glycerophospholipids including PA, PC, PE, PG, PI, PS, and LPLs. It has low solubility in water but can serve as an excellent emulsifier in aqueous solutions. Lecithin can be obtained from different sources, for example, soybean, milk, egg yolk, marine foods, rapeseed, cottonseed, and sunflower oil, with (purified and/or chemically modified) soybean lecithin being the most common commercially available form. In some embodiments, the lecithin is soy lecithin, for example, Metarin™ P (Cargill, MN).

In some embodiments, such as when the source of phospholipid comprises a mixture of glycerophospholipids and/or LPLs (e.g., lecithin), the content of each glycerophospholipid and/or LPL within the mixture can vary. Without wishing to be bound by theory, the ratio of PE:PI within the lecithin may be especially important, as certain PUFA favors certain phospholipid (e.g., EPA favors PE while AA favors PI), and thus the relative content of PE:PI within lecithin may affect the bioavailability of the PUFA component in vivo. In some embodiments, the weight ratio of PE and PI in the source of phospholipid (e.g., lecithin) ranges from about 5:1 (favoring PE) to 1:5 (favoring PI), for example, about 2:1 (favoring PE) to 1:2 (favoring PI).

In some embodiments, the source of phospholipid (e.g., lecithin) is enriched with PE and/or restricted with PI. In some embodiments, the source of phospholipid comprises up to 40%, up to 60%, up to 80%, up to 90%, up to 95%, or up to 97%, by weight of the source of phospholipid, PE, and no more than 5%, no more than 4%, no more than 3%, no more than 2%, or no more than 1%, by weight of the source of phospholipid, PI. In some embodiments, the source of phospholipid (e.g., lecithin) comprises up to 40%, up to 60%, up to 80%, or up to 85%, by weight of the source of phospholipid, PE, and no more than 10%, no more than 5%, no more than 4%, no more than 3%, no more than 2%, or no more than 1%, by weight of the source of phospholipid, PI. A source of phospholipid enriched with PE and/or restricted with PI may be derived from a natural source or may be obtained by chemical processes known to a person skilled in the art, including, for example, separation by density and chemical precipitation.

In some embodiments, the composition contains 1% to 85% of the source of phospholipid (e.g., lecithin), by weight of the composition, for example, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or 85% of the source of phospholipid, by weight of the composition. In some embodiments, the composition contains no more than 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, no more than 5%, or no more than 1% of the source of phospholipid, by weight of the composition.

Emulsifiers

In some embodiments, the composition optionally further comprises one or more additional emulsifiers. Non-limiting examples of emulsifiers include polyoxyethylene hydrogenated castor oil, polyoxyethylene sorbitan fatty acid ester, polyoxyethylene castor oil, polyethylene glycol fatty acid ester, polyoxyethylene polyoxypropylene glycol, sucrose fatty acid ester, and lecithin. In one embodiment the emulsifier is polysorbate 80, polyoxyl-35, or both.

In some embodiments, the emulsifier comprises one or more glycerol derivatives selected from the group consisting of triacylglycerol, diacylglycerol, and monoacylglycerol. In one embodiment, the glycerol derivative is castor oil. The glycerol derivative may also be a re-esterified triglyceride (rTG) enriched with the PUFA of the composition (e.g., EPA). As used herein, a “re-esterified triglyceride” is a chemically synthesized triglyceride. A re-esterified triglyceride enriched with EPA can be synthesized according to methods disclosed, for example, in J. Agric. Food Chem. (2018) 66(1):218-227. In triglyceride extracted from natural fish oil, the omega-3 PUFAs are found mainly in the sn-2 position of the glycerol molecule. In the re-esterification process, which adds, on average, one extra n-3 fatty acid to each triglyceride molecule, the positioning of EPA and/or DHA in the glyceride molecule are at random. Thus, the sn-1/3 and sn-2 positions are equally esterified by EPA and DHA in the glycerol molecule.

In some embodiments, the composition contains 1% to 20%, by weight of the composition, of the one or more emulsifiers, for example, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or 20% of the one or more emulsifiers, by weight of the composition. In some embodiments, the composition contains no more than 20%, no more than 15%, no more than 10%, no more than 9%, no more than 8%, no more than 7%, no more than 6%, no more than 5%, no more than 4%, no more than 3%, no more than 2%, or no more than 1% of the one or more emulsifiers, by weight of the composition.

In some embodiments, the composition contains 1% to 85%, by weight of the composition, of additives (e.g., phospholipids and/or emulsifiers), for example, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or 85% of the additives, by weight of the composition. In some embodiments, the composition contains no more than 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, no more than 5%, or no more than 1% of the additives, by weight of the composition.

In some embodiments, the composition contains a fixed dose of the PUFA (e.g., EPA) or derivative thereof, along with a varying dose of the source of phospholipid and a varying dose of the emulsifier compared to the PUFA or derivative thereof. In some embodiments, the weight ratio between the PUFA or derivative thereof and the source of phospholipid in the composition ranges from about 20:1 (favoring PUFA or derivative thereof) to about 1:5 (favoring source of phospholipid), for example, about 5:1 (favoring PUFA or derivative thereof) to about 1:5 (favoring source of phospholipid), about 3.75:1 (favoring PUFA or derivative thereof) to about 1:5 (favoring source of phospholipid), or about 1:1 to about 1:5 (favoring source of phospholipid). In some embodiments, the weight ratio between the PUFA or derivative thereof and the emulsifier in the composition ranges from 10:1 (favoring PUFA or derivative thereof) to 1:1.

In a preferred embodiment, provided is a lymph-releasing (LR) EPA formulation (designated herein as “LR-EtEPA”) by co-formulation of eicosapentaenoic acid ethyl ester (EtEPA, also referred to as ethyl-EPA, E-EPA, EPA-E, or IPE) with phospholipids as described herein. The composition may optionally further comprise one or more emulsifiers, for example, polysorbate 80, polyoxyl-35, or both. As illustrated herein and demonstrated in the working examples, co-formulation of EtEPA with phospholipids and/or emulsifiers (together referred to as additives or excipients) facilitates the in vivo formation of an esterified EPA substrate upon administration, thus causing the EPA to be absorbed bound to the phospholipid and re-routing the EPA to the lymphatic system for delivery to target tissues. Thus, the phospholipids and/or emulsifiers in the LR-EtEPA formulation can also be referred to as LR compounds. In one example, the source of phospholipid is lecithin, for example, soy lecithin (e.g., Metarin™ P). Representative compositions according to the above embodiments are disclosed in European Patent Application EP 3023098 A1, the entire contents of which are incorporated by reference herein, and Example 1 of the present disclosure.

In should be noted that the LR-EtEPA composition in the embodiments discussed above is illustrative, and a similar lymph-releasing composition may be formulated with another PUFA or a derivative thereof as disclosed herein, for example, an omega-6 fatty acid such as LA, GLA, DGLA, AA, AdA, and DPA6, or an omega-3 fatty acid such as ALA, SDA, ETA, DPA, and DHA, or a derivative thereof.

In some embodiments, the composition comprising one or more PUFAs or derivatives thereof, a source of phospholipid, and optionally one or more additional emulsifiers according to various embodiments disclosed herein can be formulated as one or more dosage units. The term “dosage unit” refers to a portion of a pharmaceutical composition that contains an amount of a therapeutic agent suitable for a single administration to provide a therapeutic effect. The terms “composition” and “pharmaceutical composition” are used interchangeably. Such dosage units may be administered one to a plurality (i.e., 2, 3, 4, 5, or more) of times per day, or as many times as needed to elicit a therapeutic response.

In some embodiments, the composition is orally deliverable or in a form suitable for oral administration. The term “orally deliverable” or “oral administration” includes any form of delivery of a pharmaceutical composition to a subject wherein the composition is placed in the mouth of the subject, whether or not the composition is swallowed. In some embodiments, the dosage unit is a capsule, i.e., the composition is formulated in one or more capsules, for example, soft gelatin capsules. The one or more capsules may be packaged in a blister pack or a receptacle (e.g., bottle).

In some embodiments, the PUFAs (e.g., EtEPA) or derivatives thereof and the source of phospholipid (e.g., lecithin) (and optionally one or more additional emulsifiers) can be co-formulated in the same dosage unit or can be individually formulated in separate dosage units. For a non-limiting example, EtEPA and the source of phospholipid (e.g., lecithin) may be co-formulated in the same capsule or individually formulated in separate capsules. For the latter, the capsules containing EtEPA and the capsules containing the source of phospholipid (e.g., lecithin) may be packaged in separate bottles or blister packs; alternatively, they may be packaged in the same bottle or blister pack.

In some embodiments, the composition is formulated for administration alone or with food.

In another embodiment, the composition has injectable formulations, lyophilized formulations, or liquid formulations, depending on the routes of administration. In some embodiments, the composition may have various formulations for injection and/or infusion, for example, intravenous injection, intraperitoneal injection, intertumoral injection, bone marrow injection, lymph node injection, subcutaneous injection, and cerebrospinal fluid injection. In some embodiments, the composition can be provided in a ready-to-hang formulation for enteric use in, for example, the intensive care unit (ICU) setting to treat SIRS, sepsis, and/or ARDS.

### Therapeutic Methods

In some embodiments, provided is a method of enhancing the level of PUFA in lymph fluid, or enriching lymph with PUFA, in a subject in need thereof, the method comprising administering to the subject a therapeutically effective amount of a composition comprising one or more PUFAs or derivatives thereof and a source of phospholipid according to various embodiments disclosed herein. As used herein, “lymph fluid” shall be understood to encompass both the acellular and cellular components of lymph. Because the cellular compartment of lymph is predominantly lymphocytes, these embodiments may also involve enriching immune cells with the PUFA or derivative thereof and thereby encompass conditions affecting lymphocytes.

The term “therapeutically effective amount” refers to an amount which is sufficient to effect treatment, as defined herein, when administered to a mammal in need of such treatment. The therapeutically effective amount will vary depending on the subject and disease state being treated, the severity of the affliction and the manner of administration, and may be determined routinely by one of ordinary skill in the art.

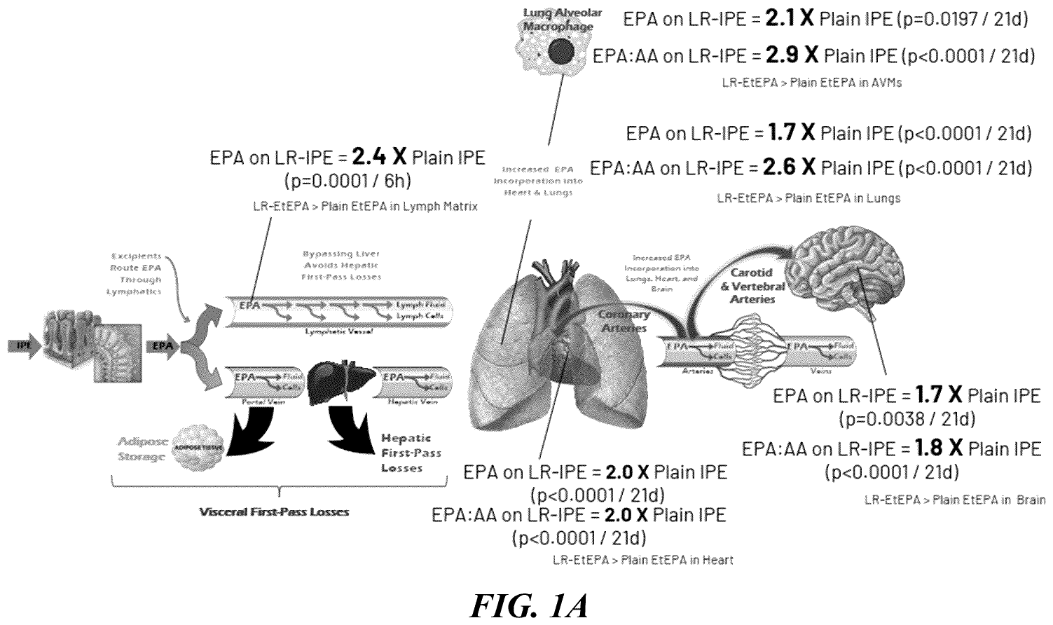

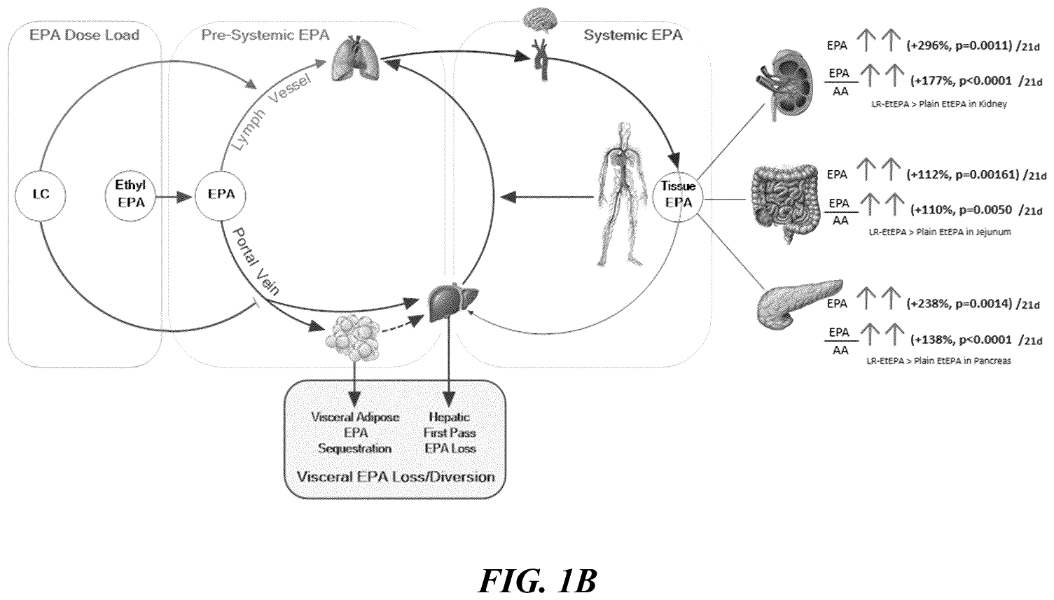

Long chain fatty acids like EPA can enter the circulation by two routes, the portal vein route and the lymphatic route. As shown in FIG. 1A, once EPA is absorbed in the intestine, the EPA circulation is bifurcated, i.e., it can either travel down the lymphatic vessel and bypass the liver and visceral adipose entirely; or it can travel down the portal vein where (1) visceral adipose can sequester EPA, and (2) the liver takes up EPA, and the fatty acid is oxidized, modified, or allowed to pass through. EPA that travels down the portal vein undergoes significant adipose sequestration and hepatic first pass losses and the resulting circulation of EPA is diminished. EPA that travels down the lymphatic vessel can avoid visceral and hepatic first pass losses resulting in the enhanced delivery of EPA to the first tissue bed it encounters, which is the lung. Delivery of fat-soluble drugs to the lung benefits from high perfusion rate, as the lung get 100% of cardiac output. After the lung, the EPA is circulated through the aorta which supplies blood and nutrients to the coronary and carotid arteries and the rest of the body. EPA that bypasses visceral adipose and the liver (and any associated first pass losses) enhances cardiopulmonary and cerebrovascular EPA uptake including coronary, carotid, and vertebral arteries.

The inclusion of phospholipids (e.g., lecithin) and/or emulsifiers with the fatty acid (e.g., EtEPA) in the composition is believed to have distinct advantages for the methods of the invention, including preferred routing through the lymphatic system and improved bioavailability of active ingredient at tissue and cell levels. For example, without wishing to be bound by theory, the co-administration of EtEPA with phospholipids results in the in vivo formation of an esterified EPA substrate thus causing the EPA to be delivered as a phospholipid. When the composition of the present technology enters the small intestine, a cross-esterification process is believed to take place between EPA and the fatty acid component of the phospholipid molecule, giving rise to PL-EPA. Said EPA-phospholipid compound is primed for uptake by cells such as enterocytes because it is in a phospholipid matrix. Thus, the EPA would get secreted from the enterocyte being bound to the phospholipid and/or to triglyceride. The presence of excess phospholipid substrate from administered phospholipids is expected to enhance incorporation into enterocyte-secreted lipoproteins as phospholipid-EPA in LR-EtEPA compared to plain EtEPA. Thus, phospholipid provision not only enhances absorption and uptake by the enterocyte, but it also promotes EPA re-assembly esterified to phospholipids, priming it for cellular uptake and integration into cell membranes. Not only so, but phospholipids also facilitate chylomicron and/or intestinal very-low density lipoprotein (I-VLDL) assembly and secretion by enterocytes. Because chylomicrons and I-VLDL are primarily secreted into the lymph, the composition of the present technology facilitates re-routing of EPA to the chylomicrons/I-VLDL and thence to the lymph, thereby avoiding hepatic and adipose first pass visceral loss associated with the portal vein route. This routing positions the heart and lungs as the first pass organs, thus increasing EPA concentrations and the EPA/AA ratio in various tissues (e.g., in lungs, heart, brain, kidney, intestines, and pancreas) (FIG. 1B).

Typically, visceral adipose traps about 50% of fatty acids that present to the portal vein. It is known that long chain polyunsaturated fatty acids (LC-PUFAs), including EPA, are taken up by adipose tissue in general, from studies of subcutaneous adipose. It is expected that visceral adipose incorporates EPA even more readily than subcutaneous adipose, since it is exposed to greater quantities of EPA in the alimentary form. Thus, visceral adipose may divert about half of the EPA presenting to the portal vein, by either storing it for an indeterminate amount of time or catabolizing it by beta-oxidation in situ. The 50% of fatty acids that bypass visceral adipose trapping present to the liver. The liver typically receives about 75% of its blood by the portal vein, and only 25% from the hepatic artery. Alimentary EPA presenting to the liver via the portal vein is subjected to first pass metabolism, which could include elongation and desaturation to form other LC-PUFAs, oxygenation to form oxylipins, or beta-oxidation for use as metabolic fuel. Between visceral adipose trapping and hepatic first pass losses, it is expected that a substantial portion of alimentary EPA is lost or diverted when it is shunted down the portal vein. Insofar as lecithin and the excipients in general shunt alimentary EPA through the lymphatic vein, visceral loss and diversion are averted, leaving more EPA available to systemic tissues after passing through the cardiopulmonary system.

As shown in the working examples, the lymph-releasing EtEPA composition of the present technology (LR-EtEPA) more than doubles (2.4 times) lymph EPA levels compared to plain EtEPA composition given at equimolar doses of EtEPA (FIG. 1A). LR-EtEPA also significantly increases EPA levels in the first pass organs, the lungs (1.7 times) and heart (2.0 times), and in the brain (1.7 times), compared to EtEPA. An increase is also observed in lung alveolar macrophages (AVMs) (2.1 times), which are the chief immune cells in the lung safeguarding against airborne pathogen and tissue injury and play an important role in exuberant inflammatory cascade in conditions like sepsis and ARDS. Thus, EPA uptake following LR-EtEPA was found to be superior to EtEPA alone when administered in equimolar doses of EtEPA, suggesting that LR-EtEPA is more potent than EtEPA at equimolar doses for raising EPA level and/or outcome in a particular tissue, and that it would require less dosing of LR-EtEPA to achieve the same amount of tissue EPA as it would for plain EtEPA. EPA/AA ratios were also increased in the same tissues/organs by LR-EtEPA, including, for example, 2.6 times as much as the level from plain EtEPA for lungs, 2.0 times for the heart, 1.8 times for the brain, and 2.9 times for AVMs.

The preclinical experiments described herein confirm that LR-EtEPA is superior to EtEPA at achieving cellular EPA uptake in three circulating blood cell types and two broad types of immune cells. The co-administration of EtEPA with glycerophospholipid resulted in increased uptake of EPA in lymph cells which are mostly composed of T lymphocytes, B lymphocytes, and natural killer (NK) cells. The rate of EPA uptake was enhanced in the alveolar macrophages, as increased EPA uptake was also observed in resident macrophages in the alveoli. One skilled in the art would expect a similar acceleration of EPA uptake for coronary macrophages or carotid macrophages.

Compared to plain EtEPA, enhanced cellular uptake of EPA with LR-EtEPA was also observed in several dense tissues including cells in the lung, heart, brain, kidneys, pancreas, jejunum, and liver. Notably, the superior cellular uptake wasn't accompanied by consistent differences in acellular blood such as plasma, suggesting that even though LR-EtEPA results in acellular blood/plasma EPA levels comparable to plain EtEPA, it is superior at delivering EPA to tissues and increasing tissue/cell-level EPA bioavailability.

Taken together, compared to administration of EtEPA alone, co-administration of phospholipids and/or emulsifiers with EtEPA in the lymph-releasing EPA formulation (LR-EtEPA) shunts EPA to the lymphatic system rather than the portal vein. In doing so, visceral/hepatic first pass loss is reduced, resulting in more efficient drug delivery. Owing to its high perfusion and diminished first pass losses, delivering drugs by the lymphatic route especially enhances lung, heart, and brain incorporation. The lungs and heart essentially become the first pass organs, which means the composition is particularly advantageous for treating cardiopulmonary diseases. The composition also enhances EPA delivery and uptake by coronary, carotid, and vertebral arteries, and hence to the heart and brain, as well as other organs/tissues of the body.

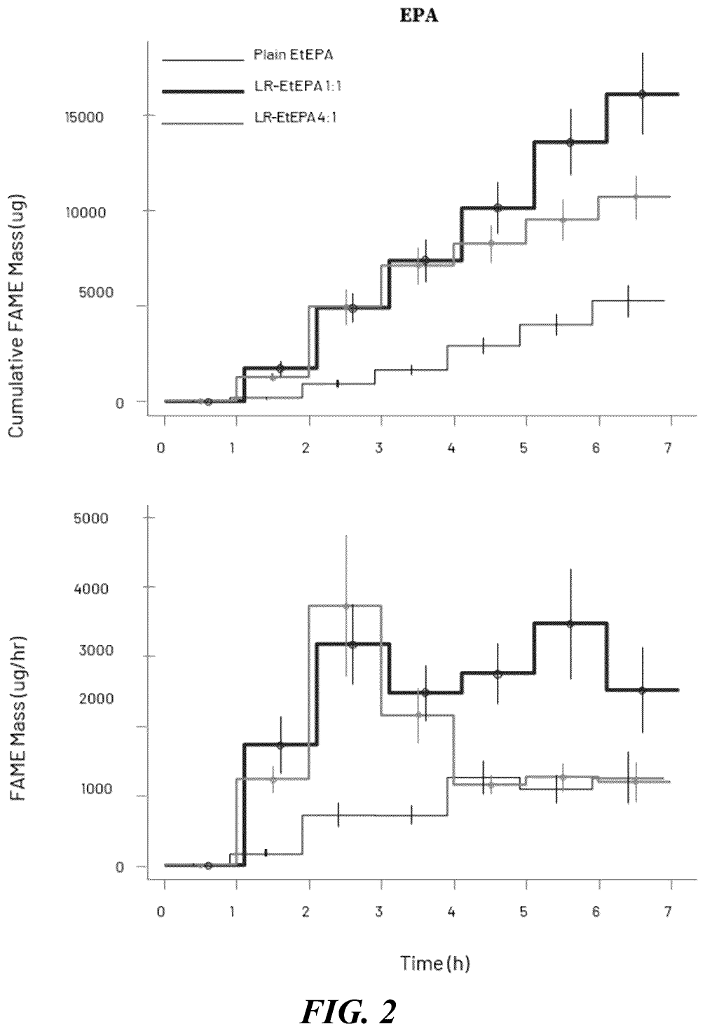

Moreover, increasing the amount of the additives (e.g., phospholipids and/or emulsifiers) appears to result in significantly more delivery of EPA to lymph (FIG. 2). By altering the ratio of EtEPA to additives from 4:1 (favoring EtEPA) to 1:1 (i.e., equal parts EtEPA and additives), the cumulative lymph delivery of EPA was significantly higher, and the increase in EPA uptake was prolonged, compared to the lower additive formulation, indicating a robust dose-dependent effect between the amount of phospholipids and the amount of EPA delivered to lymph. The lymph-releasing effects, as well as the corresponding improved bioavailability in lymph and enhanced drug delivery to tissues, of the composition and its additives are expected to apply to other PUFAs and derivatives thereof. Thus, the present technology provides a superior formulation for delivering PUFAs and derivatives thereof to various tissues and cells of the body, which presents great therapeutic potential for a variety of diseases.

It should be noted that the phospholipid of the composition need not be administered at the same rate as the fatty acid component, as intestinal absorption of fatty acids and subsequent delivery to the lymph can be discontinuous. Accordingly, in certain medical contexts, it may be advantageous to provide the phospholipid over a longer period than the fatty acid. This would also help to better distribute the volume load of the composition.

Cardiopulmonary, Cardiovascular, and Cerebrovascular Diseases

In some embodiments, provided is a method of treating and/or preventing a cardiopulmonary, cardiovascular, and/or cerebrovascular disease in a subject in need thereof by administering to the subject a composition of the present technology. As explained above, the composition of the present technology, e.g., the lymph-releasing EPA formulation (LR-EtEPA), excels at re-routing EPA to the lymphatic system and delivering EPA to tissues and cells including the heart, lungs, and brain. Notably, LR-EtEPA is superior to EtEPA alone at enhancing coronary arteries with EPA, suggesting its therapeutic potential in diseases such as atherosclerosis and vasculitis affecting the coronary arteries, carotid arteries, vertebral arteries, and the cerebrovascular system in general. Due to the therapeutic effects of EPA, it is believed that the composition of the present technology is particularly useful for delivering EPA to target tissues and cells and treating diseases including those associated with the heart, lungs, and brain.

The term “treatment” or “treating” in relation to a given disease or disorder includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder. The term “prevention” or “preventing” in relation to a given disease or disorder includes preventing the onset of disease development if none had occurred; preventing the disease or disorder from occurring in a subject that may be predisposed to the disorder or disease but has not yet been diagnosed as having the disorder or disease; and/or preventing further disease/disorder development if already present.

In some embodiments, provided is a method of treating and/or preventing a cardiovascular and/or cerebrovascular disease, or reducing risks of a cardiovascular and/or cerebrovascular disease, in a subject in need thereof, the method comprising administering to the subject a therapeutically effective amount of a composition comprising one or more PUFA or derivatives thereof and a source of phospholipid according to various embodiments disclosed herein.

The term “cardiovascular disease” refers to any disease or disorder of the heart (cardiac diseases or disorders) or blood vessels (vascular diseases or disorders) or any symptom thereof. Non-limiting examples of cardiovascular diseases include hypertriglyceridemia, hypercholesterolemia, mixed dyslipidemia, coronary heart disease, stroke, atherosclerosis, arrhythmia, hypertension, myocardial infarction, vasculitis, cardiomyopathy (e.g., viral cardiomyopathy including related to COVID-19), pericarditis, congestive heart failure, myocardial necrosis, vascular ischemia, vascular disease beyond the cardiopulmonary system, thrombotic disease, post-myocardial infarction cardiac remodeling, giant cell arteritis, polyarteritis nodosa, cryoglobulinemia, episodic small-vessel ischemia (Raynaud's disease), deep venous thrombosis, disseminated intravascular coagulation, erectile dysfunction, and other cardiovascular or related diseases.

As used herein, a “vascular disease beyond the cardiopulmonary system” includes vasculitis outside the cardiopulmonary system, including the carotid and vertebral arteries and branches therefrom and vasculitis affecting peripheral arteries (e.g., aortitis, renal vasculitis); and atherosclerosis outside the cardiopulmonary system, including the carotid, vertebral, and peripheral arteries and branches therefrom. A “thrombotic disease” includes disseminated intravascular coagulation, other diseases involving excessive platelet activation, venous thrombosis, and thrombotic events related to major adverse cardiovascular events.

In some embodiments, provided is a method of delaying an onset of a cardiovascular and/or cerebrovascular event in a subject in need thereof, the method comprising administering to the subject a therapeutically effective amount of a composition comprising PUFAs or derivatives thereof and a source of phospholipid according to various embodiments disclosed herein. The term “an onset of a cardiovascular and/or cerebrovascular event” refers to a first appearance of a sign and/or symptom of the cardiovascular and/or cerebrovascular event. In some embodiments, delaying an onset of a cardiovascular and/or cerebrovascular event prevents the subject from experiencing the cardiovascular and/or cerebrovascular event and/or developing any further symptoms of the cardiovascular and/or cerebrovascular event. Non-limiting examples of cardiovascular and/or cerebrovascular events include non-fatal myocardial infarction, stroke, cardiovascular death, unstable angina, coronary revascularization, carotid revascularization, peripheral revascularization, cerebrovascular accident, transient ischemic attached, and hospitalization for unstable angina.

In some embodiments, the subject to be treated for a cardiopulmonary, cardiovascular, and/or cerebrovascular disease has a fasting baseline triglyceride level (or a median fasting baseline triglyceride level in the case of a subject group) of about 135 mg/dL to about 500 mg/dL, for example, about 135 mg/dL to less than 500 mg/dL, about 150 mg/dL to less than 500 mg/dL, about 200 mg/dL to less than 500 mg/dL, or about 200 mg/dL to about 499 mg/dL. In some embodiments, the subject has a fasting baseline triglyceride level (or a median fasting baseline triglyceride level in the case of a subject group) of about 135 mg/dL, about 140 mg/dL, about 145 mg/dL, about 150 mg/dL, about 155 mg/dL, about 160 mg/dL, about 165 mg/dL, about 170 mg/dL, about 175 mg/dL, about 180 mg/dL, about 185 mg/dL, about 190 mg/dL, about 195 mg/dL, about 200 mg/dL, about 205 mg/dL, about 210 mg/dL, about 215 mg/dL, about 220 mg/dL, about 225 mg/dL, about 230 mg/dL, about 235 mg/dL, about 240 mg/dL, about 245 mg/dL, about 250 mg/dL, about 255 mg/dL, about 260 mg/dL, about 265 mg/dL, about 270 mg/dL, about 275 mg/dL, about 280 mg/dL, about 285 mg/dL, about 290 mg/dL, about 295 mg/dL, about 300 mg/dL, about 305 mg/dL, about 310 mg/dL, about 315 mg/dL, about 320 mg/dL, about 325 mg/dL, about 330 mg/dL, about 335 mg/dL, about 340 mg/dL, about 345 mg/dL, about 350 mg/dL, about 355 mg/dL, about 360 mg/dL, about 365 mg/dL, about 370 mg/dL, about 375 mg/dL, about 380 mg/dL, about 385 mg/dL, about 390 mg/dL, about 395 mg/dL, about 400 mg/dL, about 405 mg/dL, about 410 mg/dL, about 415 mg/dL, about 420 mg/dL, about 425 mg/dL, about 430 mg/dL, about 435 mg/dL, about 440 mg/dL, about 445 mg/dL, about 450 mg/dL, about 455 mg/dL, about 460 mg/dL, about 465 mg/dL, about 470 mg/dL, about 475 mg/dL, about 480 mg/dL, about 485 mg/dL, about 490 mg/dL, about 495 mg/dL, or about 500 mg/dL. In some embodiments, the subject has a fasting baseline triglyceride level (or a median fasting baseline triglyceride level in the case of a subject group) of about 135 mg/dL or higher, about 150 mg/dL or higher, about 200 mg/dL or higher.

In some embodiments, the subject has a fasting baseline triglyceride level (or a median fasting baseline triglyceride level in the case of a subject group) of about 500 mg/dL or higher.

In some embodiments, the subject has one or more of: a baseline non-high-density lipoprotein cholesterol (HDL-C) value of about 200 mg/dL to about 300 mg/dL; a baseline total cholesterol (TC) value of about 250 mg/dL to about 300 mg/dL; a baseline very low-density lipoprotein cholesterol (VLDL-C) value of about 140 mg/dL to about 200 mg/dL; a baseline HDL-C value of about 10 mg/dL to about 30 mg/dL; a baseline low-density lipoprotein cholesterol (LDL-C) value of about 40 mg/dL to about 100 mg/dL; and/or a baseline high-sensitivity C-reactive protein (hsCRP) level of about 2 mg/dL or less.

In some embodiments, the subject is on stable statin therapy, e.g., administered with a statin (with or without ezetimibe). In some embodiments, the statin therapy may include one or more of: atorvastatin, fluvastatin, lovastatin, pitavastatin, pravastatin, rosuvastatin, and simvastatin. In some embodiments, the statin therapy includes administration of a statin and ezetimibe. In some embodiments, the statin therapy includes administration of a statin without ezetimibe.

In some embodiments, upon administration of the composition, the subject exhibits one or more of:
(a) a reduction in triglyceride levels compared to baseline or control;
(b) a reduction in blood pressure level compared to baseline or control;
(c) a reduction in insulin resistance compared to baseline or control;
(d) a reduction in the level of an inflammatory biomarker selected from the group consisting of vascular endothelial growth factor (VEGF), tumor necrosis factor-α (TNF-α), monocyte chemoattractant protein-1 (MCP-1), interleukin-1β (IL-1β), soluble intercellular adhesion molecule-1 (sICAM-1), soluble vascular cellular adhesion molecule-1 (sVCAM-1), high sensitivity reactive protein (hsCRP), lipoprotein-associated phospholipase A2 (Lp-PLA2), and circulating monocyte compared to baseline or control;
(e) a reduction in the level of a metabolic biomarker selected from the group consisting of total cholesterol, VLDL-C, remnant lipoprotein cholesterol, LDL-C, small/dense LDL-C, HDL-C, non-HDL-C, HDL-C functionality, apolipoprotein B (Apo B), interleukin-6 (IL-6), apolipoprotein A-1 (Apo A-1), and improvements in homeostasis model assessment of insulin resistance (HOMA-IR, including HOMA2-IR), quantitative insulin sensitivity check index (QUICKI), revised quantitative insulin sensitivity check index (rQUICKI), fasting insulin resistance index (FIRI), Bennetts index, fasting insulin, insulin-to-glucose ratio, insulin sensitivity (Si) and homeostasis model assessment of beta-cell function (HOMA-B, including HOMA2-B) compared to baseline or control;
(f) a reduction in the level of an oxidative biomarker selected from the group consisting of lipid oxidation, lipid peroxidation, lipid hydroperoxidation, malondialdehyde, prostaglandin-2 alpha (PGF-2α), platelet-derived growth factor (PDGF), and antioxidant potential compared to baseline or control; and
(g) a reduction in the risk of one or more of: cardiovascular death; nonfatal myocardial infarction; transient ischemic attack; nonfatal stroke; fatal stroke; coronary revascularization; carotid revascularization; peripheral revascularization; unstable angina (e.g., unstable angina determined to be caused by myocardial ischemia by, for example, invasive or non-invasive testing, and requiring hospitalization); cardiac arrest; peripheral cardiovascular disease requiring intervention, angioplasty, bypass surgery or aneurysm repair; death; sudden cardiac death; and onset of new congestive heart failure compared to baseline or control.

Parameters described herein can be measured in accordance with any clinically acceptable methodology. For example, triglycerides, total cholesterol, HDL-C, and fasting blood sugar can be sampled from serum and analyzed using standard photometry techniques. VLDL-TG, LDL-C, and VLDL-C can be calculated or determined using serum lipoprotein fractionation by preparative ultracentrifugation and subsequent quantitative analysis by refractometry or by analytic ultracentrifugal methodology. Apo A-1, Apo B, and hsCRP can be determined from serum using standard nephelometry techniques. Small/dense LDL-C and remnant cholesterol can be determined by fast protein electrophoresis, ultracentrifugal methods, or immunolabeling methods. These techniques are described in detail in standard textbooks, for example, Tietz Fundamentals of Clinical Chemistry, 6th Ed. (Burtis, Ashwood and Borter Eds.), WB Saunders Company.

Pulmonary Disease Including Sepsis, SIRS, and/or ARDS

In some embodiments, provided is a method of treating and/or preventing a pulmonary disease in a subject in need thereof, the method comprising administering to the subject a therapeutically effective amount of a composition comprising PUFAs or derivatives thereof and a source of phospholipid according to various embodiments disclosed herein.

A “pulmonary disease” includes, but is not limited to, the following types of diseases: parenchymal diseases of the lung including acute inflammatory or thrombotic diseases (e.g., community-acquired pneumonia, COVID-19 pneumonia, sepsis, SIRS, ARDS, pulmonary embolism, diffuse interstitial pneumonia, radiation pneumonitis, pleuritis, acute eosinophilic pneumonia, chronic eosinophilic pneumonia, Loffler syndrome); chronic pulmonary diseases (e.g., sarcoidosis, interstitial lung disease, chronic obstructive pulmonary disease (COPD), reactive airway disease, asthma, bronchiectasis, bronchiolitis, cystic fibrosis, bronchial carcinoid); vascular diseases of the lung (e.g., pulmonary arterial hypertension, pulmonary vasculitis, microscopic polyangiitis, granulomatosis with polyangiitis (Wegener's disease), eosinophilic granulomatosis with polyangiitis (Churg-Strauss)); upper respiratory conditions (e.g., nasopharyngitis); and pulmonary-renal vasculitis (e.g., Goodpasture's syndrome, cryoglobulinemia, systemic lupus erythematosus (SLE), systemic sclerosis, antiphospholipid syndrome).

As explained above, the lymph-releasing compositions of the present technology are superior at delivering fat-soluble drugs such as fatty acids to the lungs because the delivery is dependent on perfusion rate and lungs have the highest perfusion rate in the body, suggesting that the lymph-releasing compositions may be particularly useful for treating pulmonary diseases.

Moreover, long-chain PUFAs (LC-PUFAs) are intimately involved in the tissue injury response, as LC-PUFAs within cell membrane phospholipids are liberated as one of the earliest steps of the inflammatory cascade. As used herein, LC-PUFAs are fatty acids having at least 18 carbon atoms. Once liberated from membrane phospholipids, LC-PUFAs, especially those that are 18 or 20 carbons long, are oxidized by several enzymes yielding a variety of bioactive metabolites known as oxylipins. Enzymes facilitating 18-, 20-, and 22-carbon LC-PUFA oxygenation include cyclooxygenase (COX), lipoxygenase (LOX), and cytochrome P450 epoxygenase (CYP). The best-known exemplar of this process is arachidonic acid (AA or ARA, also known as omega-6 eicosatetraenoic acid), a 20-carbon LC-PUFA bearing four double bonds, the last of which is in the omega-6 position. In the case of AA, the oxidation enzymes yield numerous bioactive compounds that influence the ensuing inflammatory, thrombotic, vasoconstrictive and bronchoconstrictive responses, including a series of prostanoids (e.g., prostaglandins and prostacyclin), thromboxanes, leukotrienes, and a proliferation of mono-, di-, and tri-alcohols, epoxides, ketones, and related compounds. The seemingly dominating role of AA has contributed to this oxygenation cascade also being called the “eicosanoid” cascade, referring to its strong association with 20-carbon LC-PUFAs, despite that oxygenated metabolites of 18-carbon PUFAs (octadecosanoids) typically comprise the majority of oxylipins under unbiased oxylipinomics screening. Indeed, other LC-PUFAs are also oxidized by these same enzymes to a similar or lesser extent, including oxygenated metabolites of 22-carbon PUFAs (docosanoids). However, absent exogenous dosing of the parent fatty acids, few are present at levels comparable to AA. Importantly, the oxidative metabolites of other LC-PUFAs may not have the same or any biological effects compared to those from AA. Nor are their levels easily predicted from levels of their parent fatty acid, owing to differences in oxygenating enzyme action. Given the pro-inflammatory, pro-thrombotic, vasoconstrictive, and bronchoconstrictive roles of AA-derived oxylipins, to the extent that these other octadecosanoids, eicosanoids, and docosanoids displace AA, one skilled in the art would appreciate that this could provide a therapeutic advantage at curbing the inflammatory, thrombotic, vasoconstrictive, and bronchoconstrictive responses. This could occur in conditions mediated by excessive activation of the eicosanoid/oxylipin cascade, as happens with conditions of excessive inflammation like SIRS, sepsis, or ARDS. It could also occur in conditions of excessive platelet activation from thromboxane overproduction, such as disseminated intravascular coagulation (DIC) or major atherosclerotic vascular events (MACE) or cerebrovascular events, which also involve vasoconstriction. Excessive leukotriene activation has ill effects on bronchospasm and other pathological processes, suggesting that a variety of diseases are influenced by the eicosanoid cascade.

Among potential candidates to displace AA, EPA is a particularly strong contender for distinct therapeutic advantages. Not simply an LC-PUFA, EPA is a 20-carbon PUFA like AA, and only differs from the latter insofar as it has one more double bond, toward the end of the molecule (i.e., at the omega-3 position vs the omega-6 position in AA). As such, EPA interacts with the same suite of enzymes that oxidize AA, and its metabolic byproducts (e.g., oxylipins) are analogous to those of AA. At the same time, the shape of the EPA-derived metabolites differs from those of AA by the presence of the “extra” double bond at the end. As a result, the oxylipins formed from EPA vary in their bioactivity compared to those from AA. For example, thromboxanes originating from EPA are less thrombotic, and therefore less likely to propagate runaway thrombosis. Likewise, prostanoids originating from EPA are less apt to stimulate the inflammatory and vasoconstrictive cascades and attendant ischemia. Especially important for acute inflammatory pulmonary diseases such as SIRS, sepsis, and/or ARDS, leukotrienes originating from EPA are less apt to stimulate the inflammatory and bronchospastic/bronchoconstrictive cascades. As such, if levels of EPA within the tissues mediating these conditions could be raised to compete with AA, it is expected to attenuate the adverse consequences of over-exuberant stimulation of the eicosanoid cascade from AA. This would mitigate runaway inflammatory conditions, such as the downstream cytokine storm underlying SIRS, sepsis, and/or ARDS, the pro-thrombotic state of DIC and MACE, bronchospasm/bronchoconstriction, and so forth.

Other potential candidates to displace AA include DPA (a 22-carbon LC-PUFA with five double bonds), DHA (a 22-carbon LC-PUFA with six double bonds), and DGLA (a 20-carbon LC-PUFA with three double bonds). Importantly, fatty acids can vary considerably in terms of their affinity to the oxygenation enzymes. Accordingly, DPA and DHA not only have more double bonds than AA, but they also have two additional carbons in the PUFA chain. In that regard, DHA is farther afield than EPA owing to the “extra” carbons and two “extra” double bonds compared to AA. As such, the overall suite of oxygenation metabolites from DPA and DHA is expected to have less potential for mitigating the pro-inflammatory effects of AA activation. Like AA and EPA, DGLA features 20 carbons and has one fewer double bond than AA. Its precursor, GLA (a 18-carbon LC-PUFA with 3 double bonds), can be given orally and is enriched in certain food (e.g., borage oil). Counterintuitively, DGLA is itself converted to AA in vivo. This raises the possibility that the GLA/DGLA approach would undermine its own efficacy by increasing AA levels in vivo. Conversely, EPA does not share this problem, as EPA is not converted to AA as part of its metabolism. Rather, EPA is more likely to be oxidized in parallel with AA and can also be elongated into DPA and DHA.

Given that DPA and DHA diverge more from AA chemistry and metabolism, and that DGLA itself becomes AA, EPA occupies a unique position that avoids conversion into the compound whose effects are to be avoided but remains similar enough that it can compete for enzymes that would otherwise oxidize AA. EPA itself may inhibit AA oxygenation beyond simple competitive inhibition. These chemical considerations are congruent with limited experience from clinical outcome studies. For example, combinations of EPA and DHA are also found in food, namely, fish oil. Combinations featuring marine EPA and DHA have been administered with the intent of preventing MACE events in patients with high risk for atherosclerotic MACE events. Intriguingly, two large outcomes studies featuring purified EPA as an ethyl ester indeed showed those randomly assigned EPA had fewer MACE events compared to control groups who did not receive an EPA-based intervention. On the other hand, a large outcomes study featuring EPA mixed with DHA had no such benefits. This generally accords with the notion that DHA may be too far afield from AA to provide meaningful protection to the ill effects therefrom. Similarly, studies of borage oil combined with marine oils have suggested an overall benefit in inflammatory states, albeit, with mixed results in human studies. Again, given that the GLA from borage oil is converted to DGLA and thence, AA, this option may undermine its own efficacy.

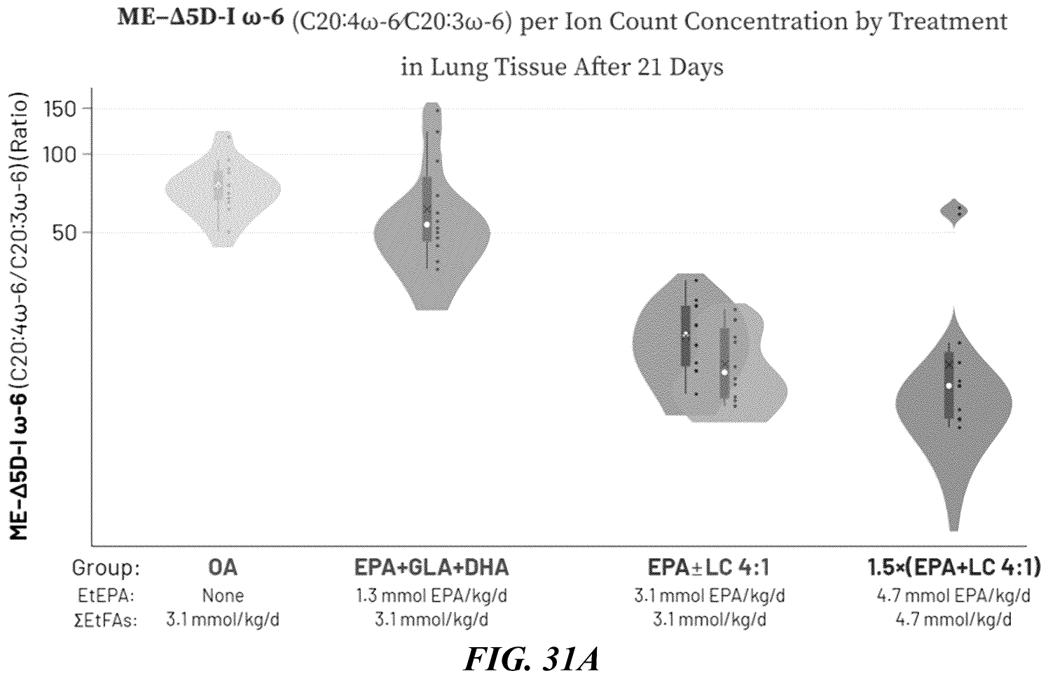

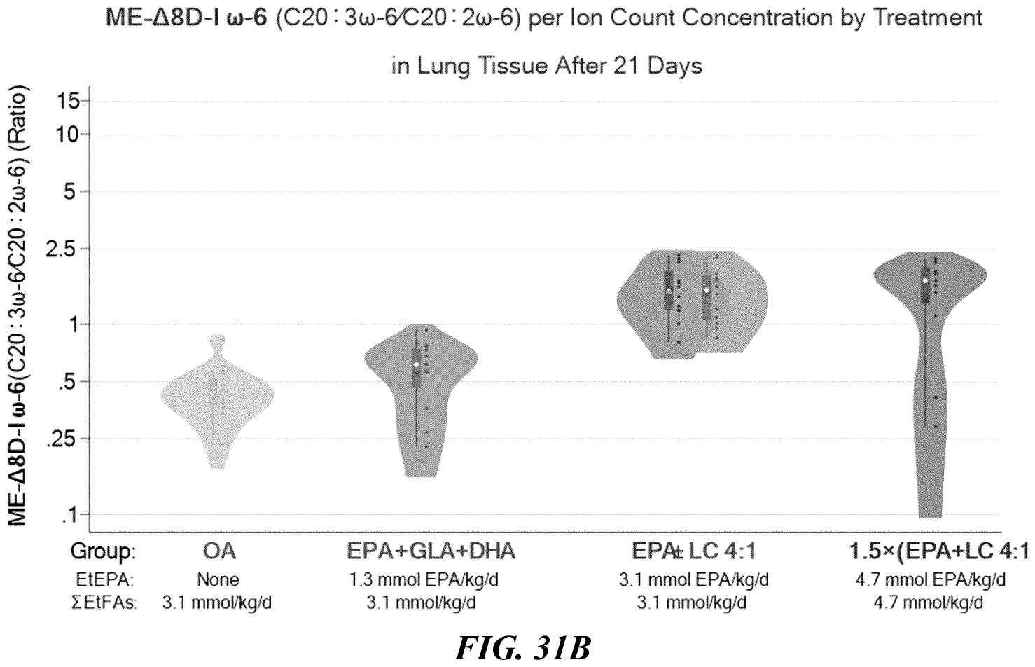

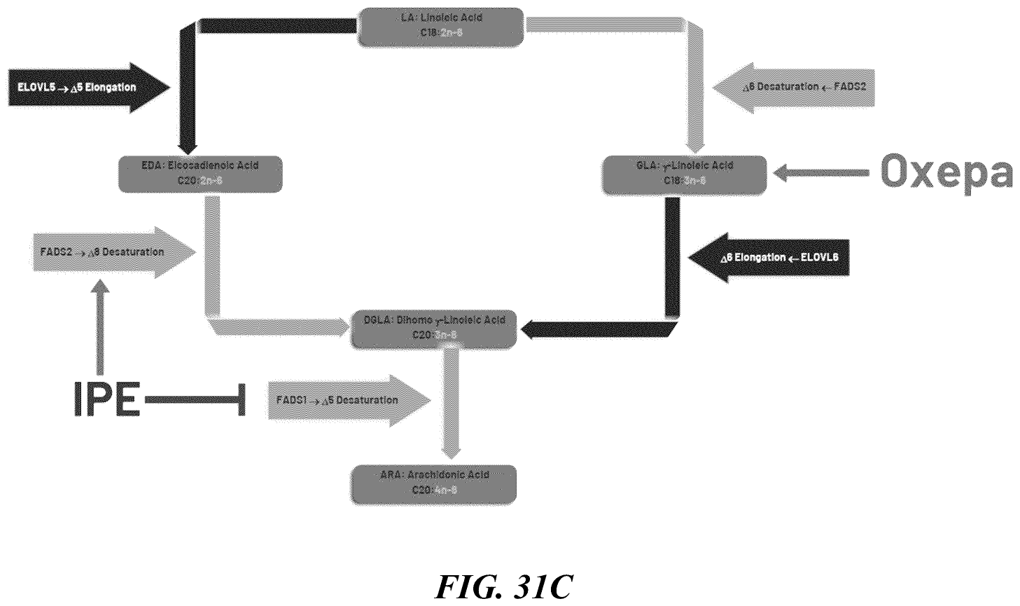

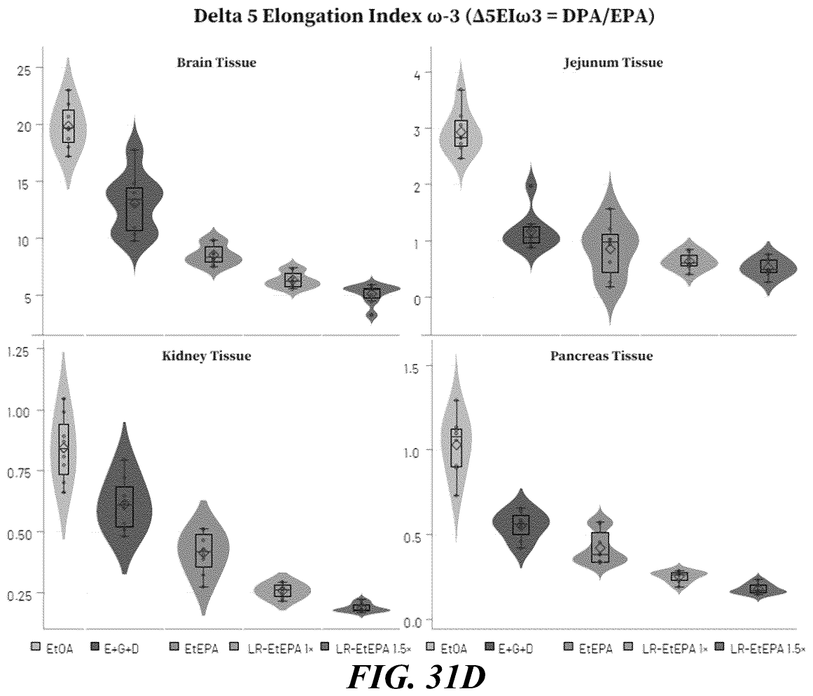

In view of (1) the specific concerns that GLA can raise AA, (2) the relative dissimilarity of DPA and DHA to AA, and (3) the relative similarity of EPA to AA in terms of analogous metabolism and competition for enzymes, compositions with high purity of EPA are expected to moderate the ill effects of over-exuberant AA oxygenation and activation seen in conditions such as SIRS, sepsis, and ARDS in a manner superior to other pharmaceutical compositions that employ a mixture of fatty acids. Oxepa® is one such pharmaceutical composition used to treat sepsis and ARDS containing a variety of excipients and mixture of fatty acids (e.g., EPA, GLA, and DHA), some of which can turn into AA in vivo and thus raise AA levels and contribute to the body's inflammatory cascade (FIG. 31C). Oxepa® also contains numerous other fatty acids, which could “dilute” the impact of the product in mitigating the ill effects of AA's oxylipins. Namely, other fatty acids in Oxepa® would naturally compete with GLA, EPA, and DHA for transport and especially for residence in cell membranes and tissue delivery. In contrast, LR-EtEPA would be dosed opposite dietary fat loads to avoid diluting its efficacy by providing competing fatty acids. A composition of high purity EPA and substantially no contamination from other fatty acids will likely not raise AA levels in vivo and instead compete with AA to mitigate its inflammatory effects.

This prompted the inclusion of an arm of the multiple-daily dose, Long-Evans rat experiment described herein that included a combination of EtEPA, ethyl-GLA (EtGLA), and ethyl-DHA (EtDHA) (henceforth, the “EPA+GLA+DHA” or “E+G+D” arm). These three fatty acids are among the mixture of fatty acids in Oxepa®. The sum total of these three fatty acids was equimolar to the dose of EtEPA in the plain EtEPA and the LR-EtEPA arms of the experiment. Thus, differences between the pure EtEPA arms and this E+G+D arm are attributable to “replacing” the GLA+DHA portions of E+G+D with EtEPA. The combination of E+G+D has medical significance, because this combination has been shown to improve oxygenation and ventilation among critically ill patients suffering from ARDS. Thus, the relative portions of these three fatty acids were used in the experiment, and the overall dose of LC-PUFAs was similar to that used to treat ARDS. Given the medical significance of these OXygenation-Promoters, this arm is also referred to as “OXP” in figures and in the discussion below. A consistent finding was that plain EtEPA and LR-EtEPA were typically more potent at offsetting/suppressing AA compared to E+G+D. Beyond ARDS, we also assembled a slightly larger set of LC-PUFAs which have been proposed for medicinal use for a variety of diseases. These are thought to be medicinal to the extent that they or their fatty acid metabolites are oxylipin precursors whose metabolites compete with AA; henceforth, they are referred to as Medicinal Oxylipin Precursors (MOPs), and consist of DGLA, EPA, DPA, and DHA. This yield three ways to compare sets of fatty acids to AA as a ratio: (1) EPA/AA, (2) OXP/AA=[DGLA+EPA+DHA]/AA, and (3) MOP/AA=[DGLA+EPA+DPA+DHA]/AA. Among these, the latter two, OXP/AA and MOP/AA, are most appropriate to compare study arms to E+G+D, because the numerators contain the three fatty acids intended to exert a therapeutic effect. Generally, LR-EtEPA was superior at raising all three ratios compared to an equimolar dose of plain EtEPA, and typically both EtEPA formulations were superior to an equimolar dose of E+G+D. Importantly, as expected E+G+D itself was often superior to the referent therapy, an equimolar dose of oleic acid (OA); thus, E+G+D demonstrated efficacy at displacing AA by raising the fatty acids comprising OXP. As such, the experiment affirmed the mechanism that motivated development of EPA+GLA+DHA as a therapeutic in the first place, namely, to increase the OXP-derived oxylipins (i.e., oxylipins from EPA, DGLA, and DHA) to thereby improve ARDS, namely, by diminishing tissue AA levels in favor of fatty acids whose oxylipins are less inflammatory, thrombotic, vasoconstrictive, and most especially, bronchoconstrictive. Accordingly, superiority of plain EtEPA and especially LR-EtEPA imply that that these would improve upon EPA+GLA+DHA, and in the case of LR-EtEPA, by a several-fold improvement in AA-displacement by medicinal oxylipin precursors.

Without wishing to be bound by theory, EPA might further decrease AA levels by altering Δ8-desaturase (D8D) and Δ5-desaturase (D5D) enzymes, which respectively facilitate DGLA synthesis and suppress subsequent catabolism yielding AA in the omega-6 PUFA pathway (FIG. 2). Through its dual function of promoting D8D (and DGLA) and inhibiting 5D5 (and AA), EPA could effectively reduce AA levels and increase metabolites that compete with AA. Additionally, fatty acids other than EPA (as found in Oxepa®) not only “dilute” the anti-inflammatory effect of EPA, but they may also compete with EPA for the cross-esterification process with the phospholipids of the composition, thereby reducing the absorption and bioavailability of EPA in lymph and at tissue level. Therefore, it is contemplated that the lymph-releasing EPA composition of the present technology (LR-EtEPA) is especially valuable and useful to treat diseases involving inflammation, vasoconstriction, and bronchoconstriction from AA-derived oxylipins through the pulmonary route, and it would have an advantage over plain EtEPA, and both would have an advantage over compositions of mixture of fatty acids such as Oxepa®.

Accordingly, in some embodiments, provided is a method of treating and/or preventing SIRS, sepsis, and/or ARDS in a subject in need thereof, the method comprising administering to the subject a therapeutically effective amount of a composition comprising PUFAs or derivatives thereof and a source of phospholipid according to various embodiments disclosed herein.

Neurological Diseases

The co-administration of PUFAs (e.g., EtEPA) with additives including phospholipids and/or additional emulsifiers facilitates in vivo absorption of the fatty acid in the form of phospholipid conjugate. Not only is such compound primed for uptake by cells because phospholipids are a major component of cell membrane, but it is also expected to have a higher efficiency as crossing the blood-brain barrier (BBB), thereby delivering the fatty acid which is the active ingredient of the composition to the brain. The BBB can be a roadblock for pharmaceutical agents to access their therapeutic target in the brain or reach a sufficient level inside the brain. Therefore, the lymph-releasing formulation of the present technology may provide a novel strategy and platform for delivering fatty acids across the BBB for them to exert their anti-inflammatory, pro-cognitive, and/or other neuroprotective effects useful in the treatment of a variety of neurological diseases and disorders.

Without wishing to be bound by theory, PUFAs including EPA are important in the regulation of phospholipase A2 (PLA2), which is an enzyme that catalyzes the cleavage of fatty acids from the sn-2 position of phospholipids and is implicated in PUFA and oxylipin pathways. PLA2 is present in the central nervous system (CNS) and is associated with neurodegenerative diseases due to its role in inflammatory responses. EPA can inhibit PLA2 and prevent production of pro-inflammatory eicosanoids. Additionally, EPA's inhibitory effect on Δ5-desaturase and Δ5-elongase as discussed above may also contribute to its anti-inflammatory function in a variety of pathological conditions of the CNS.

Accordingly, in some embodiments, provided is a method of treating and/or preventing a neurological disease in a subject in need thereof, the method comprising administering to the subject a therapeutically effective amount of a composition comprising PUFAs or derivatives thereof and a source of phospholipid according to various embodiments disclosed herein.

In some embodiments, the neurological disease comprises chorea/Huntington's chorea, sleep disorders, dementia, psychosis, anxiety, treatment-resistant depression, neuropathic pain, schizophrenia (especially in patients with tardive dyskinesia), bipolar disorder, dyslexia, dyspraxia, attention deficit hyperactivity disorder (ADHD), epilepsy, autism, Alzheimer's disease, Parkinson's Disease, senile dementia, multiple sclerosis, diabetes-induced neuropathy, macular degeneration, retinopathy of prematurity, amyotrophic lateral sclerosis (ALS), retinitis pigmentosa, cerebral palsy, muscular dystrophy, neurological cancer, cystic fibrosis, and/or neural tube defects.

Cancer

EPA is currently in clinical trials for treatment of cancer including colorectal cancer (see NCT01070355). The composition of the present technology, for example, the LR-EtEPA formulation, results in greater EPA uptake in various tissues and thus could present a preferred option of cancer treatment.

Accordingly, in some embodiments, provided is a method of treating and/or preventing cancer in a subject in need thereof, the method comprising administering to the subject a therapeutically effective amount of a composition comprising PUFAs or derivatives thereof and a source of phospholipid according to various embodiments disclosed herein.

In some embodiments, the cancer is a hematological malignancy. Non-limiting exemplary hematological malignancies include monoclonal B cell lymphocytosis, multiple myeloma, myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), blast crisis chronic myelogenous leukemia (bcCML), B cell acute lymphoid leukemia (B-ALL), T cell acute lymphoid leukemia (T-ALL), T cell lymphoma, and B cell lymphoma.

In some embodiments, the cancer is a solid tumor. Non-limiting exemplary solid tumors include lung cancer, breast cancer, liver cancer, stomach cancer, colon cancer, rectal cancer, colorectal cancer, kidney cancer, gastric cancer, gallbladder cancer, cancer of the small intestine, esophageal cancer, melanoma, bone cancer, pancreatic cancer, skin cancer, uterine cancer, ovarian cancer, testicular cancer, cancer of the thyroid gland, cancer of the adrenal gland, bladder cancer, and glioma.

Diseases Associated with the Kidneys, Pancreas, Liver, Intestines, Blood Cells, Lymph, and the Musculoskeletal System

In some embodiments, provided is a method of treating and/or preventing a disease associated with a tissue or organ in a subject in need thereof, the method comprising administering to the subject a therapeutically effective amount of a composition comprising PUFAs or derivatives thereof and a source of phospholipid according to various embodiments disclosed herein, wherein the tissue or organ is selected from the group consisting of kidney, the endocrine system, pancreas, liver, intestines, blood cells, and the musculoskeletal system.

As demonstrated in the working examples, the lymph-releasing compositions of the present technology are superior at delivering fatty acids (e.g., EPA) to various tissues of the body including the kidney, pancreas, and intestines, where the fatty acids (e.g., EPA) can mitigate injury from inflammation, vasoconstriction, bronchospasm, and/or thrombosis, potentially by displacing, competing with, or reducing harmful oxylipins. Additionally, EPA and its oxylipins are very complementary to steroids; both work by limiting oxylipin production. Alternatively, EPA could be used as a steroid-sparing agent. Steroids themselves have lots of adverse events, but EPA has few known adverse events.

In some embodiments, the disease associated with kidney comprises post-infectious glomerulonephritis, IgA nephropathy (Berger's disease), Henoch-Schönlein purpura, systemic IgA vasculitis, microscopic polyangiitis, granulomatosis with polyangiitis (Wegener's), eosinophilic granulomatosis with polyangiitis (Churg-Strauss), polyarteritis, idiopathic crescentic glomerulonephritis, anti-GBM glomerulonephritis, Goodpasure syndrome, cryoglobulin-associated glomerulonephritis, idiopathic membranoproliferative glomerulopnephritis (MPGN), hepatitis C-associated glomerulonephritis, systemic lupus erythematosus (SLE) associated glomerulonephritis, minimal change disease (nill disease, lipoid nephrosis), membranous nephropathy, focal and segmental glomerulosclerosis, amyloidosis, diabetic nephropathy, HIV-associated nephropathy, membranoproliferative glomerlonephropathy, mitigating proteinuria, mitigating chronic renal failure, and/or mitigating mortality/morbidity in severe chronic kidney disease (CKD)/end-stage renal disease (ESRD).

In some embodiments, the disease associated with the endocrine system comprises hypopituitarism, thyroiditis, and/or Paget's disease.

In some embodiments, the disease associated with pancreas comprises hyperglycemia, pre-diabetes, diabetes (Type 1 and/or Type 2), and/or pancreatitis.

In some embodiments, the disease associated with liver comprises chronic viral hepatitis, autoimmune hepatitis, alcoholic liver disease, non-alcoholic fatty liver disease, hemochromatosis, Wilson disease, primary biliary cholangitis, primary sclerosing cholangitis, and/or cholelithiasis.

In some embodiments, the disease associated with intestines comprises digestive conditions include gastroesophageal reflux disease (GERD) (by mitigating esophageal reflux via oxylipin effects), gastritis, peptic ulcer disease, obesity (mitigated obesity by inducing satiety), and cachexia (mitigated by limiting inflammation); intestinal disease conditions include intestinal angina, inflammatory bowel disease (e.g., Crohn disease, ulcerative colitis), antibiotic-associated colitis, irritable bowel syndrome, colon cancer, colon polyposis, and/or carcinoid.

In some embodiments, the disease associated with blood cells comprises iron deficiency anemia, anemia of chronic disease, hemolytic anemia, thalassemia, polycythemia vera, sickle cell disease anemia, and sickle cell disease pain/crisis; platelet disorders include immune thrombocytopenia, and pro-thrombotic conditions; white cell disorders conditions include leukemias, Non-Hodgkin lymphomas, and/or Hodgkin lymphoma.

In some embodiments, the disease associated with lymph comprises lymphedema.

In some embodiments, the disease associated with the musculoskeletal system comprises muscle conditions include statin myopathy, rhabdomyolysis, polymyalgia rheumatica, polychondritis, and Behcet syndrome; bone conditions include gouty arthritis, calcium pyrophosphate deposition, rheumatoid arthritis, Still disease, ankylosing spondylitis, psoriatic arthritis, and reactive arthritis; systemic conditions include systemic lupus erythematosus (SLE), antiphospholipid syndrome, systemic sclerosis (scleroderma), polymoysitis, dermatomyositis, Sjögren syndrome, and IgG4-related disease.

Diseases Associated with Oxidative Stress and/or Glutathione (GSH) Depletion

In some embodiments, provided is a method of treating and/or preventing a disease or disorder associated with oxidative stress, glutathione (GSH) depletion, Nrf2 activation, and/or heme-oxygenase activation (including cell lysis, hemolysis, other blood cell lysis, or conditions involving exposure to free heme during tissue injury) in a subject in need thereof, the method comprising administering to the subject a therapeutically effective amount of a composition comprising PUFAs or derivatives thereof and a source of phospholipid according to various embodiments disclosed herein.

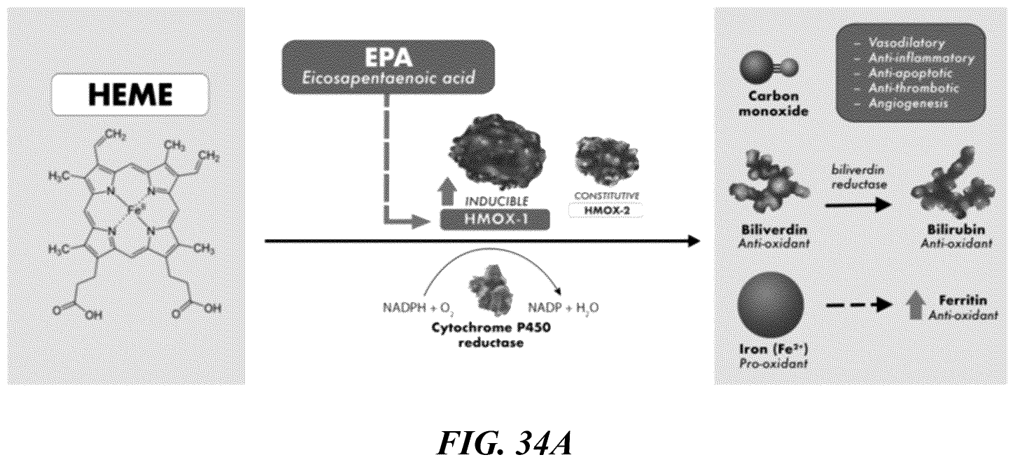

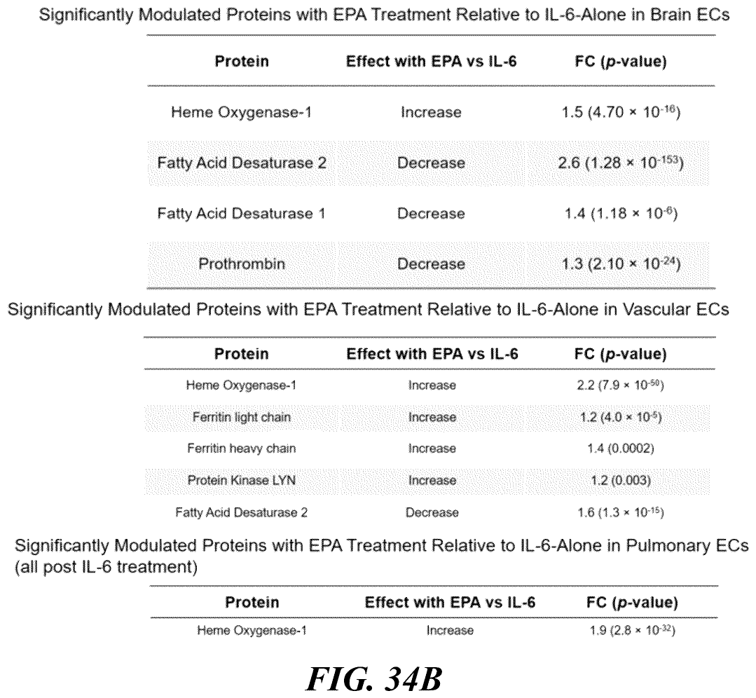

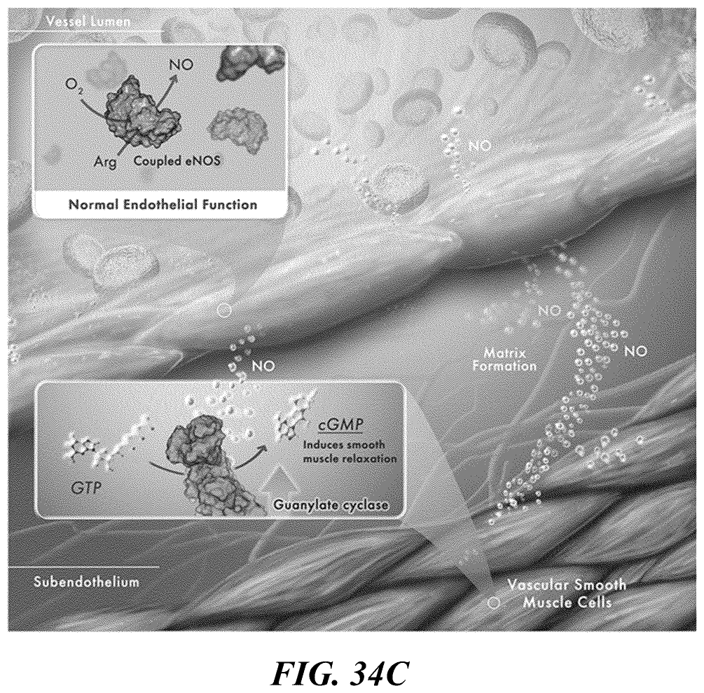

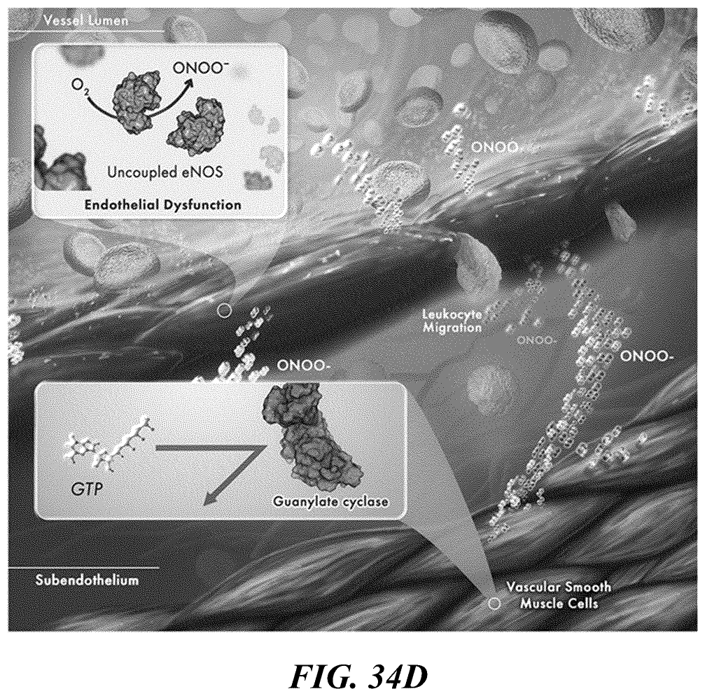

Endothelial nitric oxide (NO) is produced by the NO synthase (eNOS) dimer which couples oxidation of L-arginine with the reduction of molecular oxygen (FIGS. 34C-34D). See Forstermann and Sessa, Euro Heart J. (2011) 33:829-837, which is incorporated herein by reference in its entirety. Under disease-like conditions and increased oxidative stress (i.e., high glucose, smoking, hypertension), there is eNOS “uncoupling” that favors production of superoxide (O2-) which reacts with NO to form peroxynitrite (ONOO—), a cytotoxic radical. The ratio of [NO]/[ONOO-] is a key indicator of eNOS coupling efficiency, while loss of NO bioavailability is associated with atherothrombotic risk. It is known that EPA has favorable effects on eNOS coupling in vascular endothelial cells (ECs) compared to DHA. The benefits of EPA were associated with an improved EPA/AA ratio. See Sherratt et al., Prostaglandins Leukot Essent Fatty Acids. (2021) 173, incorporated herein by reference in its entirety. The effects of EPA on NO release were enhanced in combination with a high-intensity statin. See Mason et al., Biomed Pharmacother. (2018) 103:1231-1237, incorporated herein by reference in its entirety. The mechanism for improved eNOS function with EPA is not fully understood but may be related to increased eNOS expression and reduced production of reactive oxygen species (ROS) compared to DHA.

EPA administered as icosapent ethyl (IPE) is the first FDA and EMA approved drug to reduce cardiovascular risk among patients with elevated triglyceride levels as an add-on to maximally tolerated statin therapy. The REDUCE-IT trial showed that treatment with high dose IPE (4 g/day) reduced composite cardiovascular events by 25% in statin-treated patients with elevated baseline triglyceride levels. See Bhatt et al., N Engl J Med. (2019) 380:11-22, incorporated herein by reference in its entirety. The benefits of IPE were independent of baseline triglyceride levels but positively correlated with plasma levels of EPA. Imaging trials showed a significant regression in plaque volume and composition with IPE compared to statin alone in patients with atherothrombotic disease. See Budoff et al., Euro Heat J. (2020) 41:3925-3932; Watanabe et al., J Cardiol. (2017) 70:537-544, each of which is incorporated herein by reference in its entirety. In contrast to IPE treatment, the results of trials using mixed omega-3 fatty acids have failed to reduce cardiovascular events. This may be due to differences in formulation and potential pleiotropic benefits and membrane interactions unique to EPA. See Sherratt et al., Prostaglandins Leukot Essent Fatty Acids. (2021) 173; Mason et al., Metab Clin. (2022) 130:155161; Sherratt et al., J Lipid Res. (2021) 62; Mason et al., Arterioscler Thromb Vasc Biol. (2020) 40:1135-1147, each of which is incorporated herein by reference in its entirety.

To elucidate mechanisms of enhanced eNOS coupling and NO bioavailability of n-3 fatty acids, the effects of EPA and DHA have been compared on the expression of eNOS and proteins that regulated reactive oxygen species (ROS) in human ECs during inflammation (see Example 5).

GSH is an antioxidant capable of preventing damage to important cellular components caused by sources such as ROS, free radicals, peroxides, and heavy metals. As shown in Example 5, the protein for glutathione reductase (GSR) was significantly increased by EPA. In addition to heme oxygenase-1 (HO-1), antioxidant response element (ARE) may induce glutathione S-transferases (GST). The GSR activity may be evaluated using mRNA expression of GST. Both GSR and GST induction may aid the antioxidant effects of GSH. Therefore, GSH may be affected by two separate proteins, which are regulated independently.

Because of EPA's effect on GSR, the composition of the present technology comprising EPA may exhibit a particularly robust effect on the overall antioxidant effects by enhancing GSH metabolism. The composition may show a broad effect on treating or preventing tissue injuries caused by diseases including, but not limited to, pulmonary inflammation, anemia, sickle cell disease, and glomerulonephritis. The composition may also treat diseases/conditions related to GSH deficiency, such as kwashiorkor, seizure, Alzheimer's disease, Parkinson's disease, liver disease, cystic fibrosis, anemia, sickle cell disease, human immunodeficiency virus (HIV) infection/acquired immunodeficiency syndrome (AIDS), cancer, heart attack, stroke, and diabetes.

Accordingly, in some embodiments, provided is a method of treating and/or preventing pulmonary inflammation, anemia, sickle cell disease, and/or glomerulonephritis in a subject in need thereof, the method comprising administering to the subject a therapeutically effective amount of a composition comprising PUFAs or derivatives thereof and a source of phospholipid according to various embodiments disclosed herein.

In some embodiments, provided is a method of treating and/or preventing a disease or disorder associated with GSH depletion in a subject in need thereof, the method comprising administering to the subject a therapeutically effective amount of a composition comprising PUFAs or derivatives thereof and a source of phospholipid according to various embodiments disclosed herein.

In some embodiments, the disease or disorder associated with GSH depletion is at least one selected from the group consisting of a neurodegenerative disorder, a pulmonary disease, an immune disease, a cardiovascular disease, a renal disease, a liver disease, an endocrine disease, a red blood cell disease, a gastrointestinal disease, a rheumatologic and/or musculoskeletal disorder, a dermatologic disease, an obstetric and/or gynecological disease, and an age-related disorder.

In some embodiments, the neurodegenerative disorder comprises Alzheimer's disease, Parkinson's disease, Huntington's disease, amyotrophic lateral sclerosis (ALS), and/or Friedreich's ataxia.

In some embodiments, the pulmonary disease comprises community-acquired pneumonia, sepsis, SIRS, ARDS, chronic obstructive pulmonary disease (COPD), asthma, interstitial lung disease, cystic fibrosis, pulmonary vasculitis (e.g., granulomatosis with polyangiitis (GP), eosinophilic granulomatosis with polyangiitis (EGPA), microscopic polyangiitis (MPA)), pulmonary-renal vasculitis (e.g., Goodpasture's syndrome, cryoglobulinemia, systemic lupus erythematosus (SLE), systemic sclerosis, antiphospholipid syndrome), pulmonary inflammation, non-small cell lung cancer (especially COX-2 over-expressing cancer), and/or chronic/episodic hemolytic anemia (e.g., hereditary spherocytosis, thalassemia, secondary hemolysis from other diseases, and transfusion reactions).

In some embodiments, the immune disease is an autoimmune disease and/or HIV infection/AIDS. Non-limiting exemplary autoimmune diseases include type 1 diabetes, lupus, systemic lupus erythematosus, rheumatoid arthritis, psoriasis, psoriatic arthritis, multiple sclerosis, inflammatory bowel disease, Crohn's disease, ulcerative colitis, Addison's disease, Graves' disease, Sjögren's syndrome, Hashimoto's thyroiditis, myasthenia gravis, autoimmune vasculitis, pernicious anemia, and celiac disease.

In some embodiments, the cardiovascular disease comprises hypertension, myocardial infarction, and/or cholesterol oxidation.

In some embodiments, the renal disease comprises renal vasculitis (e.g., Berger's disease), proteinuria, chronic kidney disease (CKD), and/or end stage renal disease (ESRD).

In some embodiments, the liver disease comprises non-alcoholic fatty liver disease.

In some embodiments, the endocrine disease comprises hyperglycemia, pre-diabetes, and/or Paget's disease.

In some embodiments, the red blood cell disease comprises anemia and/or sickle cell disease.

In some embodiments, the gastrointestinal disease comprises pancreatitis, inflammatory bowel disease, irritable bowel syndrome, obesity, cachexia, esophageal reflux, and/or biliary cirrhosis.

In some embodiments, the rheumatologic and/or musculoskeletal disorder comprises statin myopathy.

In some embodiments, the dermatologic disease comprises allergic dermatitis, general dermatitis, menopausal hot flush, and/or medicinal hot flush.

In some embodiments, the obstetric and/or gynecological disease comprises menorrhagia, preeclampsia, and/or dysmenorrhea.

In some embodiments, the age-related disorder comprises cataracts, macular degeneration, hearing impairment, and/or glaucoma.

In some embodiments, the method further comprised administering to the subject a N-acetylcysteine (NAC) related agent that can raise GSH. Non-limiting examples of NAC related agents include cystine, methionine, N-acetylcysteine, and L-2-oxothiazolidine-4-carboxylate. Using one of these NAC related agents with the presently disclosed composition comprising EPA may further potentiate GSH raising.

In some embodiments, upon administration of the composition, the subject exhibits an increase in GSR activity.

In some embodiments, upon administration of the composition, the subject exhibits an increase in GST activity.

Inflammation and Diseases Induced by Air Pollution

Long-term and/or short-term exposure to air pollution contributes to an inflammatory response of the body and the pathogenesis of cardiovascular diseases and disorders. It is approximated that air pollution causes seven million deaths per year worldwide with over half of the deaths attributable to the progression or development of a cardiovascular disease or disorder. Given the known anti-inflammatory effects of EPA, as well as the superior tissue delivery capability of the lymph-releasing formulation of the present technology particularly to the cardiopulmonary system, it is contemplated that the composition of the present technology is useful for treating diseases associated with air pollution.

Accordingly, in some embodiments, provided is a method of treating and/or preventing oxidative stress, endothelial dysfunction, narrowing and/or thickening of arteries, and/or inflammation induced by inhalation of particulate matter in a subject in need thereof, the method comprising administering to the subject a therapeutically effective amount of a composition comprising PUFAs or derivatives thereof and a source of phospholipid according to various embodiments disclosed herein. The term “particulate matter” refers to a mixture of species generated from numerous emission sources. The particulate matter may be emitted directly into the air in the form of soot, smoke, and/or dust. The particulate matter can be formed in the atmosphere from the reactions of gases including by not limited to nitric oxides (NOx), sulfur oxides (SOx), reactive organic gases (ROG), and/or ammonia.

As used herein, the term “oxidative stress” refers to the increased formation of reactive oxygenated species (ROS) and/or decreased antioxidative potential (i.e., capacity to reduce or impair the generation of ROS) in an afflicted person. The term “endothelial dysfunction” refers to damage or degradation of the endothelial lining caused by numerous factors, including but not limited to, high blood pressure, high blood glucose levels, and/or increased blood lipid levels. Endothelial dysfunction can then lead to reduced function in endothelium-dependent vasodilation, pro-coagulation, and proinflammatory response. The term “narrowing” of arteries refers to a condition characterized by a decreased or a complete reduction in blood flow and oxygen transport to target tissues and organs of an afflicted person that occurs, for example, from the formation of plaque within the arterial wall and/or as a result of inflammation causing a swelling of the arterial wall. An occlusion (i.e., blockage) of the arteries prevents sufficient blood flow and thereby, oxygen transport to target tissues and organs, which can lead to a wide range of illness such as, but not limited to, hypoxia, myocardial infraction, stroke, and/or pulmonary embolism. The term “thickening” of arteries herein refers to an actual thickening of the arterial wall (i.e., an increase in the ratio of the wall thickness-to-radius of the artery) and/or an actual enlargement of the arterial wall (i.e., dilatation). The thickening of the arterial wall can lead to a weakened and narrowed arterial wall, which overtime can cause irregular blood flow and oxygen transport. On some occasions, the thickening of the arterial wall can result in an actual rupture of the wall, preventing blood flow and oxygen transport. Both a partial and complete block in blood flow and oxygen transport to target tissues can result in subsequent organ and tissue damage and/or death. The narrowing and thickening of the arterial wall can occur independently or dependently of each other.

The term “inflammation” refers to individual tissue (e.g., pulmonary) and/or systemic inflammation. For example, pulmonary inflammation is characterized by inflammation of the pulmonary system, resulting in restricted oxygen flow due to a narrowing of the air passageways of an afflicted person. The term “pulmonary system” refers to those organs and/or structures responsible for taking in oxygen into and/or expelling carbon dioxide from the body. The organs and/or structures include, but are not limited to, those associated with the nasal, pharyngeal, and laryngeal passageways, the trachea, bronchi, bronchioles, and/or alveoli. In one embodiment, the alveoli in the lungs become inflamed, which can decrease the follow of oxygen through the alveoli to the bloodstream. The narrowing of the air passageways causes episodic dyspnea, coughing, and/or wheezing, all of which are associated with asthma, and in severe cases, causes death. Systemic inflammation is characterized by the widespread inflammation throughout the body of an afflicted person. Systemic inflammation leads to the degradation of both the structure and function of essential organs, such as the muscle, heart, and liver, compromises the immune system, and also causes multi-organ failure and death.

In some embodiments, provided is a method of treating and/or preventing oxidative stress, endothelial dysfunction, narrowing and/or thickening of arteries, and/or inflammation induced by long-term and/or short-term exposure to air pollution in a subject in need thereof, the method comprising administering to the subject a therapeutically effective amount of a composition comprising PUFAs or derivatives thereof and a source of phospholipid according to various embodiments disclosed herein. The term “long-term” in the present context refers to exposure to air pollution for a period of time greater than or equal to one year. The term “short-term” refers to exposure to air pollution for a period of time less than one year.

In some embodiments, provided is a method of treating and/or preventing an atherosclerotic cardiovascular disease, or reducing the risk of an atherosclerotic cardiovascular disease, in a subject in need thereof, the method comprising administering to the subject a therapeutically effective amount of a composition comprising PUFAs or derivatives thereof and a source of phospholipid according to various embodiments disclosed herein. The term “atherosclerotic cardiovascular disease” refers to any condition characterized by plaque accumulation on vessel walls and vascular inflammation.

Polluted air contains particulate matter, which can be a mixture of particulates of varying sizes. The various sizes of particulate matter are classified as coarse, fine, and ultrafine. In some embodiments, coarse particulate matter refers to particulates having a mean or median diameter, on a volume basis, less than about 10 μm and greater than about 2.5 μm (PM2.5-10). In some embodiments, fine particulate matter refers to particulates having a mean or median diameter, on a volume basis, of about 2.5 μm (PM2.5). In some embodiments, ultrafine particulate matter refers to particulates having a mean or median diameter, on a volume basis, less than about 0.1 μm (PM0.1). The particulate matter described in any of the embodiments herein may be less than about 10 μm and greater than about 2.5 μm in diameter, less than or equal to about 2.5 μm in diameter, or less than about 0.1 μm in diameter.

In some embodiments, upon administration of the composition of the composition described herein in any of its embodiments, the subject may exhibit beneficial effects in heart rate and/or rhythm following administration. In some embodiments, the beneficial effects include a reduction in arrhythmia suppression levels, ventricular arrhythmia rates, or heart rate, or an increase in heart rate variability.

In another embodiment, the disclosure provides a method of suppressing an inflammatory response caused by the inhalation of particulate matter in the lungs. In some embodiments, the inflammatory response is observed in not only the lungs, but also other organs, to included but not limited to the brain, heart, coronaries, liver, kidneys, spleen, pancreas, and intestine.

In any of the above embodiments disclosed herein, the therapeutically effective dose of the composition of the present technology is between 1 mg and 20 g of EPA (as the term “EPA” is defined and exemplified herein) per day, for example, about 50 mg, about 100 mg, about 500 mg, about 750 mg, about 1 g, about 2 g, about 3 g, about 4 g, about 5 g, about 6 g, about 7 g, about 8 g, about 9 g, about 10 g, about 11 g, about 12 g, about 13 g, about 14 g, about 15 g, about 16 g, about 17 g, about 18 g, about 19 g, or about 20 g of EPA per day. In some embodiments, the therapeutically effective dose is between 2 g and 12 g of EPA per day. In some embodiments, the therapeutically effective dose is between 4 g and 6 g of EPA per day. In another embodiment, the therapeutically effective dose is 4 g of EPA per day.

In some embodiments, the composition administered to the subject in an amount sufficient to provide a daily dose of EPA (as the term “EPA” is defined and exemplified herein) about 1 mg to about 20,000 mg, about 25 mg to about 10,000 mg, about 50 mg to about 5000 mg, about 75 mg to about 2500 mg, or about 100 mg to about 1000 mg, for example, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5025 mg, about 5050 mg, about 5075 mg, about 5100 mg, about 5125 mg, about 5150 mg, about 5175 mg, about 5200 mg, about 5225 mg, about 5250 mg, about 5275 mg, about 5300 mg, about 5325 mg, about 5350 mg, about 5375 mg, about 5400 mg, about 5425 mg, about 5450 mg, about 5475 mg, about 5500 mg, about 5525 mg, about 5550 mg, about 5575 mg, about 5600 mg, about 5625 mg, about 5650 mg, about 5675 mg, about 5700 mg, about 5725 mg, about 5750 mg, about 5775 mg, about 5800 mg, about 5825 mg, about 5850 mg, about 5875 mg, about 5900 mg, about 5925 mg, about 5950 mg, about 5975 mg, about 6000 mg, about 6025 mg, about 6050 mg, about 6075 mg, about 6100 mg, about 6125 mg, about 6150 mg, about 6175 mg, about 6200 mg, about 6225 mg, about 6250 mg, about 6275 mg, about 6300 mg, about 6325 mg, about 6350 mg, about 6375 mg, about 6400 mg, about 6425 mg, about 6450 mg, about 6475 mg, about 6500 mg, about 6525 mg, about 6550 mg, about 6575 mg, about 6600 mg, about 6625 mg, about 6650 mg, about 6675 mg, about 6700 mg, about 6725 mg, about 6750 mg, about 6775 mg, about 6800 mg, about 6825 mg, about 6850 mg, about 6875 mg, about 6900 mg, about 6925 mg, about 6950 mg, about 6975 mg, about 7000 mg, about 7025 mg, about 7050 mg, about 7075 mg, about 7100 mg, about 7125 mg, about 7150 mg, about 7175 mg, about 7200 mg, about 7225 mg, about 7250 mg, about 7275 mg, about 7300 mg, about 7325 mg, about 7350 mg, about 7375 mg, about 7400 mg, about 7425 mg, about 7450 mg, about 7475 mg, about 7500 mg, about 7525 mg, about 7550 mg, about 7575 mg, about 7600 mg, about 7625 mg, about 7650 mg, about 7675 mg, about 7700 mg, about 7725 mg, about 7750 mg, about 7775 mg, about 7800 mg, about 7825 mg, about 7850 mg, about 7875 mg, about 7900 mg, about 7925 mg, about 7950 mg, about 7975 mg, about 8000 mg, about 8025 mg, about 8050 mg, about 8075 mg, about 8100 mg, about 8125 mg, about 8150 mg, about 8175 mg, about 8200 mg, about 8225 mg, about 8250 mg, about 8275 mg, about 8300 mg, about 8325 mg, about 8350 mg, about 8375 mg, about 8400 mg, about 8425 mg, about 8450 mg, about 8475 mg, about 8500 mg, about 8525 mg, about 8550 mg, about 8575 mg, about 8600 mg, about 8625 mg, about 8650 mg, about 8675 mg, about 8700 mg, about 8725 mg, about 8750 mg, about 8775 mg, about 8800 mg, about 8825 mg, about 8850 mg, about 8875 mg, about 8900 mg, about 8925 mg, about 8950 mg, about 8975 mg, about 9000 mg, about 9025 mg, about 9050 mg, about 9075 mg, about 9100 mg, about 9125 mg, about 9150 mg, about 9175 mg, about 9200 mg, about 9225 mg, about 9250 mg, about 9275 mg, about 9300 mg, about 9325 mg, about 9350 mg, about 9375 mg, about 9400 mg, about 9425 mg, about 9450 mg, about 9475 mg, about 9500 mg, about 9525 mg, about 9550 mg, about 9575 mg, about 9600 mg, about 9625 mg, about 9650 mg, about 9675 mg, about 9700 mg, about 9725 mg, about 9750 mg, about 9775 mg, about 9800 mg, about 9825 mg, about 9850 mg, about 9875 mg, about 9900 mg, about 9925 mg, about 9950 mg, about 9975 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg.

In some embodiments, the composition is administered to the subject for a period of time between about 3 days to about 1 year, for example, about 3 days, about 4 days, about 5 days, about 6 days, about 1 week, about 1.5 weeks, about 2 weeks, about 2.5 weeks, about 3 weeks, about 1 month, about 2 months, about 3 months, about 4 months, about 5 months, about 6 months, about 7 months, about 8 months, about 9 months, about 10 months, about 11 months, or about 1 year.

In some embodiments, the composition is administered to the subject once a day, twice a day, three times a day, or four times a day for a period of about 3 days, about 5 days, about 7 days, about 10 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years.

In some embodiments, the composition is administered to the subject once or more times (e.g., twice, three times, four times, or more) a day. In some embodiments, the composition is administered to the subject every day, every other day, every third day, weekly, biweekly (i.e., every other week), every third week, monthly, every other month, or every third month. In some embodiments, the composition can be administered continuously or intermittently, for example, in one or more cycles. In those embodiments, within each cycle, the composition can be administered at various lengths and/or frequencies as described above.

In some embodiments, the composition may be administered over a pre-determined time. Alternatively, the composition may be administered until a particular therapeutic benchmark is reached. In some embodiments, the methods provided herein include a step of evaluating one or more therapeutic benchmarks in a biological sample, such as, but not limited to, a lipid biomarker, a metabolic biomarker, an inflammatory biomarker, a cancer biomarker, to determine whether to continue administration of the composition.

In some embodiments, the composition may be administered in various routes as determined by one skilled in the art as suitable for an indication of interest. In some embodiments, the composition is administered by oral or enteric administration, intravenous injection, intraperitoneal injection, intertumoral injection, bone marrow injection, lymph node injection, subcutaneous injection, and/or cerebrospinal fluid injection.

In some embodiments, the composition is administered with or without food. In some embodiments, the composition is administered to a subject in a fasting state, for example, having not consumed any food in the preceding 12 hours, 8 hours, 4 hours, or 2 hours. In some embodiments, the composition is administered to a subject in a fed state, for example, within 2 hours, 1 hour, 45 minutes, or 30 minutes of having a meal.

### EXAMPLES

### Example 1: Exemplary Lymph-Releasing (LR) EtEPA (LR-EtEPA) Composition

This example provides an exemplary LR-EtEPA composition, which is formulated as 1 g soft gelatin capsules, with EtEPA being the active ingredient and soy lecithin being the source of phospholipid. Polysorbate 80 and polyoxyl-35 (purified from castor bean oil) are included as emulsifiers. The content of each component is listed in Table 1:

TABLE 1
Exemplary LR-EtEPA Formulation

| | % Comp. | mg/cap | MW | Moles | Role |
|---|---|---|---|---|---|
| EtEPA | 77.12 | 800 | 330.512 | 0.002420487 | Active |
| Soy Lecithin | 4.55 | 47.15 | 643.9 | 7.32276E−05 | Additive |
| (Metarin ™ P) | | | | | |
| Polysorbate 80 | 8.41 | 87.23 | 1310 | 6.65877E−05 | Additive |
| Polyoxyl-35 | 8.41 | 87.23 | 172.0099 | 0.000507121 | Additive |
| Water | 1.52 | 15.72 | 18.02 | 0.000872203 | Additive |
| Total | 100.00 | 1037.33 | | | |

Soy lecithin (Metarin™ P) is a mixture of phospholipids, the minimum/maximum contents of each of which are specified in Table 2:

TABLE 2
Metarin ™ P Formulation

| Phospholipids | Min | Max |
|---|---|---|
| Phosphatidylcholine | 19% | 27% |
| Lysophosphatidylcholine | | 4% |
| Phosphatidylethanolamine | 16% | 22% |
| Phosphatidylinositol | 11% | 18% |
| Phosphatidic acid | 1% | 9% |

The relative weight ratio of EtEPA and lecithin may be further adjusted from the above formulation per Table 3 below:

TABLE 3
Relative Weight Ratios of EtEPA and Lecithin (LC)

| | | | | EPA mass (mg) | |
|---|---|---|---|---|---|
| EtEPA:LC | LC:EtEPA | | Formulation | in Lymph Matrix | Fold X [ref] |
| 15:1 | 1:15 | (0.067) | 4:1 EtEPA:Excip | 10 | 1X [ref] |
| 3.75:1 | 1:3.75 | (0.267) | 1:1 EtEPA:Excip | 17 | 1.7X |

| | | | | |
|---|---|---|---|---|
| 1:1 | 1:1 | 1:1 LC:EtEPA | 42 | 4.2X |
| 1:2 | 2:1 | 2:1 LC:EtEPA | 77.67 | 77.7X |
| 1:5 | 5:1 | 5:1 LC:EtEPA | 182.67 | 183X |

The model indicates that further increases in the lecithin ratio to EtEPA are likely to continue to increase the amount of EPA delivered to the lymph, consistent with the very large capacity of chylomicrons and intestinal VLDL to accommodate large boluses of fat. Accordingly, the 1:1 ratio of EtEPA:Excipients given in the single-dose experiment (i.e. 1:3.75 LC:EtEPA) likely represent the low end of the potential for LR-EtEPA to enrich lymph with EPA, and thence, pulmonary, cardiac, and brain tissue, as well as systemic tissues.

### Example 2: Comparison of Single-Dose Equimolar EtEPA and LR-EPA

The present experiment was a single-dose study where equal molar doses of eicosapentaenoic acid ethyl ester (EtEPA) and the lymph-releasing EtEPA formulation (LR-EtEPA) were administered to Sprague-Dawley rats to compare their bioavailability in various tissues after dosing. The rats had catheters placed in the mesenteric lymph vessel and the portal vein for sampling during the day of dosing. Rats were allowed to recover, and on the day of dosing, were dosed by gavage with equimolar doses of EtEPA at 1.55 mg/kg body weight. They were randomly assigned to three EtEPA dosing groups: (1) unadulterated EtEPA (n=12); (2) EtEPA and low-dose lymph-releasing (LR) compounds at a 4:1 ratio by weight (i.e., 4 parts EtEPA per 1 part LR compounds) (n=12); and (3) EtEPA and high-dose LR compounds at a 1:1 ratio by weight (i.e., 1 part EtEPA per 1 part LR compounds) (n=12). The LR compounds (i.e., phospholipids and/or emulsifiers) used in this experiment are shown in Example 1. Lymph and portal vein blood were collected before dosing and then hourly for 6 hours, whereupon the animals were sacrificed, and other tissues collected. Results were analyzed by non-parametric pharmacokinetic (PK) parameters, and the chief comparison between groups was incremental (i.e., net) area under the curve (incAUC). Levels of EPA and other fatty acids of interest were determined by mass spectrometry protocols optimized for the type of lipid studied. Phospholipid esters were assayed by UPLC-Qoadrupole/Orbitrap Q Exactive MS. Cholesteryl esters were assayed by tandem MS (LC-MS/MS). Triacylglycerol esters were assayed by GC-High Resolution MS. Fatty acid methyl esters were assayed by GC-MS.

As shown in FIG. 1A, there is a favorable 140% increase of total EPA free acid levels in lymph fluid for LR-EtEPA over EtEPA over six hours post dose. This result is highly statistically significant.

Keeping EtEPA doses equimolar between the treatment groups and varying only the amount of additives (e.g., phospholipids and/or emulsifiers), the difference between a one-to-one mixture of EPA and additives by weight and a four-to-one mixture was also statistically significant at a p-value of 0.002 (FIG. 2). Increasing amounts of additives relative to EtEPA enhances uptake through the lymph fluid.

Assuming equal EPA release by enterocytes, every mole of increased lymphatic EPA from LR-EtEPA vs EtEPA indicates a mole that was diverted from the portal system by the additives. Not only so, but this also occurred in a dose-responsive manner, such that keeping the EtEPA dose constant, increasing the dose of the additive alone effected higher lymphatic EPA levels. Thus, diversion toward the lymphatic system is further supported by dose-response effects of the phospholipids (e.g., lecithin) and/or emulsifiers.

### Example 3: Comparison of Multiple-Dose Equimolar EtEPA and LR-EPA

This experiment consisted of multiple-daily dosing of Long-Evans rats by gavage in two cohorts that varied by length of exposure: (1) a 7-day exposure, and (2) a 21-day exposure. Rats were randomized to the following treatment groups to receive equimolar amounts of interventional fatty acids (3.1265 mmol FA/kg/day):
1. Ethyl oleic acid (OA): 3.1265 mmol OA/kg/day;
2. Ethyl-GLA, ethyl-EPA, and ethyl-DHA (GLA+EPA+DHA or G+E+D): 3.1265 mmol total FA/kg/day;
3. Ethyl-EPA (EtEPA, E-EPA, or IPE): 3.1265 mmol EPA/kg/day;
4. Ethyl-EPA+lecithin (LC) at a 4:1 weight ratio (EPA+LC 4:1, LR-EtEPA, or 1×LR-EtEPA): 3.1265 mmol EPA/kg/day; and
5. 1.5× (Ethyl-EPA+LC at a 4:1 weight ratio) (1.5× (EPA+LC 4:1) or 1.5× LR-EtEPA): 4.68975 mmol EPA/kg/day.

For reference, the 1.033 g IPE/kg/day dose in the Long-Evans rat is comparable to a 10 g/day dose in a human. Since Vascepa® (>96% IPE) is dosed at 4 g/day clinically, this is 2.5-fold the typical dose. This dose was selected as a reasonable dose to consider for the treatment of diseases including SIRS, sepsis, and ARDS. Upon sacrifice at the end of the dosing period, several tissues were collected. Levels of EPA and other fatty acids were determined by the same methods described above in Experiment 2. The LR compounds (i.e., phospholipids and/or emulsifiers) used in this experiment are shown in Example 1.

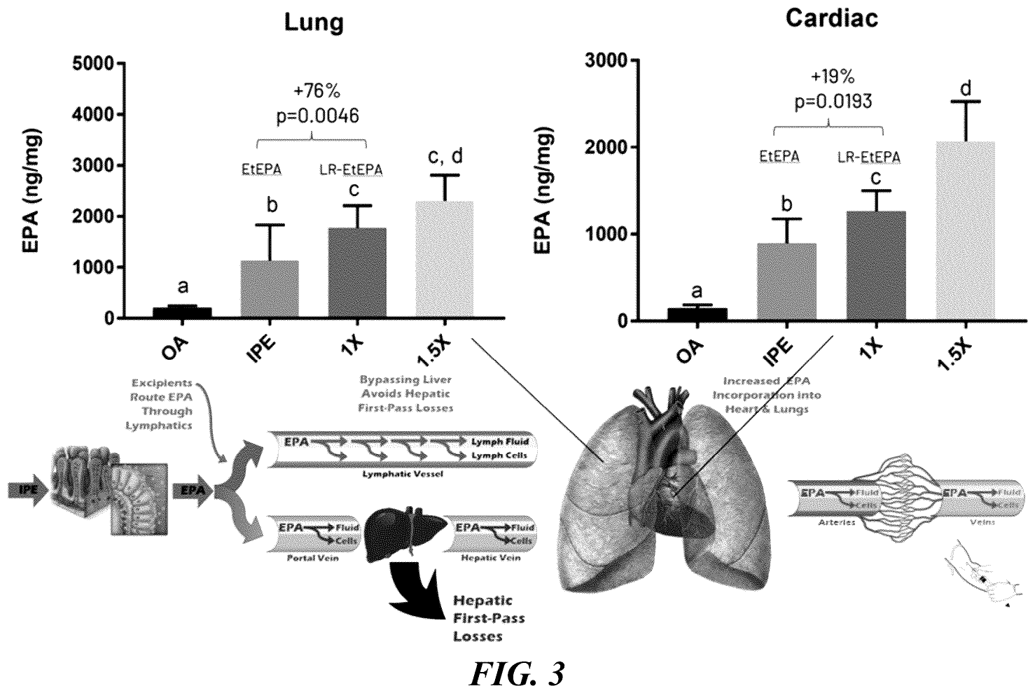

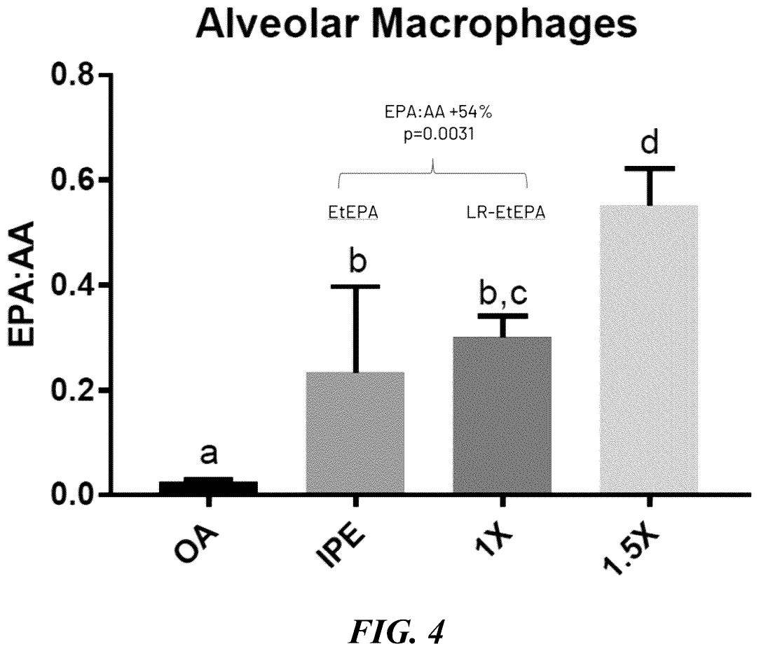

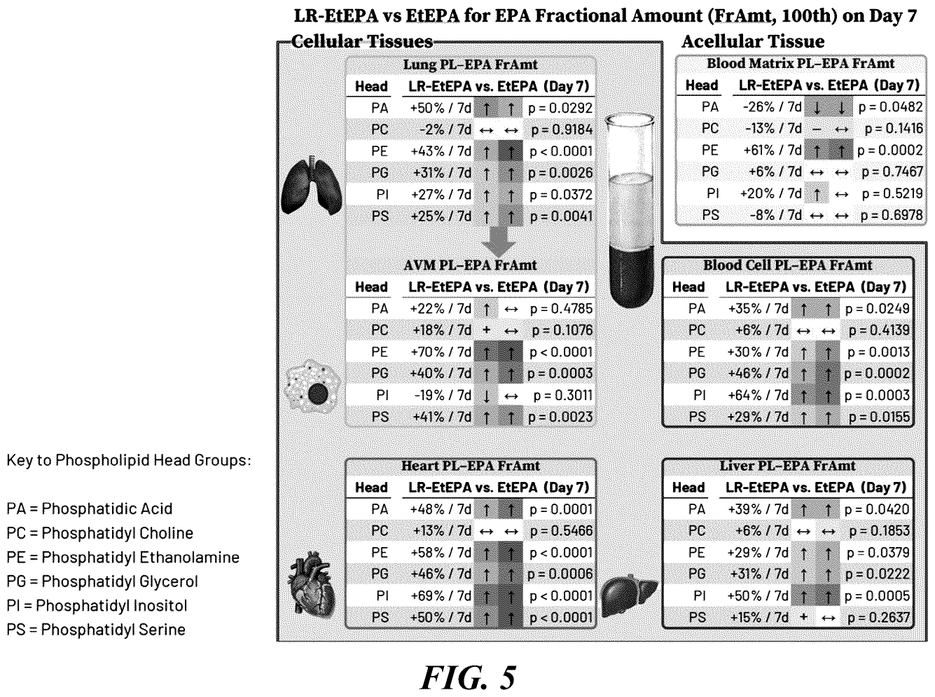

As shown in FIGS. 3-4, LR-EtEPA was superior to plain EtEPA in enriching lung and heart tissues with EPA at equimolar doses of IPE. In the lung, after 7 days of daily dosing by gavage, there is a 76% increase in total EPA favoring LR-EtEPA which is statistically significant (FIG. 3), and there's a 41% increase in the EPA to arachidonic acid (AA) ratio (data not shown). The heart had a statistically significant 19% increase in EPA uptake (FIG. 3), and there was a 30% increase in the EPA to AA ratio (data not shown). LR-EtEPA also raised the EPA/AA ratio in lung alveolar macrophages (AVMs) compared to an equimolar dose of plain EtEPA (FIG. 4). Thus, the enhanced uptake through the lymph fluid in turn enhances cardiac and pulmonary uptake of EPA.

Macrophages are a key immune cell in the lung and plays an important role in inflammatory diseases, including but not limited to, SIRS, sepsis, ARDS, interstitial lung disease, and pneumonia. LR-EtEPA robustly increased the EPA to AA ratio in the immune cells after 7 days (FIG. 4). EPA levels were slightly decreased but there was a much greater decrease in AA. Without wishing to be bound by theory, EPA appears to be displacing AA from the immune cells resulting in a higher EPA to AA ratio. One skilled in the art would expect this result to be very favorable for treating inflammatory diseases of the lung such as SIRS, sepsis, ARDS, and pneumonia.

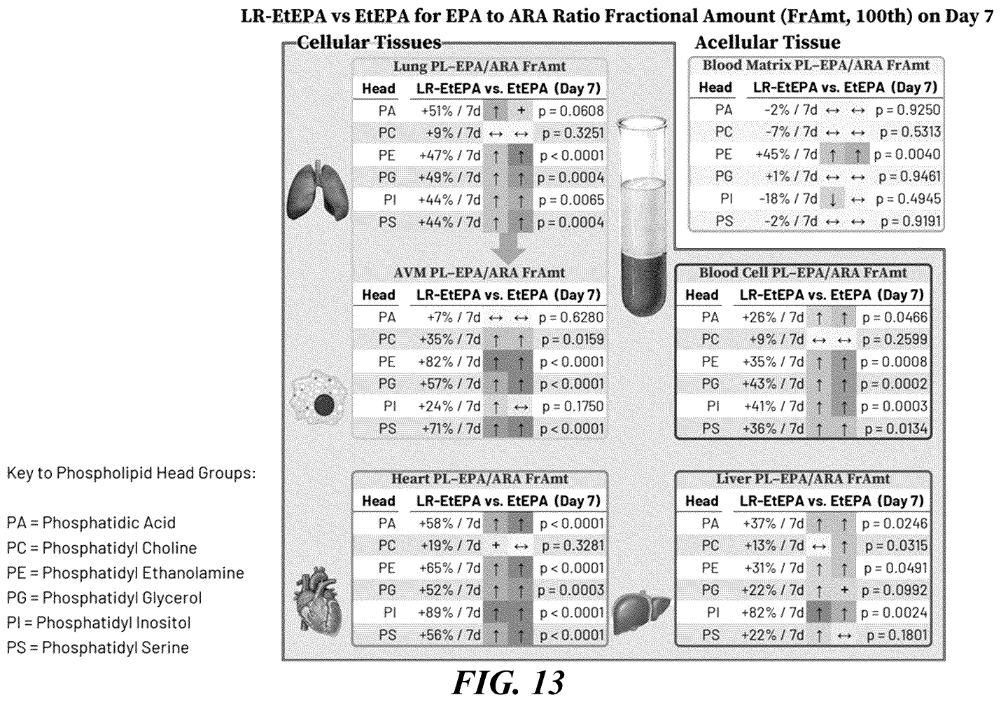

The phospholipid-EPA (PL-EPA) composition can be subdivided into phospholipid levels according to the six classic types, including phosphatidylcholine and phosphatidylethanolamine, which are the two most abundant of the phospholipids in cell membranes in mammals. Most of the uptake is as phospholipids in the membranes. The data is represented in a “heat map” (FIG. 5). The heat map is using intensity of color to express greater differences for LR-EtEPA versus EtEPA. FIG. 5 represents the percent increase or decrease for LR-EtEPA and the second column of the heat map is looking at p-values. The most robust findings have the darkest color in both of the columns. In the lung, a robust numerical increase is shown for LR-EtEPA as percent differences and very robust p-values. Notably, phosphatidylcholine (PC) appears to lag the others at seven days. It may be the case that PC “catches up” at 21 days. Figuratively speaking, PC is the largest “pool” of the phospholipid types and one skilled in the art would expect PC to lag. A large pool of PC means that the turnover rate of the pool will be slower. Similarly, a heat map for PL-EPA to AA (or ARA) ratio in presented in FIG. 13.

Within the lungs, PL-EPA levels in the alveolar macrophages were analyzed. Several of the phospholipid types increased both in number and by p value. A similar result was observed in heart tissue. PC lagged at seven days, but for other phospholipids, robust increases were seen. There are also significant increases for several of the phospholipid types in lung. The cellular component of blood had very robust increases in EPA phospholipids according to the heat map as evidenced by the percent changes and the p values. By comparison, significant changes occurring in the acellular part of blood (i.e., plasma) are sparse, consistent with a more robust effect on cellular vs acellular tissue. As a prominent exception, phosphatidylethanolamine (PE) was robustly increased in acellular blood matrix. Increased PE has biological significance, especially as blood PE is delivered to cells and cell membranes, because the PE moiety is more likely found in the inner cell membrane; as such, PE is more likely to have component fatty acids liberated by phospholipases, so that PE enriched with EPA is likely more efficacious than PC enriched with EPA for offsetting the ill effects of AA-derived oxylipins.

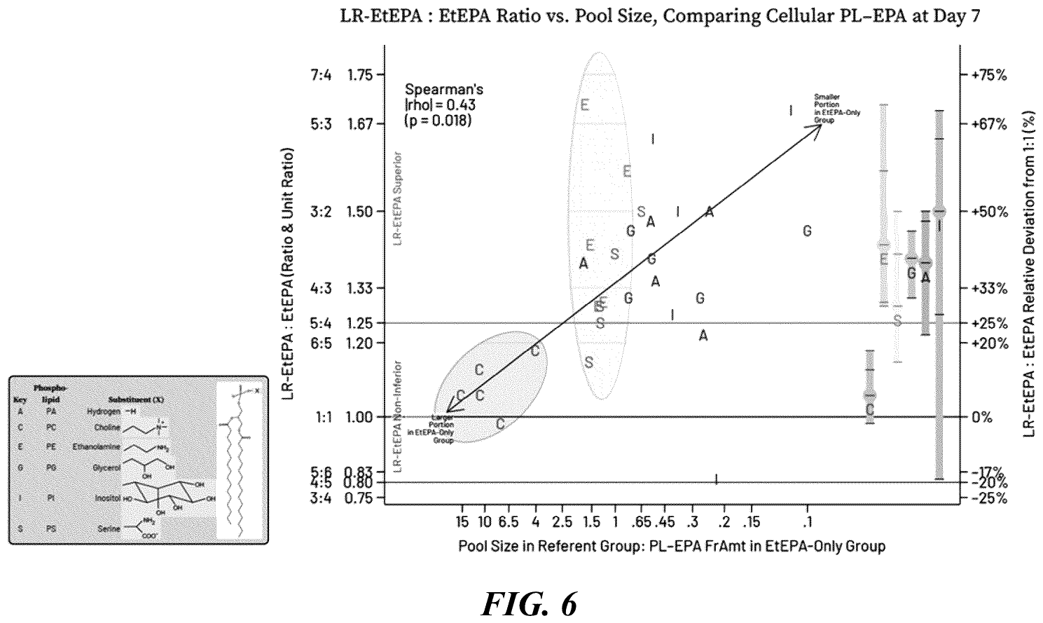

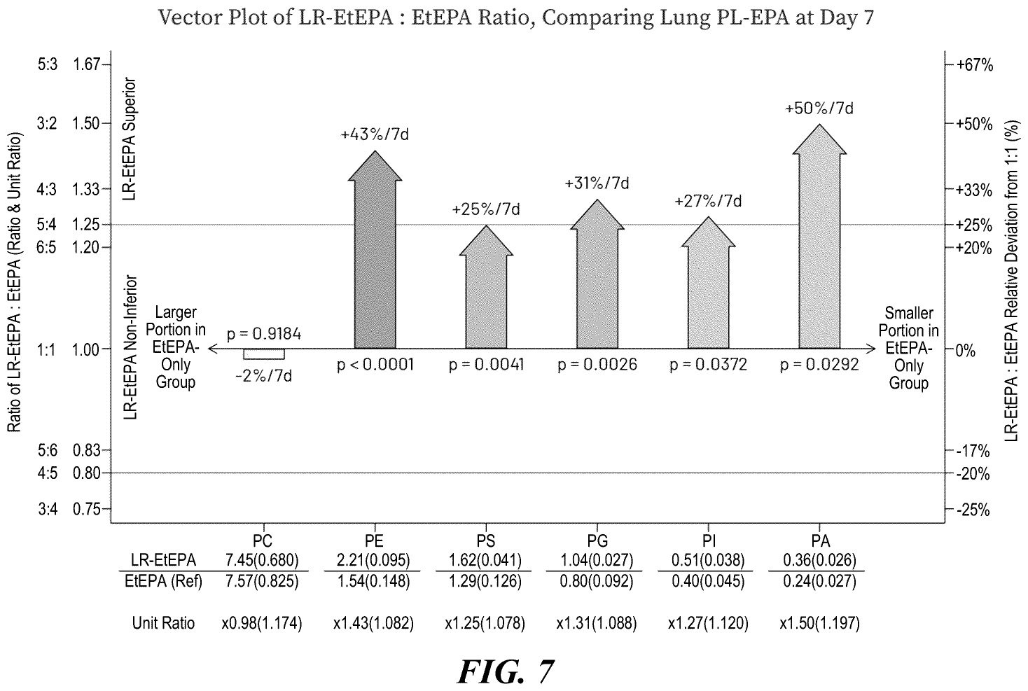

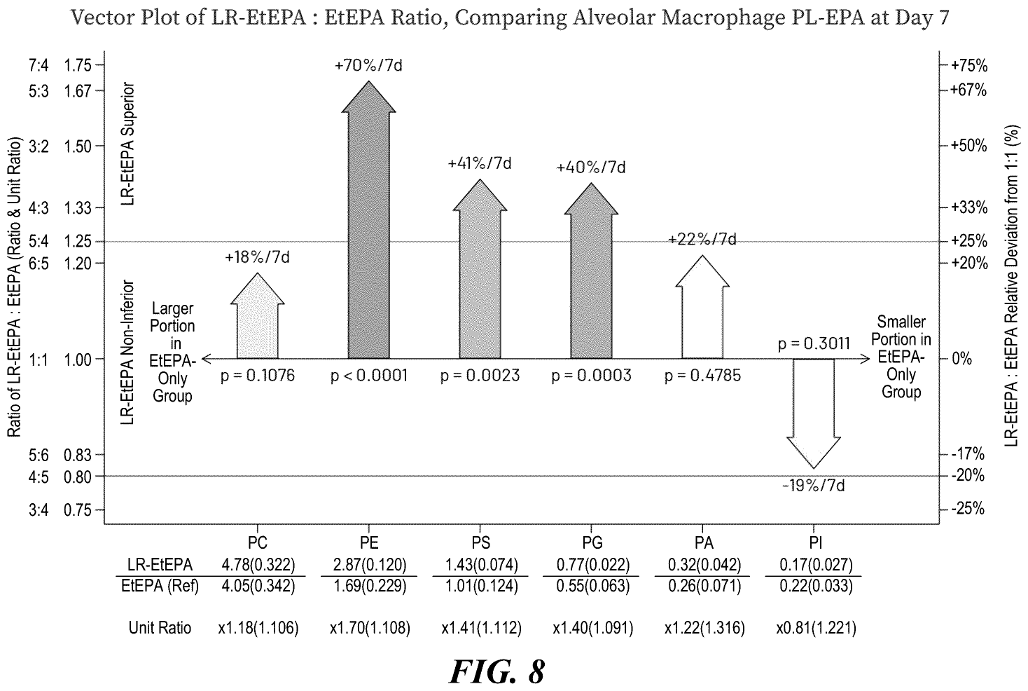

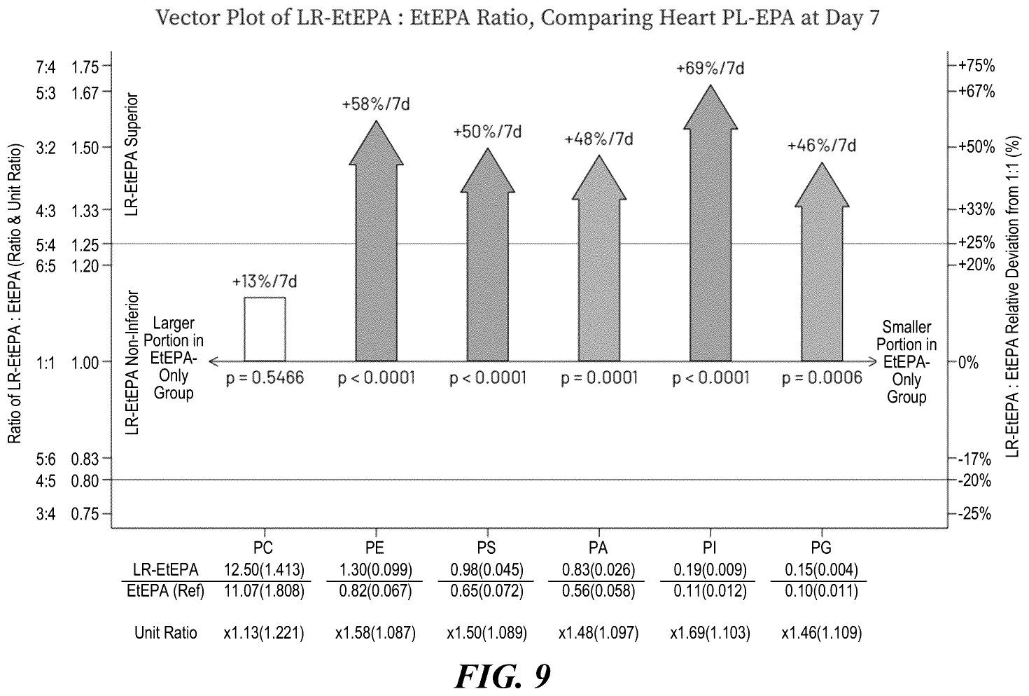

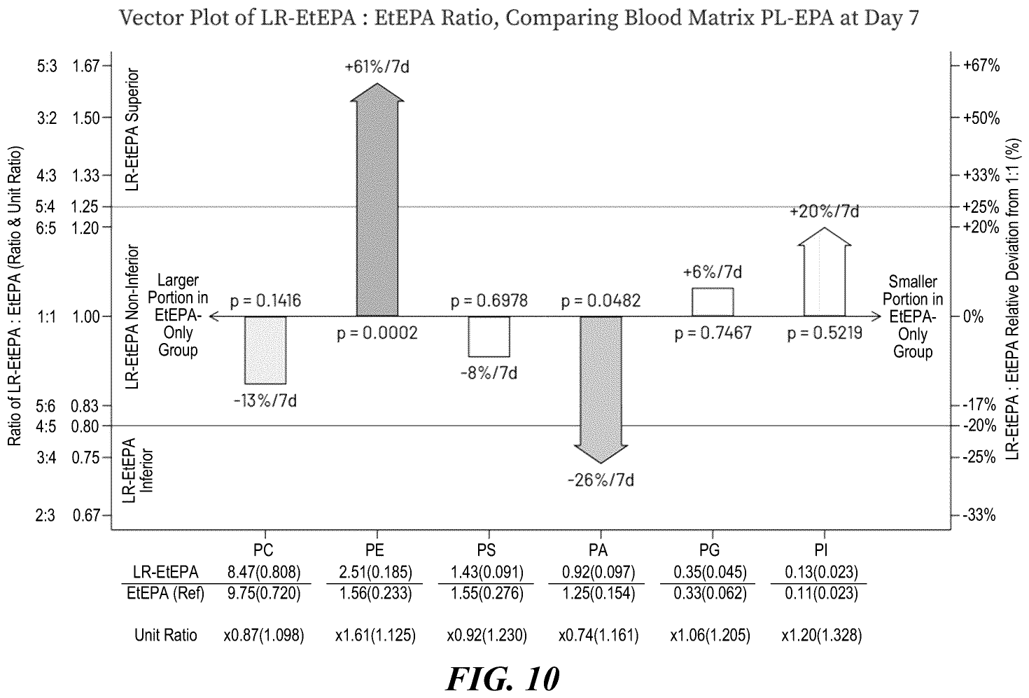

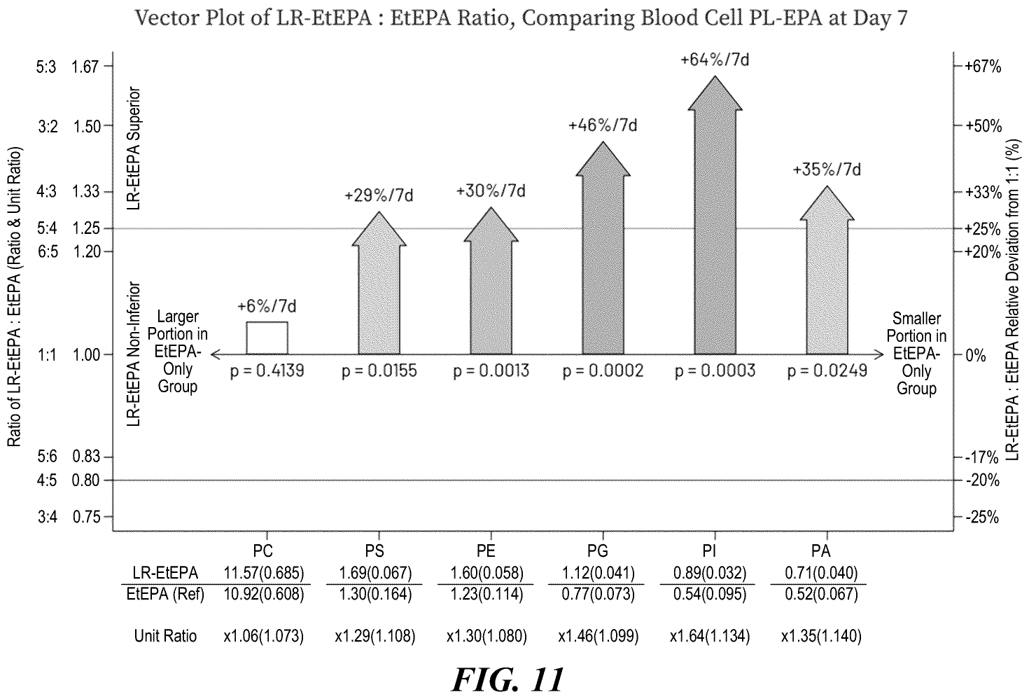

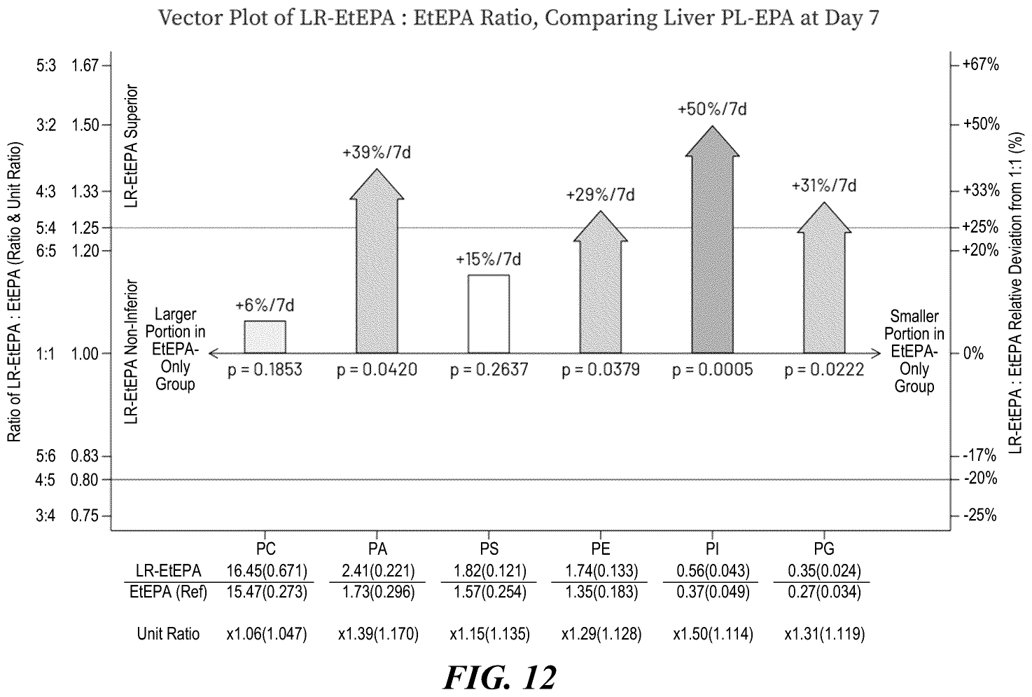

In FIG. 6, the five types of cellular PL-EPA were represented in a vector plot. Specifically, the five cellular PL-EPA types are lung-PL, AVM-PL, heart-PL, blood cell-PL, and liver-PL. The ratio of LR-EtEPA to EtEPA was plotted on the y axis. If the ratio is above 1.25, it is considered evidence of LR-EtEPA's biological superiority. If the ratio is between 0.8 and 1.25, LR-EtEPA is considered to be non-inferior. Most of the phospholipids are in the range of analytical superiority. Phosphatidylcholine is lagging behind at 7 days. By contrast, phosphatidyl ethanolamine was superior for LR-EtEPA in all five tissue/cellular-PL types. See also FIGS. 7-12.

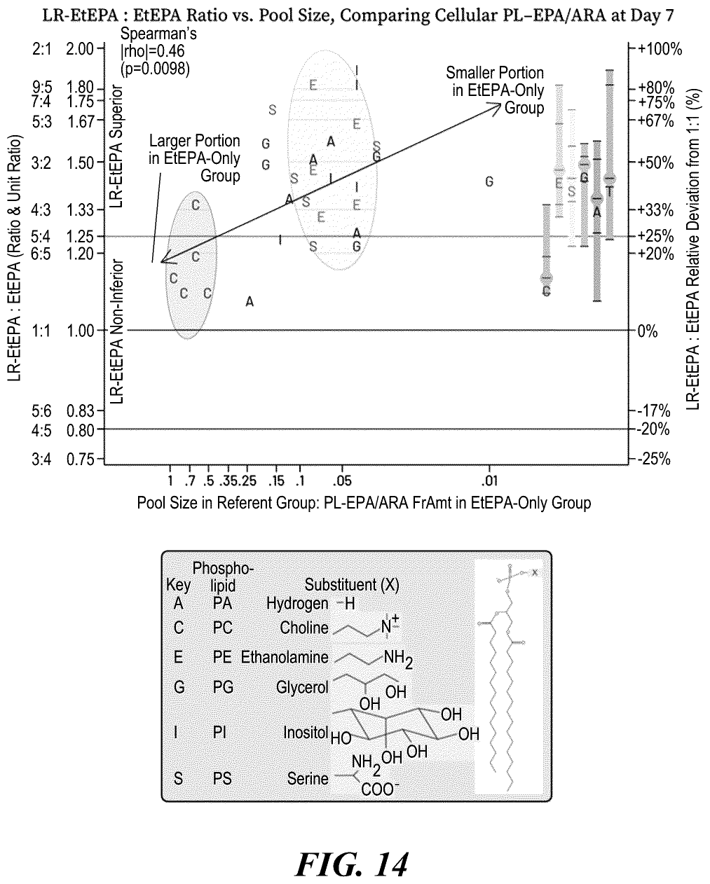

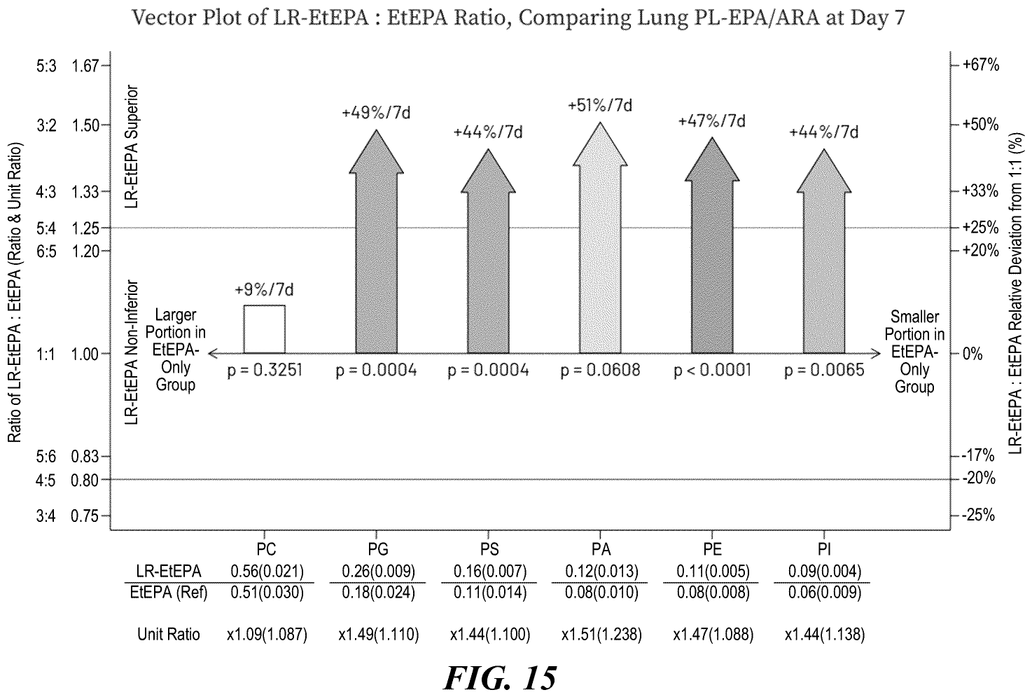

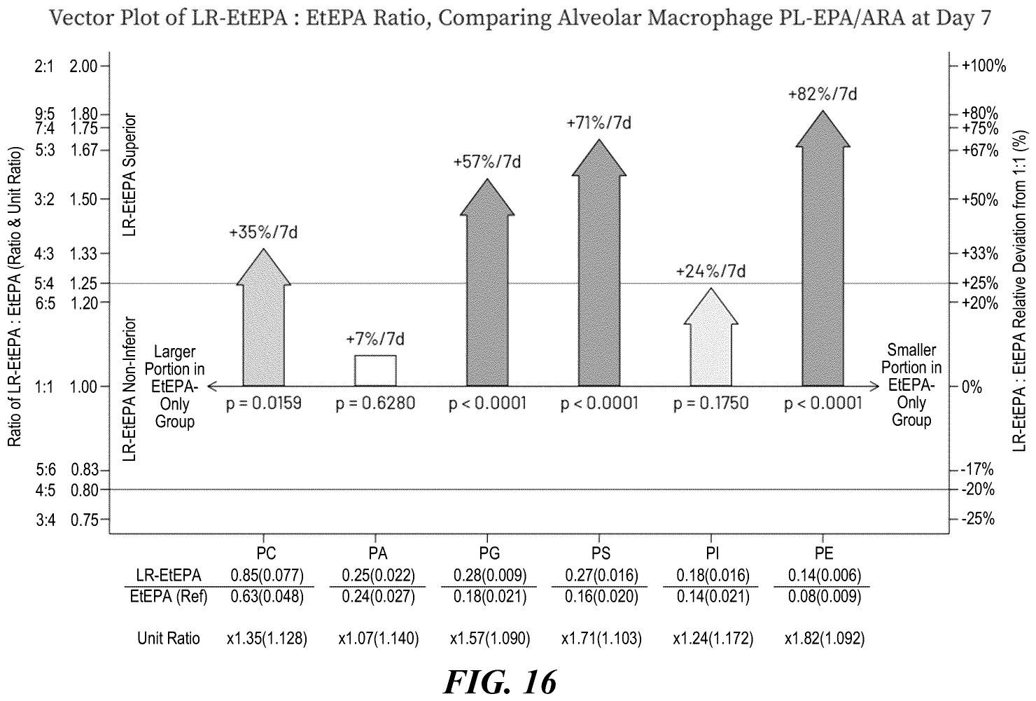

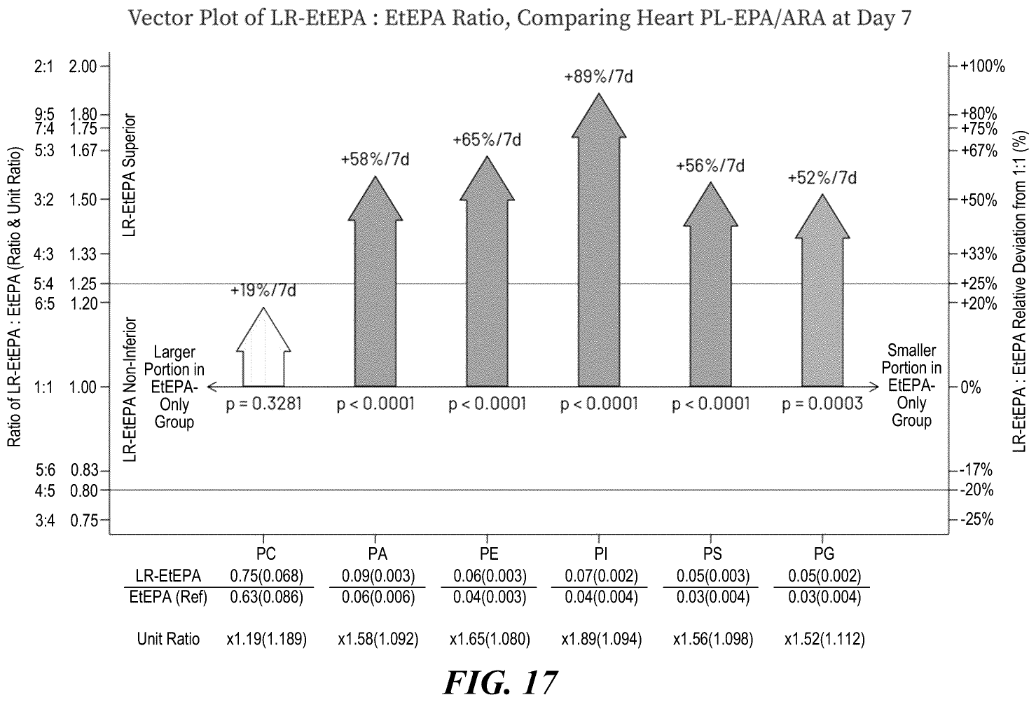

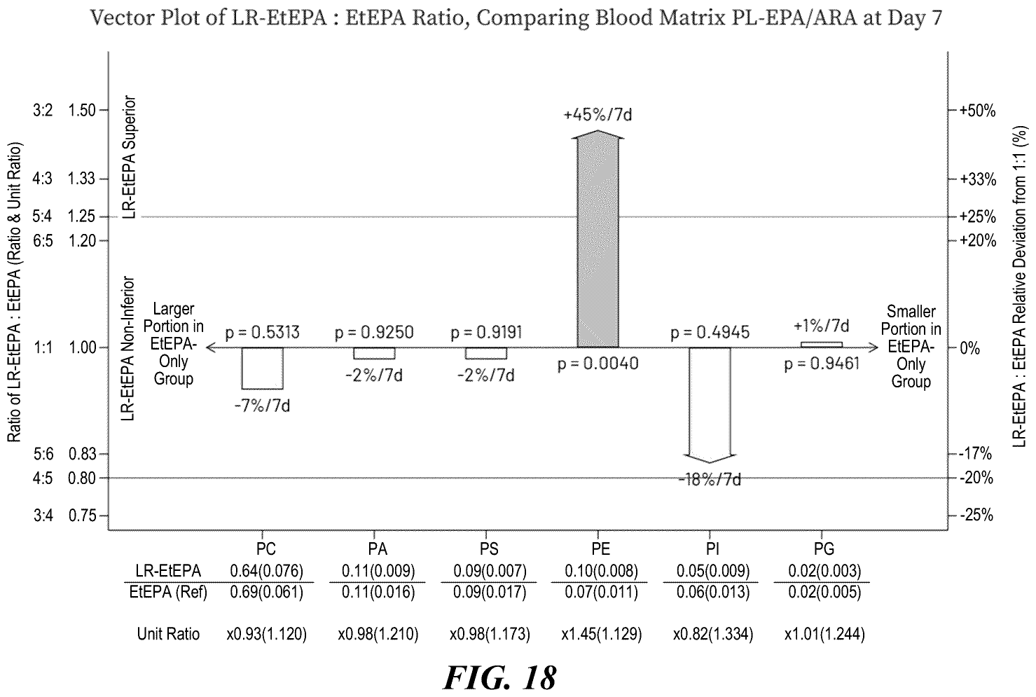

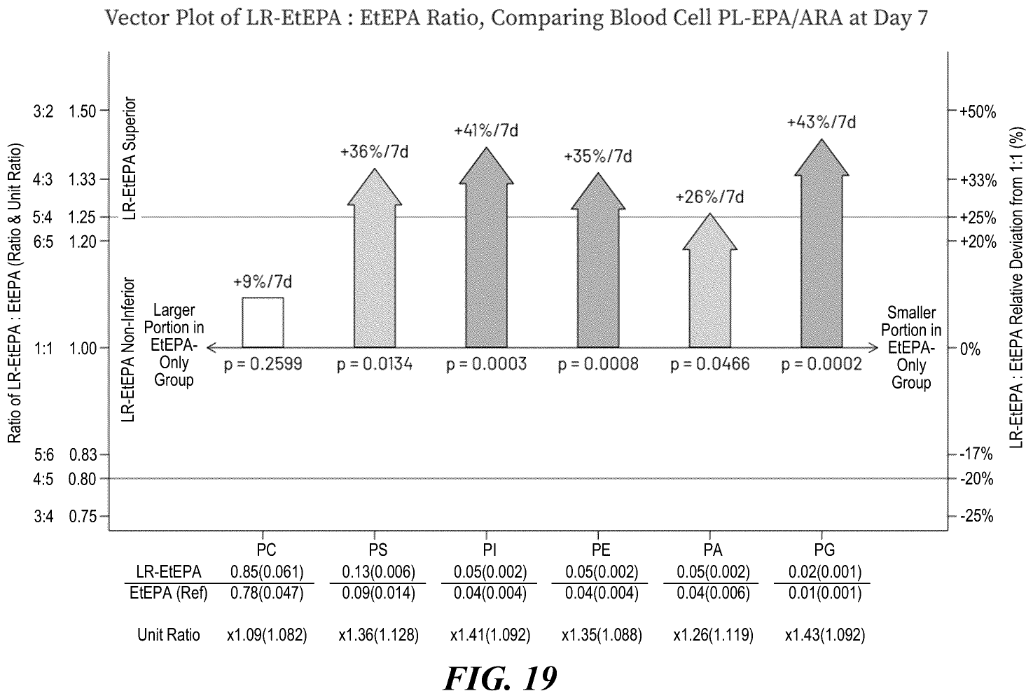

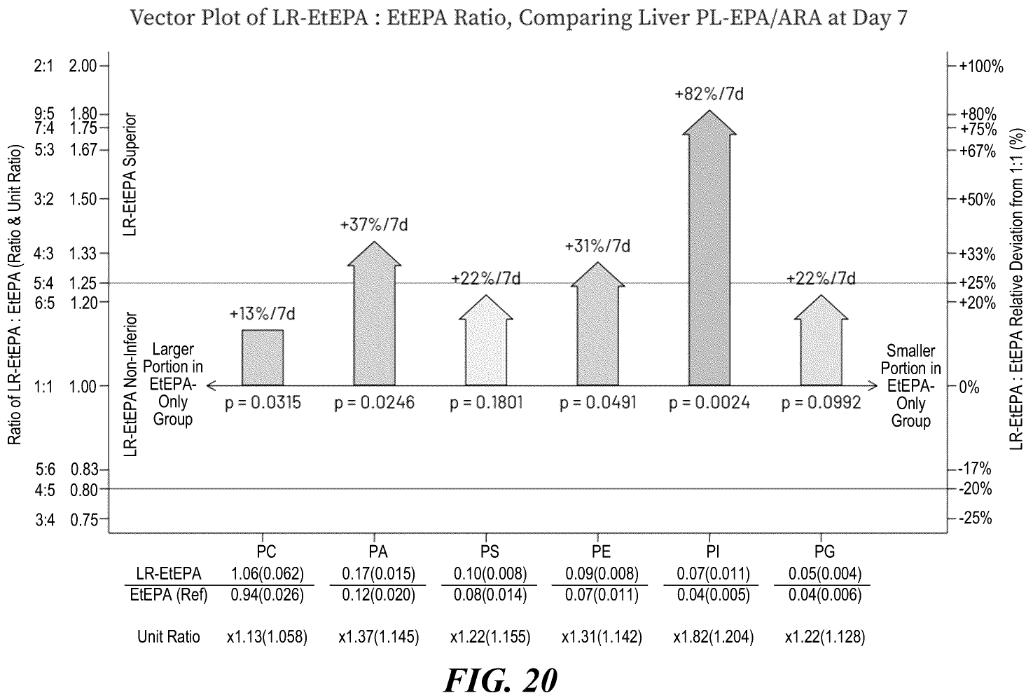

FIG. 14 is a vector plot illustrating the ratio of EPA to AA (or ARA). The plot demonstrates the very robust benefits favoring biological superiority of LR-EtEPA for a variety of cells. At Day 7, PC lags behind but due to the strong relationship between the pool size of the phospholipids, one might expect that PC will eventually “catch up” after 7 days. See also FIGS. 15-20.

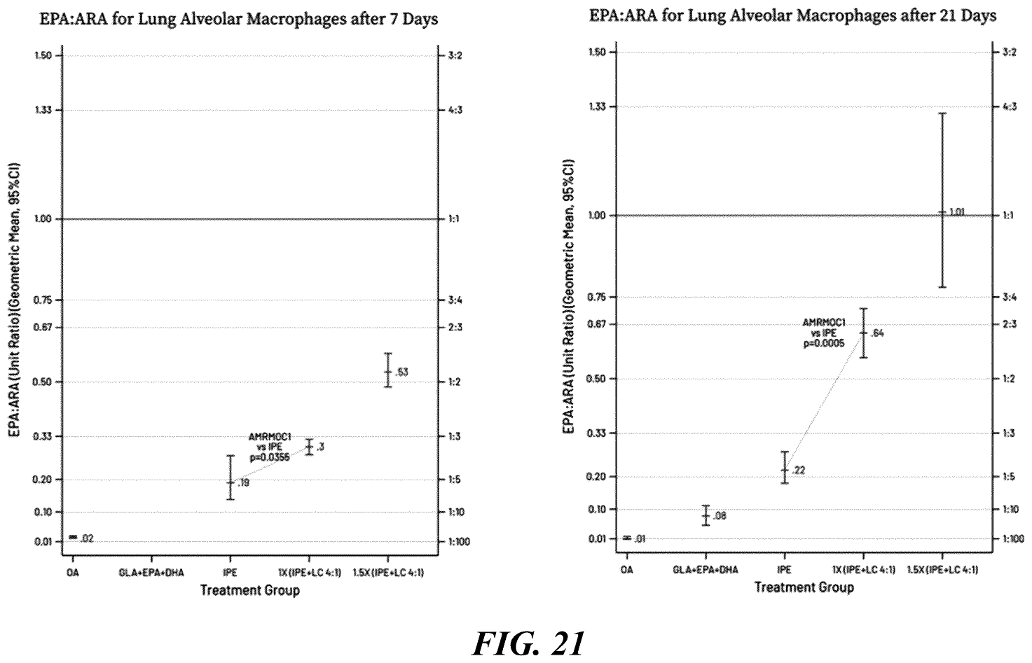

As shown in FIG. 21, the EPA:ARA ratio in lung AVMs at 21 days is several folds higher on LR-EtEPA vs IPE alone, even more so compared to at 7 days, supporting the superior therapeutic effects of the claimed composition/methods. Importantly, tripling the exposure duration has shown to increase AVM EPA:ARA in the LR-EtEPA groups (1× and 1.5×). In the 1× dose (equimolar EPA to IPE group), the AVM EPA:ARA went from 0.3 at 7 days to 0.64 at 21 days. Similarly, in the 1.5× dose (1.5×EPA vs IPE group), the AVM EPA:ARA went from 0.53 at 7 days to 1.0/=at 21 days. This demonstrates LR-EtEPA's ability to transfer EPA to cells, and particularly, to cells in the lung/heart system. The result further shows that a longer exposure provides more time for EPA to integrate into AVMs at their usual turnover rate.

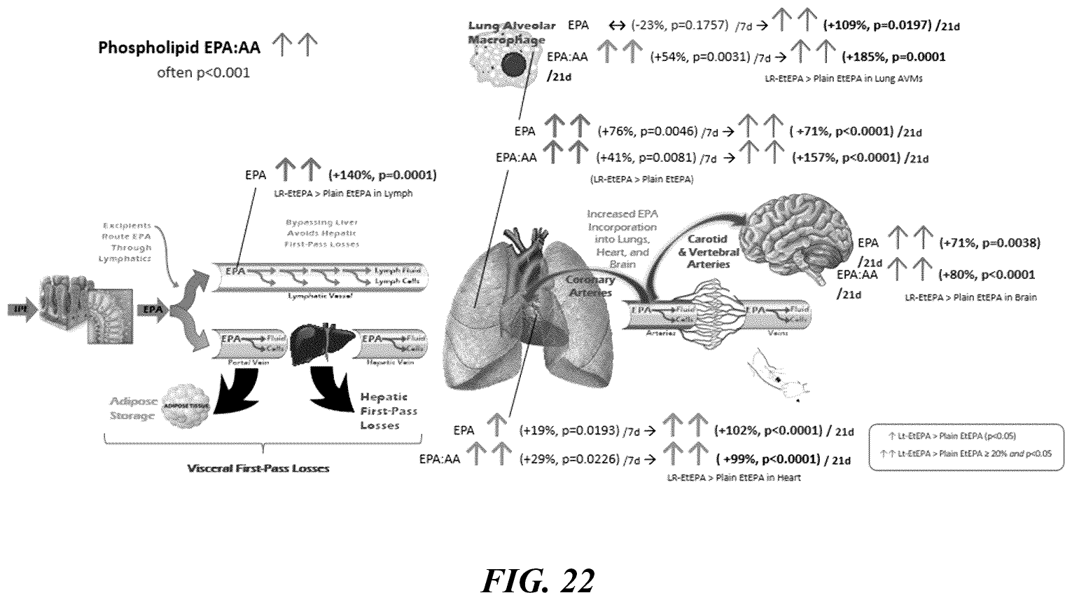

FIG. 22 summarizes the results from the study that compares LR-EtEPA to EtEPA in rats to (i) understand the delivery route to plasma and tissue for both drugs, and (ii) confirm superior cell/tissue uptake of LR-EtEPA compared to plain EtEPA. It shows that despite dosing EPA at equimolar doses, LR-EtEPA generally doubles EPA by the lungs, alveolar macrophages, heart, and brain.

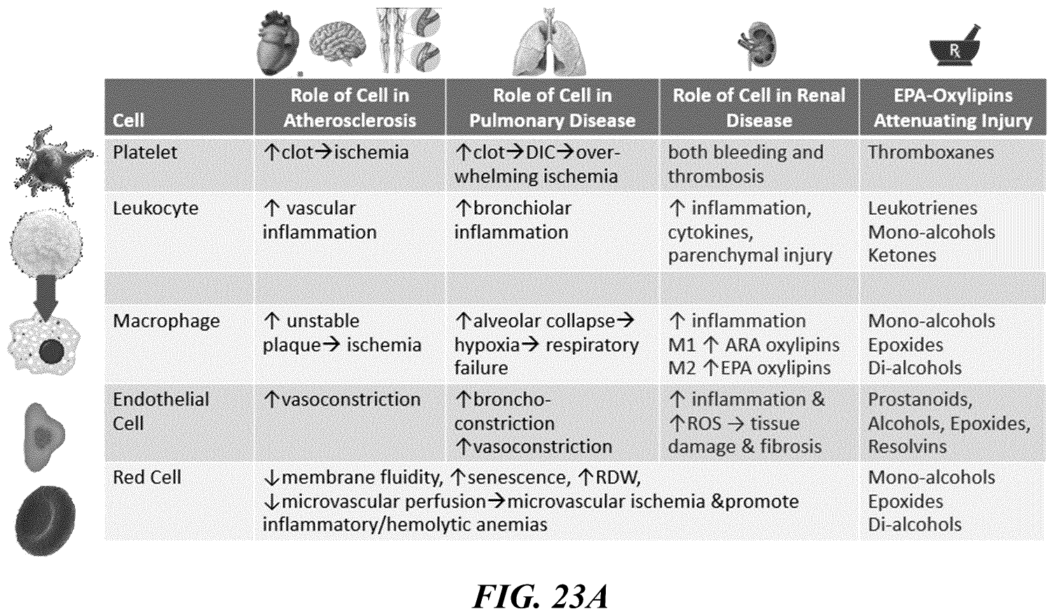

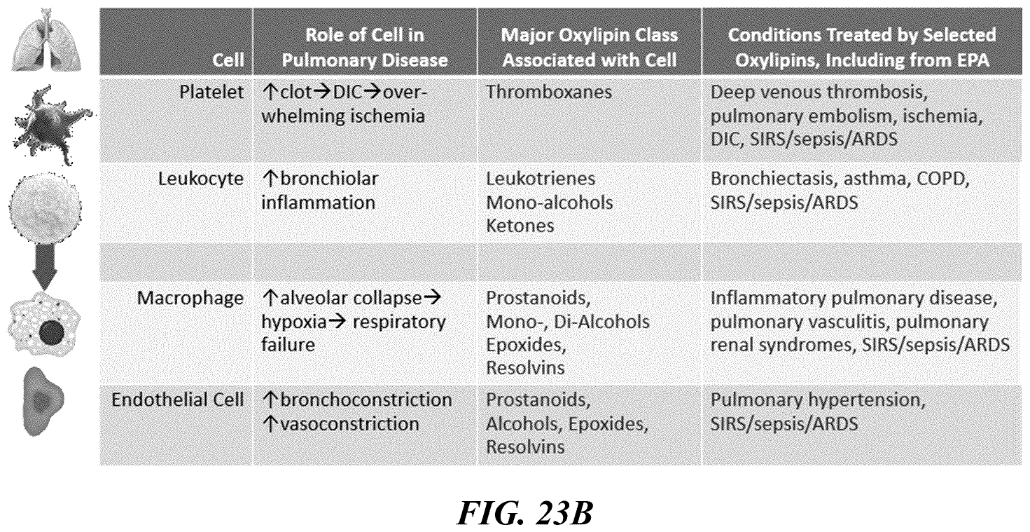

Taken together, the data provided herein demonstrate that LR-EtPA is biologically superior to EtEPA as a pharmaceutical composition for achieving EPA uptake. Biological superiority is herein assessed by the FDA analytical criteria. When comparing two formulations, analytical non-inferiority is defined as a ratio between 0.8 on the low end and 1.25 on the high end. If a 90% confidence interval is outside of that range, the result is supportive of either analytical superiority or inferiority. If the 90% confidence interval is within that range, the result is on only supportive of analytical non-inferiority. By these analysis criteria, LR-EtEPA is superior to EtEPA in targeting tissues and cells with EPA enrichment. As a consequence, one skilled in the art would expect LR-EtEPA to be superior to EtEPA in treating diseases that benefit from enhanced EPA update, including by the mechanisms shown in FIGS. 23A-23B.

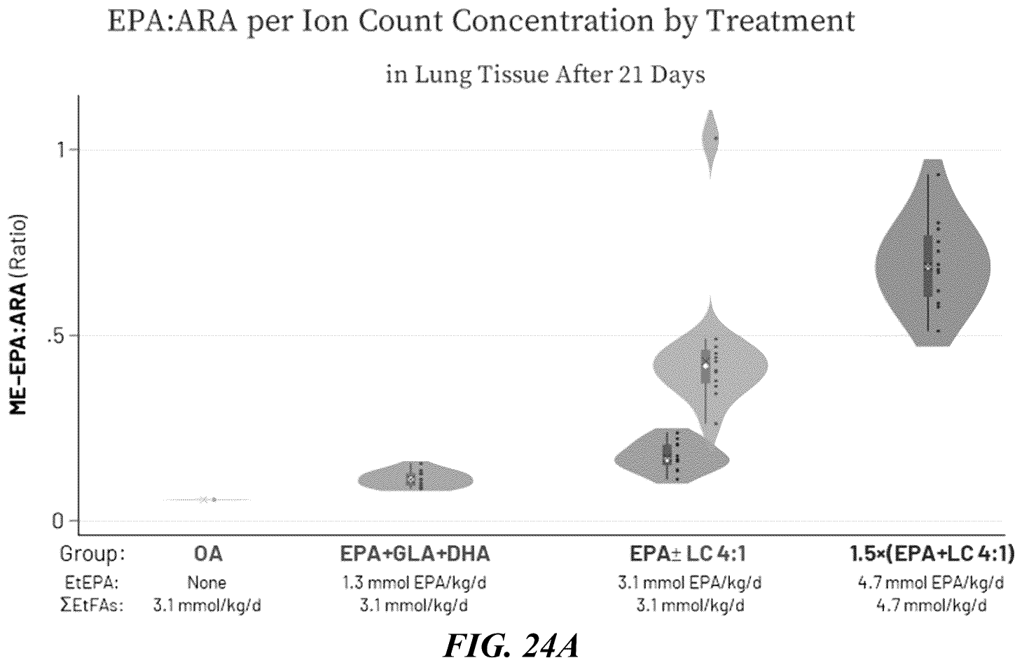

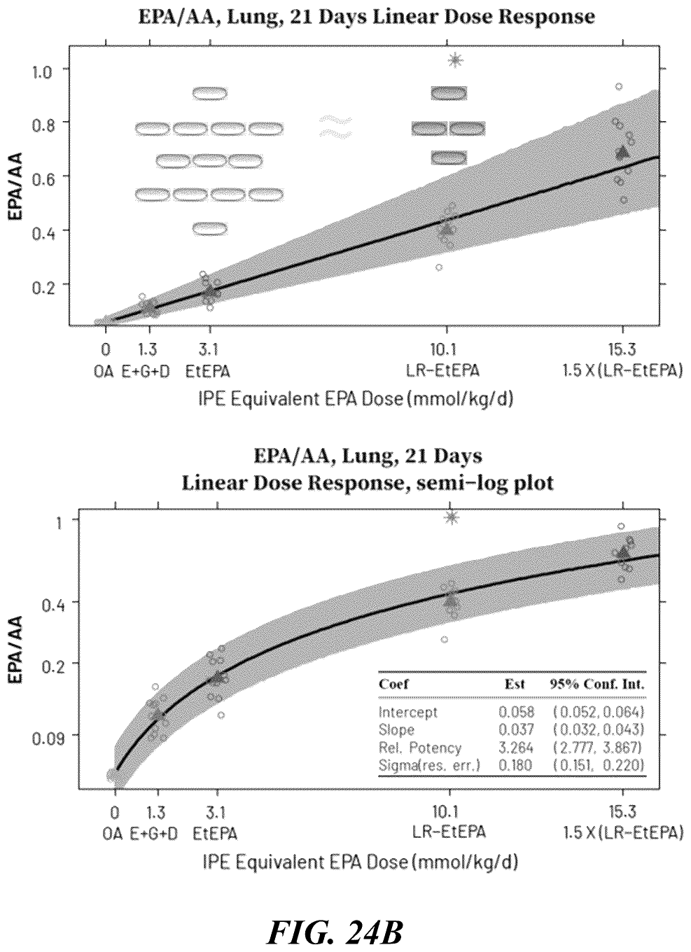

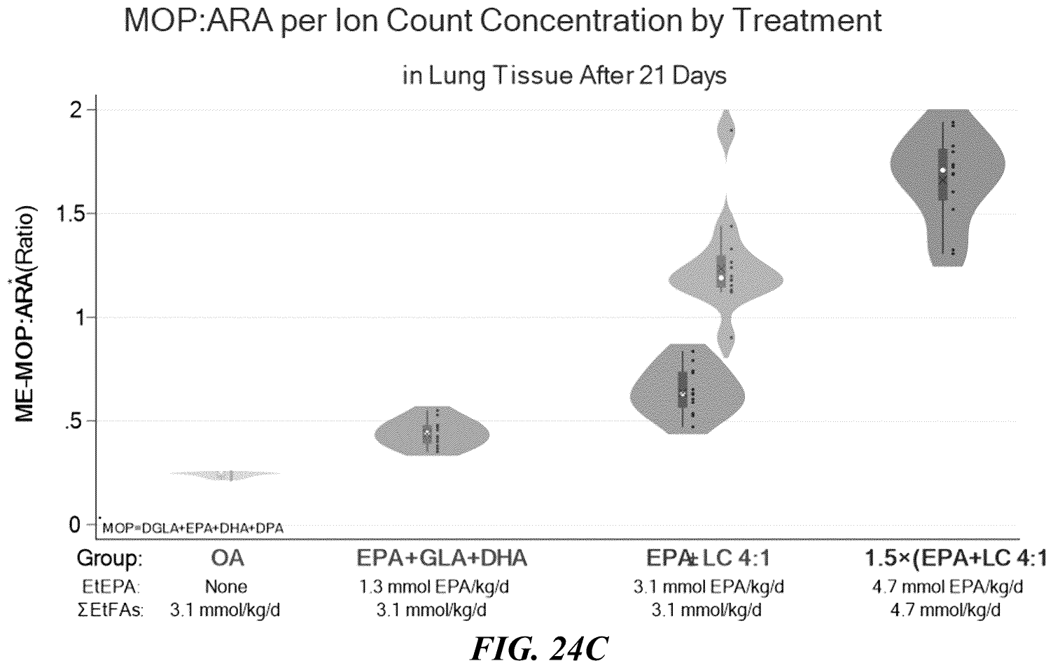

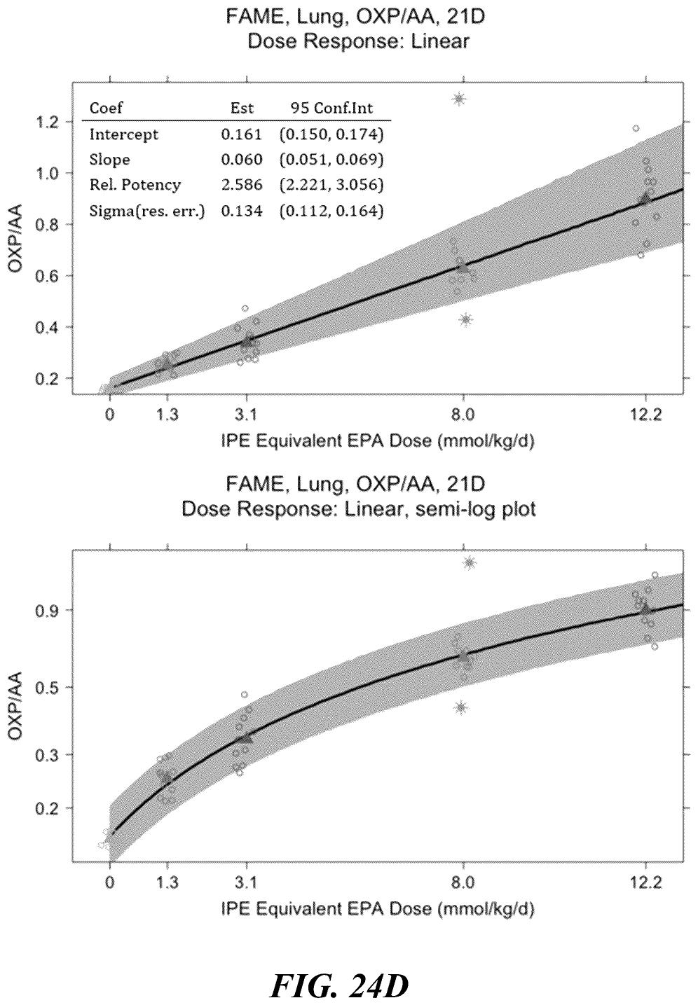

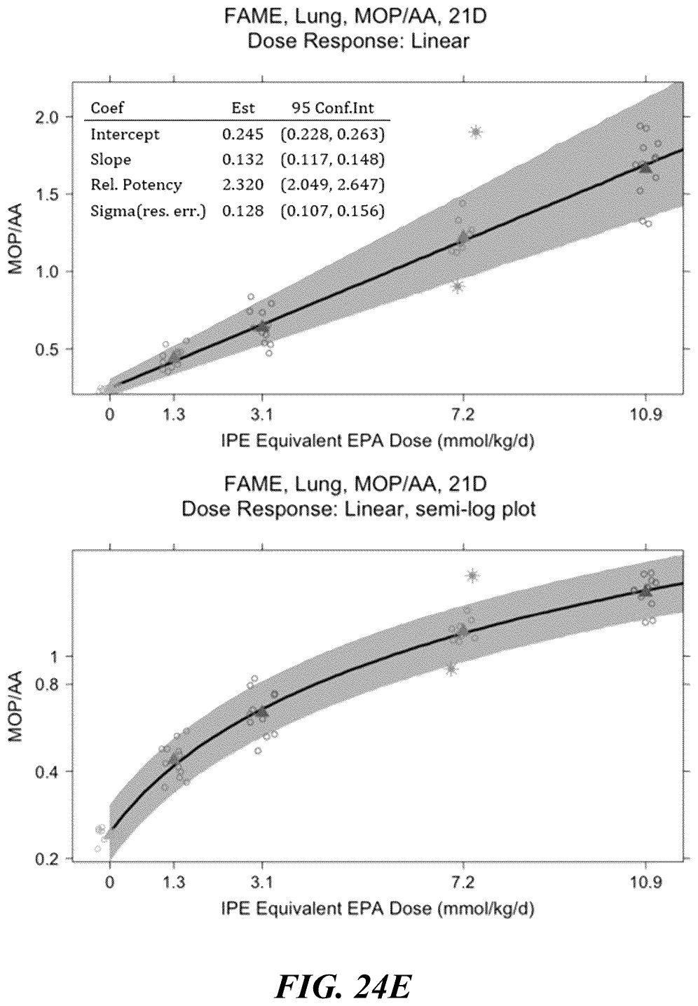

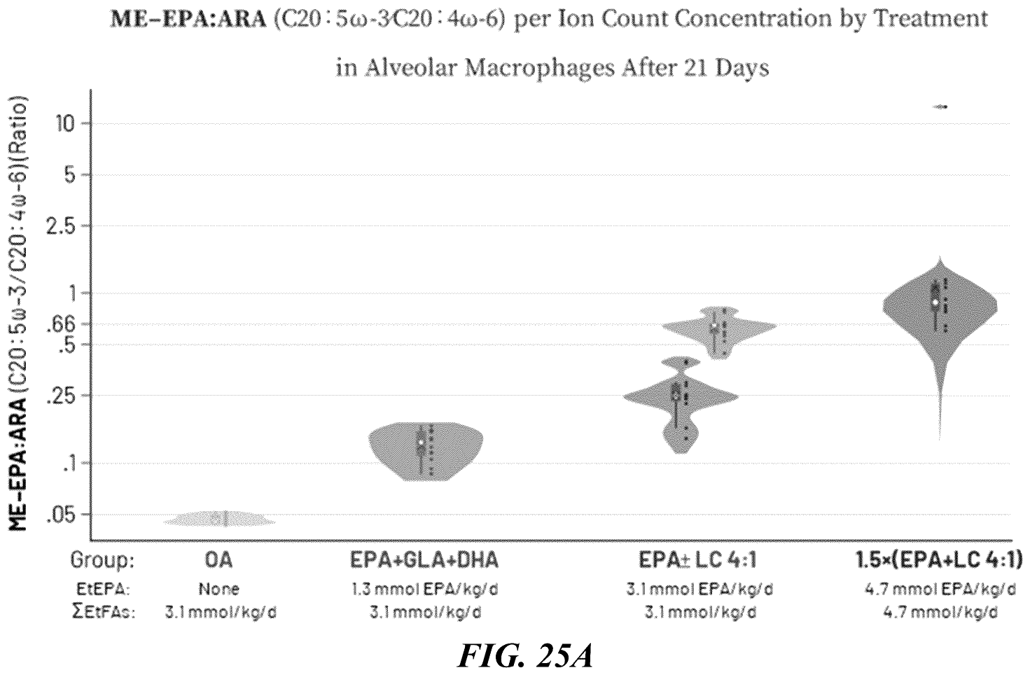

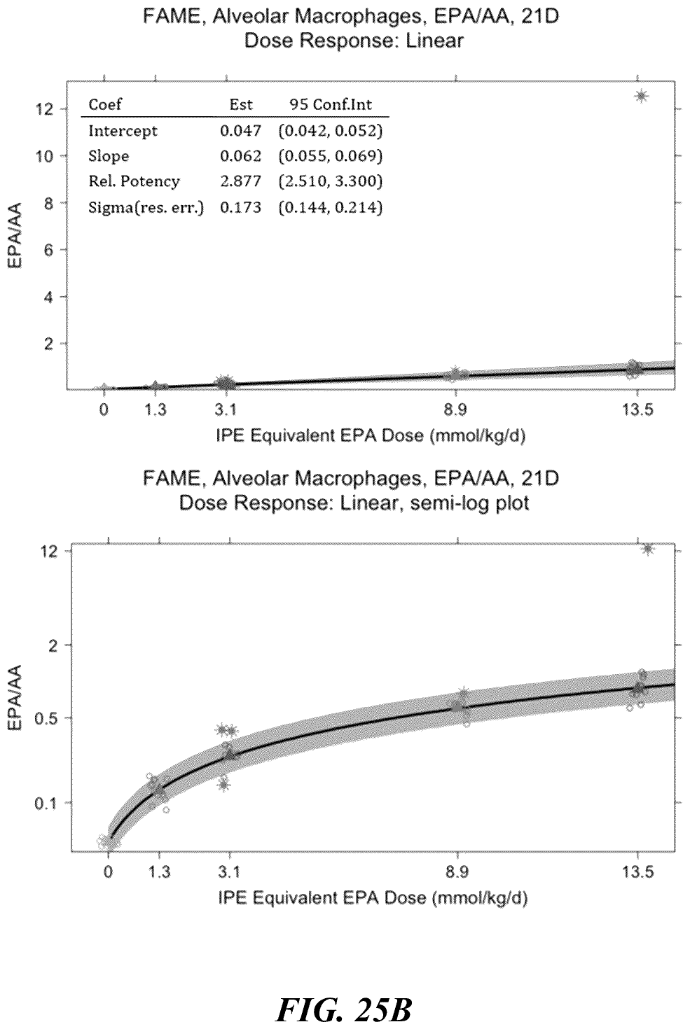

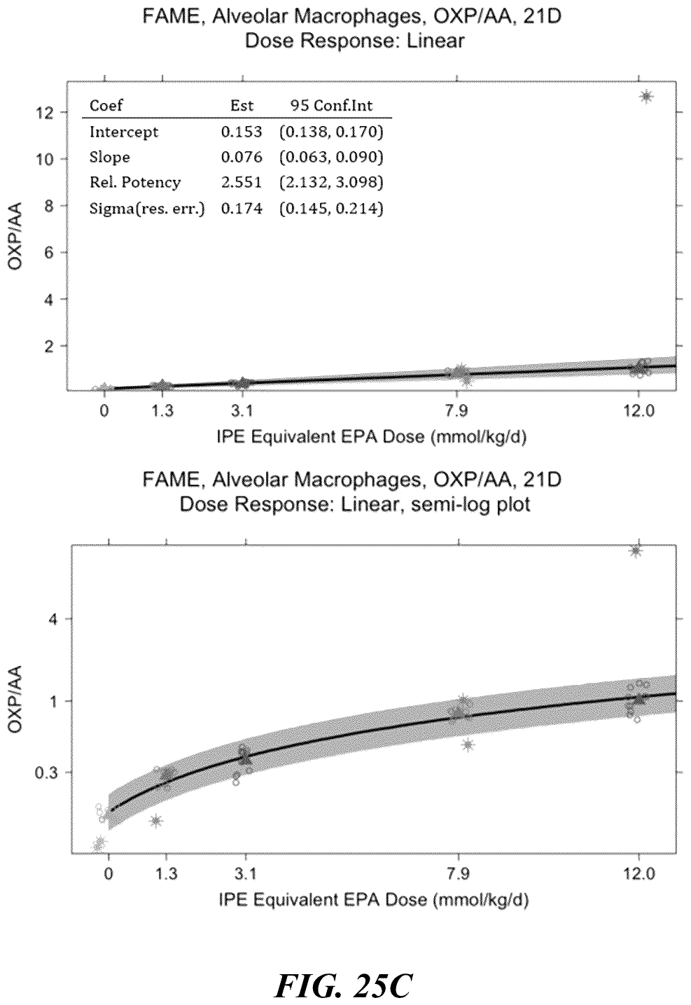

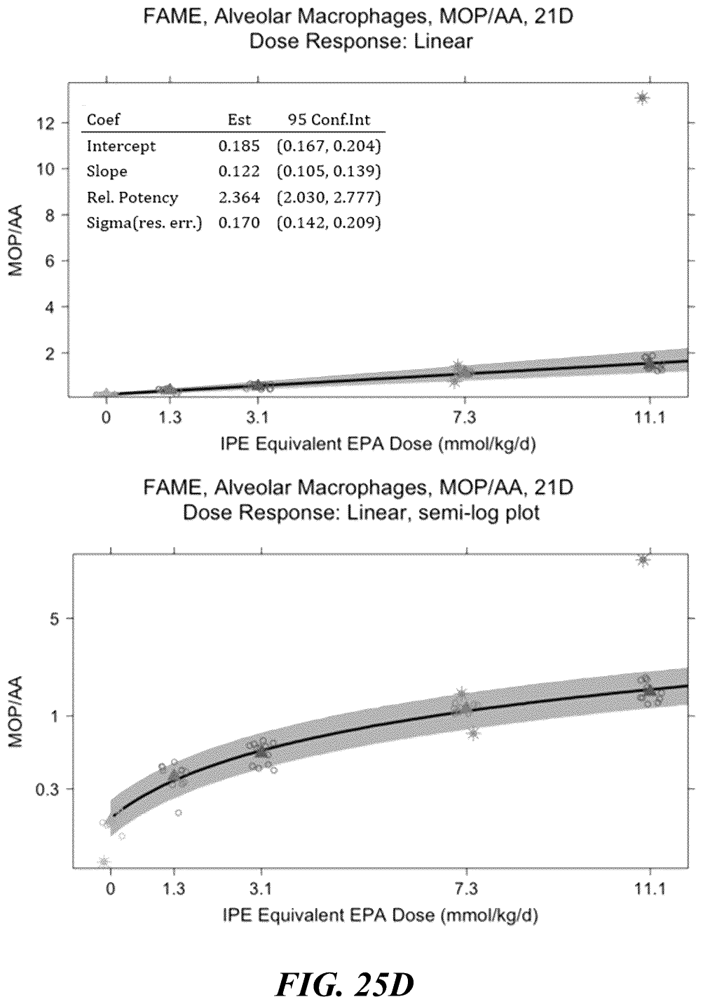

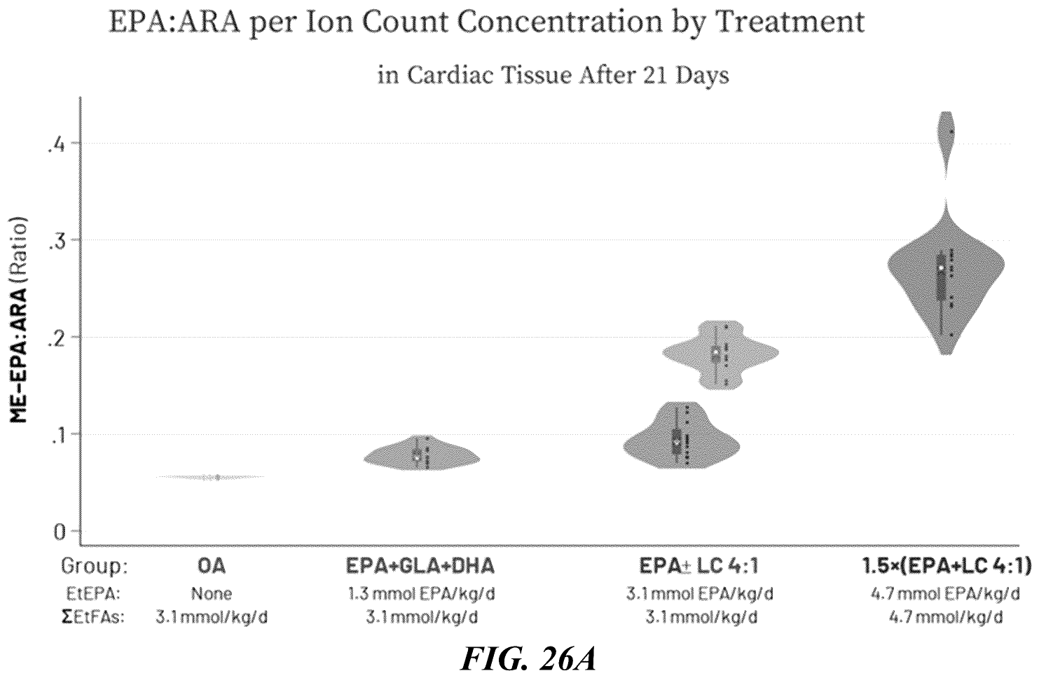

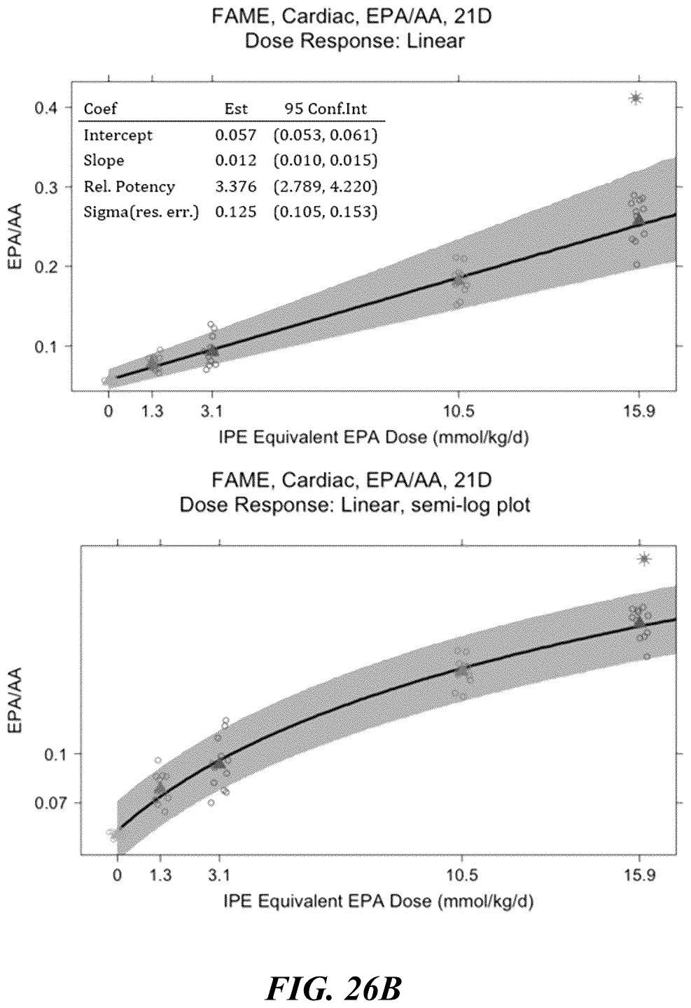

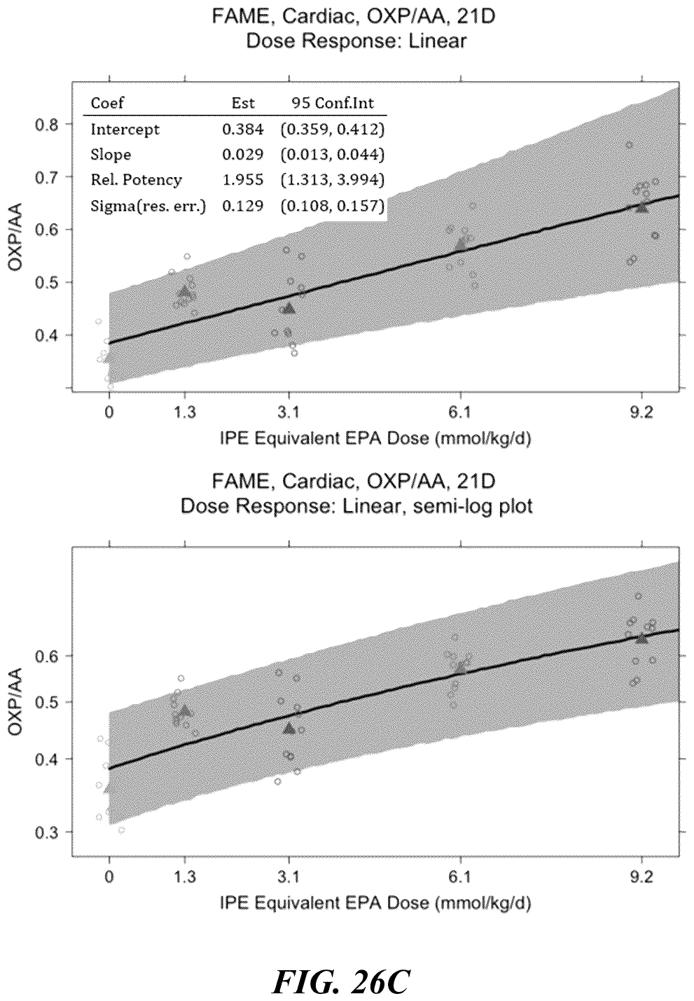

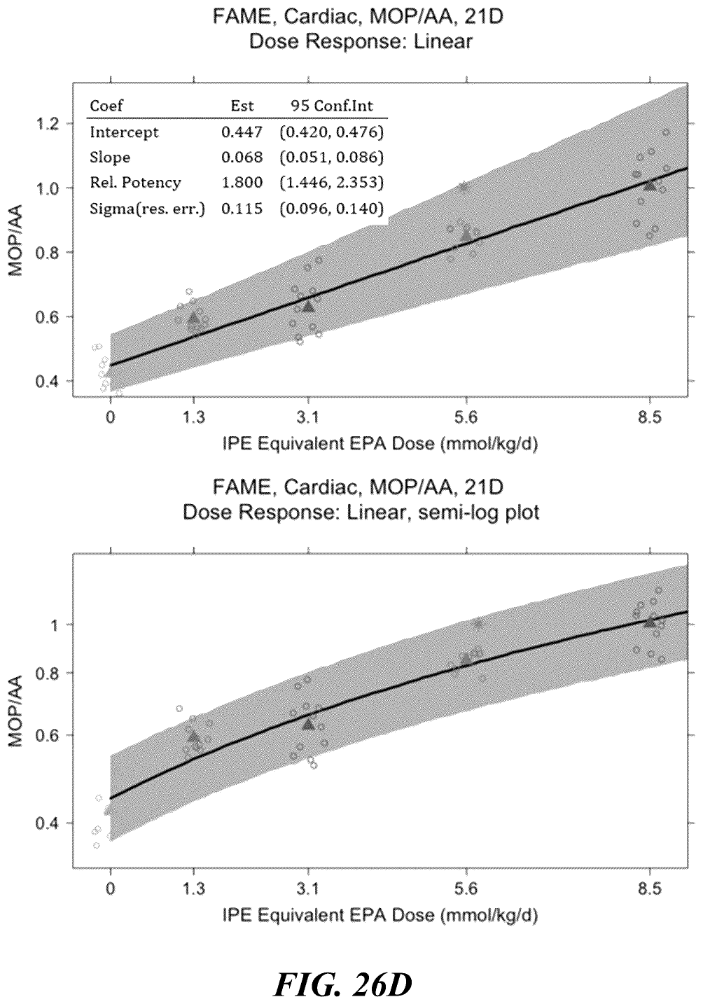

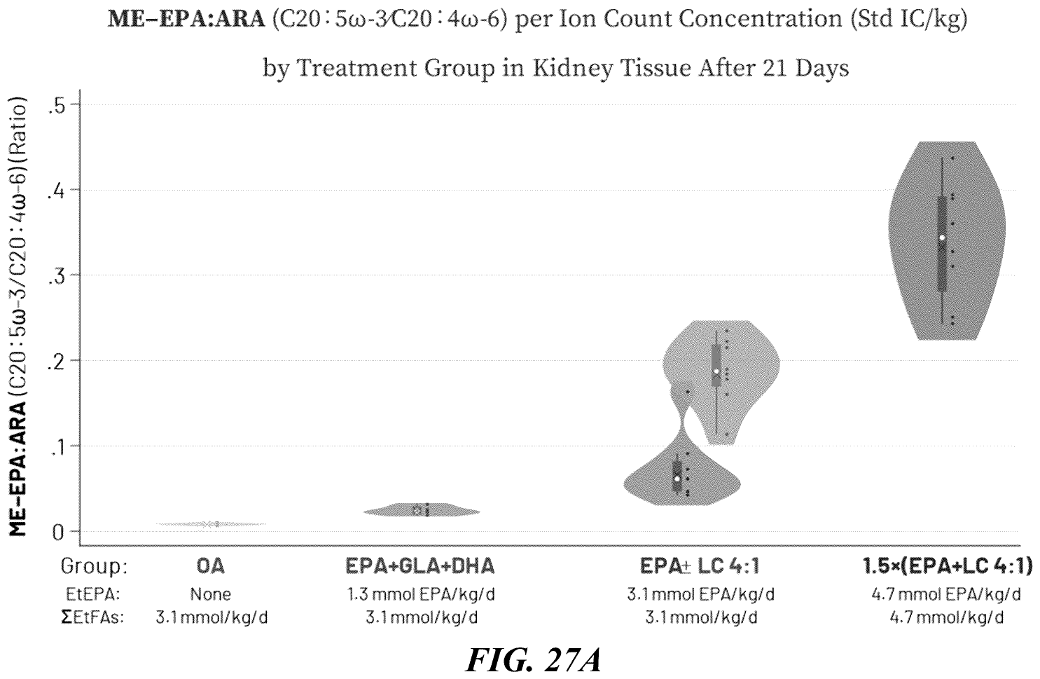

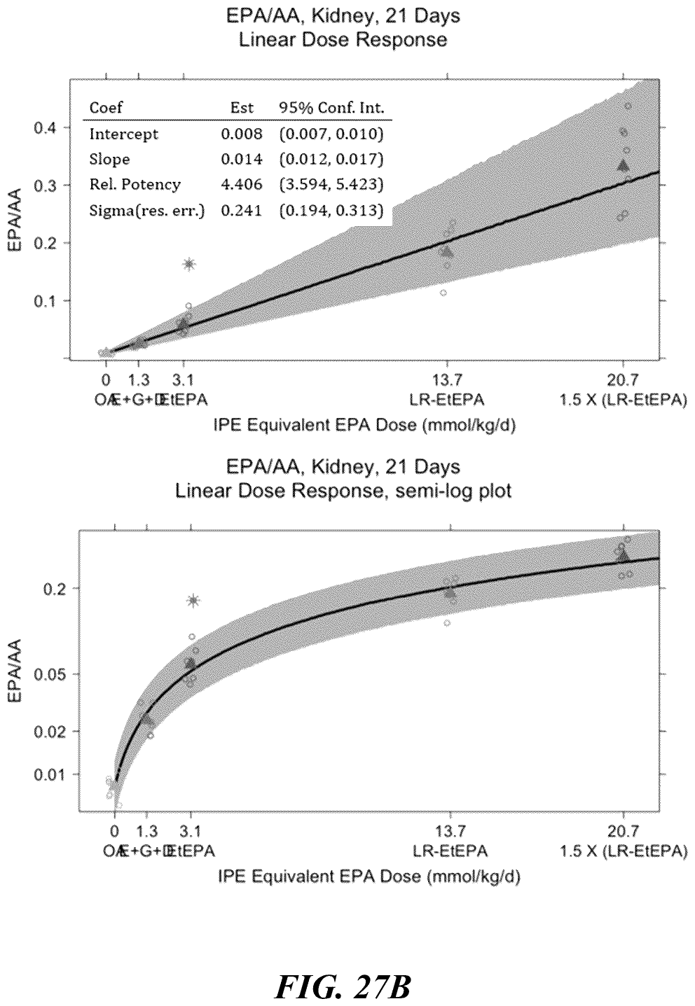

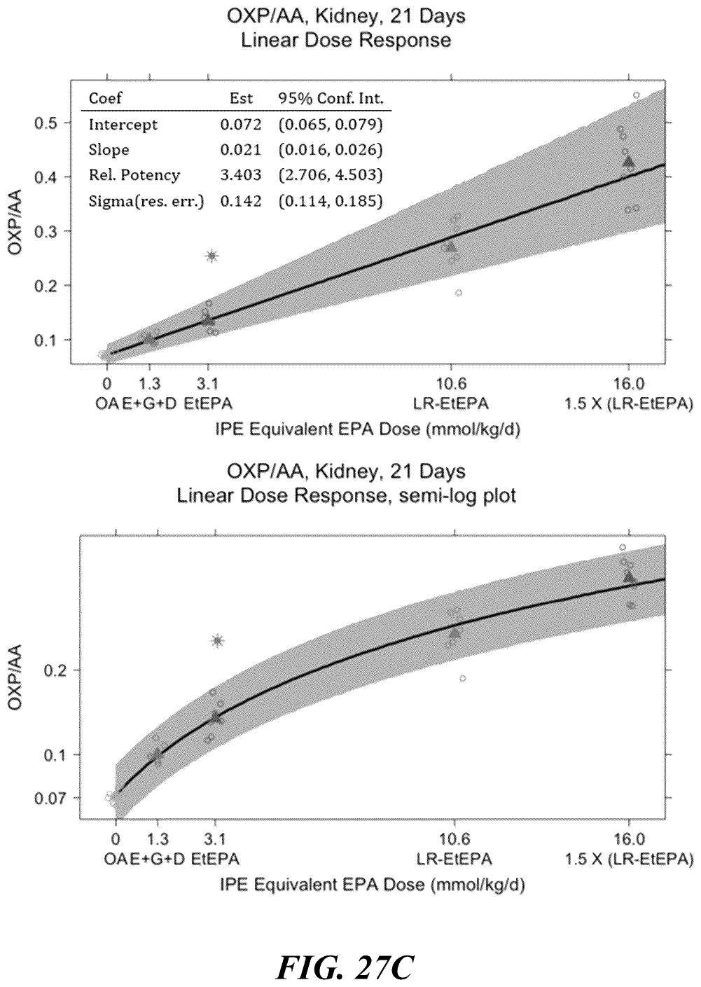

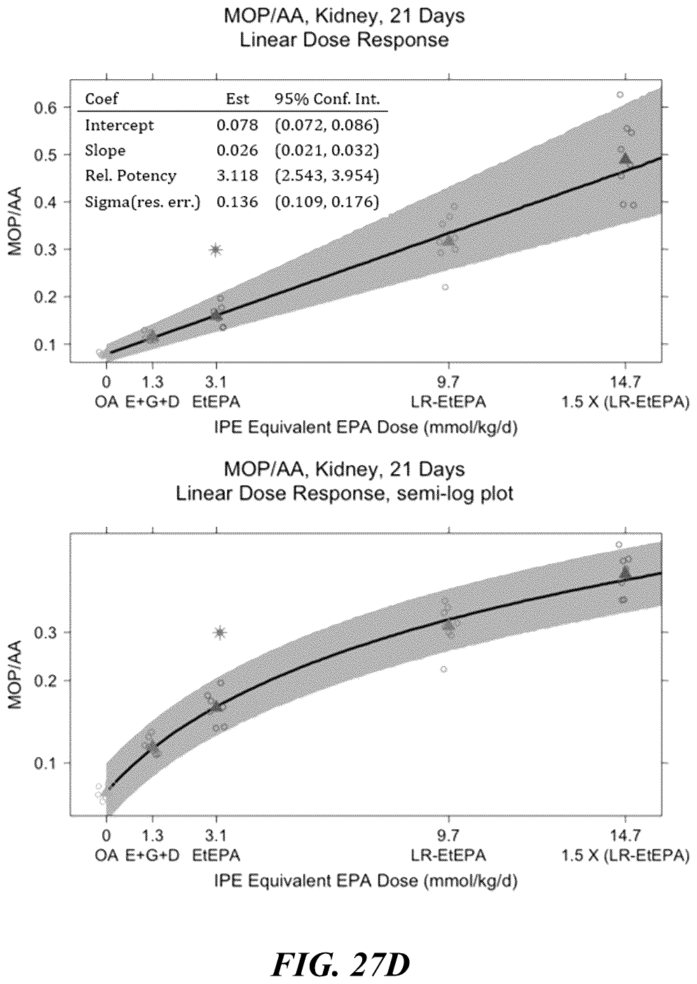

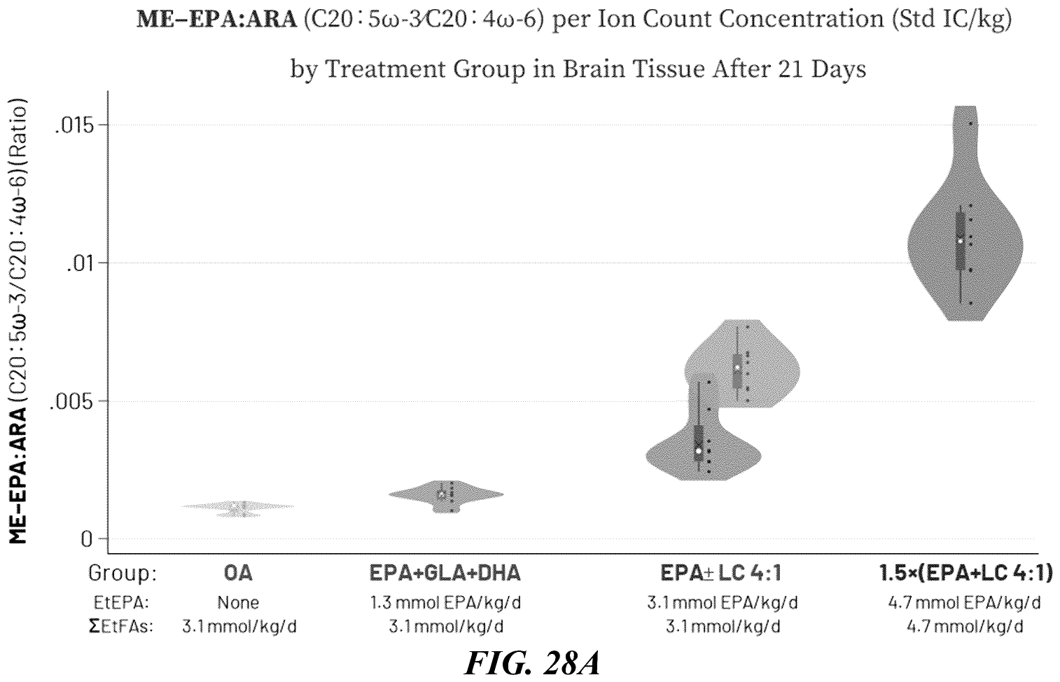

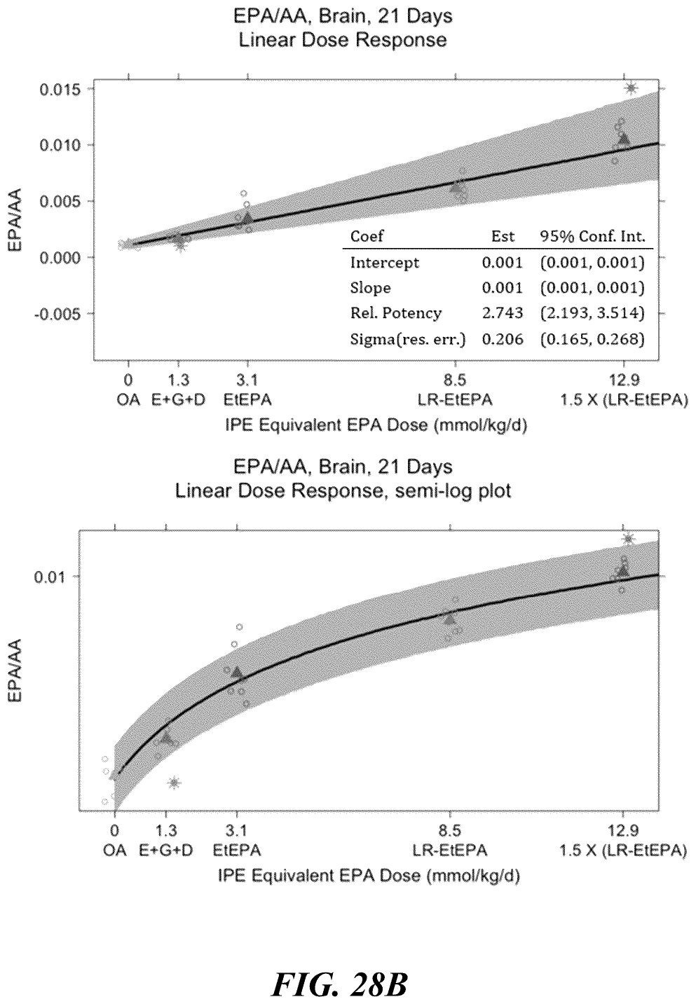

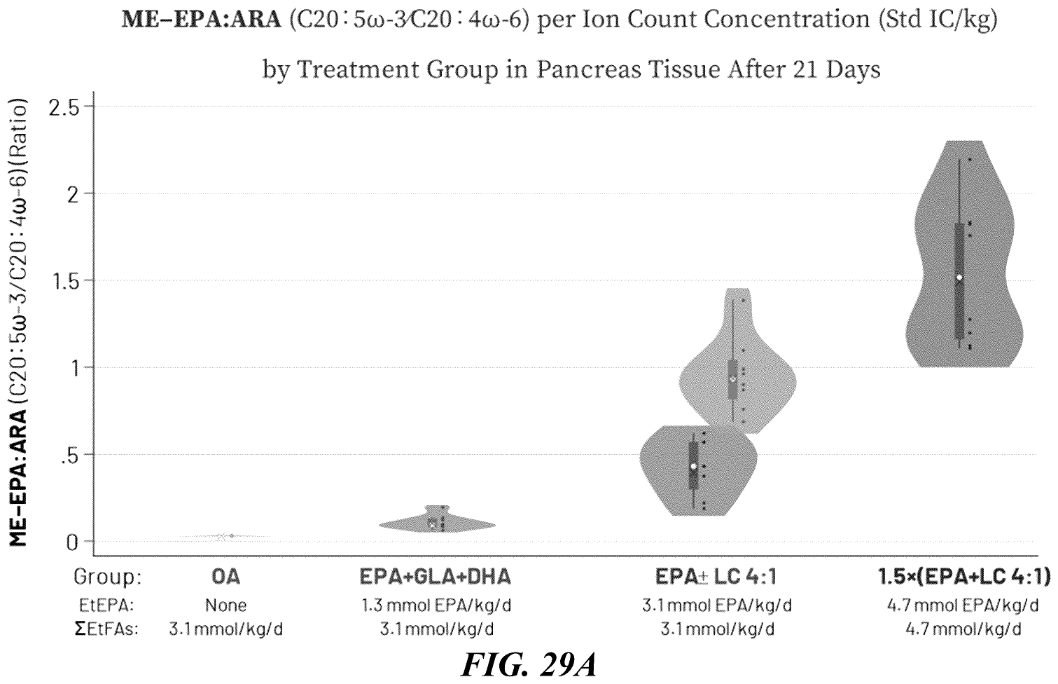

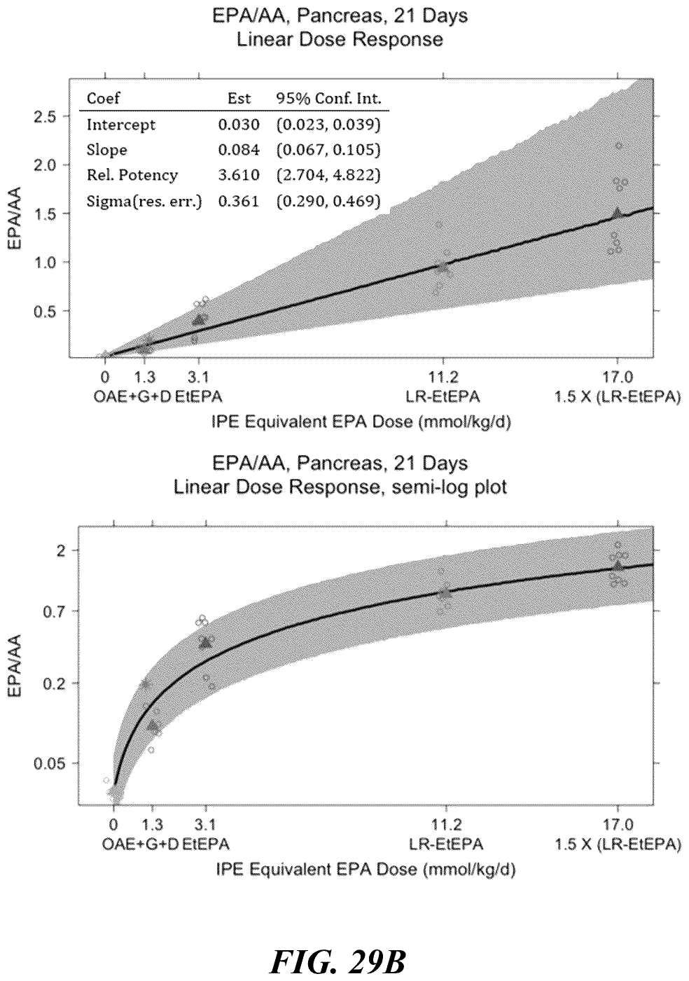

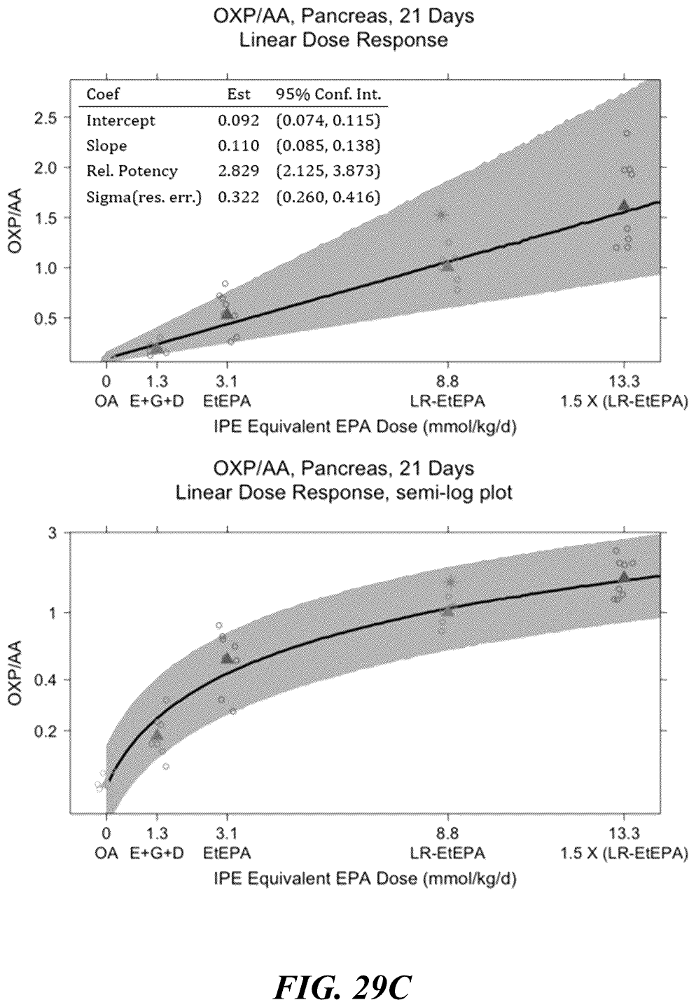

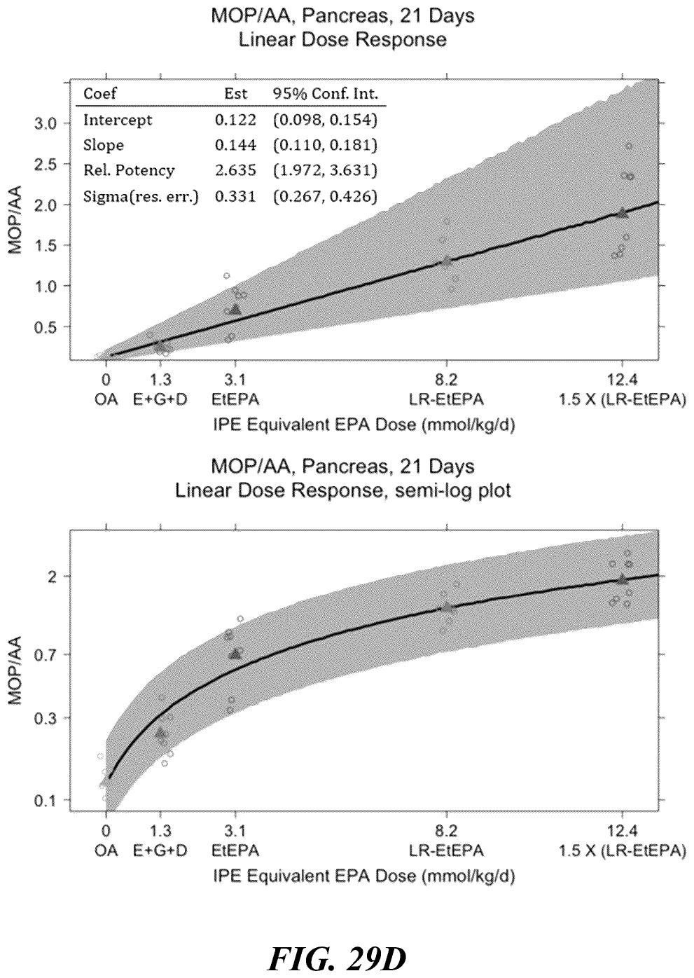

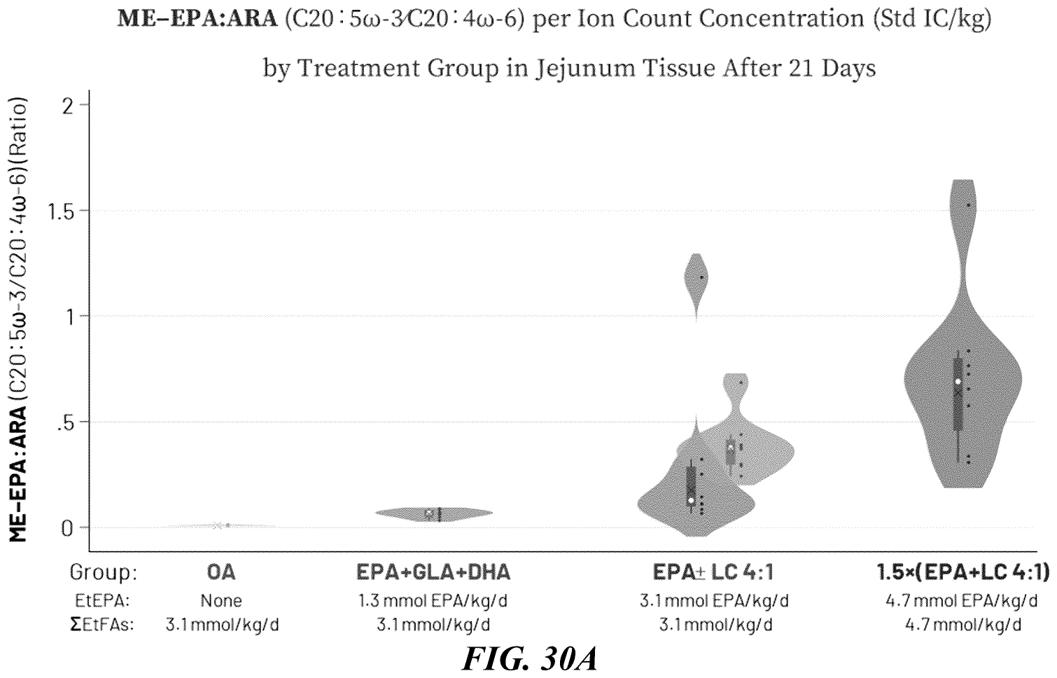

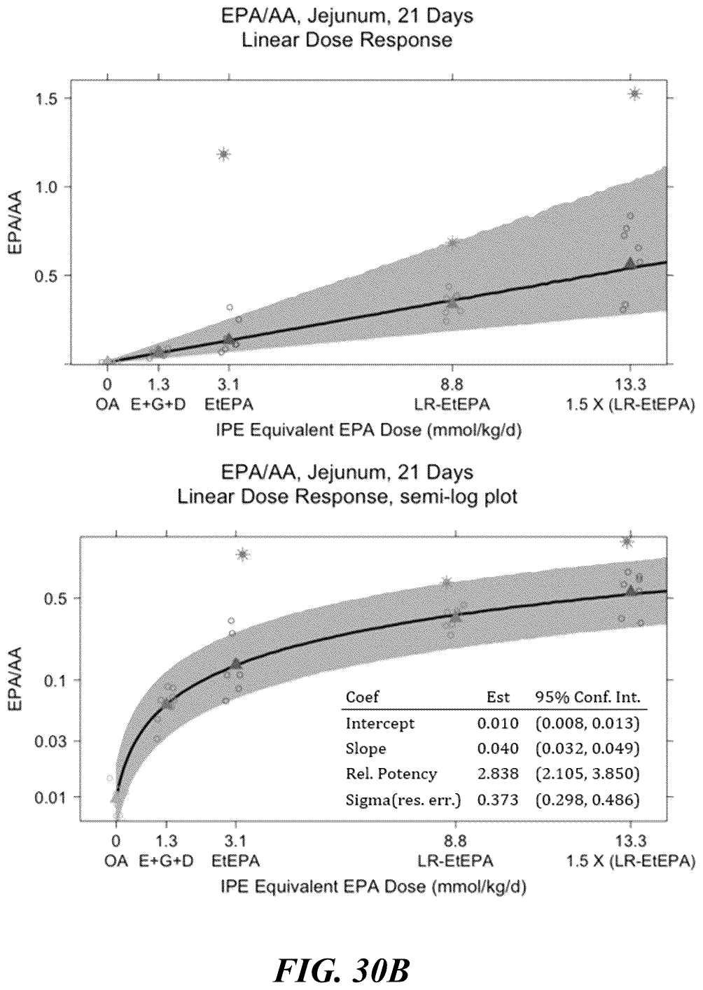

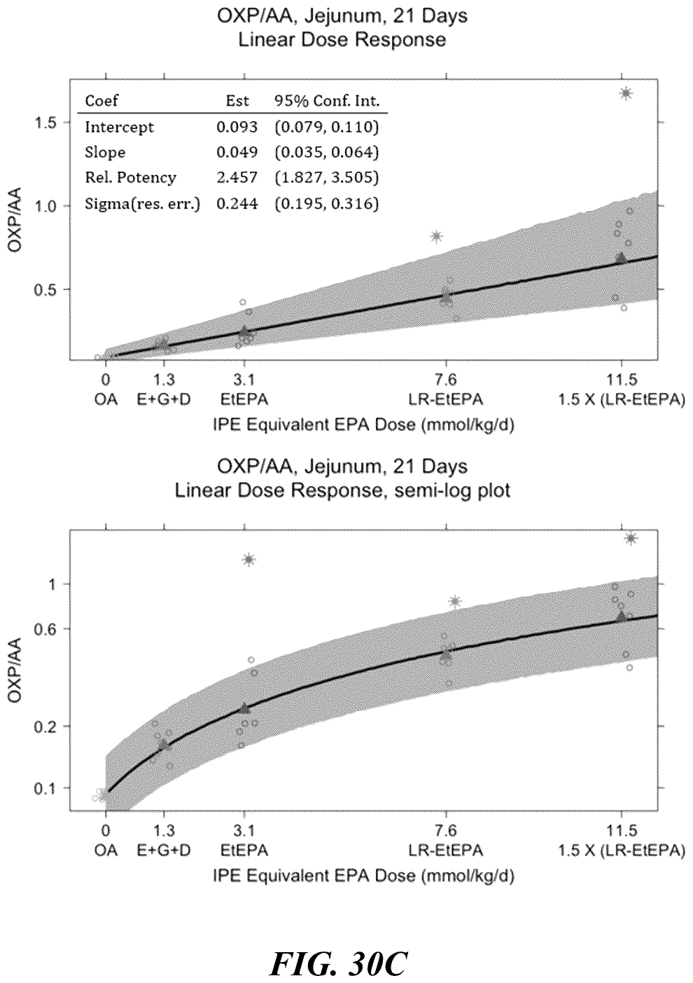

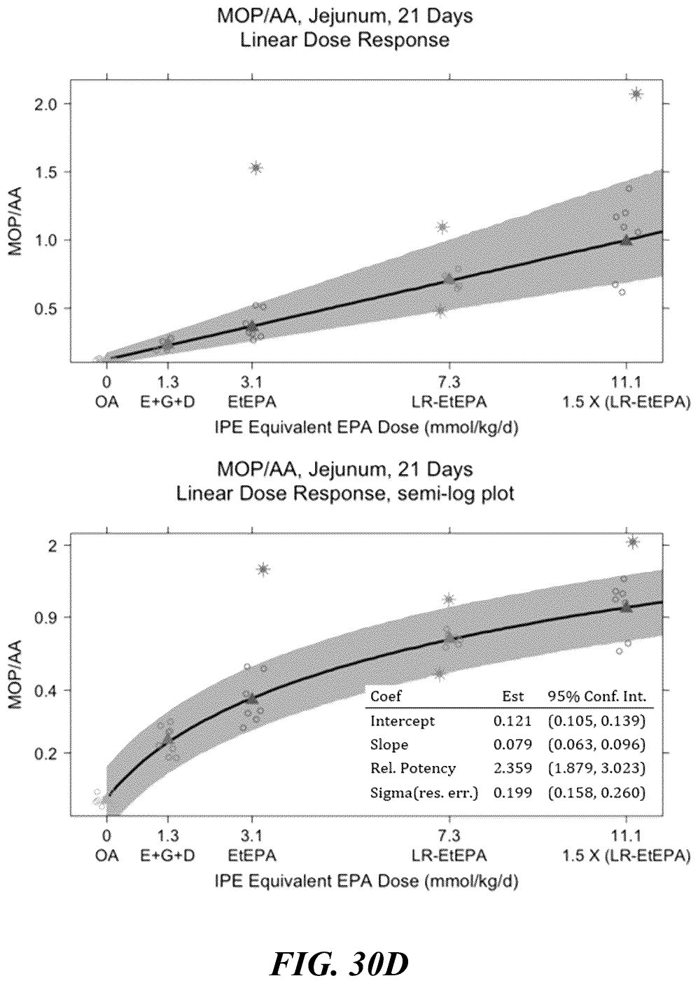

The relative potency of the LR-EtEPA formulation compared to plain EtEPA was also examined in dose response plots as shown in FIGS. 24A-30D. These and subsequent figures involving all five treatment arms present the formulations in the same order, from left to right: (1) OA, (2) EPA+GLA+DHA, (3) EtEPA, (4) LR-EtEPA with EtEPA equimolar to plain EtEPA (1× LR-EtEPA), and (5) LR-EtEPA at 1.5-fold the molar dose of plain EtEPA (1.5× LR-EtEPA). Importantly, the molar amounts of LC-PUFAs were equimolar for treatments (1) through (4), and only treatment (5) has a different dose (1.5×). The latter was done to gauge dose/response for LR-EtEPA, for example, to understand whether further dose response might be available at higher doses following 21-days of treatment; typically, further dose responsiveness was affirmed.

For each “violin” plot (e.g., FIG. 24A for the lung tissue), the rectangles inside each violin represent box plots of the interquartile range (IQR, 25^{th} and 75^{th} percentiles) with median as an uncolored dot within the IQR. The whiskers of the box plots represent 10^{th} and 90^{th} percentiles, and the dots to the right of the box plots are individual subject responses. The curved region outside the box plots represents the density of the distribution (a kernel density plot). The x-axis is proportional to the EtEPA dose given, so that OA is zero, 1.5× LR-EtEPA 1 is 4.7 mmol EPA/kg/day, and the other treatments fall in between at appropriate distances. Note that plain EtEPA and LR-EtEPA are slightly offset from 3.1 mmol EPA/kg/day so as not to overprint. Importantly, these treatments involve an identical amount of EtEPA dosed, so the null hypothesis is that they should overprint if LR-EtEPA does not differ from plain EtEPA.

The primary finding from these violin plots is therefore whether LR-EtEPA and plain EtEPA are distinguishable. Since the x-axis also represents the dose as a continuous variable, the violin plots are also dose-response plots. A key secondary finding is whether the outcome is altered going from left to right; if so, this is evidence for a dose response. A key tertiary finding is whether the dose response is continuous vs there is a discontinuity. The y-axis is the EPA:ARA ratio in Long-Evans rat lung tissue after daily treatment by gavage for 21 days. The findings are interpreted as follows: (1) LR-EtEPA and plain EtEPA are perfectly distinguished, meaning their actual distributions are non-overlapping (i.e., the minimum in the LR-EtEPA group is higher than the maximum in the plain EtEPA group); (2) there is strong evidence for a positive dose response, such that EPA:ARA in lung tissue increases with ascending doses of EtEPA; (3) The overall dose-response relationship features a large discontinuity at the dose of 3.1 mmol EPA/kg/day; this break reflects the non-overlapping distributions of LR-EtEPA and plain EtEPA.

These three features were typical of data from the experiment, so much so that it motivated formal modeling of this “broken” dose response relationship, including the discontinuity. Three statistical models were evaluated: (1) rectilinear, (2) exponential, and (3) E-Max (sigmoidal curve or Hill function). For all tissues examined, the linear model proved best. This implies that results are on the linear region of the underlying dose-response curve (i.e., there was no evidence of a plateau at up to 4.7 mmol EPA/kg/day). Accordingly, all dose response curves were modeled as linear. Normally, a linear function would be modeled as y=mx+b, where y is outcome, m is slope, x is EtEPA dose, and b is y-intercept. To model the discontinuity simply involves including a multiplier to the slope which is only operative for the lymph-releasing formulations (A). For OA, EPA+GLA+DHA, and plain EtEPA, λ=0, whereas for 1× LR-EtEPA and 1.5× LR-EtEPA, λ=1. The slope factor multiplying the slope is referred to as theta (8, or relative potency). Therefore, the equation modeled is:

y=mxθ^{λ}+b

The factor θ expresses the relative potency of the lymph-releasing formulations, as a multiple or fold-change beyond that of the other three formulations. Accordingly, the null hypothesis is that θ=1, meaning LR-EtEPA is “1-fold” the potency of OA, EPA+GLA+DHA, and plain EtEPA (i.e., LR-EtEPA is neither better nor worse or non-inferior). If θ were significantly <1, it means that the outcome is significantly diminished by LR-EtEPA. Finally, θ significantly >1 means that LR-EtEPA raises the outcome versus plain/unadulterated formulations of EtEPA.

After estimating θ in the linear model, the dose response curve is recast in the corresponding dose response curve for each tissue examined (e.g., FIG. 24B from the lung tissue) to present IPE equivalent EPA dose (mmol/kg/day) instead of actual dose. For the three formulations with plain fatty acids (i.e., OA, EPA+GLA+DHA, and EtEPA), the actual doses are given; however, for the LR-EtEPA formulations, the dose is the equivalent dose of plain EtEPA. Since the actual doses of EtEPA are identical, if there were no benefit (i.e., θ=1), the 1× LR-EtEPA dose would superimpose upon the plain EtEPA dose. If θ>1, then the LR-EtEPA will be separated from plain EtEPA, and the distance between the two reflects the factor θ in terms of folds above plain EtEPA.

Accordingly, FIG. 24B is interpreted as follows for lung tissue EPA/ARA following 21 days of treatment: (1) there is a strong linear dose response, described by the line \ y=mxθ^{λ}+b=0.037[dose]3.264^{λ} where λ=0 for plain EtEPA formulations and λ=1 for lymph-releasing formulations. Importantly, 8 is significantly >1, indicated by the 95% confidence intervals (CI) that exclude 1 by a wide margin (95% CI 2.777 to 3.867). The θ of 3.264 means that for a given dose of plain EtEPA, LR-EtEPA achieves ˜3.3-fold the effect of EtEPA on the outcome, EPA/ARA (EPA/AA in the figure). Put in simple terms, to achieve the same increase in EPA/ARA of the LR-EtEPA 1× group, one would have to dose about 3⅓ times the dose of Plain EtEPA. Purely for illustrative purposes, this is demonstrated by Vascepa® capsules on the graph. The usual human dose for Vascepa® is four capsules daily. Again, for illustration only, if one took four capsules of LR-EtEPA 1× to raise the EPA/ARA, one would have to take ˜3.3-fold that amount in Plain EtEPA capsules to raise EPA/ARA the same amount. Thus, 4×3.264≈13 capsules are shown so the reader can compare to the four capsules of the new formulation. The typical human dose is shown here because the human equivalent dose the rats received would results in many more capsules depicted (e.g., 33 capsules for Plain EtEPA vs. 10 capsules for LR-EtEPA, a dose of LC-PUFAs that would be optimal to treat ARDS). Stated another way, taking four capsules of LR-EtEPA would be equivalent to taking 33 capsules of Plain EtEPA to raise EPA/ARA the same amount.

Having such a prodigious difference between the actual and equivalent dose could be medically beneficial in two ways: (1) perhaps one need not dose as much as much for conditions that benefit from modestly raising EPA/ARA, or (2) one could achieve greater rises in EPA/ARA by taking a “usual” dose of the more potent formulation. Of course, this would vary depending on the condition and the degree to which the outcome benefits the condition. Importantly, the ability to enhance the efficacy of plain EtEPA on a given outcome by multiple folds is significant itself, but there is much room for extending these effects by increasing the amount of selected excipients. For example, as shown in Example 2, a 1:1 mix was used, whereby equal amounts of EtEPA and the excipients were used. As per FIG. 2, changing the mix of EtEPA from 4:1 to 1:1 greatly increased the amount of EPA in lymph fluid. Extended to other tissues, increasing the amount of the excipients would also enhance efficacy, especially increasing and/or refining the phospholipid composition.

Table 4 below shows the relative potency (8) for several tissues assessed by EPA/ARA, OXP/ARA (i.e., [EPA+DGLA+DHA]/ARA), and MOP/ARA (i.e., [EPA+DPA+DGLA+DHA]/ARA). Each of these provides an index for the ratio of LC-PUFAs that yield oxylipins that are less apt to worsen inflammation, thrombosis, vasoconstriction, and/or bronchoconstriction compared to ARA. Accordingly, higher ratios indicate greater potential to offset the deleterious effects of ARA-derived oxylipins.

TABLE 4
Relative Potency of LR-EtEPA at Raising
Fatty Acids in Various Tissues

| | | Relative | |
|---|---|---|---|
| Parameter | Tissue | Potency (θ) | 95% Conf. Int. |
| EPA/ARA | Lung | 3.264 | (2.777, 3.867) |
| EPA/ARA | Alveolar Macrophages | 2.877 | (2.510, 3.300) |
| EPA/ARA | Cardiac | 3.376 | (2.789, 4.220) |
| EPA/ARA | Kidney | 4.406 | (3.594, 5.423) |
| EPA/ARA | Brain | 2.743 | (2.193, 3.514) |
| EPA/ARA | Pancreas | 3.61 | (2.704, 4.822) |
| EPA/ARA | Jejunum | 2.838 | (2.105, 3.850) |
| OXP/ARA | Lung | 2.586 | (2.221, 3.056) |
| OXP/ARA | Alveolar Macrophages | 2.551 | (2.132, 3.098) |
| OXP/ARA | Cardiac | 1.955 | (1.313, 3.994) |
| OXP/ARA | Kidney | 3.403 | (2.706, 4.503) |
| OXP/ARA | Pancreas | 2.829 | (2.125, 3.873) |
| OXP/ARA | Jejunum | 2.457 | (1.827, 3.505) |
| MOP/ARA | Lung | 2.32 | (2.049, 2.647) |
| MOP/ARA | Alveolar Macrophages | 2.364 | (2.030, 2.777) |
| MOP/ARA | Cardiac | 1.8 | (1.446, 2.353) |
| MOP/ARA | Kidney | 3.118 | (2.543, 3.954) |
| MOP/ARA | Pancreas | 2.635 | (1.972, 3.631) |
| MOP/ARA | Jejunum | 2.359 | (1.879, 3.023) |

Remarkably, the relative potency was routinely well above one for all tissues examined, including lungs, AVMs, heart, kidney, brain, pancreas, and jejunum, and was often several fold the slope for the plain EtEPA formulations, and for several configurations of LC-PUFAs compared to ARA. The relative potency for EPA/ARA in the brain was almost 3-fold, indicating one would need to take 3 times the dose of plain EtEPA as LR-EtEPA to raise brain EPA/ARA as much as LR-EtEPA did. Note that it is not always the case that a line can be fit for the dose-response, as was the case for brain OXP/ARA and MOP/ARA (data not shown).

The six tissues shown here were selected because they represent the spectrum of tissue perfusion. Fatty acids are distributed in accordance with the tissue perfusion rates of various organs, so sampling across the range of perfusion rates would allow us to understand and model how LR-EtEPA would fare with different tissues. Surprisingly, across a range of tissue perfusion rates, the relative potency remained considerably higher than one. Indeed, the lowest was 1.8, which is still almost double the relative potency of plain EtEPA. In this list, the brain is toward the low end of the perfusion spectrum, typically receiving 0.5 mL/min per mL of tissue in a 70-kg adult male. See Derendorf and Schmidt, eds., Rowland and Tozer's Clinical Pharmacokinetics and Pharmacodynamics: Concepts and Applications (5th Ed. 2020 Wolters Kluwer, Philadelphia). The lungs represent the other end of the perfusion spectrum, receiving 10 mL/min per mL of tissue. Indeed, the lung is the best-perfused organ in the body, receiving 100% of the cardiac output in the parenchyma, and also had a high relative potency for EPA/ARA, 8=3.264, as discussed above. Also, at the high end of the spectrum, the kidneys are very well perfused at 4 m L/min per mL of tissue, and had the highest relative potency, with 8=4.406 for EPA/ARA (95% CI 3.594 to 5.423). This means that for renal diseases that benefit from a more favorable mix of oxylipins, including inflammatory kidney disease, or kidney disease sensitive to vasoactive stimuli, the LR-EtEPA formulation is capable of raising EPA/ARA more than four-fold the same amount of EtEPA given as plain EtEPA. Given the large range of tissue perfusion rates that yielded relative potencies that were folds above plain EtEPA, the LR-EtEPA formulation is suitable for tissues that range in perfusion rates between 0.01 m L/min per mL of tissue to >10 m L/min per mL of tissue in a healthy adult male. Accordingly, LR-EtEPA is suitable to raise EPA/ARA across this range of tissues, if not OXP/ARA and MOP/ARA among conditions that could benefit from lessened inflammation, thrombosis, vasoconstriction, and tissue-specific benefits mentioned in the section above detailing oxylipins. Insofar as the lymph-releasing properties of the composition apply to several LC-PUFAs and oxygenated LC-PUFAs, the technology would be suitable beyond EPA therapy per se.

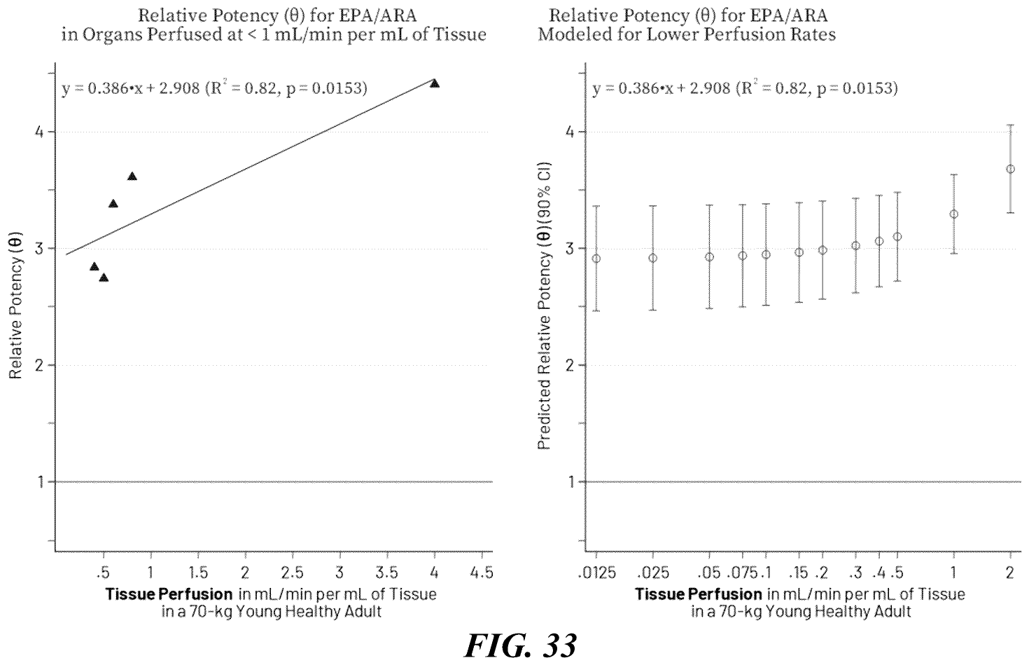

To assure that the lowest-perfused tissues would also have a relative potency (8) above 1, regression modeling was used to model the relationship between tissue perfusion and measured 8 for tissues that were already in the lower half of the perfusion range (i.e., kidney, pancreas, heart, brain, and jejunum), regressing for EPA/ARA, OXP/ARA, and MOP/ARA simultaneously, with separate slopes for each outcome to specify equations appropriate to each outcome. Results for EPA/ARA are shown in FIG. 33 (left panel). The best-fit line was y=0.386x+2.908, with R²=0.82 and p=0.0153, indicating a very strong goodness of fit. Next, we modeled lower perfusion rates, which would be more suited to skin, adipose, and inactive muscle (FIG. 33, right panel). This implies that even these lower-perfusion tissues would achieve relative potencies around 3 after 21 days of therapy. Importantly, the 90% CI's exclude 1, and by a large margin, reassuring that variances in tissue perfusion (e.g., during disease or for lower-perfusion organs) are unlikely to “erase” the relative potency advantage of LR-EtEPA compared to plain EtEPA. This was important to model, because tissues that are less avidly perfused would generally require longer to reach steady state, in which case, more than 21 days of therapy would be needed to get the maximal effect, absent a loading dose. This model reassures that 21 days is likely adequate for LR-EtEPA to reach a relative potency that is a multiple of that of Plain EtEPA. In summary, the perfusion-relative potency relationship was strong and permitted modeling of lower-perfusion tissues, indicating that said tissues would also likely manifest the potency advantage of LR-EtEPA within 21 days.

Another aspect of the present study looked at the effect of different treatment arms on fatty acid metabolism, including fatty acid-derived intermediaries and oxylipins as they may be important in a variety of biological processes including inflammation, vasoconstriction, bronchospasm, and/or thrombosis. DGLA was one of the fatty acids evaluated. Surprisingly, in alveolar macrophages and cardiac tissue, formulations containing equimolar amounts of EtEPA did not differ from E+G+D in their effect on DGLA (data not shown). This was unexpected because E+G+D included DGLA precursor, GLA; one would expect E+G+D to therefore prove superior to EtEPA in raising DGLA. This implies that pure EtEPA itself at least matches E+G+D with respect to DGLA. Even more surprising, in lung tissue, the equimolar EtEPA formulations were actually superior to E+G+D, meaning they raised DGLA significantly more than E+G+D, despite that EtEPA formulations lack GLA. This suggests that EPA is altering the kinetics of DGLA. To test this, the ratio of the product/precursor is an index of successful DGLA conversion to AA (i.e., the inverse of the ratio presented above). A high AA/DGLA indicates efficient conversion of DGLA to its daughter molecule AA. The conversion of DGLA to AA is facilitated by the Δ5-desaturase enzyme (c.f., FADS1, the fatty acid desaturase 1 gene). When LC-PUFAs are converted to other PUFAs, this occurs by both elongation (adding carbon units) and desaturation (i.e., adding new double bonds). Of these processes, desaturation represents the rate-limiting steps vs. elongation. Therefore, the conversion of fatty acids is largely a matter of the activity of a series of desaturase enzymes. Accordingly, the AA/DGLA ratio is a functional Δ5-desaturase index for these omega-6 fatty acids (Δ5D-I-ω6). Viewed as a functional index of Δ5-desaturase, if EPA suppresses Δ5-desaturase, the consequence would be a less efficient synthesis of AA from DGLA, and consequently, a significant drop in the Δ5-desaturase index. Indeed, viewed this way, the AA/DGLA ratio provides a more intuitive way to look at the impact of EPA on DGLA kinetics (FIG. 31A). In accordance with the functional assay represented by Δ5D-I-ω6, in a separate experiment involving a provocative inflammatory challenge (by IL-6 administration), EPA strongly suppressed Δ5-desaturase, that is, the protein product of fatty acid desaturase 1 (FADS1), in endothelial cells (FIG. 34B). Similarly, EPA strongly suppressed 06-desaturase, that is, the protein product of fatty acid desaturase 2 (FADS2) in endothelial cells (FIG. 34B). These results affirm that functional tests of desaturase enzymes are accompanied by diminished protein levels of desaturases, and in the case of Δ5-desaturase, affirm the ability of EPA to suppress this enzyme as assayed by Δ5D-I-ω6 (FIG. 31A). The substantial suppression of Δ5D-I-ω6 by isolated EtEPA formulations indicates that EPA is altering DGLA kinetics by inhibiting DGLA's catabolism. This in turn raises the DGLA pool size. It is novel and surprising that EtEPA can actually outperform the clinical approach of dosing GLA to increase the DGLA pool, especially because lung tissue is the target of therapy for DGLA administration as Oxepa® (i.e., to treat ARDS). To rule out altered DGLA synthesis, the Δ8-desaturates enzyme is important (c.f., FADS2, the fatty acid desaturase 2 gene). The Δ8-desaturase enzyme catalyzes DGLA synthesis from the precursor eicosadienoic acid (EDA, a 20-carbon omega-6 LC-PUFA with 2 double bonds). Like Δ5D-I-ω6, the Δ8-desaturase index for these omega-6 fatty acids (Δ8D-I-ω6) is a functional index of enzyme activity. Also surprisingly, the isolated EPA formulations actually raised Δ8D-I-ω6, consistent with sufficient induction to raise DGLA synthesis (FIG. 31B). Thus, EPA is capable of raising the DGLA pool “coming and going,” meaning it promotes DGLA synthesis and suppresses its catabolism by conversion to AA. This would be a boon for treating conditions involving pathological levels of deleterious AA oxylipins, because not only would the supply of AA be diminished as an oxylipin precursor, but the greater DGLA pool would provide competing oxylipins that are less detrimental. Beyond disadvantaging AA by substrate competition by DGLA for oxygenating enzymes, specific DGLA oxylipins inhibit COX and LOX enzymes that oxygenate AA into oxylipins (e.g., PGE1 and 15-HETrE). The apparent effect of IPE and Oxepa® on DGLA kinetics is shown in FIG. 31C. The significance of the ability of EtEPA to raise the DGLA pool and kinetics via Δ5D-I-ω6 and Δ8D-I-ω6 is that this implies that EtEPA is not at all disadvantaged compared to the current practice of dosing GLA. To the contrary, these results confirm that EtEPA is superior at raising DGLA in the lung.

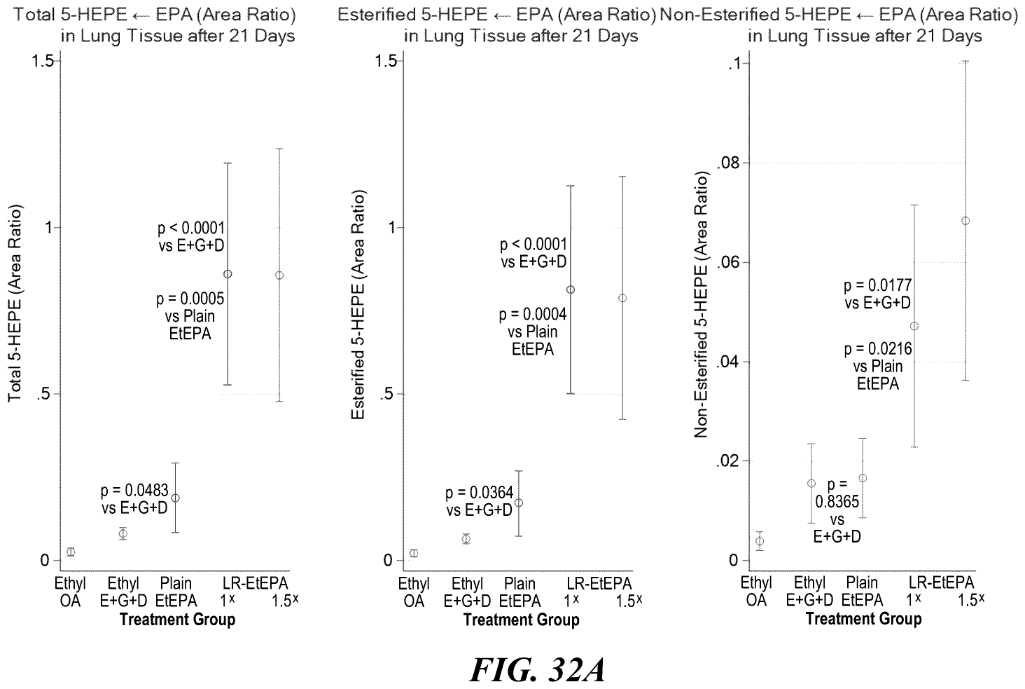

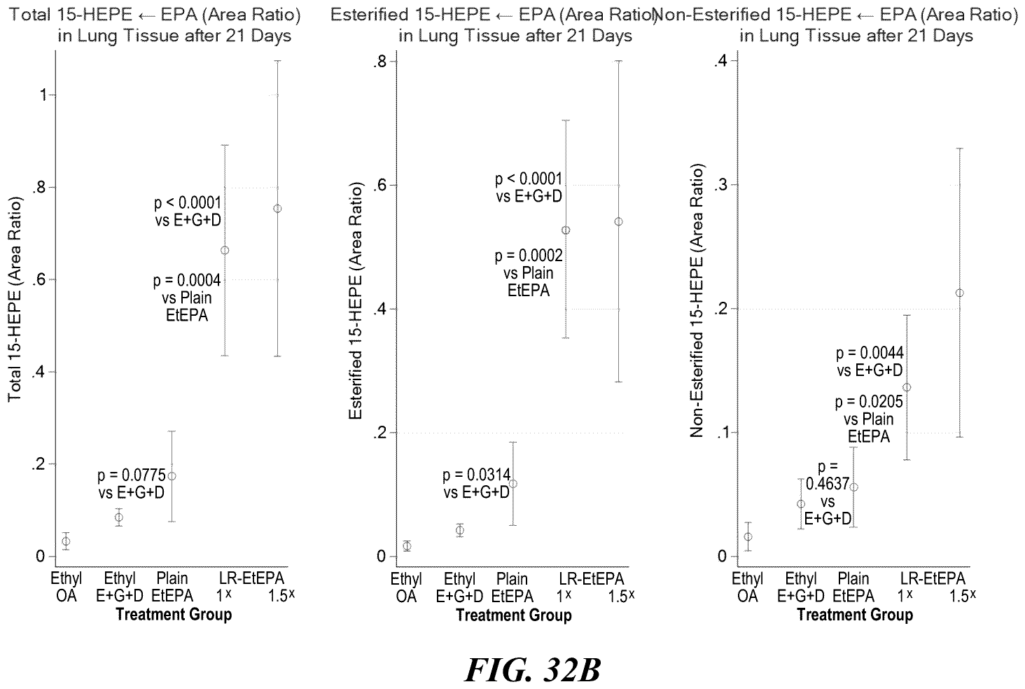

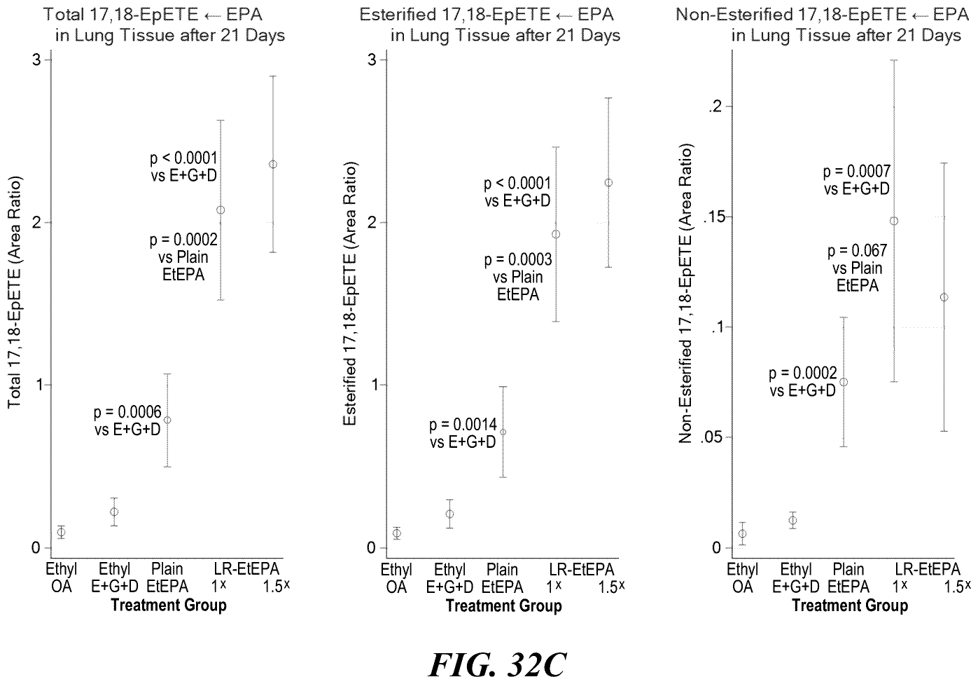

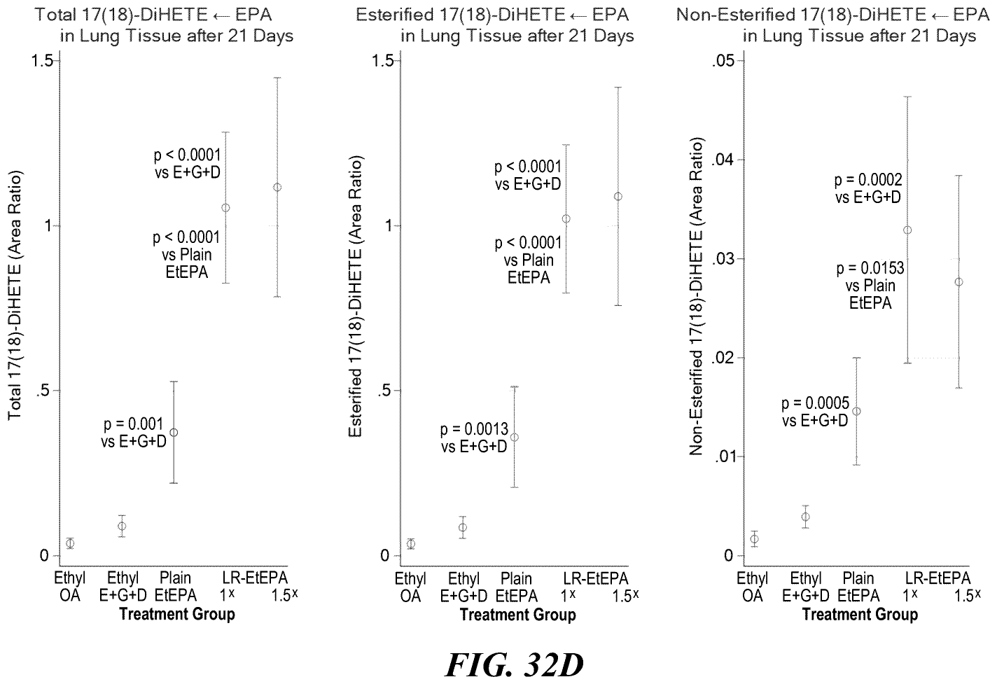

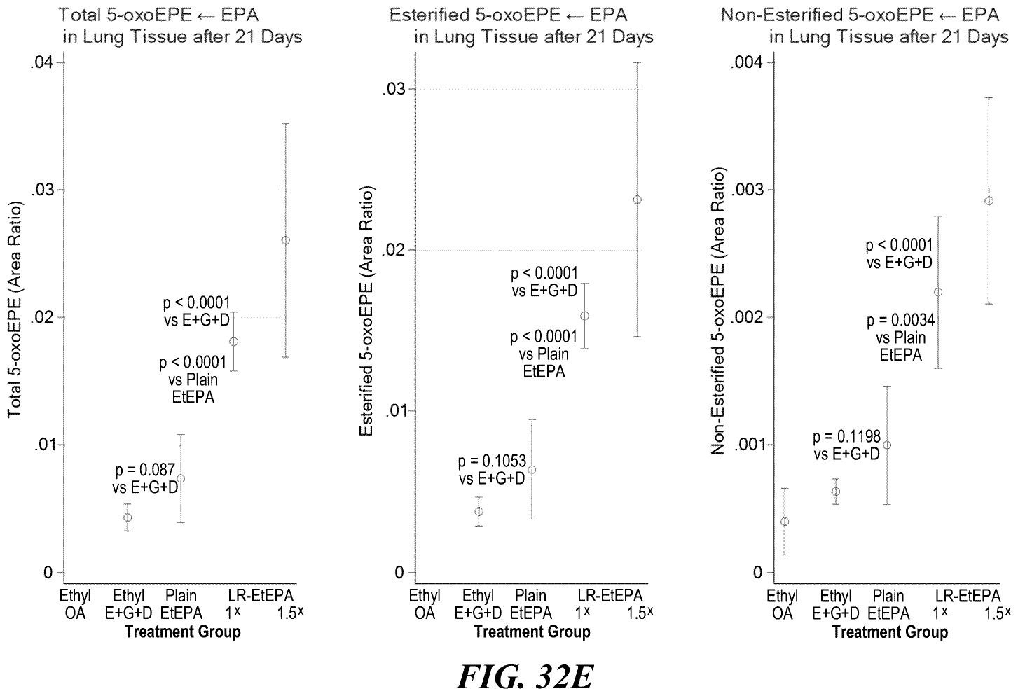

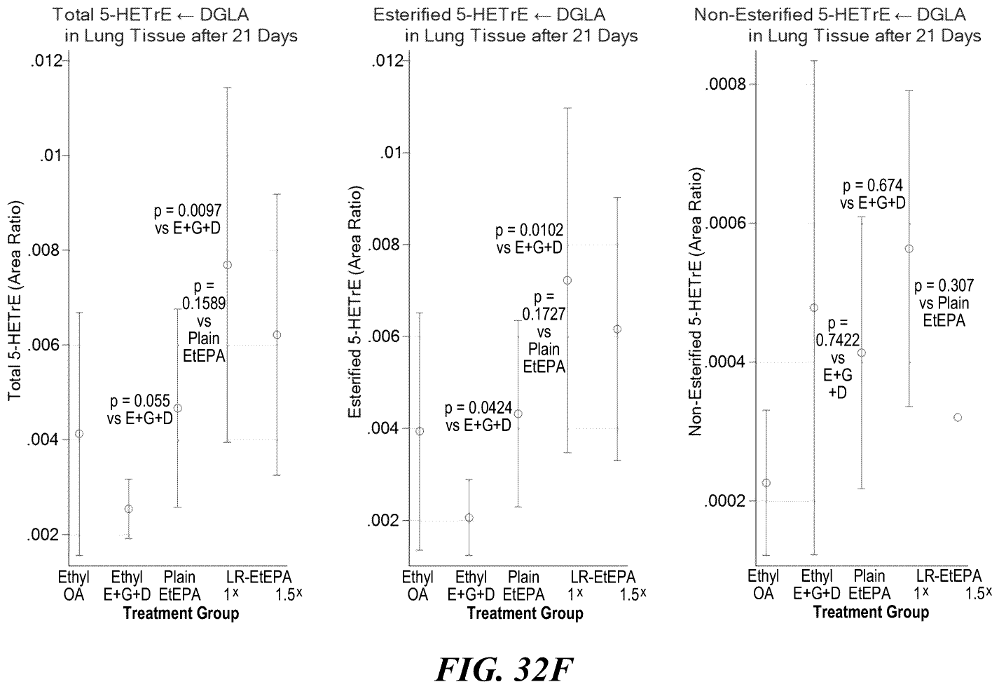

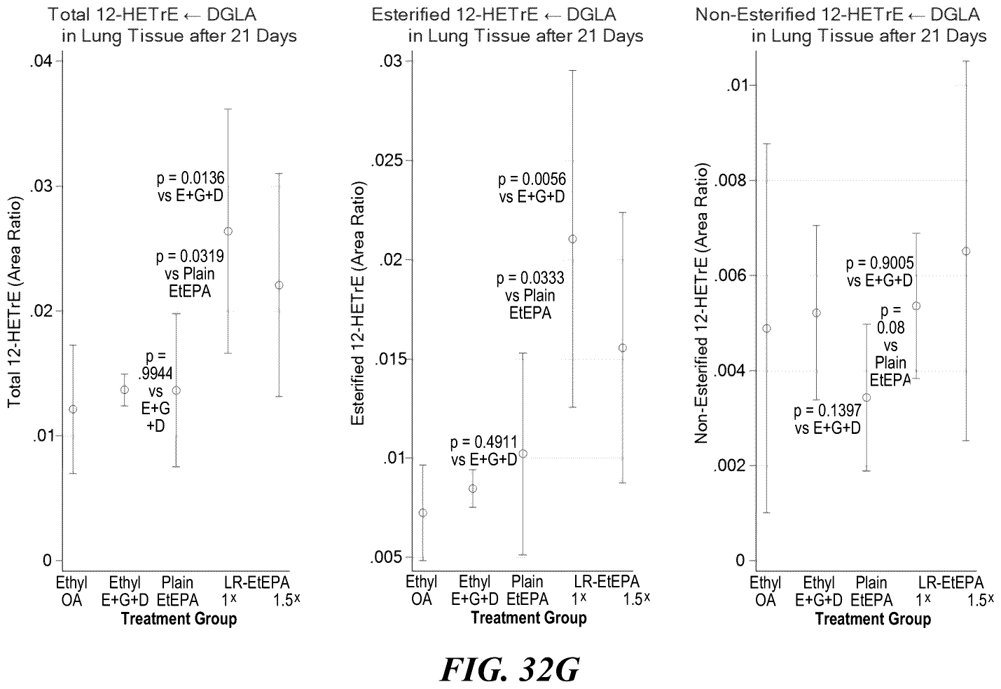

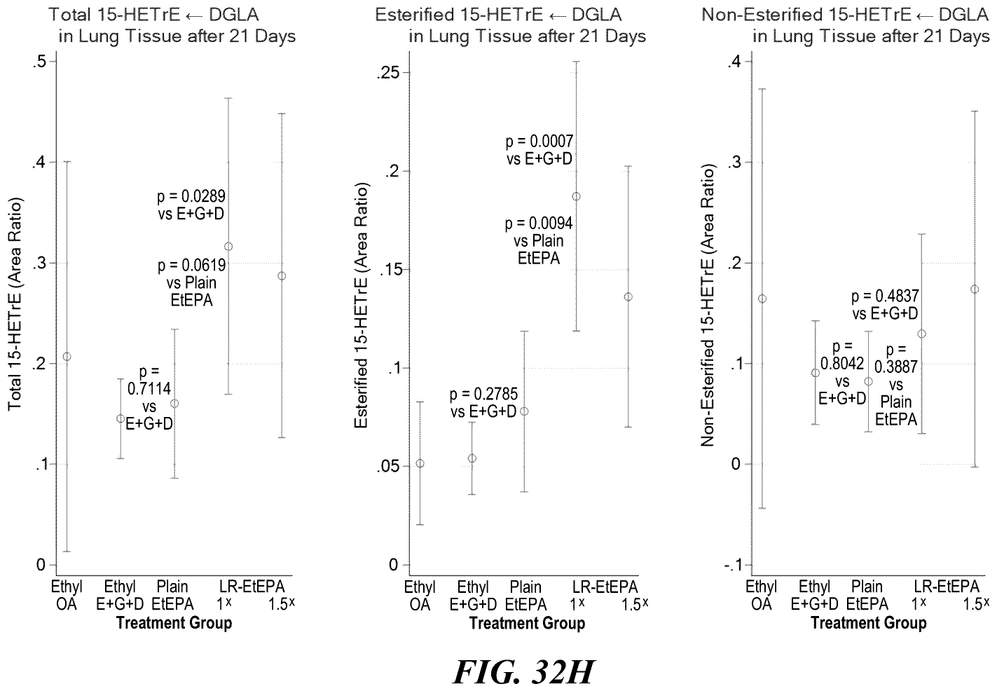

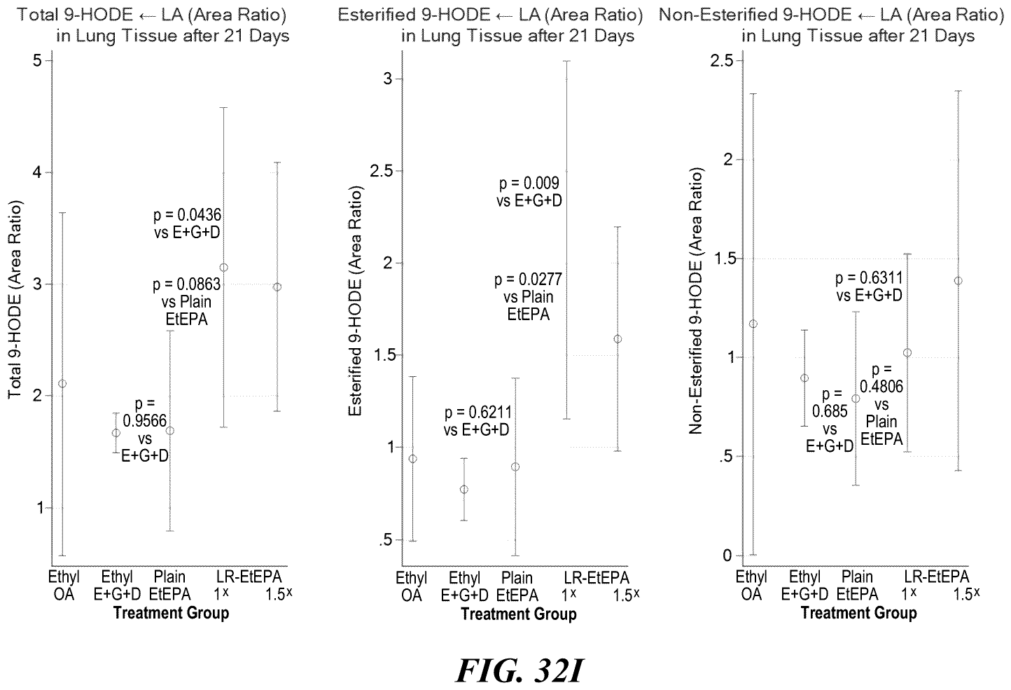

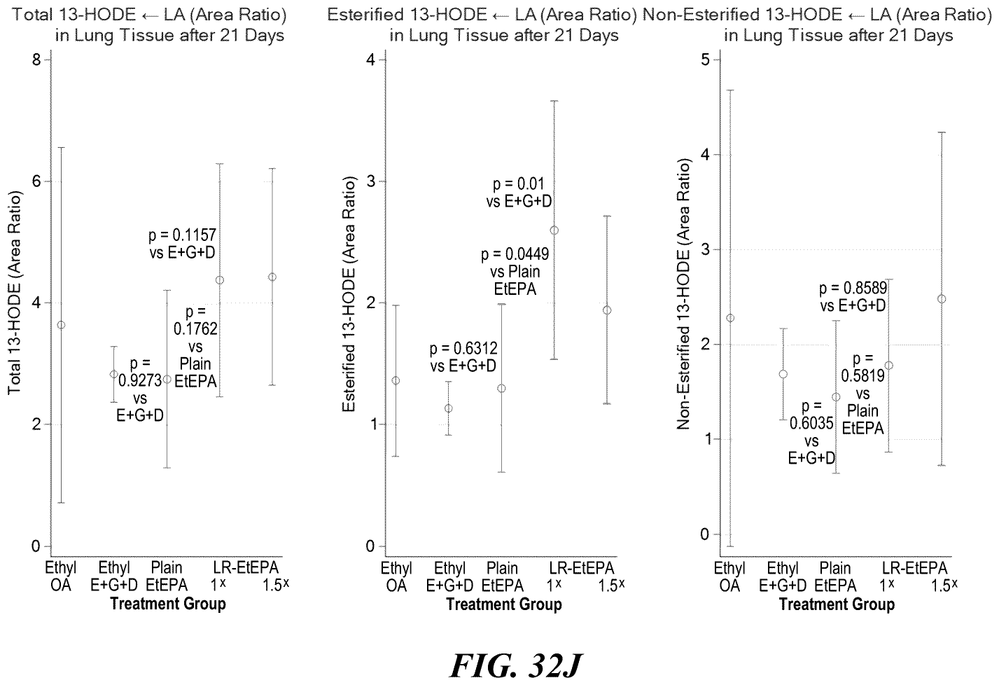

Again, raising DGLA in the lungs is expected to be beneficial insofar as (1) this raises the less-detrimental (i.e., less inflammatory, less thrombotic, less bronchonstrictive) oxylipins derived from DGLA, and (2) DGLA-derived oxylipins, like EPA-derived oxylipins, can offset ARA-derived oxylipins by competing for oxygenating enzymes (synthesis) and receptors (function). Though the idea that EPA would raise DGLA was itself surprising, even more unexpected was the observation that even in unprovoked lung tissue, DGLA-derived oxylipins were subject to a dose-responsive relationship to EPA (FIGS. 32F-32H). In unprovoked lung tissue, the DGLA-oxylipin 15-HETrE was assessed as total oxylipins, non-esterified oxylipins, and as esterified oxylipins. Total 15-HETrE was substantially increased by LR-EtEPA compared to E+G+D (p=0.0289) and to plain EtEPA (p=0.0619). This analysis only included half of the subjects in the main study, so the loss of statistical power from half the sample likely accounts for the borderline p-value when comparing to plain EtEPA. Moreover, esterified 15-HETrE was also substantially higher in LR-EtEPA compared to E+G+D (p=0.0007) and to plain EtEPA (p=0.0094). Likewise, total and esterified 12-HETrE is derived from DGLA, and total and esterified 12-HETrE were significantly greater than E+G+D and compared to plain EtEPA. These results reassure that the unexpectedly elevated DGLA from LR-EtEPA also translate to elevated oxylipins derived from DGLA, implying that these involve functional differences related to the favorable properties of these oxylipins and oxylipins from DGLA in general.

This means that both plain EtEPA and LR-EtEPA are distinguished from the approach currently in practice for ARDS and would outperform the current approach. To understand how LR-EtEPA is superior to plain EtEPA, one must turn to the more compelling outcomes, such as OXP/AA and MOP/AA, because these outcomes are more broadly related to the mechanism of action by which E+G+D treats ARDS, for example. Indeed, plain EtEPA is superior at raising OXP/AA (i.e., [EPA+DGLA+DHA]/AA) in the lung vs E+G+D. Importantly, in turn, LR-EtEPA was about twice as effective at raising OXP/AA vs. plain EtEPA at an equimolar dose (FIGS. 24C-24D). The relative potency of the LR formulation was estimated as 2.6-fold plain EtEPA (95% CI 2.2 to 3.1). This implies one would have to dose 2.6-fold the dose of plain EtEPA just to compete with the same molar dose of LR-EtEPA. This disparity is even greater for E+G+D, which again, was given an advantage in this experiment by withholding a plethora of competing fatty acids that are co-administered with this approach in practice. Similar results were seen in the chief immune cell of the lung, AVMs (FIG. 25C). Thus, in treating ARDS, the OXP/AA index indicates the LR-EtEPA is superior to both plain EtEPA and E+G+D in raising the LC-PUFAs that drive the mechanism of action of Oxepa®. Moving outside of ARDS therapy, the MOP/AA index is broadly applicable to several other diseases, including diseases outside of the pulmonary system. Here, MOP incorporates several LC-PUFAs proposed to be medically beneficial as medicinal oxylipin precursors: EPA+DGLA+DPA+DHA. Thus, the MOP/AA ratio indicates the ability of E+G+D, pain EtEPA, and especially LR-EtEPA to exert therapeutic effects by limiting the “damage” from AA and its detrimental oxylipins in conditions that are worsened by the inability to regulate said oxylipins. In the lung, the MOP/AA ratio was higher in plain EtEPA vs. E+G+D, and more importantly, LR-EtEPA was superior to plain EtEPA (FIG. 24E). The relative potency for LR formulations was 2.3-fold that of plain EtEPA (95% CI 2.1 to 2.6) for lung tissue (FIG. 24E) and was 2.4-fold (95% CI 2.0 to 2.8) for alveolar macrophages (FIG. 25D). These data indicate several advantages over Oxepa® for treating ARDS. These include (1) the ability to limit fatty acids to medicinal oxylipin precursors by timing doses opposite of diets featuring other, “competing” fatty acids, so the benefits of MOPs are not diluted by said fatty acids, (2) the ability to limit deleterious conversion of DGLA to AA by withholding GLA, (3) the ability to increase the lecithin portion, especially for critical care applications, such as the 1:1 ratio of excipients to EtEPA shown in FIG. 3, which significantly raised EPA, and did so quickly (i.e., within hours of the first/only dose), (4) the ability to rely upon robust suppression of the rate-limiting Δ5-desaturase enzyme and robust induction of the rate-limiting Δ8-desaturase enzyme, which thereby raise DGLA at the expense of AA by robustly altering its kinetics, rather than relying on feed-forward dosing of the DGLA precursor substrate. Especially pertinent to Oxepa® and ARDS, the observation that LR-EtEPA is greatly superior to plain EtEPA at raising OXP/AA (and for that matter, MOP/AA) and E+G+D implies greater efficacy for LR-EtEPA.

Moreover, dosing LC-PUFAs often results in suppression of certain enzymes that facilitate conversion of one LC-PUFA to its canonical metabolite LC-PUFAs. These conversions are facilitated by two broad categories of enzymes: (1) desaturase enzymes, which add double bonds (rendering the LC-PUFA less saturated/more desaturated); and (2) elongase enzymes (rendering the LC-PUFA longer, usually by two carbon units). Of these enzyme families, the desaturases are thought to catalyze the rate-limiting steps. As an example, EPA is synthesized from omega-3 eicosatetraenoic acid (ETA, a 20-carbon omega-3 LC-PUFA with four double bonds), which conversion is catalyzed by Δ5-desaturase. In turn, EPA is converted to omega-3 docosapentaenoic acid (DPA, a 22-carbon omega-3 LC-PUFA with five double bonds), which conversion is catalyzed by Δ5-elongase (regulated by elements of the ELOVL gene family). A functional index of the efficiency of EPA conversion to DPA is the product/precursor ratio, the Δ5-elongase index for omega-3's (Δ5EI-ω3). A more efficient reaction might raise the product and/or lower the precursor, yielding a higher ratio. Conversely, a less efficient reaction might lower the product and/or raise the precursor, either in absolute terms or relative to each other. Surprisingly, there appears to be a strong dose-response relationship between EPA and Δ5EI-ω3 suppression. This was manifest as substantial suppression of the product/precursor ratio, namely, a precipitous drop in the DPA/EPA ratio (FIG. 31D). This effect was very consistent between several tissues examined. Specifically, LR-EtEPA is consistently suppressed compared to plain EtEPA and E+G+D; more broadly, there is an unmistakable, if unexpected, dose-response trend across the ascending doses of EtEPA represented by the five groups from left to right on the figure. As with many other outcomes, the differences between LR-EtEPA and plain EtEPA arise despite that they both involve the same molar dose of EtEPA given; therefore, the differences in Δ5EI-ω3 must be attributable to the different composition of LR-EtEPA. The significant of Δ5-elongase suppression is that this would tend to increase the amount of EPA in the tissues, for example, in the LC-PUFA reservoir in the cell membrane. This likely facilitates EPA retention in the cell membrane, so that a larger reservoir is on hand in a given tissue when a cellular injury occurs. In case of cellular injury more EPA would be available as a substrate for oxylipin production, yielding more EPA-derived oxylipins compared to the detrimental oxylipins that arise from ARA. In summary, apparent Δ5-elongase suppression by LR-EtEPA is an unexpected added benefit of the formulation.

### Example 4: A Provoked-Inflammation Model to Elucidate the Effects of Icosapent Ethyl on Tissue Heme Oxygenase Expression

Icosapent ethyl (IPE, also referred to as ethyl-EPA, EtEPA, E-EPA, or EPA-E) is an ethyl ester of eicosapentaenoic acid, for which daily gram doses improves dyslipidemia and prevents major atherosclerotic vascular disease events. Currently, a highly purified form of IPE, Vascepa®, is used to treat hypertriglyceridemia and prevent major atherosclerotic vascular events (MACE). The previous examples have shown that co-administering IPE with small amounts of a mixture of excipients featuring soy lecithin, polysorbate 80, and polyoxyl-35 hastens EPA's appearance in the circulation. Preliminary results suggest the excipients may achieve this by altering the pre-circulatory route to the circulation, increasing EPA tissue distribution to select tissues accordingly. Particularly, in the rat, the excipients enhanced EPA delivery to the lymphatic system upon acute dosing, and thence, significantly increased EPA levels in lung and heart tissue upon daily dosing.

Separately, heme oxygenase 1 gene (HMOX1) is a Nrf2-regulated gene and codes the enzyme heme oxygenase 1 (HO-1). HO-1 has important antioxidant, anti-inflammatory, antiapoptotic, and immunomodulatory effects in vascular cells and tissues, whereby HO-1 can mitigate adverse tissue injury responses. These effects are distinct from beneficial effects from oxylipins and are mediated by protein products whose gene expression is induced by Nrf2, rather than mediated directly by fatty acid products derived from LC-PUFAs. As such, the antioxidant, anti-inflammatory, antiapoptotic, and immunomodulatory effects in vascular cells and other tissues from HO-1 and/or other Nrf2-induced antioxidants are distinct from beneficial effects from oxlylipins, and constitute a separate mechanism of action. Thus, the antioxidant effects of HO-1 and co-regulated antioxidant gene products comprise a distinct mechanism from EtEPA and derivatives therefrom which moderate cellular injury and thereby improve disease. Importantly, enhancing delivery of LC-PUFAs and their oxylipins, including but not limited to EPA and its oxylipins, would improve the ability to exploit HO-1 and co-regulated antioxidant gene products to mitigate cellular injury. This is particularly important for conditions of cell lysis, especially hemolysis and other tissue damage that releases free heme into the injured tissue, as HO-1 is well known to limit toxicity from free-heme. Thus, cellular injury from hemolysis, tumor lysis, or from ischemia, as well as SIRS, sepsis, and ARDS are expected to be remediated by the ability of LC-PUFAs to protect from free-heme toxicity and from antioxidant effects from HO-1 itself and other antioxidants co-regulated on demand by Nrf2. Indeed, several LC-PUFAs and their oxylipins likely promote this injury response by activating Nrf2, including other omega-3 LC-PUFAs besides EPA, such as DHA. All such LC-PUFAs would benefit from a multi-fold improvement in delivering said LC-PUFA to the site of cellular injury, as happens with the lymph-releasing formulation, aided by the rapid integration of PL-EPA into cell membranes. Without being limited to EPA for this purpose among different choices among various LC-PUFAs, if multiple LC-PUFAs activate Nrf-2 and engender a robust response from HO-1 and related antioxidants, then the optimal choice may be the LC-PUFA that produces the suite of oxylipins that best competes with the ill effects of ARA-derived oxylipins. This is because cellular injury, including injury from free heme, is accompanied by the inflammatory and pro-thrombotic cascades. As such, EPA may well be in a superior position to leverage HO-1 benefits owing to its ability to (1) strongly suppress ARA synthesis by inhibiting Δ5-desaturase, (2) enhance the DGLA pool and its attendant oxylipins by inhibiting Δ5-desaturase as well as inducing DGLA production by Δ8-desaturase, and (3) by limiting EPA loss/conversion to DPA by inhibiting Δ5-elongase, and (4) by promoting production of oxylipins from other LC-PUFAs, such as HODEs from LA, all of which would limit the ill effects of ARA-derived oxylipins by (1) suppressing ARA synthesis, (2) introducing substrates that compete with ARA for oxygenating enzymes, and (3) producing oxylipins that inhibit oxygenation enzymes. As described before, EPA and DGLA are particularly well suited to compete with ARA owing to their 20-carbon structure, whereas longer LC-PUFAs may be farther afield and may produce a more limited variety of oxylipins. To with, compared to the 22-carbon DHA, EPA has a greater complement of anti-thrombotic oxylipins, as it produces copious amounts of prostanoids, particularly, thromboxanes that are less apt to cause thrombosis vs. thromboxanes from ARA, prostacyclins, and prostaglandins. Furthermore, EPA produces leukotrienes that can offset the ill-effects of leukotrienes from ARA. These suites of “medicinal” oxylipins would be lacking in DHA. As such, all things being equal, EPA may have the advantages of offsetting the ill effects of ARA while preserving the ability to promote HO-1 and related antioxidants. That said, the benefits of LR-EtEPA in delivering LC-PUFAs to the site of cellular injury need not be restricted to EPA, even if EPA is the preferred embodiment.

Classically, HMOX1 is an inducible gene coding HO-1, induced by a variety of cell-injury and other stimuli. Other experiments suggest EPA free acid can upregulate HMOX1 and HO-1 (FIG. 34A). To the extent that IPE also induces HMOX1, this itself could mediate some of IPE's benefits, particularly for preventing MACE. Moreover, insofar as the lymph-releasing IPE formulation enhances EPA levels within the lungs, heart, brain, kidneys, pancreas, and jejunum, LR-EtEPA could be particularly useful in diseases affecting these organs, including MACE and lung diseases, and any disease involving cellular lysis, including hemolysis or lysis of other blood cells, or lysis associated with organ infarction or cancer.

In contrast to HMOX1, heme oxygenase 2 gene (HMOX2) is a separate gene with different regulators and encodes the protein heme oxygenase-2 (HO-2). Importantly, HMOX2 is thought to be produced constitutively, and few molecules have been found that alter its expression. A notable exception to this is that HMOX2 is induced by corticosteroids. Steroid-induction might be mediated by suppressing COX and LOX, and thereby strongly suppresses arachidonic acid (AA) oxidation, in turn, limiting exposure to bioactive AA-derived oxylipins, a canonical steroid effect. Similarly, LR-EtEPA suppresses AA and raises EPA and EPA/AA, which effects are analogous to those of steroids, insofar as the net effect of to diminish AA oxidation. This is achieved as EPA competes with arachidonic acid and is metabolized to distinct, analogous oxylipins, many of which have anti-inflammatory effects. In other words, large-dose IPE is expected to have anti-inflammatory effects via oxylipin metabolites that resemble critical aspects of corticosteroid effects. As such, IPE may offer an alternative to steroids to induce HMOX2, or may act as a steroid-sparing agent.

Other genes are tightly co-regulated with HO-1 by the same antioxidant response element (ARE): NQ01 and GST. COX2 is an enzyme involved in converting EPA into bioactive oxylipins that may be less inflammatory compared to analogous oxylipins originating from AA. Angiotensin-converting enzyme (ACE) is produced in the lungs and regulates angiotensin I to the vasoconstrictive angiotensin II, thereby affecting blood pressure. In unpublished proteomics work, ACE was inhibited by EPA (FIG. 34B).

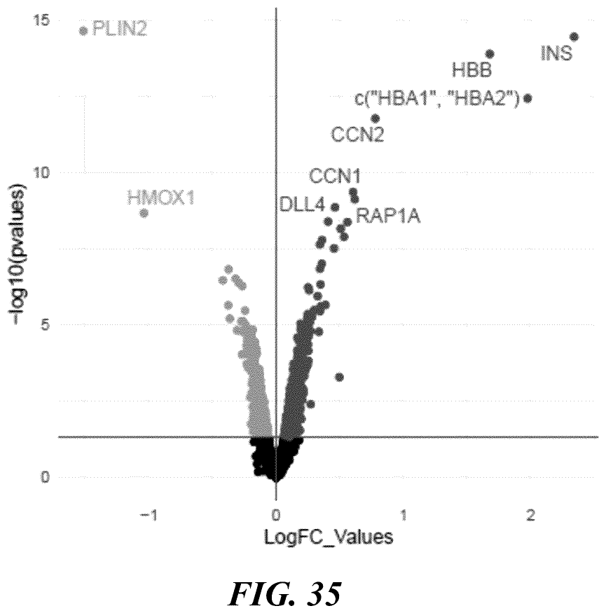

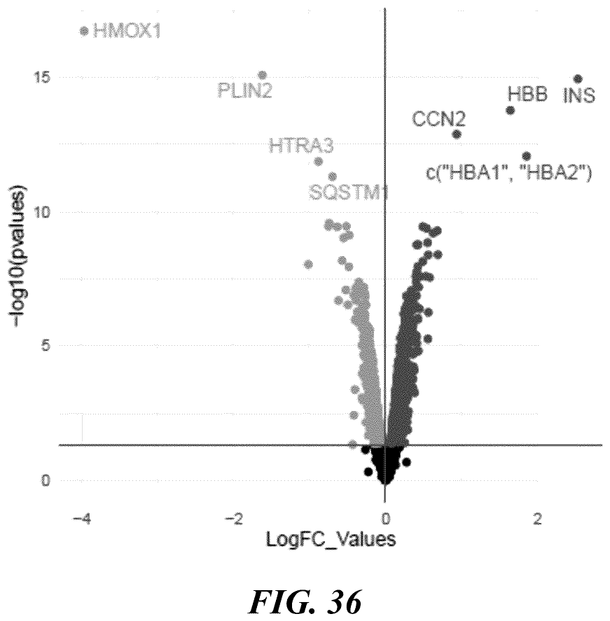

In a series of tissue culture experiments cells were provoked with IL-6 to induce an inflammatory and cellular injury response, and EPA added to determine whether it moderated proteins involved in injury responses. Notably, EPA increased HO-1 upon provoking inflammation in pulmonary endothelial cells (1.9-fold for EPA+IL-6 vs IL-6 alone, p=2.8×10⁻³²), vascular endothelial cells (2.2-fold for EPA+IL-6 vs IL-6 alone, p=7.9×10⁻⁶⁰), and brain endothelial cells (1.5-fold for EPA+IL-6 vs IL-6 alone, p=4.70×10⁻¹⁶) (see FIGS. 35-36). Notably, in vascular and brain endothelial cells, adding EPA significantly lowered fatty acid desaturase 2 (FADS2), the gene that encodes 06-desaturase, and fatty acid desaturase 1 (FADS1), the gene that encodes Δ5-desaturase. Thus, the proteomics results not only affirm the ability of EPA to promote HO-1, but also confirm the ability of EPA to inhibit A5-, and 06-desaturases, corroborating the aforementioned ability of EtEPA to suppress the Δ5-desaturase index, for example.

As an example of a condition that would especially benefit from enhancing HO-1 and related antioxidants, consider SIRS, sepsis, and ARDS. For reference, the 1.033 g IPE/kg/day dose in the Long-Evans rat is comparable to a 10 g/day dose in a human. Since Vascepa® is dosed at 4 g/day clinically, this is 2.5-fold the typical dose. This dose was selected as a reasonable dose to consider for the treatment of SIRS, sepsis and ARDS. The data from this experiment support the concept that EtEPA and especially LR-EtEPA would improve ARDS by altering the cellular injury response at several points: (1) by raising EPA/AA, OXP/AA, or MOP/AA as measures of enhanced levels of oxylipin precursors with less deleterious oxylipins vs. AA-derived oxylipins, (2) by inhibiting synthesis of AA from DGLA by suppressing FADS2 and Δ5-desaturase, (3) by promoting synthesis of DGLA by inducing FADS1 and Δ8-desaturase, (4) by diminishing AA-derived oxylipins and/or augmenting DGLA-derived oxylipins from both (2) and (3) above, (5) by diminishing AA-derived oxylipins and augmenting EPA-derived oxylipins by providing copious amounts of EPA, and particular to the HO-1 example in this section, (6) by inducing the antioxidant response element and other pathways that induce HMOX1 and/or HMOX2, NQ01, GST, and GHS.

### Example 5: EPA Increased Endothelial Nitric Oxide Synthase (eNOS) Levels and Proteins Associated with Cellular Responses to Oxidative Stress During Inflammation

The objective of this study is to measure and compare the effects of EPA and DHA on eNOS levels and expression of proteins that regulate ROS in human vascular ECs under conditions of inflammation.

Human umbilical vein endothelial cells (HUVECs) were isolated into primary cultures from female donors by Clonetics (San Diego, California) and purchased as proliferating cells. All cell culture donors were healthy, with no pregnancy or prenatal complications. The cultured cells were incubated in 95% air/5% CO2 at 37° C. and passaged by an enzymatic (trypsin) procedure. The confluent cells (4 to 5×10⁵ cells/35 mm dish) were placed with minimum essential medium containing 3 mM L-arginine and 0.1 mM BH4 [(6R)-5,6,7,8-tetrahydrobiopterin]. Before experimental use, the cells (from second or third passage) were rinsed twice with Tyrode-HEPES buffer with 1.8 mM CaCl₂).

EPA and DHA were purchased from Sigma-Aldrich (St. Louis, MO) and prepared initially in redistilled ethanol. Primary and secondary stock solutions were prepared and stored under nitrogen at −20° C.

HUVECs were treated with vehicle, EPA, or DHA (10 μM) for 2 hours, and then challenged with IL-6 (12 ng/mL) for 24 h hours. After incubation, cells were pelleted and frozen at −80° C. until proteomic analysis was performed.

Relative protein expression levels among the various treatments were measured using LC/MS proteomic techniques. Following protein digest, peptides are separated over a reverse phase column and then identified based on their mass.

Cell pellets were lysed using methanol/chloroform extraction. Proteins were denatured, reduced, alkylated, and trypsin digested. Samples were then prepared for Tandem Mass Tag (TMT) 10plex labeling. A bicinchoninic acid (BCA) assay was performed on each sample to quantify the total protein in each sample, which is important to confirm equal amounts of each sample are added to the multiplex sample. Each peptide in a sample was given a unique, low molecular weight (typically 126-130 Da), and then the samples were combined.

Each multiplexed sample was then fractionated to increase the overall protein coverage using high pH reversed phase fractionation and analyzed by LC/MS using a Dionex UltiMate 3000 RSLC in tandem with a Q-Exactive/Lumos Orbitrap Mass Spectrometer. The chromatography was performed using a 2-hour gradient on a Thermo Pepmap C18 column (100 Å pore size, 3.0 μm particle size, 100 μM×150 mm) set at 50° C. Mobile phase A was water with 0.1% formic acid, and mobile phase B was acetonitrile with 0.1% formic acid.

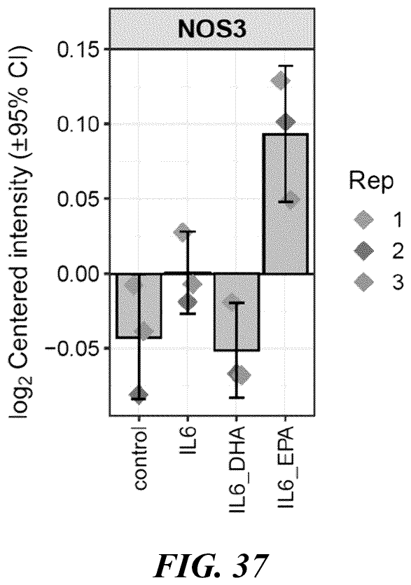

Proteins that showed a fold change >1.0 and p<0.05 for the relevant comparisons were considered significant and further analyzed. A bioinformatic package was applied to all the mass spectrometry intensity data known as Differential Enrichment analysis of Proteomics data (DEP) to process proteomic data (FIGS. 35-37).

TABLE 5
Summary of Key Protein Changes with EPA and DHA

| Protein | EPA | DHA |
|---|---|---|
| eNOS | 1.1-fold increase, | n.s |
| | p = 0.014 | |
| DDAH-1 | 1.1-fold increase, | n.s. |
| | p = 0.010 | |
| DDAH-2 | 1.1-fold increase, | n.s. |
| | p = 0.0009 | |
| TXN | 1.2-fold increase, | 1.2-fold increase, |
| | p = 1.35 × 10⁻⁵ | p = 4.95 × 10⁻⁶ |
| GSR | 1.1-fold increase, | 1.2-fold increase, |
| | p = 0.0008 | p = 3.32 × 10⁻⁶ |
| PRDX1 | 1.1-fold increase, | 1.1-fold increase, |
| | p = 0.0004 | p = 0.005 |
| PRDX2 | 1.1-fold increase, | n.s. |
| | p = 0.047 | |
| PRDX3 | n.s. | 1.1-fold increase, |
| | | p = 0.0004 |
| PRDX4 | n.s. | 1.5-fold increase |
| | | p = 2.14 × 10⁻⁷ |
| PRDX5 | 1.1-fold increase, | 1.1-fold increase, |
| | p = 0.032 | p = 0.006 |
| PRDX6 | n.s. | 1.3-fold decrease |
| | | p = 0.0002 |

In summary, EPA and DHA significantly stimulated or repressed the expression of 544/472 and 864/767 proteins, respectively, compared with IL-6 alone. Specifically, EPA increased expression of eNOS by 1.1-fold (p=0.014) as compared to DHA. EPA, but not DHA, increased dimethylarginine dimethylaminohydrolase (DDAH-1) and DDAH-2 levels, enzymes that hydrolyze endogenous eNOS inhibitors. Additionally, EPA and DHA increased proteins that limit oxidative stress, including glutathione reductase (GSR), thioredoxin (TXN), and peroxiredoxin (PRDX) species.

Accordingly, the inventor has shown that under inflammatory conditions, EPA significantly increased expression of eNOS and proteins that reduce ROS as compared to DHA in vascular endothelial cells. These changes in protein expression during inflammation may contribute to preserved arterial function and reduced cardiovascular risk.

### Example 6: EPA Modulates Expression of Inflammatory Proteins in Pulmonary Endothelial Cells Following Exposure to Air Pollution Particle Matter

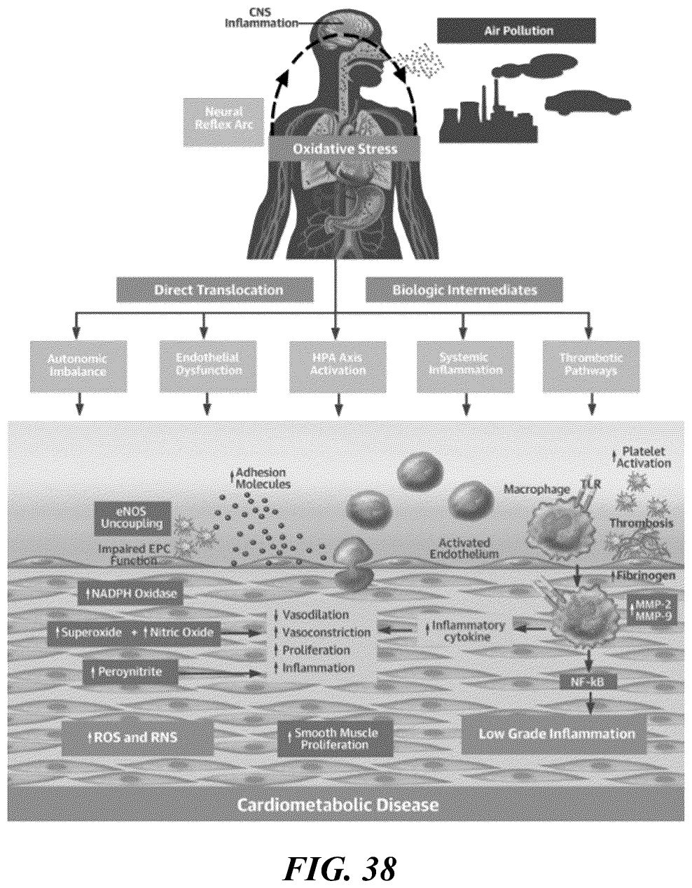

As illustrated in FIG. 38, air pollution is a major contributor to global mortality and various chronic conditions, including cardiovascular diseases. See Rajagopalan et al., J. Am. Coll. Cardiol. (2018) 72:2054-2070, incorporated herein by reference in its entirety. Exposure to fine particulate matter (PM) in air pollution (≤2.5 μm in mean diameter) causes both acute and chronic tissue injury as a result of penetration and toxic chemical constituents. See Franklin et al., Curr. Probl. Cardiol. (2015) 40:207-2382, incorporated herein by reference in its entirety. In pulmonary endothelial cells, PMs cause nitric oxide (NO) synthase (eNOS) uncoupling resulting in loss of function, apoptosis, and aberrant immune responses. Cherng et al., Environ. Health Perspect. (2011) 119:98-103; Hansen et al., Toxicol. Appl. Pharmacol. (2007) 219:24-32; Pope et al., Circ. Res. (2016) 119:1204-1214; and Courtois et al., Environ. Health Perspect. (2008) 116:1294-1299, each of which is incorporated herein by reference in its entirety. Systemic inflammatory damage is also associated with PM exposure as evidenced by increased neutrophil degranulation. See Lacy, Allergy Asthma Clin. Immunol. (2006) 2:98; Hoenderdos et al., Thorax. (2016) 71:1030-1038, each of which is incorporated herein by reference in its entirety. When challenged with pro-inflammatory stimuli, endothelial cells produce signals (IL-8) that induce such neutrophil degranulation. See Gill et al., FASEB J. (1998) 12:673-684, incorporated herein by reference in its entirety. Finally, loss of NO-dependent vasomotor control has been reported in human and animal models of PM exposure. See Courtois et al., Environ. Health Perspect. (2008) 116:1294-1299; Nurkiewicz et al., Environ. Health Perspect. (2004) 112:1299-1306; Brook et al., Circulation (2002) 105:1534-1536, each of which is incorporated herein by reference in its entirety.

EPA administered as icosapent ethyl (IPE) is the first FDA approved drug to reduce cardiovascular risk among patients with elevated triglyceride levels as an add-on to maximally tolerated statin therapy. The REDUCE-IT trial showed that treatment with high dose IPE (4 g/day) reduced composite cardiovascular events by 25% in statin-treated patients with elevated baseline triglyceride levels. See Bhatt et al., N. Engl. J. Med. (2019) 380:11-22, incorporated herein by reference in its entirety. The benefits of IPE were independent of baseline triglyceride levels but positively correlated with plasma levels of EPA. Imaging trials showed a significant regression in plaque volume and composition with IPE compared to statin alone in patients with atherothrombotic disease. See Budoff et al., Euro. Heart J. (2020) 41:3925-3932; Watanabe et al., J. Cardiol. (2017) 70:537-544, each of which is incorporated herein by reference in its entirety. In contrast to IPE treatment, the results of trials using mixed omega-3 fatty acids have failed to reduce cardiovascular events. This may be due to differences in formulation and potential pleiotropic benefits and membrane interactions unique to EPA. See Mason et al., Metab. Clin. (2022) 130:155161; Sherratt et al., Prostaglandin Leukot. Essent. Fatty Acid (2021)173; Sherratt et al., J. Lipid Res. (2021) 62; Mason et al., Arterioscler. Thromb. Vasc. Biol. (2020) 40:1135-1147, each of which is incorporated herein by reference in its entirety.

This example evaluated the ability of EPA to modulate expression of inflammatory proteins and related pathways, including neutrophil degranulation, in pulmonary endothelial cells following exposure to air pollution PMs of different sizes.

Primary human lung microvascular endothelial cells (HMVEC-Ls, PECs) were purchased from Lonza (Manassas, VA). Cells were cultured in complete endothelial cell growth medium and maintained at 37° C. in a 95% air/5% CO₂ humidified incubator. Cells were supplied with fresh medium every other day and propagated by an enzymatic (trypsin) procedure. Cell culture medium also contained 2% FBS to facilitate fatty acid treatment.

EPA was purchased from Sigma-Aldrich (Saint Louis, MO) and solubilized in redistilled ethanol under nitrogen atmosphere and stored at −20° C.

Urban air particulate matter and fine particulate matter were purchased from Sigma-Aldrich (SRM 1648a), catalog #: NIST1648a3110 and NIST27863110. The mean particle diameters were 5.85 and 2.8 μm, respectively.

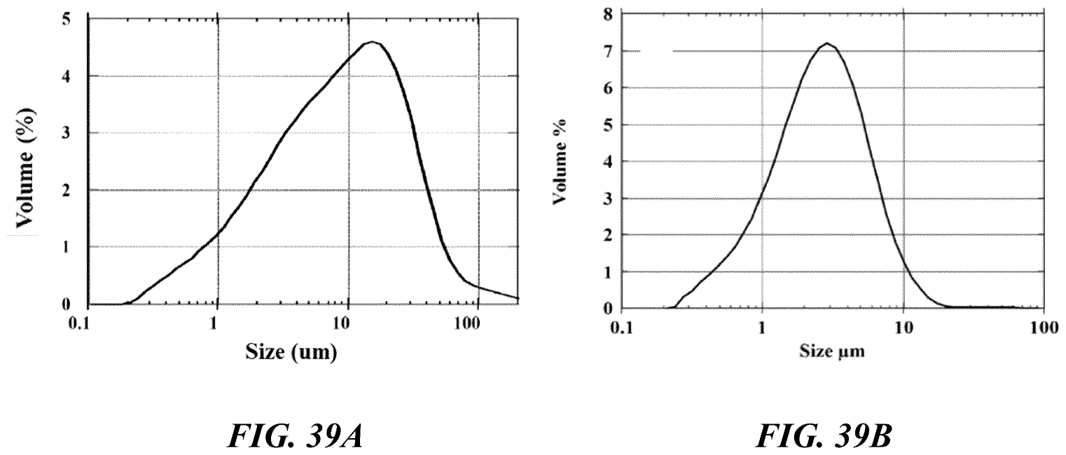

FIGS. 39A-39B show particle size distribution for SRM 1648a and SRM2786 after 10-minute and 1-hour sonication in water, respectively. Solid line represents the volume in % (see NIST Certificate of Analysis, SRM1648a and NIST Certificate of Analysis, SRM2786).

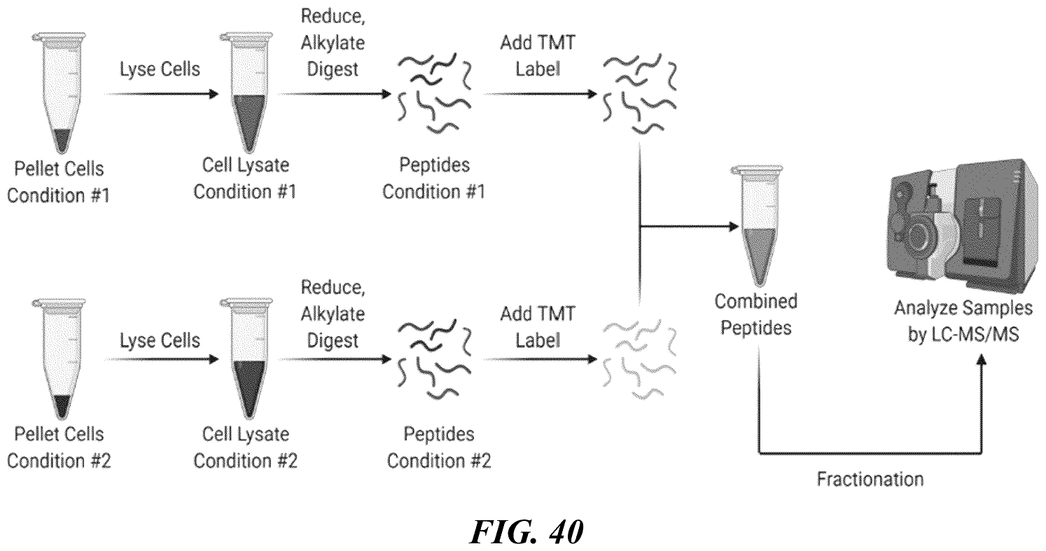

PECs were pre-treated with EPA (40 μM) for 2 hours in 2% FBS-containing medium and then challenged with urban particulate matter (50 μg/mL) for 2 hours. After 2 hours, media was washed out and HBSS buffer was added. After incubation, cells were pelleted and frozen at −80° C. until proteomic analysis was performed. Relative protein expression levels among the various treatments were measured using LC/MS proteomic techniques (FIG. 40).

Cell pellets were lysed using methanol/chloroform extraction. Proteins were then denatured, reduced, alkylated, and trypsin digested. Samples were labeled by Tandem Mass Tag (TMT) 10plex labeling to multiplex the samples and ensure that the injection, LC, and MS conditions are identical for each sample. A bicinchoninic acid (BCA) assay was performed to quantify the total protein in each sample. Each peptide in a sample was given a unique, low molecular weight (typically 126-130 Da), and then the samples were combined.

Each multiplexed sample was then fractionated to increase the overall protein coverage using high pH reversed phase fractionation and analyzed by LC/MS using a Dionex UltiMate 3000 RSLC in tandem with a Q-Exactive/Lumos Orbitrap Mass Spectrometer.

Proteins that showed a fold change >1.0 and p<0.05 for the relevant comparisons were considered significant and further analyzed. A ComBat function was applied to all mass spectrometry intensity data to correct for batch effects. The bioinformatic package Differential Enrichment analysis of Proteomics data (DEP) was used to process proteomic data and generate the figures.

In the present study, the inventors have discovered the following results:

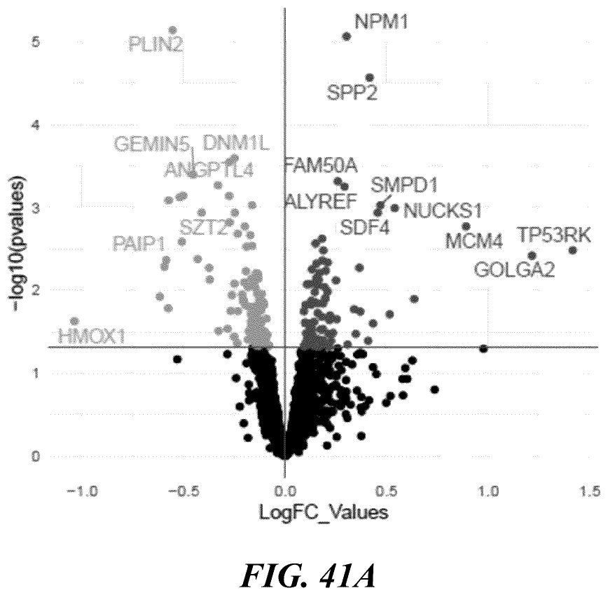

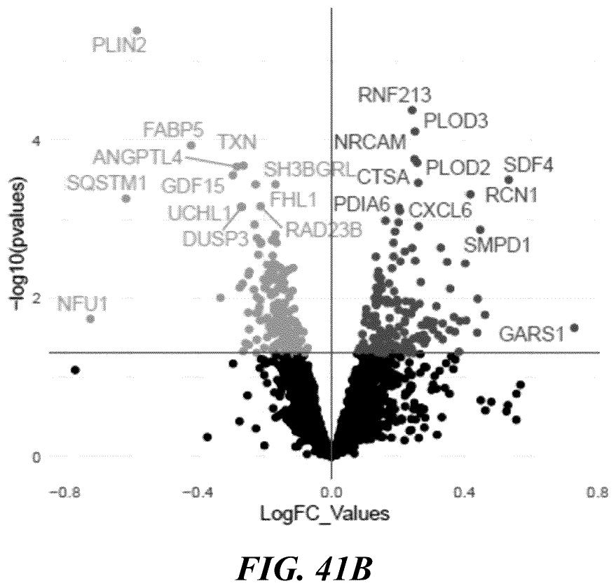

First, as shown in FIGS. 41A-41B, EPA significantly modulated expression of 205 and 347 proteins relative to fine and urban PMs, respectively.

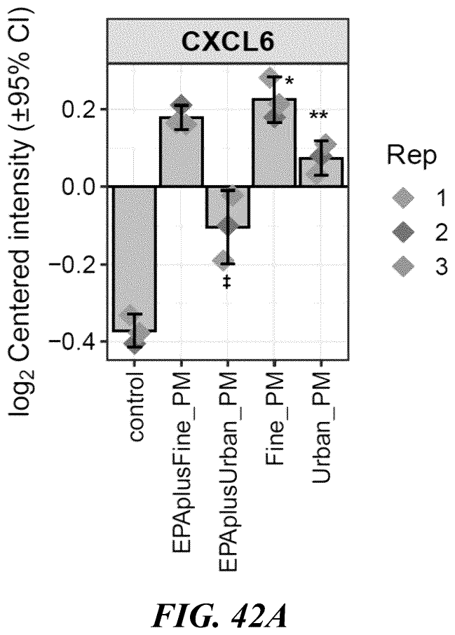

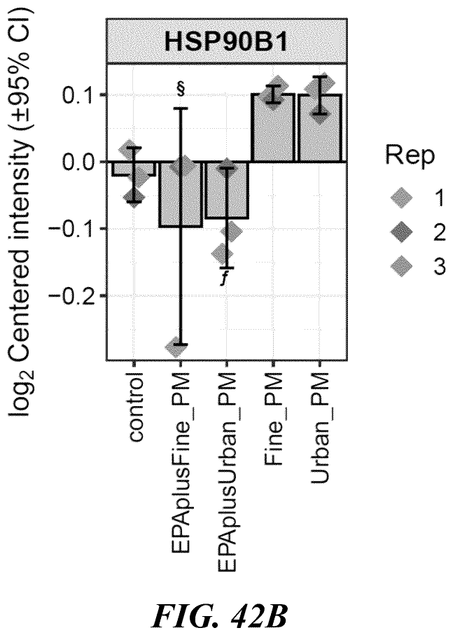

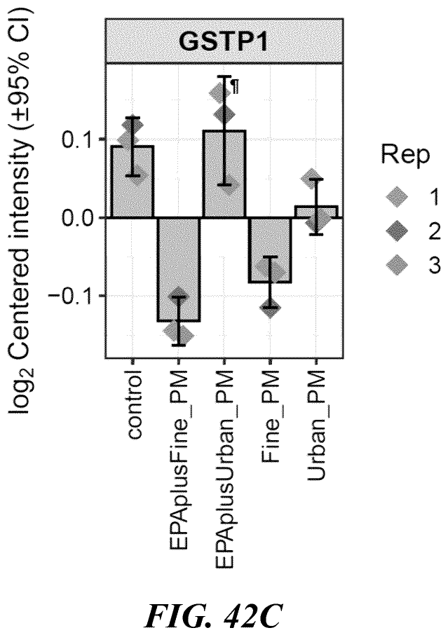

FIGS. 42A-42C show normalized intensity values of each replicate in the treatment groups (N=3). CXCL6, -X-C motif chemokine 6; HSP90B1, heat shock protein 90-β, GSTP1, glutathione S-transferase P. § p=0.015 vs Fine PM; fp=0.011 vs Urban PM; †p=0.030 vs Fine PM; *p=8.88×10⁻⁹ vs control; **p=3.79×10⁻⁷ vs control; ‡p=0.0008 vs Urban PM; ¶p=0.036 vs Urban PMs.

Second, as summarized in Table 6 below, among pathways modulated by both PMs was neutrophil degranulation (Gene Ontology ID: 0043312), where fine and urban PMs modulated 36 and 13 proteins, respectively. Common to fine and urban PMs were eight proteins, including 1.5-fold and 1.3-fold increases in C-X-C motif chemokine 6, respectively.

TABLE 6
Summary of changes to Neutrophil Degranulation Pathway (GO: 0043312)

| | # of Proteins | # of Proteins | |
|---|---|---|---|
| | Increased Expression | Decreased Expression | Pathway |
| Comparison | by Treatment | by Treatment | adjusted-p value |
| Control vs Fine PM | 8 | 28 | 1.62 × 10⁻¹⁰ |
| Control vs Urban PM | 6 | 7 | 0.071 |
| Fine PM vs EPA + Fine PM | 11 | 11 | 2.08 × 10⁻⁶ |
| Urban PM vs EPA + Urban PM | 16 | 19 | 4.43 × 10⁻⁹ |

The protein increases/decreases are determined based on the treatment effects (i.e., the 2nd treatment in each comparison).

Third, EPA treatment modulated 22 and 35 proteins associated with neutrophil degranulation compared to fine and urban PMs, respectively.

Fourth, relative to fine PMs, EPA increased expression of heat shock protein 90-β and decreased expression of interleukin enhancer-binding factor 2.

Fifth, compared to urban PMs, EPA decreased expression of C-X-C motif chemokine 6 and increased expression of glutathione S-transferase P.

In summary, EPA favorably modulated expression of various cytoprotective as well as inflammatory proteins in pulmonary ECs during exposure to multiple air pollution PMs. These findings support a potential cardiovascular benefit for EPA under inflammatory conditions caused by air pollution PMs.

The present technology includes, but is not limited to, the following specific embodiments:

1. A composition comprising: (a) at least 15%, by weight, one or more polyunsaturated fatty acids (PUFAs) or derivatives thereof; and (b) 1% to 85%, by weight, a source of phospholipid.

2. The composition of embodiment 1, further comprising (c) 1% to 20%, by weight, one or more emulsifiers.

3. The composition of embodiment 1 or 2, wherein the one or more PUFAs or derivatives thereof are selected from the group consisting of linoleic acid (LA), gamma-linoleic acid (GLA), dihomo-gamma-linoleic acid (DGLA), arachidonic acid (AA), adrenic acid (AdA), omega-6 docosapentaenoic acid (DPA6), alpha-lineoleic acid (ALA), stearidonic acid (SDA), omega-3 eicosatetraenoic acid (ETA), eicosapentaenoic acid (EPA), docosapentaenoic acid (DPA), docosahexaenoic acid (DHA), an LA derivative, a GLA derivative, a DGLA derivative, an AA derivative, an AdA derivative, a DPA6 derivative, an ALA derivative, an SDA derivative, an ETA derivative, an EPA derivative, a DPA derivative, and a DHA derivative.

4. The composition of any one of embodiments 1-3, wherein the PUFA derivative comprises an oxylipin.

5. The composition of embodiment 3, wherein the LA derivative is selected from the group consisting of 9-hydroperoxy-octadecadienoic acid (9-HpODE), 13-hydroperoxy-octadecadienoic acid (13-HpODE), 9-hydroxy-octadecadienoic acid (9-HODE), 13-hydroxy-octadecadienoic acid (13-HODE), 9,10,13 trihydroxy-octadecenoic acid (9,10,13 TriHOME), 9,12,13 trihydroxy-octadecenoic acid (9,12,13 TriHOME), 9-oxo-octadecadienoic acid (9-oxo-ODE), 13-oxo-octadecadienoic acid (13-oxo-ODE), 9,10-epoxy-octadecenoic acid (9,10-EpOME), 12,13-epoxy-octadecenoic acid (12,13-EpOME), 9,10-dihydroxy-octadecenoic acid (9,10-DiHOME), and 12,13-dihydroxy-octadecenoic acid (12,13-DiHOME).

6. The composition of embodiment 3, wherein the GLA derivative is selected from the group consisting of 6-hydroxy-octatrienoic acid (6-HOTrE or 6-hydroxy-GLA), 7-hydroxy-octatrienoic acid (7-HOTrE or 7-hydroxy-GLA), 9-hydroxy-octatrienoic acid (9-HOTrE or 9-hydroxy-GLA), 10-hydroxy-octatrienoic acid (10-HOTrE or 10-hydroxy-GLA), 12-hydroxy-octatrienoic acid (12-HOTrE or 12-hydroxy-GLA), 13-hydroxy-octatrienoic acid (13-HOTrE or 13-hydroxy-GLA), 6,13-dihydroxy-octadienoic acid (6,13-DiHODE or 6,13-dihydroxy-GLA), and trihydroxy GLA derivatives (trihydroxy-GLAs).

7. The composition of embodiment 3, wherein the DGLA derivative is selected from the group consisting of prostaglandin D1 (PGD1), prostaglandin E1 (PGE1), 15-hydroxy-PGE1, 19-hydroxy-PGE1, 13,14-dihydroxy-PGE1, 13,14-dihydroxy-15-keto-PGE1, prostaglandin F1α (PGF1α), 6-keto-PGF1α, 15-keto-PGF1α, 13,14-dihydroxy-PGF1α, 15,19-dihydroxy-PGF1α, 13,14-dihydroxy-15-keto PGF1α, prostacyclin 11 (prostaglandin 11 or PGI1), thromboxane A1 (TXA1), thromboxane B1 (TXB1), leukotriene B3 (LTB3), leukotriene C3 (LTC3), leukotriene D3 (LTD3), leukotriene E3 (LTE3), 5-hydroperoxy-eicosatrienoic acid (5-HpETrE), 8-hydroperoxy-eicosatrienoic acid (8-HpETrE), 12-hydroperoxy-eicosatrienoic acid (12-HpETrE), 15-hydroperoxy-eicosatrienoic acid (15-HpETrE), 5-hydroxy-eicosatrienoic acid (5-HETrE), 8-hydroxy-eicosatrienoic acid (8-HETrE), 12-hydroxy-eicosatrienoic acid (12-HETrE), 15-hydroxy-eicosatrienoic acid (15-HETrE), 8,9-epoxy-eicosadienoic acid (8,9-EpEDE), 11,12-epoxy-eicosadienoic acid (11,12-EpEDE), 14,15-epoxy-eicosadienoic acid (14,15-EpEDE), 8,9-dihydroxy-eicosadienoic acid (8,9-DiHEDE), 11,12-dihydroxy-eicosadienoic acid (11,12-DiHEDE), and 14,15-dihydroxy-eicosadienoic acid (14,15-DiHEDE).

8. The composition of embodiment 3, wherein the AA derivative is selected from the group consisting of 6-keto-prostaglandin F1 alpha (6k-PGF1α), thromboxane B2 (TXB2), 11-dehydro-thromboxane B2 (11-dTXB2), prostaglandin F2 alpha (PGF2a), prostaglandin E2 (PGE2), prostaglandin A2 (PGA2), prostaglandin D2 (PGD2), 2,3-dinor 11 beta-prostaglandin F2 alpha (2,3-dinor11bPGF2a), prostaglandin J2 (PGJ2), 15-deoxy-delta-12,14-prostaglandin J2 (15d-PGJ2), leukotriene B4 (LTB4), 20-hydroxy-leukotriene B4 (20-OH-LTB4), leukotriene C4 (LTC4), leukotriene D4 (LTD4), leukotriene E4 (LTE4), 5-hydroperoxy-eicosatetraenoic acid (5-HpETE), 8-hydroperoxy-eicosatetraenoic acid (8-HpETE), 9-hydroperoxy-eicosatetraenoic acid (9-HpETE), 11-hydroperoxy-eicosatetraenoic acid (11-HpETE), 12-hydroperoxy-eicosatetraenoic acid (12-HpETE), 15-hydroperoxy-eicosatetraenoic acid (15-HpETE), 5-hydroxy-eicosatetraenoic acid (5-HETE), 8-hydroxy-eicosatetraenoic acid (8-HETE), 9-hydroxy-eicosatetraenoic acid (9-HETE), 11-hydroxy-eicosatetraenoic acid (11-HETE), 12-hydroxy-eicosatetraenoic acid (12-HETE), 15-hydroxy-eicosatetraenoic acid (15-HETE), 18-hydroxy-eicosatetraenoic acid (18-HETE), 19-hydroxy-eicosatetraenoic acid (19-HETE), 20-hydroxy-eicosatetraenoic acid (20-HETE), 5-oxo-eicosatetraenoic acid (5-oxo-ETE), 8-oxo-eicosatetraenoic acid (8-oxo-ETE), 11-oxo-eicosatetraenoic acid (11-oxo-ETE), 12-oxo-eicosatetraenoic acid (12-oxo-ETE), 15-oxo-eicosatetraenoic acid (15-oxo-ETE), lipoxin A4 (LXA4), lipoxin A5 (LXA5), hepoxilin A3 (HxA3), hepoxilin B3 (HxB3), trioxilin A3 (TrxA3), trioxilin B3 (TrxB3), eoxin A4 (ExA4), eoxin C4 (ExC4), eoxin D4 (ExD4), eoxin E4 (ExE4), 5,6-epoxy-eicosatrienoic acid (5,6-EpETrE, or 5,6-EET), 8,9-epoxy-eicosatrienoic acid (8,9-EpETrE, or 8,9-EET), 11,12-epoxy-eicosatrienoic acid (11,12-EpETrE, or 11,12-EET), 14,15-epoxy-eicosatrienoic acid (14,15-EpETrE, or 14,15-EET), 5,12-dihydroxy-eicosatetraenoic acid (5,12-DiHETE), 5,6-dihydroxy-eicosatrienoic acid (5,6-DiHETrE, or 5,6-DiHET), 8,9-dihydroxy-eicosatrienoic acid (8,9-DiHETrE, or 8,9-DiHET), 11,12-dihydroxy-eicosatrienoic acid (11,12-DiHETrE, or 11,12-DiHET), 14,15-dihydroxy-eicosatrienoic acid (14,15-DiHETrE, or 14,15-DiHET), and 12-hydroxyheptadecatrenoic acid (12-HHTrE).

9. The composition of embodiment 3, wherein the AdA derivative is selected from the group consisting of dihomo-prostaglandin E2 (dihomo-PGE2), dihomo-prostaglandin D2 (dihomo-PGD2), dihomo-prostaglandin F1α (dihomo-PGF2a), dihomo-prostacyclin 12 (dihomo-prostaglandin 12 or dihomo-PGI2), dihomo-thromboxane A2 (dihomo-TXA2), dihomo-thromboxane B2 (dihomo-TXB2), 7-hydroperoxy-docosatetraenoic acid (dihomo-7-HpETE), 10-hydroperoxy-docosatetraenoic acid (dihomo-10-HpETE), 11-hydroperoxy-docosatetraenoic acid (dihomo-11-HpETE), 13-hydroperoxy-docosatetraenoic acid (dihomo-13-HpETE), 14-hydroperoxy-docosatetraenoic acid (dihomo-14-HpETE), 17-hydroperoxy-docosatetraenoic acid (dihomo-17-HpETE), 7-hydroxy-docosatetraenoic acid (dihomo-7-HETE), 10-hydroxy-docosatetraenoic acid (dihomo-10-HETE), 11-hydroxy-docosatetraenoic acid (dihomo-11-HETE), 13-hydroxy-docosatetraenoic acid (dihomo-13-HETE), 14-hydroxy-docosatetraenoic acid (dihomo-14-HETE), 17-hydroxy-docosatetraenoic acid (dihomo-17-HETE), 7,11-dihydroxy-docosatetraenoic acid (dihomo-7,11-DiHETE), 7,14-dihydroxy-docosatetraenoic acid (dihomo-7,14-DiHETE), 7,17-dihydroxy-docosatetraenoic acid (dihomo-7,17-DiHETE), 10,17-dihydroxy-docosatetraenoic acid (dihomo-10,17-DiHETE), 11,17-dihydroxy-docosatetraenoic acid (dihomo-11,17-DiHETE), 13,15-dihydroxy-docosatetraenoic acid (dihomo-13,15-DiHETE), 13,17-dihydroxy-docosatetraenoic acid (dihomo-13,17-DiHETE), 16,17-dihydroxy-docosatetraenoic acid (dihomo-16,17-DiHETE), 7,8-epoxy-docosatrienoic acid (dihomo-7,8-EpETrE), 10,11-epoxy-docosatrienoic acid (dihomo-10,11-EpETrE), 13,14-epoxy-docosatrienoic acid (dihomo-13,14-EpETrE), 16,17-epoxy-docosatrienoic acid (dihomo-16,17-EpETrE), 7,8-dihydroxy-docosatrienoic acid (dihomo-7,8-DiHETrE), 10,11-dihydroxy-docosatrienoic acid (di homo-10,11-DiHETrE), 13,14-dihydroxy-docosatrienoic acid (dihomo-13,14-DiHETrE), 16,17-dihydroxy-docosatrienoic acid (dihomo-16,17-DiHETrE), 7,16,17-trihydroxy-docosatetraenoic acid (dihomo-7,16,17-trihydroxy-ETrE), and 14-hydroxy-7,10,12-nonadecatrienoic acid (14-HNTrE).

10. The composition of embodiment 3, wherein the DPA6 derivative is selected from the group consisting of 7-hydroperoxy-DPA6, 8-hydroperoxy-DPA6, 10-hydroperoxy-DPA6, 11-hydroperoxy-DPA6, 13-hydroperoxy-DPA6, 14-hydroperoxy-DPA6, 17-hydroperoxy-DPA6, 7-hydroxy-DPA6, 8-hydroxy-DPA6, 10-hydroxy-DPA6, 11-hydroxy-DPA6, 13-hydroxy-DPA6, 14-hydroxy-DPA6, 17-hydroxy-DPA6, 4,5-dihydroxy-DPA6, 7,14-dihydroxy-DPA6, 7,17-dihydroxy-DPA6, 8,14-dihydroxy-DPA6, 10,17-di hydroxy-DPA6, 13,17-di hydroxy-DPA6, 16,17-di hydroxy-DPA6, 4,5,17-trihydroxy-DPA6, 7,16,17-trihydroxy-DPA6, and 10,13,17-trihydroxy-DPA6.

11. The composition of embodiment 3, wherein the ALA derivative is selected from the group consisting of 9-hydroperoxy-octatrienoic acid (9-HpOTrE), 13-hydroperoxy-octatrienoic acid (13-HpOTrE), 9-hydroxy-octatrienoic acid (9-HOTrE), 13-hydroxy-octatrienoic acid (13-HOTrE), 9,16-dihydroxy-octatrienoic acid (9,16-DiHOTrE), 9-oxo-octatrienoic acid (9-oxo-OTrE), 13-oxo-octatrienoic acid (13-oxo-OtrE), 9,10-epoxy-octadienoic acid (9,10-EpODE), 12,13-epoxy-octadienoic acid (12,13-EpODE), 15,16-epoxy-octadienoic acid (15,16-EpODE), 9,10-dihydroxy-octadienoic acid (9,10-DiHODE), 12,13-dihydroxy-octadienoic acid (12,13-DiHODE), and 15,16-dihydroxy-octadienoic acid (15,16-DiHODE).

12. The composition of embodiment 3, wherein the SDA derivative is selected from the group consisting of 6-hydroperoxy-octatetraenoic acid (6-HpOTE or 6-hydroperoxy-SDA), 7-hydroperoxy-octatetraenoic acid (7-HpOTE or 7-hydroperoxy-SDA), 9-hydroperoxy-octatetraenoic acid (9-HpOTE or 9-hydroperoxy-SDA), 10-hydroperoxy-octatetraenoic acid (10-HpOTE or 10-hydroperoxy-SDA), 12-hydroperoxy-octatetraenoic acid (12-HpOTE or 12-hydroperoxy-SDA), 13-hydroperoxy-octatetraenoic acid (13-HpOTE or 13-hydroperoxy-SDA), 15-hydroperoxy-octatetraenoic acid (15-HpOTE or 15-hydroperoxy-SDA), 16-hydroperoxy-octatetraenoic acid (16-HpOTE or 16-hydroperoxy-SDA), 6-hydroxy-octatetraenoic acid (6-HOTE or 6-hydroxy-SDA), 7-hydroxy-octatetraenoic acid (7-HOTE or 7-hydroxy-SDA), 9-hydroxy-octatetraenoic acid (9-HOTE or 9-hydroxy-SDA), 10-hydroxy-octatetraenoic acid (10-HOTE or 10-hydroxy-SDA), 12-hydroxy-octatetraenoic acid (12-HOTE or 12-hydroxy-SDA), 13-hydroxy-octatetraenoic acid (13-HOTE or 13-hydroxy-SDA), 15-hydroxy-octatetraenoic acid (15-HOTE or 15-hydroxy-SDA), 16-hydroxy-octatetraenoic acid (16-HOTE or 16-hydroxy-SDA), 6,13-dihydroxy-octadecatrienoic acid (6,13-DiHOTrE or 6,13-dihydroxy-SDA), 6,16-dihydroxy-octadecatrienoic acid (6,16-DiHOTrE or 6,16-dihydroxy-SDA), 6,7-dihydroxy-octadecadienoic acid (6,7-DiHODE or 6,7-dihydroxy-SDA), 9,10-dihydroxy-octadecadienoic acid (9,10-DiHODE or 9,10-di hydroxy-SDA), 12,13-dihydroxy-octadecadienoic acid (12,13-DiHODE or 12,13-dihydroxy-SDA), 15,16-dihydroxy-octadecadienoic acid (15,16-DiHODE or 15,16-dihydroxy-SDA), and trihydroxy-SDAs bearing a hydroxyl group at any three positions among carbons C6, C7, C9, C10, C12, C13, C15 or C16 of SDA (trihydroxy-SDAs).

13. The composition of embodiment 3, wherein the ETA derivative is selected from the group consisting of Δ17,18 prostaglandin D1 (Δ17,18 PGD1 or ω-3 PGD1), Δ17,18 prostaglandin E1 (Δ17,18 PGE1 or ω-3 PGE1), and Δ17,18 prostaglandin F1α (Δ17,18 PGF1α or ω-3 PGF1α), Δ17,18 prostacyclin 11 (Δ17,18 PGI1 or ω-3 PGE1), Δ17,18 12-hydroperoxy-eicosatetraenoic acid (Δ17,18 12-HpETE or ω-3 12-HpETE), Δ17,18 15-hydroperoxy-eicosatetraenoic acid (Δ17,18 15-HpETE or ω-3 15-HpETE), Δ16,17 18-hydroperoxy-eicosatetraenoic acid (Δ16,17 18-HpETE), Δ17,18 12-hydroxy-eicosatetraenoic acid (Δ17,18 12-HETE or ω-3 12-HETE), Δ17,18 15-hydroxy-eicosatetraenoic acid (Δ17,18 15-HETE or ω-3 15-HETE), Δ16,17 18-hydroxy-eicosatetraenoic acid (Δ16,17 18-HETE), Δ17,18 19-hydroxy-eicosatetraenoic acid (Δ17,18 19-HETE or ω-3 19-HETE), Δ17,18 20-hydroxy-eicosatetraenoic acid (Δ17,18 20-HETE or ω-3 20-HETE), Δ17,18 11,12 epoxy-eicosatrienoic acid (Δ17,18 11,12-EpETrE or ω-3 11,12-EpETrE), Δ17,18 14,15 epoxy-eicosatrienoic acid (Δ17,18 14,15-EpETrE or ω-3 14,15-EpETrE), and 17,18 epoxy-eicosatrienoic acid (17,18-EpETrE), Δ17,18 11,12 dihydroxy-eicosatrienoic acid (Δ17,18 11,12-DiHETrE or ω-3 11,12-DiHETrE), Δ17,18 14,15 dihydroxy-eicosatrienoic acid (Δ17,18 14,15-DiHETrE or ω-3 14,15-DiHETrE), and 17,18 dihydroxy-eicosatrienoic acid (17,18-DiHETrE).

14. The composition of embodiment 3, wherein the EPA derivative is selected from the group consisting of 6-keto-prostaglandin F2 alpha (6k-PGF2a), thromboxane B3 (TXB3), 11-dehydro-thromboxane B3 (11-dTXB3), prostaglandin F3 alpha (PGF3a), prostaglandin E3 (PGE3), prostaglandin A3 (PGA3), prostaglandin D3 (PGD3), 2,3-dinor 11 beta-prostaglandin F3 alpha (2,3-dinor11bPGF3a), prostaglandin J3 (PGJ3), 15-deoxy-delta-12,14-prostaglandin J3 (15d-PGJ3), leukotriene B5 (LTB5), 20-hydroxy-leukotriene B5 (20-OH-LTB5), leukotriene C5 (LTC5), leukotriene D5 (LTD5), leukotriene E5 (LTE5), 5-hydroperoxy-eicosapentaenoic acid (5-HpEPE), 8-hydroperoxy-eicosapentaenoic acid (8-HpEPE), 9-hydroperoxy-eicosapentaenoic acid (9-HpEPE), 11-hydroperoxy-eicosapentaenoic acid (11-HpEPE), 12-hydroperoxy-eicosapentaenoic acid (12-HpEPE), 15-hydroperoxy-eicosapentaenoic acid (15-HpEPE), 18-hydroperoxy-eicosapentaenoic acid (18-HpEPE), 5-hydroxy-eicosapentaenoic acid (5-HEPE), 8-hydroxy-eicosapentaenoic acid (8-HEPE), 9-hydroxy-eicosapentaenoic acid (9-HEPE), 11-hydroxy-eicosapentaenoic acid (11-HEPE), 12-hydroxy-eicosapentaenoic acid (12-HEPE), 15-hydroxy-eicosapentaenoic acid (15-HE PE), 18-hydroxy-eicosapentaenoic acid (18-HEPE), 19-hydroxy-eicosapentaenoic acid (19-HE PE), 20-hydroxy-eicosapentaenoic acid (20-HEPE), 5-oxo-eicosapentaenoic acid (5-oxo-EPE), 12-oxo-eicosapentaenoic acid (12-oxo-EPE), 15-oxo-eicosapentaenoic acid (15-oxo-EPE), 5,6-epoxy-eicosatetraenoic acid (5,6-EpETE), 8,9-epoxy-eicosatetraenoic acid (8,9-EpETE), 11,12-epoxy-eicosatetraenoic acid (11,12-EpETE), 14,15-epoxy-eicosatetraenoic acid (14,15-EpETE), 5,6-dihydroxy-eicosatetraenoic acid (5,6-diHETE), 8,9-dihydroxy-eicosatetraenoic acid (8,9-diHETE), 11,12-dihydroxy-eicosatetraenoic acid (11,12-diHETE), 14,15-dihydroxy-eicosatetraenoic acid (14,15-diHETE), 17,18-dihydroxy-eicosatetraenoic acid (17,18-diHETE), 17,18-epoxy-eicosatetraenoic acid (17,18-EpETE), lipoxin A5 (LxAS), lipoxin B5 (LxBS), 15-epi-lipoxin A4, resolvin E1 (RvE1), resolvin E2 (RvE2), resolvin E3 (RvE3), and resolvin E4 (RvE4).

15. The composition of embodiment 3, wherein the DPA derivative is selected from the group consisting of 7-hydroperoxy-docosapentaeonic acid (7-hydroperoxy-DPA), 10-hydroperoxy-docosapentaeonic acid (10-hydroperoxy-DPA), 11-hydroperoxy-docosapentaeonic acid (11-hydroperoxy-DPA), 13-hydroperoxy-docosapentaeonic acid (13-hydroperoxy-DPA), 14-hydroperoxy-docosapentaeonic acid (14-hydroperoxy-DPA), 16-hydroperoxy-docosapentaeonic acid (16-hydroperoxy-DPA), 17-hydroperoxy-docosapentaeonic acid (17-hydroperoxy-DPA), 7-hydroxy-docosapentaeonic acid (7-hydroxy-DPA), 10-hydroxy-docosapentaeonic acid (10-hydroxy-DPA), 11-hydroxy-docosapentaeonic acid (11-hydroxy-DPA), 13-hydroxy-docosapentaeonic acid (13-hydroxy-DPA), 14-hydroxy-docosapentaeonic acid (14-hydroxy-DPA), 16-hydroxy-docosapentaeonic acid (16-hydroxy-DPA), 17-hydroxy-docosapentaeonic acid (17-hydroxy-DPA), 7,17-dihydroxy-docosapentaeonic acid (7,17-dihydroxy-DPA), 8,14-dihydroxy-docosapentaeonic acid (8,14-dihydroxy-DPA), 10,17-dihydroxy-docosapentaeonic acid (10,17-dihydroxy-DPA), 10,20-dihydroxy-docosapentaeonic acid (10,20-dihydroxy-DPA), 13,20-dihydroxy-docosapentaeonic acid (13,20-dihydroxy-DPA), 16,17-dihydroxy-docosapentaeonic acid (16,17-dihydroxy-DPA), 13-oxo-docosapentaeonic acid (13-oxo-DPA or 13-EFOX-D5), MaR1n-3 DPA, MaR2n-3 DPA, MaR3n-3 DPA, PD1n-3 DPA, PD2n-3 DPA, 7,13,20-trihydroxy-n-3-docosapentaeonic acid (resolvin T1 or RvT1), 7,12,13-trihydroxy-n-3-docosapentaeonic acid (resolvin T2 or RvT2), 7,8,13-trihydroxy-n-3-docosapentaeonic acid (resolvin T3 or RvT3), 7,16,17-trihydroxy-n-3-docosapentaeonic acid (7,16,17-tri hydroxy-DPA), RvD1n-3 DPA, RvD2n-3 DPA, and RvD2n-3 DPA.

16. The composition of embodiment 3, wherein the DHA derivative is selected from the group consisting of 4-hydroperoxy-docosahexaenoic acid (4-HpDoHE), 7-hydroperoxy-docosahexaenoic acid (7-HpDoHE), 8-hydroperoxy-docosahexaenoic acid (8-HpDoHE), 10-hydroperoxy-docosahexaenoic acid (10-HpDoHE), 11-hydroperoxy-docosahexaenoic acid (11-HpDoHE), 13-hydroperoxy-docosahexaenoic acid (13-HpDoHE), 14-hydroperoxy-docosahexaenoic acid (14-HpDoHE), 16-hydroperoxy-docosahexaenoic acid (16-HpDoHE), 17-hydroperoxy-docosahexaenoic acid (17-HpDoHE), 4-hydroxy-docosahexaenoic acid (4-HDoHE), 7-hydroxy-docosahexaenoic acid (7-HDoHE), 8-hydroxy-docosahexaenoic acid (8-HDoHE), 10-hydroxy-docosahexaenoic acid (10-HDoHE), 11-hydroxy-docosahexaenoic acid (11-HDoHE), 13-hydroxy-docosahexaenoic acid (13-HDoHE), 14-hydroxy-docosahexaenoic acid (14-HDoHE), 16-hydroxy-docosahexaenoic acid (16-HDoHE), 17-hydroxy-docosahexaenoic acid (17-HDoHE), 20-hydroxy-docosahexaenoic acid (20-HDoHE), 21-hydroxy-docosahexaenoic acid (21-HDoHE), 22-hydroxy-docosahexaenoic acid (22-HDoHE), 7,14-dihydroxy-docosahexaenoic acid (7,14-DiHDoHE), 7,17-dihydroxy-docosahexaenoic acid (7,17-DiHDoHE), 8,14-dihydroxy-docosahexaenoic acid (8,14-DiHDoHE), 10,17-dihydroxy-docosahexaenoic acid (10,17-DiHDoHE), 10,20-dihydroxy-docosahexaenoic acid (10,20-DiHDoHE), 14,20-dihydroxy-docosahexaenoic acid (14,20-DiHDoHE), 14,21-dihydroxy-docosahexaenoic acid (14,21-DiHDoHE), 7-oxo-docosahexaenoic acid (7-oxo-DoHE), 4,5-epoxy-docosapentaenoic acid (4,5-EpDPE), 7,8-epoxy-docosapentaenoic acid (7,8-EpDPE), 10,11-epoxy-docosapentaenoic acid (10,11-EpDPE), 13,14-epoxy-docosapentaenoic acid (13,14-EpDPE), 16,17-epoxy-docosapentaenoic acid (16,17-EpDPE), 19,20-epoxy-docosapentaenoic acid (19,20-EpDPE), 4,5-dihydroxy-docosapentaenoic acid (4,5-DiHDPE), 7,8-dihydroxy-docosapentaenoic acid (7,8-DiHDPE), 10,11-dihydroxy-docosapentaenoic acid (10,11-DiHDPE), 13,14-dihydroxy-docosapentaenoic acid (13,14-DiHDPE), 16,17-dihydroxy-docosapentaenoic acid (16,17-DiHDPE), 19,20-dihydroxy-docosapentaenoic acid (19,20-DiHDPE), 4,5-epoxy-17-0H-docosahexaenoic acid, (4,5-epoxy-17-hydroxy-DHA), 7,8-epoxy-17-0H-docosahexaenoic acid (7,8-epoxy-17-hydroxy-DHA), maresin 1 (MaR1), maresin 2 (MaR2), protectin 1 (PD1), protectin X (PDX), aspirin-triggered PD1 (AT-PD1), resolvin D1 (RvD1), resolvin D2 (RvD2), resolvin D3 (RvD3), resolvin D4 (RvD4), resolvin D5 (RvD5), resolvin D6 (RvD6), aspirin-triggered resolvin D1 (AT-RvD1), aspirin-triggered resolvin D2 (AT-RvD2), aspirin-triggered resolvin D3 (AT-RvD3), aspirin-triggered resolvin D4 (AT-RvD4), aspirin-triggered resolvin D5 (AT-RvD5), and aspirin-triggered resolvin D6 (AT-RvD6).

17. The composition of embodiment 1 or 2, wherein the one or more PUFAs or derivatives thereof are selected from the group consisting of tetracosatetraenoic acid (TTE), tetracosapentaenoic acid (TPA), tetracosahexaenoic acid (THA), a TTE derivative, a TPA derivative, and a THA derivative.

18. The composition of embodiment 1 or 2, wherein the one or more PUFAs or derivatives thereof comprises EPA in free acid form or a pharmaceutically acceptable ester, conjugate, or salt thereof.

19. The composition of embodiment 18, wherein the EPA is eicosapentaenoic acid ethyl ester (EtEPA).

20. The composition of embodiment 18 or 19, wherein the EPA or EtEPA comprises at least 66%, 75%, 80%, 90%, 95%, or 96%, by weight, of all PUFAs present in the composition.

21. The composition of embodiment 18 or 19, wherein the composition comprises no more than 20%, by weight of all PUFAs present in the composition, one or more of the following: (a) one or more omega-6 PUFAs or derivatives thereof selected from the group consisting of LA, GLA, DGLA, AdA, DPA6, an LA derivative, a GLA derivative, a DGLA derivative, an AdA derivative, and a DPA6 derivative; (b) one or more omega-3 PUFAs or derivatives thereof selected from the group consisting of ALA, SDA, ETA, DPA, an ALA derivative, an SDA derivative, an ETA derivative, and a DPA derivative; and (c) one or more oxylipins.

22. The composition of any one of embodiments 18-21, wherein the composition comprises about 500 mg to about 1 g of the EPA or EtEPA.

23. The composition any one of embodiments 1-22, wherein the source of phospholipid comprises a glycerophospholipid, a lysophospholipid, or a mixture thereof.

24. The composition of embodiment 23, wherein the source of phospholipid is lecithin.

25. The composition of embodiment 24, wherein the lecithin comprises up to 40%, up to 60%, up to 80%, up to 90%, up to 95%, or up to 97%, by weight of the lecithin, phosphatidylethanolamine, and no more than 10%, no more than 5%, no more than 4%, no more than 3%, no more than 2%, or no more than 1%, by weight of the lecithin, phosphatidylinositol.

26. The composition of embodiment 24, wherein the lecithin comprises: (a) 19%-27%, by weight, phosphatidylcholine; (b) no more than 4%, by weight, lysophosphatidylcholine; (c) 16%-22%, by weight, phosphatidylethanolamine; (d) 11%-18%, by weight, phosphatidylinositol; and (e) 1%-9%, by weight, phosphatidic acid.

27. The composition of any one of embodiments 1-26, wherein a weight ratio of the one or more PUFAs or derivatives thereof and the source of phospholipid ranges from about 5:1 to about 1:5, from about 3.75:1 to about 1:5, or from about 1:1 to about 1:5.

28. The composition of embodiment 20, wherein a weight ratio of the EPA or EtEPA and the source of phospholipid ranges from about 1:1 to about 1:5.

29. The composition of any one of embodiments 2-28, wherein the one or more emulsifiers comprise polysorbate 80, polyoxyl-35, or both.

30. The composition of any one of embodiments 2-28, wherein the one or more emulsifiers comprise one or more glycerol derivatives selected from the group consisting of triacylglycerol, diacylglycerol, and monoacylglycerol.

31. The composition of embodiment 30, wherein the glycerol derivative is castor oil.

32. The composition of embodiment 30, wherein the glycerol derivative is re-esterified triglyceride (rTG) enriched with the PUFA.

33. A kit comprising: (a) a first composition comprising one or more polyunsaturated fatty acids (PUFAs) or derivatives thereof; and (b) a second composition comprising a source of phospholipid.

34. The kit of embodiment 33, wherein the first and/or second composition further comprises one or more emulsifiers.

35. The kit of embodiment 33 or 34, wherein the one or more PUFAs or derivatives thereof are selected from the group consisting of linoleic acid (LA), gamma-linoleic acid (GLA), dihomo-gamma-linoleic acid (DGLA), arachidonic acid (AA), adrenic acid (AdA), omega-6 docosapentaenoic acid (DPA6), alpha-lineoleic acid (ALA), stearidonic acid (SDA), omega-3 eicosatetraenoic acid (ETA), eicosapentaenoic acid (EPA), docosapentaenoic acid (DPA), docosahexaenoic acid (DHA), an LA derivative, a GLA derivative, a DGLA derivative, an AA derivative, an AdA derivative, a DPA6 derivative, an ALA derivative, an SDA derivative, an ETA derivative, an EPA derivative, a DPA derivative, and a DHA derivative.

36. The kit of embodiment 33 or 34, wherein the one or more PUFAs or derivatives thereof are selected from the group consisting of tetracosatetraenoic acid (TTE), tetracosapentaenoic acid (TPA), tetracosahexaenoic acid (THA), a TTE derivative, a TPA derivative, and a THA derivative.

37. The kit of any one of embodiments 33-36, wherein the PUFA derivative comprises an oxylipin.

38. The kit of embodiment 33 or 34, wherein the one or more PUFAs or derivatives thereof comprises EPA in free acid form or a pharmaceutically acceptable ester, conjugate, or salt thereof.

39. The kit of embodiment 38, wherein the EPA is eicosapentaenoic acid ethyl ester (EtEPA).

40. The kit of embodiment 38 or 39, wherein the EPA or EtEPA comprises at least 66%, 75%, 80%, 90%, 95%, or 96%, by weight, of all PUFAs present in the first composition.

41. The kit of embodiment 38 or 39, wherein the first composition comprises no more than 20%, by weight of all PUFAs present in the first composition, one or more of the following: (a) one or more omega-6 PUFAs or derivatives thereof selected from the group consisting of LA, GLA, DGLA, AdA, DPA6, an LA derivative, a GLA derivative, a DGLA derivative, an AdA derivative, and a DPA6 derivative; (b) one or more omega-3 PUFAs or derivatives thereof selected from the group consisting of ALA, SDA, ETA, DPA, an ALA derivative, an SDA derivative, an ETA derivative, and a DPA derivative; and (c) one or more oxylipins.

42. The kit of any one of embodiments 38-41, wherein the first composition comprises about 500 mg to about 1 g of the EPA or EtEPA.

43. The kit any one of embodiments 33-42, wherein the source of phospholipid comprises a glycerophospholipid, a lysophospholipid, or a mixture thereof.

44. The kit of embodiment 43, wherein the source of phospholipid is lecithin.

45. The kit of embodiment 44, wherein the lecithin comprises up to 40%, up to 60%, up to 80%, up to 90%, up to 95%, or up to 97%, by weight of the lecithin, phosphatidylethanolamine, and no more than 10%, no more than 5%, no more than 4%, no more than 3%, no more than 2%, or no more than 1%, by weight of the lecithin, phosphatidylinositol.

46. The kit of embodiment 44, wherein the lecithin comprises: (a) 19%-27%, by weight, phosphatidylcholine; (b) no more than 4%, by weight, lysophosphatidylcholine; (c) 16%-22%, by weight, phosphatidylethanolamine; (d) 11%-18%, by weight, phosphatidylinositol; and (e) 1%-9%, by weight, phosphatidic acid.

47. The kit of any one of embodiments 33-46, wherein a weight ratio of the one or more PUFAs or derivatives thereof and the source of phospholipid ranges from about 5:1 to about 1:5, from about 3.75:1 to about 1:5, or from about 1:1 to about 1:5.

48. The kit of embodiment 40, wherein a weight ratio of the EPA or EtEPA and the source of phospholipid ranges from about 1:1 to about 1:5.

49. The kit of any one of embodiments 34-48, wherein the one or more emulsifiers comprise polysorbate 80, polyoxyl-35, or both.

50. The kit of any one of embodiments 34-48, wherein the one or more emulsifiers comprise one or more glycerol derivatives selected from the group consisting of triacylglycerol, diacylglycerol, and monoacylglycerol.

51. The kit of embodiment 50, wherein the glycerol derivative is castor oil.

52. The kit of embodiment 50, wherein the glycerol derivative is re-esterified triglyceride (rTG) enriched with the PUFA.

53. A lymph-releasing eicosapentaenoic acid ethyl ester (LR-EtEPA) composition comprising: (a) at least 15%, by weight, EtEPA; and (b) 1% to 85%, by weight, a source of phospholipid.

54. The LR-EtEPA composition of embodiment 53, further comprising (c) 1% to 20%, by weight, one or more emulsifiers.

55. The LR-EtEPA composition of embodiment 53 or 54, wherein the EtEPA comprises at least 66%, 75%, 80%, 90%, 95%, or 96%, by weight, of all fatty acids present in the composition.

56. The LR-EtEPA composition of any one of embodiments 53-55, wherein the composition comprises about 500 mg to about 1 g of the EtEPA.

57. The LR-EtEPA composition any one of embodiments 53-56, wherein the source of phospholipid comprises a glycerophospholipid, a lysophospholipid, or a mixture thereof.

58. The LR-EtEPA composition of embodiment 57, wherein the source of phospholipid is lecithin.

59. The LR-EtEPA composition of embodiment 58, wherein the lecithin comprises up to 40%, up to 60%, up to 80%, up to 90%, up to 95%, or up to 97%, by weight of the lecithin, phosphatidylethanolamine, and no more than 10%, no more than 5%, no more than 4%, no more than 3%, no more than 2%, or no more than 1%, by weight of the lecithin, phosphatidylinositol.

60. The LR-EtEPA composition of embodiment 58, wherein the lecithin comprises: (a) 19%-27%, by weight, phosphatidylcholine; (b) no more than 4%, by weight, lysophosphatidylcholine; (c) 16%-22%, by weight, phosphatidylethanolamine; (d) 11%-18%, by weight, phosphatidylinositol; and (e) 1%-9%, by weight, phosphatidic acid.

61. The LR-EtEPA composition of any one of embodiments 53-60, wherein a weight ratio of the EtEPA and the source of phospholipid ranges from about 5:1 to about 1:5, from about 3.75:1 to about 1:5, or from about 1:1 to about 1:5.

62. The LR-EtEPA composition of any one of embodiments 54-61, wherein the one or more emulsifiers comprise polysorbate 80, polyoxyl-35, or both.

63. The LR-EtEPA composition of any one of embodiments 54-61, wherein the one or more emulsifiers comprise one or more glycerol derivatives selected from the group consisting of triacylglycerol, diacylglycerol, and monoacylglycerol.

64. The LR-EtEPA composition of embodiment 63, wherein the glycerol derivative is castor oil.

65. The LR-EtEPA composition of embodiment 63, wherein the glycerol derivative is re-esterified triglyceride (rTG) enriched with the PUFA.

66. The LR-EtEPA composition of any one of embodiments 53-65, wherein the EtEPA and the source of phospholipid are co-formulated in a same dosage unit or individually formulated in separate dosage units.

67. The LR-EtEPA composition of embodiment 66, wherein the dosage unit is a capsule.

68. A method of treating or preventing a disease in a subject in need thereof, the method comprising administering to the subject a therapeutically effective amount of the composition of any one of embodiments 1-32, the kit of any one of embodiments 33-52, or the LR-EtEPA composition of any one of embodiments 53-67.

69. The method of embodiment 68, wherein the disease is a cardiovascular disease.

70. The method of embodiment 69, wherein the cardiovascular disease is selected from the group consisting of hypertriglyceridemia, hypercholesterolemia, mixed dyslipidemia, coronary heart disease, stroke, atherosclerosis, arrhythmia, hypertension, myocardial infarction, vasculitis, cardiomyopathy (e.g., viral cardiomyopathy including related to COVID-19), pericarditis, congestive heart failure, myocardial necrosis, vascular ischemia, vascular disease beyond the cardiopulmonary system, thrombotic disease, post-myocardial infarction cardiac remodeling, giant cell arteritis, polyarteritis nodosa, cryoglobulinemia, episodic small-vessel ischemia (Raynaud's disease), deep venous thrombosis, disseminated intravascular coagulation, and erectile dysfunction.

71. The method of embodiment 69 or 70, wherein the subject has a fasting baseline triglyceride level of about 135 mg/dL to about 500 mg/dL.

72. The method of any one of embodiments 69-71, wherein the subject has one or more of: a baseline non-high-density lipoprotein cholesterol (HDL-C) value of about 200 mg/dL to about 300 mg/dL; a baseline total cholesterol (TC) value of about 250 mg/dL to about 300 mg/dL; a baseline very low-density lipoprotein cholesterol (VLDL-C) value of about 140 mg/dL to about 200 mg/dL; a baseline HDL-C value of about 10 mg/dL to about 30 mg/dL; a baseline low-density lipoprotein cholesterol (LDL-C) value of about 40 mg/dL to about 100 mg/dL; and a baseline high-sensitivity C-reactive protein (hsCRP) level of about 2 mg/dL or less.

73. The method of any one of embodiments 69-72, wherein the subject is on stable statin therapy.

74. The method of embodiment 73, wherein the stable statin therapy comprises a statin and optionally ezetimibe.

75. The method of embodiment 74, wherein the statin is selected from the group consisting of atorvastatin, fluvastatin, lovastatin, pitavastatin, pravastatin, rosuvastatin, and simvastatin.

76. The method of embodiment 68, wherein the disease is a pulmonary disease.

77. The method of embodiment 76, wherein the pulmonary disease is selected from the group consisting of community-acquired pneumonia, COVID-19 pneumonia, systemic inflammatory response syndrome (SIRS), sepsis, SIRS, acute respiratory distress syndrome (ARDS), pulmonary embolism, diffuse interstitial pneumonia, radiation pneumonitis, pleuritis, acute eosinophilic pneumonia, chronic eosinophilic pneumonia, Loffler syndrome, sarcoidosis, interstitial lung disease, chronic obstructive pulmonary disease (COPD), reactive airway disease, asthma, bronchiectasis, bronchiolitis, cystic fibrosis, bronchial carcinoid, pulmonary arterial hypertension, pulmonary vasculitis, microscopic polyangiitis, granulomatosis with polyangiitis (Wegener's disease), eosinophilic granulomatosis with polyangiitis (Churg-Strauss), nasopharyngitis, Goodpasture's syndrome, cryoglobulinemia, systemic lupus erythematosus (SLE), systemic sclerosis, and antiphospholipid syndrome.

78. The method of embodiment 68, wherein the disease is a neurological disease.

79. The method of embodiment 78, wherein the neurological disease is selected from the group consisting of Huntington's disease, sleep disorders, dementia, psychosis, anxiety, treatment-resistant depression, neuropathic pain, schizophrenia, bipolar disorder, dyslexia, dyspraxia, attention deficit hyperactivity disorder (ADHD), epilepsy, autism, Alzheimer's disease, Parkinson's Disease, senile dementia, multiple sclerosis, diabetes-induced neuropathy, macular degeneration, retinopathy of prematurity, amyotrophic lateral sclerosis (ALS), retinitis pigmentosa, cerebral palsy, muscular dystrophy, neurological cancer, cystic fibrosis, and neural tube defects.

80. The method of embodiment 68, wherein the disease is cancer.

81. The method of embodiment 80, wherein the cancer is a hematological malignancy selected from the group consisting of monoclonal B cell lymphocytosis, multiple myeloma, myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), blast crisis chronic myelogenous leukemia (bcCML), B cell acute lymphoid leukemia (B-ALL), T cell acute lymphoid leukemia (T-ALL), T cell lymphoma, and B cell lymphoma.

82. The method of embodiment 80, wherein the cancer is a solid tumor selected from the group consisting of lung cancer, breast cancer, liver cancer, stomach cancer, colon cancer, rectal cancer, colorectal cancer, kidney cancer, gastric cancer, gallbladder cancer, cancer of the small intestine, esophageal cancer, melanoma, bone cancer, pancreatic cancer, skin cancer, uterine cancer, ovarian cancer, testicular cancer, cancer of the thyroid gland, cancer of the adrenal gland, bladder cancer, and glioma.

83. The method of embodiment 68, wherein the disease is a disease associated with kidney selected from the group consisting of post-infectious glomerulonephritis, IgA nephropathy (Berger's disease), Henoch-Schönlein purpura, systemic IgA vasculitis, microscopic polyangiitis, granulomatosis with polyangiitis (Wegener's), eosinophilic granulomatosis with polyangiitis (Churg-Strauss), polyarteritis, idiopathic crescentic glomerulonephritis, anti-GBM glomerulonephritis, Goodpasure syndrome, cryoglobulin-associated glomerulonephritis, idiopathic membranoproliferative glomerulopnephritis (MPGN), hepatitis C-associated glomerulonephritis, systemic lupus erythematosus (SLE) associated glomerulonephritis, minimal change disease (nill disease, lipoid nephrosis), membranous nephropathy, focal and segmental glomerulosclerosis, amyloidosis, diabetic nephropathy, HIV-associated nephropathy, membranoproliferative glomerlonephropathy, mitigating proteinuria, mitigating chronic renal failure, and/or mitigating mortality/morbidity in severe chronic kidney disease (CKD)/end-stage renal disease (ESRD).

84. The method of embodiment 68, wherein the disease is a disease associated with pancreas selected from the group consisting of hyperglycemia, pre-diabetes, diabetes (Type 1 and/or Type 2), and pancreatitis.

85. The method of embodiment 68, wherein the disease is a disease associated with liver selected from the group consisting of chronic viral hepatitis, autoimmune hepatitis, alcoholic liver disease, non-alcoholic fatty liver disease, hemochromatosis, Wilson disease, primary biliary cholangitis, primary sclerosing cholangitis, and cholelithiasis.

86. The method of embodiment 68, wherein the disease is a disease associated with intestines selected from the group consisting of gastroesophageal reflux disease (GERD), gastritis, peptic ulcer disease, obesity, cachexia, intestinal angina, Crohn disease, ulcerative colitis, antibiotic-associated colitis, irritable bowel syndrome, colon cancer, colon polyposis, and carcinoid.

87. The method of embodiment 68, wherein the disease is a disease associated with blood cells selected from the group consisting of iron deficiency anemia, anemia of chronic disease, hemolytic anemia, thalassemia, polycythemia vera, sickle cell disease anemia, sickle cell disease pain, immune thrombocytopenia, leukemias, Non-Hodgkin lymphoma, and Hodgkin lymphoma.

88. The method of embodiment 68, wherein the disease is a disease associated with oxidative stress, glutathione (GSH) depletion, Nrf2 activation, and/or heme-oxygenase activation.

89. The method of embodiment 88, wherein the disease is anemia, sickle cell disease, and/or glomerulonephritis.

90. The method of embodiment 88 or 89, wherein the method further comprises administering to the subject a N-acetylcysteine (NAC) related agent.

91. The method of embodiment 90, wherein the NAC related agent is selected from the group consisting of cystine, methionine, N-acetylcysteine, and L-2-oxothiazolidine-4-carboxylate.

92. The method of embodiment 68, wherein the disease is oxidative stress, endothelial dysfunction, narrowing and/or thickening of arteries, and/or inflammation induced by inhalation of particulate matter.

93. The method of embodiment 68, wherein the disease is oxidative stress, endothelial dysfunction, narrowing and/or thickening of arteries, and/or inflammation induced by long-term and/or short-term exposure to air pollution.

94. The method of any one of embodiments 68-93, wherein the composition, kit, or LR-EtEPA composition is administered to the subject to provide a daily dose of about 1 g to about 20 g of EtEPA.

95. The method of embodiment 94, wherein the composition, kit, or LR-EtEPA composition is administered to the subject to provide a daily dose of about 4 g of EtE PA.

96. The method of any one of embodiments 68-95, wherein the composition, kit, or LR-EtEPA composition is administered to the subject once or twice per day.

97. The method of any one of embodiments 68-96, wherein the composition, kit, or LR-EtEPA composition is administered to the subject with or without food.

98. The composition of any one of embodiments 1-32, the kit of any one of embodiments 33-52, or the LR-EtEPA composition of any one of embodiments 53-67, for use in a method of treating or preventing a disease in a subject in need thereof, wherein a therapeutically effective amount of the composition, kit, or LR-EtEPA composition is administered to the subject.

99. The composition, kit, or LR-EtEPA composition of embodiment 98, wherein the disease is a cardiovascular disease.

100. The composition, kit, or LR-EtEPA composition of embodiment 99, wherein the cardiovascular disease is selected from the group consisting of hypertriglyceridemia, hypercholesterolemia, mixed dyslipidemia, coronary heart disease, stroke, atherosclerosis, arrhythmia, hypertension, myocardial infarction, vasculitis, cardiomyopathy (e.g., viral cardiomyopathy including related to COVID-19), pericarditis, congestive heart failure, myocardial necrosis, vascular ischemia, vascular disease beyond the cardiopulmonary system, thrombotic disease, post-myocardial infarction cardiac remodeling, giant cell arteritis, polyarteritis nodosa, cryoglobulinemia, episodic small-vessel ischemia (Raynaud's disease), deep venous thrombosis, disseminated intravascular coagulation, and erectile dysfunction.

101. The composition, kit, or LR-EtEPA composition of embodiment 99 or 100, wherein the subject has a fasting baseline triglyceride level of about 135 mg/dL to about 500 mg/dL.

102. The composition, kit, or LR-EtEPA composition of any one of embodiments 99-101, wherein the subject has one or more of: a baseline non-high-density lipoprotein cholesterol (HDL-C) value of about 200 mg/dL to about 300 mg/dL; a baseline total cholesterol (TC) value of about 250 mg/dL to about 300 mg/dL; a baseline very low-density lipoprotein cholesterol (VLDL-C) value of about 140 mg/dL to about 200 mg/dL; a baseline HDL-C value of about 10 mg/dL to about 30 mg/dL; a baseline low-density lipoprotein cholesterol (LDL-C) value of about 40 mg/dL to about 100 mg/dL; and a baseline high-sensitivity C-reactive protein (hsCRP) level of about 2 mg/dL or less.

103. The composition, kit, or LR-EtEPA composition of any one of embodiments 99-102, wherein the subject is on stable statin therapy.

104. The composition, kit, or LR-EtEPA composition of embodiment 103, wherein the stable statin therapy comprises a statin and optionally ezetimibe.

105. The composition, kit, or LR-EtEPA composition of embodiment 104, wherein the statin is selected from the group consisting of atorvastatin, fluvastatin, lovastatin, pitavastatin, pravastatin, rosuvastatin, and simvastatin.

106. The composition, kit, or LR-EtEPA composition of embodiment 98, wherein the disease is a pulmonary disease.

107. The composition, kit, or LR-EtEPA composition of embodiment 106, wherein the pulmonary disease is selected from the group consisting of community-acquired pneumonia, COVID-19 pneumonia, systemic inflammatory response syndrome (SIRS), sepsis, SIRS, acute respiratory distress syndrome (ARDS), pulmonary embolism, diffuse interstitial pneumonia, radiation pneumonitis, pleuritis, acute eosinophilic pneumonia, chronic eosinophilic pneumonia, Loffler syndrome, sarcoidosis, interstitial lung disease, chronic obstructive pulmonary disease (COPD), reactive airway disease, asthma, bronchiectasis, bronchiolitis, cystic fibrosis, bronchial carcinoid, pulmonary arterial hypertension, pulmonary vasculitis, microscopic polyangiitis, granulomatosis with polyangiitis (Wegener's disease), eosinophilic granulomatosis with polyangiitis (Churg-Strauss), nasopharyngitis, Goodpasture's syndrome, cryoglobulinemia, systemic lupus erythematosus (SLE), systemic sclerosis, and antiphospholipid syndrome.

108. The composition, kit, or LR-EtEPA composition of embodiment 98, wherein the disease is a neurological disease.

109. The composition, kit, or LR-EtEPA composition of embodiment 108, wherein the neurological disease is selected from the group consisting of Huntington's disease, sleep disorders, dementia, psychosis, anxiety, treatment-resistant depression, neuropathic pain, schizophrenia, bipolar disorder, dyslexia, dyspraxia, attention deficit hyperactivity disorder (ADHD), epilepsy, autism, Alzheimer's disease, Parkinson's Disease, senile dementia, multiple sclerosis, diabetes-induced neuropathy, macular degeneration, retinopathy of prematurity, amyotrophic lateral sclerosis (ALS), retinitis pigmentosa, cerebral palsy, muscular dystrophy, neurological cancer, cystic fibrosis, and neural tube defects.

110. The composition, kit, or LR-EtEPA composition of embodiment 98, wherein the disease is cancer.

111. The composition, kit, or LR-EtEPA composition of embodiment 110, wherein the cancer is a hematological malignancy selected from the group consisting of monoclonal B cell lymphocytosis, multiple myeloma, myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), blast crisis chronic myelogenous leukemia (bcCML), B cell acute lymphoid leukemia (B-ALL), T cell acute lymphoid leukemia (T-ALL), T cell lymphoma, and B cell lymphoma.

112. The composition, kit, or LR-EtEPA composition of embodiment 110, wherein the cancer is a solid tumor selected from the group consisting of lung cancer, breast cancer, liver cancer, stomach cancer, colon cancer, rectal cancer, colorectal cancer, kidney cancer, gastric cancer, gallbladder cancer, cancer of the small intestine, esophageal cancer, melanoma, bone cancer, pancreatic cancer, skin cancer, uterine cancer, ovarian cancer, testicular cancer, cancer of the thyroid gland, cancer of the adrenal gland, bladder cancer, and glioma.

113. The composition, kit, or LR-EtEPA composition of embodiment 98, wherein the disease is a disease associated with kidney selected from the group consisting of post-infectious glomerulonephritis, IgA nephropathy (Berger's disease), Henoch-Schönlein purpura, systemic IgA vasculitis, microscopic polyangiitis, granulomatosis with polyangiitis (Wegener's), eosinophilic granulomatosis with polyangiitis (Churg-Strauss), polyarteritis, idiopathic crescentic glomerulonephritis, anti-GBM glomerulonephritis, Goodpasure syndrome, cryoglobulin-associated glomerulonephritis, idiopathic membranoproliferative glomerulopnephritis (MPGN), hepatitis C-associated glomerulonephritis, systemic lupus erythematosus (SLE) associated glomerulonephritis, minimal change disease (nill disease, lipoid nephrosis), membranous nephropathy, focal and segmental glomerulosclerosis, amyloidosis, diabetic nephropathy, HIV-associated nephropathy, membranoproliferative glomerlonephropathy, mitigating proteinuria, mitigating chronic renal failure, and/or mitigating mortality/morbidity in severe chronic kidney disease (CKD)/end-stage renal disease (ESRD).

114. The composition, kit, or LR-EtEPA composition of embodiment 98, wherein the disease is a disease associated with pancreas selected from the group consisting of hyperglycemia, pre-diabetes, diabetes (Type 1 and/or Type 2), and pancreatitis.

115. The composition, kit, or LR-EtEPA composition of embodiment 98, wherein the disease is a disease associated with liver selected from the group consisting of chronic viral hepatitis, autoimmune hepatitis, alcoholic liver disease, non-alcoholic fatty liver disease, hemochromatosis, Wilson disease, primary biliary cholangitis, primary sclerosing cholangitis, and cholelithiasis.

116. The composition, kit, or LR-EtEPA composition of embodiment 98, wherein the disease is a disease associated with intestines selected from the group consisting of gastroesophageal reflux disease (GERD), gastritis, peptic ulcer disease, obesity, cachexia, intestinal angina, Crohn disease, ulcerative colitis, antibiotic-associated colitis, irritable bowel syndrome, colon cancer, colon polyposis, and carcinoid.

117. The composition, kit, or LR-EtEPA composition of embodiment 98, wherein the disease is a disease associated with blood cells selected from the group consisting of iron deficiency anemia, anemia of chronic disease, hemolytic anemia, thalassemia, polycythemia vera, sickle cell disease anemia, sickle cell disease pain, immune thrombocytopenia, leukemias, Non-Hodgkin lymphoma, and Hodgkin lymphoma.

118. The composition, kit, or LR-EtEPA composition of embodiment 98, wherein the disease is a disease associated with oxidative stress, glutathione (GSH) depletion, Nrf2 activation, and/or heme-oxygenase activation.

119. The composition, kit, or LR-EtEPA composition of embodiment 118, wherein the disease is anemia, sickle cell disease, and/or glomerulonephritis.

120. The composition, kit, or LR-EtEPA composition of embodiment 118 or 119, wherein the method further comprises administering to the subject a N-acetylcysteine (NAC) related agent.

121. The composition, kit, or LR-EtEPA composition of embodiment 120, wherein the NAC related agent is selected from the group consisting of cystine, methionine, N-acetylcysteine, and L-2-oxothiazolidine-4-carboxylate.

122. The composition, kit, or LR-EtEPA composition of embodiment 98, wherein the disease is oxidative stress, endothelial dysfunction, narrowing and/or thickening of arteries, and/or inflammation induced by inhalation of particulate matter.

123. The composition, kit, or LR-EtEPA composition of embodiment 98, wherein the disease is oxidative stress, endothelial dysfunction, narrowing and/or thickening of arteries, and/or inflammation induced by long-term and/or short-term exposure to air pollution.

124. The composition, kit, or LR-EtEPA composition of any one of embodiments 98-123, wherein the composition, kit, or LR-EtEPA composition is administered to the subject to provide a daily dose of about 1 g to about 20 g of EtEPA.

125. The composition, kit, or LR-EtEPA composition of embodiment 124, wherein the composition, kit, or LR-EtEPA composition is administered to the subject to provide a daily dose of about 4 g of EtEPA.

126. The composition, kit, or LR-EtEPA composition of any one of embodiments 98-125, wherein the composition, kit, or LR-EtEPA composition is administered to the subject once or twice per day.

127. The composition, kit, or LR-EtEPA composition of any one of embodiments 98-126, wherein the composition, kit, or LR-EtEPA composition is administered to the subject with or without food.

1. A composition comprising:
(a) one or more polyunsaturated fatty acids (PUFAs) or derivatives thereof; and
(b) a source of phospholipid.

2. The composition of claim 1, further comprising (c) one or more emulsifiers.

3. The composition of claim 1, wherein the one or more PUFAs or derivatives thereof are selected from the group consisting of linoleic acid (LA), gamma-linoleic acid (GLA), dihomo-gamma-linoleic acid (DGLA), arachidonic acid (AA), adrenic acid (AdA), omega-6 docosapentaenoic acid (DPA6), alpha-lineoleic acid (ALA), stearidonic acid (SDA), omega-3 eicosatetraenoic acid (ETA), eicosapentaenoic acid (EPA), docosapentaenoic acid (DPA), docosahexaenoic acid (DHA), an LA derivative, a GLA derivative, a DGLA derivative, an AA derivative, an AdA derivative, a DPA6 derivative, an ALA derivative, an SDA derivative, an ETA derivative, an EPA derivative, a DPA derivative, and a DHA derivative.

4. The composition of claim 3, wherein the PUFA derivative comprises an oxylipin.

5.-17. (canceled)

18. The composition of claim 1, wherein the one or more PUFAs or derivatives thereof comprises EPA in free acid form or a pharmaceutically acceptable ester, conjugate, or salt thereof.

19. The composition of claim 18, wherein the EPA is eicosapentaenoic acid ethyl ester (EtEPA).

20. The composition of claim 18, wherein the composition comprises about 500 mg to about 1 g of the EPA in an amount of at least 66%, 75%, 80%, 90%, 95%, or 96%, by weight, of all PUFAs present in the composition.

21. The composition of claim 1, wherein the composition comprises no more than 20%, by weight of all PUFAs present in the composition, one or more of the following:
(a) one or more omega-6 PUFAs or derivatives thereof selected from the group consisting of LA, GLA, DGLA, AdA, DPA6, an LA derivative, a GLA derivative, a DGLA derivative, an AdA derivative, and a DPA6 derivative;
(b) one or more omega-3 PUFAs or derivatives thereof selected from the group consisting of ALA, SDA, ETA, DPA, an ALA derivative, an SDA derivative, an ETA derivative, and a DPA derivative; and
(c) one or more oxylipins.

22. (canceled)

23. The composition of claim 1, wherein the source of phospholipid comprises a glycerophospholipid, a lysophospholipid, or a mixture thereof.

24. The composition of claim 23, wherein the source of phospholipid is lecithin.

25. The composition of claim 24, wherein the lecithin is comprised of (i) up to 40%, up to 60%, up to 80%, up to 90%, up to 95%, or up to 97%, by weight of the lecithin, phosphatidylethanolamine, and (ii) no more than 10%, no more than 5%, no more than 4%, no more than 3%, no more than 2%, or no more than 1%, by weight of the lecithin, phosphatidylinositol.

26. The composition of claim 24, wherein the lecithin comprises:
(a) 19%-27%, by weight, phosphatidylcholine;
(b) no more than 4%, by weight, lysophosphatidylcholine;
(c) 16%-22%, by weight, phosphatidylethanolamine;
(d) 11%-18%, by weight, phosphatidylinositol; and
(e) 1%-9%, by weight, phosphatidic acid.

27. The composition of claim 1, wherein a weight ratio of the one or more PUFAs or derivatives thereof and the source of phospholipid ranges from about 5:1 to about 1:5, from about 3.75:1 to about 1:5, or from about 1:1 to about 1:5.

28. The composition of claim 18, wherein a weight ratio of the EPA and the source of phospholipid ranges from about 1:1 to about 1:5.

29. The composition of claim 2, wherein the one or more emulsifiers comprise polysorbate 80, polyoxyl-35, or both.

30. The composition of claim 2, wherein the one or more emulsifiers comprise one or more glycerol derivatives selected from the group consisting of triacylglycerol, diacylglycerol, and monoacylglycerol.

31. The composition of claim 30, wherein the glycerol derivative is castor oil.

32. The composition of claim 30, wherein the glycerol derivative is re-esterified triglyceride (rTG) enriched with the PUFA.

33.-52. (canceled)

53. A lymph-releasing eicosapentaenoic acid ethyl ester (LR-EtEPA) composition comprising:
(a) EtEPA; and
(b) a source of phospholipid.

54.-67. (canceled)

68. A method of treating or preventing a disease in a subject in need thereof, the method comprising administering to the subject a therapeutically effective amount of a composition comprising
(a) one or more polyunsaturated fatty acids (PUFAs) or derivatives thereof; and
(b) a source of phospholipid.

69.-97. (canceled)
